(19) United States
(12) Patent Application Publication
(10) Pub. No.: US 2024/0343735 A1
(43) Pub. Date: Oct. 17, 2024

(54) Hydrochloride Salt of Inupadenant, Pharmaceutical Compositions and Methods of Use Thereof

(71) Applicant: iTeos Belgium SA, Gosselies (BE)

(72) Inventors: Marcel de Matas, Gosselies (BE); Paul Dickinson, Gosselies (BE); Manfred Schneider, Gosselies (BE); Sally Ross, Cheadle (GB); Esha Gangolli, Acton (MA); Shouraydeep Srivastava, Andover, MA (US); Joanne Lager, Hollis, NH (US); Edwin Aret, Weert (NL); Chantalle Van Berkel, Weert (NL); Pratheepan Madasamy, Acton, MA (US); Chiara Martinoli, Milan (IT); Maura Rossetti, Waterloo (BE)

(73) Assignee: iTeos Belgium SA, Gosselies (BE)

(21) Appl. No.: 18/618,211

(22) Filed: Mar. 27, 2024

Related U.S. Application Data
(63) Continuation of application No. PCT/US2022/045923, filed on Oct. 6, 2022.
(60) Provisional application No. 63/341,621, filed on May 13, 2022.
(60) Provisional application No. 63/309,163, filed on Feb. 11, 2022.
(60) Provisional application No. 63/253,537, filed on Oct. 7, 2021.

(51) Int. Cl. C07D 487/14; A61K 9/48; A61K 31/519; A61P 35/00

(52) U.S. Cl. CPC C07D 487/14; A61K 9/4858; A61K 9/4866; A61K 31/519; A61P 35/00

(57) ABSTRACT

A hydrochloride salt of inupadenant, pharmaceutical compositions thereof, methods of making pharmaceutical compositions of a hydrochloride salt of inupadenant (e.g., crystalline hydrochloride salt of inupadenant) and methods of using the pharmaceutical compositions in combination with carboplatin and pemetrexed in the treatment of non-small cell lung cancer (NSCLC), including squamous NSCLC and nonsquamous NSCLC.

### CROSS-REFERENCE TO RELATED APPLICATIONS

This application is a continuation of International Application No. PCT/US2022/045923, filed Oct. 6, 2022, which claims the benefit of and priority to U.S. Patent Application No. 63/341,621, filed on May 13, 2022, U.S. Patent Application No. 63/309,163, filed on Feb. 11, 2022, and U.S. Patent Application No. 63/253,537, filed on Oct. 7, 2021, each of which are incorporated by reference herein in their entirety.

### FIELD OF INVENTION

The present invention relates to solid forms of inupadenant and inupadenant hydrochloride and pharmaceutical compositions comprising inupadenant or inupadenant hydrochloride. The solid forms and pharmaceutical compositions of the invention are particularly useful for oral dosing in the treatment of cancers, for example non-small cell lung cancer (NSCLC) including nonsquamous NSCLC.

### BACKGROUND OF THE INVENTION

Many of the immunosuppressive mechanisms in tumors are common to physiological immunoregulation in normal tissues. Such immunoregulation is very important in keeping the immune system under control in order to block a self-reactive immune response and to prevent an ongoing immune response from causing critical tissue damage. The lack of physiological immunoregulation often results in overwhelming immune activation that accompanies autoimmunity. For example, CTLA-4 is a physiological mechanism that negatively regulates T cell activity by blocking a costimulatory signal through CD28-B7 interaction. The lack of CTLA-4 causes non-specific T cell activation, and CTLA-4-deficient mice die in several weeks with massive lymphocytic tissue infiltration. PD-1 also provides a T cell inhibitory signal upon interaction with its ligands, PD-L1 and PD-L2. Deficiency of PD-1 in mice is known to cause various types of autoimmune disorders depending on the genetic strains.

Besides cell surface transducers of immunosuppressive signal, e.g., CTLA-4 and PD-1, immunosuppression in the tumor microenvironment involves anti-inflammatory cytokines (IL-10, TGF-b), enzymes (indoleamine-2, 3-dioxygenase), and professional immunoregulatory cells (regulatory T cells, myeloid-derived suppressor cells MDSCs). These immunosuppressive mechanisms play an important role in controlling immune response in normal tissues. Since tumors take advantage of such physiological immunoregulatory mechanisms to protect their tissue from immune attack, these mechanisms intended to prevent inflammatory complication, now turn out to be major obstacles hampering spontaneous cancer regression and immunological cancer treatment. The identification of immunosuppressive mechanisms in tumors pointed out molecular targets to restore the antitumor immune response. Thus, these negative immunoregulatory mechanisms, so-called immune checkpoints, became a focus in drug discovery. Antibodies against PD-1, PD-L1 or CTLA-4 have been approved as anticancer therapies for a large number of indications, such as metastatic melanoma, non-small cell lung cancer (NSCLC), renal cell carcinoma, Hodgkin's Lymphoma, head and neck cancer, urothelial carcinoma, and hepatocellular carcinoma, as well as treatment for patients with solid tumors that have one of two specific genetic features known as mismatch repair deficiency and high microsatellite instability (irrespective of cancer type).

Extracellular adenosine has been known as an inhibitor of immune functions. While intracellular adenosine is involved in energy metabolism, nucleic acid metabolism, and the methionine cycle, extracellular adenosine plays an important role in intercellular signaling. Its signal is transmitted by G protein-coupled adenosine receptors on the cell surface, and it affects diverse physiological functions including neurological, cardiovascular, and immunological systems.

Tumors contain high levels of extracellular adenosine, suggesting that tumor cells may benefit from its immunosuppressive effect and catabolic energy production (Allard et al., Curr. Opin. Pharmacol., 2016, 29, 7-16; Otta A., Frontiers in Immunology, 2016, 7: 109). This high level of extracellular adenosine is probably due to overexpression of the enzyme CD73, which is responsible for production of extracellular adenosine. CD73 is overexpressed by a large number of tumors, with all the following tumors expressing medium or high levels of CD73 in >50% of tumor surface by immunohistochemistry (www.proteinatlas.org): breast, carcinoid, cervical, colorectal, endometrial, glioma, head and neck, liver, lung, melanoma, ovarian, pancreatic, prostate, renal, gastric, thyroid, and urothelial.

Of the four known types of adenosine receptors, A2A adenosine receptor (A2AR) is the predominantly expressed subtype in most immune cells. Stimulation of A2AR generally provides an immunosuppressive signal that inhibits activities of T cells (proliferation, cytokine production, cytotoxicity), NK cells (cytotoxicity), NKT cells (cytokine production, CD40L upregulation), macrophages/dendritic cells (antigen presentation, cytokine production), and neutrophils (oxidative burst). The presence of high levels of extracellular adenosine in tumors was found to play a significant role in the evasion of antitumor immune response. Especially, it was shown that A2AR-deficient mice could spontaneously regress the inoculated tumor, whereas no wild-type mice showed similar tumor regression. A2AR antagonists were also beneficial in tumor-bearing wild-type animals. Importantly, depletion of T cells and NK cells impaired the retardation of tumor growth by A2AR antagonists, suggesting improvement of antitumor cellular immune response. Effector functions of T cells and NK cells are susceptible to A2AR stimulation. In addition, when activated in the presence of A2AR agonist, the effector function of T cells is persistently impaired even after removal of A2AR agonist. This result suggests that the adenosine-rich environment in tumors may induce T cells that are anergic to the tumor cells. Therefore, given that A2A receptor is expressed in most immune cells and particularly effector immune cells such as T cells and NK cells and given that A2A receptor is engaged in tissues where adenosine is produced, it is thought that A2A inhibitors can be helpful in all cancer indications. Consequently, there is a need for A2A inhibitors that are able to restore immune functions in the tumor environment.

Adenosine is known to be an endogenous modulator of a number of other physiological functions. For example, at the central nervous system (CNS) level, adenosine in known to induce sedative, anxiolytic and antiepileptic effects. Thus, A2A inhibitors were previously developed for the treatment of depression and neurodegenerative diseases such as Parkinson's disease or Alzheimer's disease (Pinna A., CNS Drugs, 2014, 28, 455). One of the most advanced A2A inhibitors developed for the treatment of CNS diseases is Preladenant (Hodgson R A et al., J. Pharmacol. Exp. Ther., 2009, 330(1), 294-303; Hauser R A et al., JAMA Neurol., 2015, 72(12), 1491-1500). However, such previously developed A2A inhibitors were designed to cross the blood brain barrier, in order to target A2A receptor in the CNS.

Given the higher level of adenosine in tumors when compared to the brain, much higher amounts of compounds will be needed to achieve the desired effect on immune function restoration for treating cancers. Thus, in order to avoid deleterious side effects, one should provide A2A inhibitors which have a limited, if any, CNS penetrance, contrary to all previously developed A2A inhibitors.

The Applicant provided a series of non-brain penetrant thiocarbamate derivatives that are A2A inhibitors in international patent application PCT/EP2018/058301. These compounds are useful in restoring immune functions in tumor environment. Nevertheless, these compounds present very low solubility in aqueous buffers, low intestinal solubility and thus low oral bioavailability. As such, the development and manufacture of pharmaceutical formulations of non-brain penetrant A2A inhibitors with the necessary bioavailability, chemical and physical stability, and material properties required to produce safe and efficacious oral drug products for the treatment of cancers and other diseases and disorders remains a significant challenge.

Consequently, there is a need for pharmaceutical formulations of non-brain penetrant A2A inhibitors that display desirable physicochemical properties, are amenable to simple oral administration protocols that would facilitate patient compliance, and exhibit bioavailability profiles that provide sufficient drug exposure to treat cancers in human patients.

As evidenced in the experimental section below, the Applicant hereby provides pharmaceutical compositions that enable suitable oral bioavailability for inupadenant.

Additionally, the Applicant provides a method for treating cancers (e.g., NSCLC, including nonsquamous NSCLC) in human patients by administering to the patients the pharmaceutical compositions described herein, comprising the hydrochloride salt of inupadenant.

Further, the anticancer effect of anticancer agents, such as for example immunotherapeutic agents, chemotherapeutic agents or antiangiogenic agents or combinations thereof, may remain insufficient. This may be due, at least in part, to the tumor's immune escape mechanisms as those described above. Herein, the Applicant further provides methods of treating cancer with a pharmaceutical composition of inupadenant as a hydrochloride salt in combination with one or more of carboplatin and pemetrexed.

### SUMMARY OF THE INVENTION

In one aspect, provided herein is amorphous inupadenant hydrochloride.

In certain embodiments, amorphous inupadenant hydrochloride has an X-ray powder diffraction pattern substantially the same as shown in either diffraction pattern of FIG. 1.

In certain embodiments, the amorphous inupadenant hydrochloride has a differential scanning calorimetry (DSC) thermogram comprising an exotherm with a peak onset temperature between about 155° C. and about 165° C. In certain embodiments, the amorphous inupadenant hydrochloride has a DSC thermogram comprising an endotherm with a peak onset between about 230° C. and about 250° C. In certain embodiments, amorphous inupadenant hydrochloride is characterized by a DSC thermogram substantially the same as shown in FIG. 2A.

In certain embodiments, amorphous inupadenant hydrochloride is characterized by a glass transition temperature with a midpoint at about 110° C. In certain embodiments, amorphous inupadenant hydrochloride is characterized by a glass transition temperature with a midpoint at a temperature between about 140° C. and about 160° C.

In certain embodiments, a weight loss of less than or equal to about 3.5% occurs upon heating the amorphous inupadenant hydrochloride from about 31° C. to about 83° C. In certain embodiments, amorphous inupadenant hydrochloride has a thermogravimetric analysis (TGA) thermogram substantially the same as shown in FIG. 3.

In another aspect, provided herein is crystalline inupadenant hydrochloride.

In another aspect, provided herein is Form 2 inupadenant hydrochloride.

In another aspect, provided herein is inupadenant hydrochloride hydrate.

In another aspect, provided herein is crystalline inupadenant hydrochloride hydrate.

In certain embodiments, crystalline inupadenant hydrochloride, Form 2 inupadenant hydrochloride, inupadenant hydrochloride hydrate, and/or crystalline inupadenant hydrochloride hydrate has an X-ray powder diffraction (XRPD) pattern comprising a peak at about 8.9° 2θ. In certain embodiments, crystalline inupadenant hydrochloride has an XRPD pattern comprising a peak at about 9.3° 2θ. In certain embodiments, the crystalline inupadenant hydrochloride has an XRPD pattern comprising peaks at about 8.9° and about 9.3° 2θ. In certain embodiments, the XRPD pattern further comprises one or more peaks selected from about 14.8° and about 26.7° 2θ. In certain embodiments, the XRPD pattern further comprises one or more peaks selected from about 5.3°, about 18.10, and about 22.6° 2θ. In certain embodiments, the XRPD pattern further comprises a peak at about 32.2° 2θ. In certain embodiments, the XRPD pattern further comprises one or more peaks selected from about 19.3°, about 23.5°, and about 27.5° 2θ. In certain embodiments, the XRPD pattern further comprises one or more peaks selected from about 28.2° and about 29.3° 2θ. In certain embodiments, the XRPD pattern further comprises one or more peaks selected from about 6.7°, about 12.6°, about 13.4°, about 15.4°, about 16.1°, about 16.5°, about 17.4°, about 19.8°, about 24.3°, and about 24.9° 2θ. In certain embodiments, the XRPD pattern further comprises one or more peaks selected from about 30.4°, about 31.0°, and about 31.6° 2θ. In certain embodiments, the XRPD pattern further comprises one or more peaks selected from about 14.5°, about 22.2°, about 23.1°, about 25.4°, and about 25.9° 2θ. In certain embodiments, the XRPD pattern further comprises a peak at about 34.4° 2θ. In certain embodiments, the XRPD pattern further comprises one or more peaks selected from about 21.5°, about 22.8°, about 25.7°, and about 27.1° 2θ. In certain embodiments, the XRPD pattern further comprises one or more peaks selected from about 33.0° and about 33.9° 2θ. In certain embodiments, the XRPD pattern further comprises one or more peaks selected from about 25.0° and about 25.8° 2θ. In certain embodiments, the XRPD pattern further comprises one or more peaks selected from about 22.0° and about 25.2° 2θ. In certain embodiments, the XRPD pattern further comprises one or more peaks selected from about 9.4°, about 13.3°, about 15.5°, about 17.5°, about 23.5°, about 24.4°, and about 26.1° 2θ. In certain embodiments, the XRPD pattern further comprises one or more peaks selected from about 28.3°, about 30.5°, about 31.1°, and about 33.7° 2θ. In certain embodiments, the XRPD pattern further comprises one or more peaks selected from about 12.7°, about 16.8°, about 19.9°, about 20.4°, about 22.10, about 25.5°, and about 27.6°±2θ. In certain embodiments, the XRPD pattern further comprises one or more peaks selected from about 14.6°, about 16.6°, about 22.4°, and about 24.0° 2θ. In certain embodiments, the XRPD pattern further comprises one or more peaks selected from about 31.7° and about 34.5° 2θ. In certain embodiments, the XRPD pattern further comprises one or more peaks selected from about 18.2° and about 18.8° 2θ. In certain embodiments, the XRPD pattern further comprises one or more peaks selected from about 9.5° and about 23.2° 2θ. In certain embodiments, the XRPD pattern further comprises a peak at about 18.9° 2θ. In certain embodiments, the XRPD pattern further comprises one or more peaks selected from about 13.5°, about 16.3°, about 16.7°, about 20.0°, about 21.7°, about 23.7°, about 24.1°, about 25.1°, about 26.0°, and about 27.7° 2θ. In certain embodiments, the XRPD pattern further comprises one or more peaks selected from about 28.6°, about 30.6°, and about 33.1° 2θ. In certain embodiments, the XRPD pattern further comprises one or more peaks selected from about 15.6° and about 27.0° 2θ. In certain embodiments, the XRPD pattern further comprises a peak at about 29.5° 2θ. In certain embodiments, the XRPD pattern further comprises a peak at about 19.4° 2θ. In certain embodiments, the XRPD pattern further comprises one or more peaks selected from about 12.9°, about 15.3°, about 16.0°, about 18.4°, and about 27.4° 2θ. In certain embodiments, the XRPD pattern further comprises a peak at about 29.9° 2θ. In certain embodiments, the XRPD pattern further comprises one or more peaks selected from about 3.3°, about 6.5°, about 22.3°, about 23.9°, about 24.8°, and about 26.8° 2θ. In certain embodiments, the XRPD pattern further comprises one or more peaks selected from about 26.8°, about 29.10, about 29.3°, and about 32.9° 2θ.

In certain embodiments, Form 2 inupadenant hydrochloride has an XRPD pattern substantially the same as shown in FIG. 5. In certain embodiments, crystalline inupadenant hydrochloride has an XRPD pattern substantially the same as shown in FIG. 6. In certain embodiments, crystalline inupadenant hydrochloride has an XRPD pattern substantially the same as shown in FIG. 7. In certain embodiments, crystalline inupadenant hydrochloride has an XRPD pattern substantially the same as shown in FIG. 8.

In certain embodiments, crystalline inupadenant hydrochloride, Form 2 inupadenant hydrochloride, inupadenant hydrochloride hydrate, and/or crystalline inupadenant hydrochloride hydrate has a DSC thermogram comprising one or more endotherms with peak onsets selected from about 70° C., about 140° C., and about 240° C. In certain embodiments, crystalline inupadenant hydrochloride, Form 2 inupadenant hydrochloride, inupadenant hydrochloride hydrate, and/or crystalline inupadenant hydrochloride hydrate has a DSC thermogram substantially the same as shown in FIG. 9.

In certain embodiments, the crystalline inupadenant hydrochloride, the Form 2 inupadenant hydrochloride, the inupadenant hydrochloride hydrate, and/or the crystalline inupadenant hydrochloride hydrate exhibits a change in mass of less than or equal to about 5% wt occurs when varying the relative humidity between 0% and about 95%, when measured at 25° C. In certain embodiments, the crystalline inupadenant hydrochloride, the Form 2 inupadenant hydrochloride, the inupadenant hydrochloride hydrate, and/or the crystalline inupadenant hydrochloride hydrate has a water sorption isotherm, when measured at 25° C., substantially the same as shown in FIG. 10A.

In certain embodiments, the crystalline inupadenant hydrochloride, the Form 2 inupadenant hydrochloride, the inupadenant hydrochloride hydrate, and/or the crystalline inupadenant hydrochloride hydrate has a molar ratio of inupadenant to water of about 1:1 to about 1:1.5. In certain embodiments, a weight loss of between about 1% wt and about 3.2% wt occurs upon heating the crystalline inupadenant hydrochloride, the Form 2 inupadenant hydrochloride, the inupadenant hydrochloride hydrate, and/or the crystalline inupadenant hydrochloride hydrate from about 31° C. to about 83° C. In certain embodiments, the crystalline inupadenant hydrochloride, the Form 2 inupadenant hydrochloride, the inupadenant hydrochloride hydrate, and/or the crystalline inupadenant hydrochloride hydrate has a TGA thermogram substantially the same as shown in FIG. 11A. In certain embodiments, the crystalline inupadenant hydrochloride, the Form 2 inupadenant hydrochloride, the inupadenant hydrochloride hydrate, and/or the crystalline inupadenant hydrochloride hydrate has a water content of about 2.7% wt to about 3.5% wt.

In certain embodiments, the amorphous inupadenant hydrochloride, the crystalline inupadenant hydrochloride, the Form 2 inupadenant hydrochloride, the inupadenant hydrochloride hydrate, and/or the crystalline inupadenant hydrochloride hydrate has a molar ratio of inupadenant to hydrochloride of about 1:1. In certain embodiments, the amorphous inupadenant hydrochloride, the crystalline inupadenant hydrochloride, the Form 2 inupadenant hydrochloride, the inupadenant hydrochloride hydrate, and/or the crystalline inupadenant hydrochloride hydrate has a molar ratio of inupadenant to hydrochloride of 1:1.

In another aspect, provided herein is a pharmaceutical composition generally comprising: amorphous inupadenant hydrochloride and/or crystalline inupadenant hydrochloride; and at least one pharmaceutically acceptable excipient.

In certain embodiments, the at least one pharmaceutically acceptable excipient comprises a lipid carrier.

In various embodiments, a pharmaceutical composition described herein comprises: crystalline inupadenant hydrochloride; and a lipid carrier.

In certain embodiments, a pharmaceutical composition described herein can further comprise a copovidone.

In various embodiments, a pharmaceutical composition described herein comprises:
(a) inupadenant hydrochloride;
(b) a lipid carrier; and
(c) a copovidone.

In certain embodiments, a pharmaceutical composition described herein can further comprise a polyethylene glycol (PEG). In certain embodiments, a pharmaceutical composition described herein can further comprise an antioxidant.

In various embodiments, a pharmaceutical composition described herein comprises:
(a) inupadenant hydrochloride;
(b) a lipid carrier;
(c) a copovidone;
(d) a polyethylene glycol; and
(e) an antioxidant.

In certain embodiments, the inupadenant hydrochloride is present in a pharmaceutical composition described herein as amorphous inupadenant hydrochloride and/or crystalline inupadenant hydrochloride. In certain embodiments, the amorphous inupadenant hydrochloride is an amorphous inupadenant hydrochloride described herein. In certain embodiments, the inupadenant hydrochloride is a crystalline inupadenant hydrochloride described herein. In certain embodiments, the inupadenant hydrochloride is an inupadenant hydrochloride hydrate. In certain embodiments, the inupadenant hydrochloride hydrate is a crystalline inupadenant hydrochloride hydrate described herein. In certain embodiments, the inupadenant hydrochloride is one or more of Form 1 inupadenant hydrochloride and/or Form 2 inupadenant hydrochloride.

In certain embodiments, a pharmaceutical composition described herein comprises about 4% (w/w) to about 6% (w/w) inupadenant hydrochloride. In certain embodiments, a pharmaceutical composition described herein comprises about 4.5% (w/w) to about 5.5% (w/w) inupadenant hydrochloride. In certain embodiments, a pharmaceutical composition described herein comprises about 4.8% (w/w) to about 5.2% (w/w) inupadenant hydrochloride. In certain embodiments, a pharmaceutical composition described herein comprises about 5.1% (w/w) inupadenant hydrochloride. In these and other embodiments, inupadenant hydrochloride is one or more of amorphous inupadenant hydrochloride, crystalline inupadenant hydrochloride, inupadenant hydrochloride hydrate, crystalline inupadenant hydrochloride hydrate, Form 1 inupadenant hydrochloride, and Form 2 inupadenant hydrochloride.

In certain embodiments, a pharmaceutical composition described herein comprises about 5 mg to about 60 mg inupadenant hydrochloride. In certain embodiments, a pharmaceutical composition described herein comprises about 5 mg to about 15 mg inupadenant hydrochloride. In certain embodiments, a pharmaceutical composition described herein comprises about 15 mg to about 25 mg inupadenant hydrochloride. In certain embodiments, a pharmaceutical composition described herein comprises about 35 mg to about 45 mg inupadenant hydrochloride. In certain embodiments, a pharmaceutical composition described herein comprises about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 35 mg, about 36 mg, about 37 mg, about 38 mg, about 39 mg, about 40 mg, about 41 mg, about 42 mg, about 43 mg, about 44 mg or about 45 mg inupadenant hydrochloride. In certain embodiments, a pharmaceutical composition described herein comprises about 9.6 mg inupadenant hydrochloride. In certain embodiments, a pharmaceutical composition described herein comprises about 9.8 mg inupadenant hydrochloride. In certain embodiments, a pharmaceutical composition described herein comprises about 10.0 mg inupadenant hydrochloride. In certain embodiments, a pharmaceutical composition described herein comprises about 10.2 mg inupadenant hydrochloride. In certain embodiments, a pharmaceutical composition described herein comprises about 10.4 mg inupadenant hydrochloride. In certain embodiments, a pharmaceutical composition described herein comprises about 10.6 mg inupadenant hydrochloride. In certain embodiments, a pharmaceutical composition described herein comprises about 10.8 mg inupadenant hydrochloride. In certain embodiments, a pharmaceutical composition described herein comprises about 11.0 mg inupadenant hydrochloride. In certain embodiments, a pharmaceutical composition described herein comprises about 11.2 mg inupadenant hydrochloride. In certain embodiments, a pharmaceutical composition described herein comprises about 11.4 mg inupadenant hydrochloride. In certain embodiments, a pharmaceutical composition described herein comprises about 11.6 mg inupadenant hydrochloride. In certain embodiments, a pharmaceutical composition described herein comprises about 20.0 mg inupadenant hydrochloride. In certain embodiments, a pharmaceutical composition described herein comprises about 20.2 mg inupadenant hydrochloride. In certain embodiments, a pharmaceutical composition described herein comprises about 20.4 mg inupadenant hydrochloride. In certain embodiments, a pharmaceutical composition described herein comprises about 20.6 mg inupadenant hydrochloride. In certain embodiments, a pharmaceutical composition described herein comprises about 20.8 mg inupadenant hydrochloride. In certain embodiments, a pharmaceutical composition described herein comprises about 21.0 mg inupadenant hydrochloride. In certain embodiments, a pharmaceutical composition described herein comprises about 21.2 mg inupadenant hydrochloride. In certain embodiments, a pharmaceutical composition described herein comprises about 21.4 mg inupadenant hydrochloride. In certain embodiments, a pharmaceutical composition described herein comprises about 21.6 mg inupadenant hydrochloride. In certain embodiments, a pharmaceutical composition described herein comprises about 21.8 mg inupadenant hydrochloride. In certain embodiments, a pharmaceutical composition described herein comprises about 22.0 mg inupadenant hydrochloride. In certain embodiments, a pharmaceutical composition described herein comprises about 41.0 mg inupadenant hydrochloride. In certain embodiments, a pharmaceutical composition described herein comprises about 41.2 mg inupadenant hydrochloride. In certain embodiments, a pharmaceutical composition described herein comprises about 41.4 mg inupadenant hydrochloride. In certain embodiments, a pharmaceutical composition described herein comprises about 41.6 mg inupadenant hydrochloride. In certain embodiments, a pharmaceutical composition described herein comprises about 41.8 mg inupadenant hydrochloride. In certain embodiments, a pharmaceutical composition described herein comprises about 42.0 mg inupadenant hydrochloride. In certain embodiments, a pharmaceutical composition described herein comprises about 42.2 mg inupadenant hydrochloride. In certain embodiments, a pharmaceutical composition described herein comprises about 42.4 mg inupadenant hydrochloride. In certain embodiments, a pharmaceutical composition described herein comprises about 42.6 mg inupadenant hydrochloride. In certain embodiments, a pharmaceutical composition described herein comprises about 42.8 mg inupadenant hydrochloride. In certain embodiments, a pharmaceutical composition described herein comprises about 43.0 mg inupadenant hydrochloride. In these and other embodiments, inupadenant hydrochloride is one or more of amorphous inupadenant hydrochloride, crystalline inupadenant hydrochloride, inupadenant hydrochloride hydrate, crystalline inupadenant hydrochloride hydrate, Form 1 inupadenant hydrochloride, and Form 2 inupadenant hydrochloride.

In certain embodiments, a pharmaceutical composition described herein comprises about 4% (w/w) to about 6% (w/w) crystalline inupadenant hydrochloride hydrate. In certain embodiments, a pharmaceutical composition described herein comprises about 4.5% (w/w) to about 5.5% (w/w) crystalline inupadenant hydrochloride hydrate. In certain embodiments, the crystalline inupadenant hydrochloride hydrate is one or more of Form 1 inupadenant hydrochloride and Form 2 inupadenant hydrochloride.

In certain embodiments, a pharmaceutical composition described herein comprises about 5 mg to about 60 mg crystalline inupadenant hydrochloride hydrate. In certain embodiments, a pharmaceutical composition described herein comprises about 5 mg to about 15 mg crystalline inupadenant hydrochloride hydrate. In certain embodiments, a pharmaceutical composition described herein comprises about 15 mg to about 25 mg crystalline inupadenant hydrochloride hydrate. In certain embodiments, a pharmaceutical composition described herein comprises about 35 mg to about 45 mg crystalline inupadenant hydrochloride hydrate. In certain embodiments, a pharmaceutical composition described herein comprises about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 35 mg, about 36 mg, about 37 mg, about 38 mg, about 39 mg, about 40 mg, about 41 mg, about 42 mg, about 43 mg, about 44 mg or about 45 mg crystalline inupadenant hydrochloride hydrate.

In these and other embodiments, a pharmaceutical composition described herein comprises about 70% (w/w) to about 90% (w/w) of the lipid carrier. In certain embodiments, a pharmaceutical composition described herein comprises about 70% (w/w) to about 85% (w/w) of the lipid carrier. In these and other embodiments, a pharmaceutical composition described herein comprises about 75% (w/w) to about 85% (w/w) of the lipid carrier.

In these and other embodiments, a pharmaceutical composition described herein comprises about 150 mg to about 800 mg of the lipid carrier. In these and other embodiments, a pharmaceutical composition described herein comprises about 150 mg to about 250 mg of the lipid carrier. In these and other embodiments, a pharmaceutical composition described herein comprises about 300 mg to about 400 mg of the lipid carrier. In these and other embodiments, a pharmaceutical composition described herein comprises about 600 mg to about 700 mg of the lipid carrier.

In these and other embodiments, the lipid carrier is lauroyl polyoxyl-32 glycerides.

In these and other embodiments, a pharmaceutical composition described herein comprises about 1.0% (w/w) to about 1.5% (w/w) of the copovidone. In these and other embodiments, a pharmaceutical composition described herein comprises about 2.0 mg to about 20 mg of the copovidone.

In these and other embodiments, a pharmaceutical composition described herein comprises about 10% (w/w) to about 20% (w/w) of the PEG. In these and other embodiments, a pharmaceutical composition described herein comprises about 12% (w/w) to about 18% (w/w) of the PEG. In these and other embodiments, a pharmaceutical composition described herein comprises about 20 mg to about 200 mg of the PEG. In these and other embodiments, a pharmaceutical composition described herein comprises about 20 mg to about 120 mg of the PEG.

In these and other embodiments, the antioxidant is butylated hydroxytoluene (BHT). In these and other embodiments, a pharmaceutical composition described herein comprises about 0.05% (w/w) to about 0.15% (w/w) BHT. In these and other embodiments, a pharmaceutical composition described herein comprises about 0.09% (w/w) to about 0.11% (w/w) BHT. In these and other embodiments, a pharmaceutical composition described herein comprises about 0.1% (w/w) BHT.

In these and other embodiments, a pharmaceutical composition described herein comprises about 0.1 mg to about 1.00 mg BHT. In these and other embodiments, a pharmaceutical composition described herein comprises about 0.15 mg to about 0.25 mg BHT. In these and other embodiments, a pharmaceutical composition described herein comprises about 0.35 mg to about 0.45 mg BHT. In these and other embodiments, a pharmaceutical composition described herein comprises about 0.75 mg to about 0.85 mg BHT.

In various embodiments, a pharmaceutical composition described herein comprises:
(a) about 2% (w/w) to about 15% (w/w) inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, a pharmaceutical composition described herein comprises:
(a) about 4% (w/w) to about 6% (w/w) inupadenant hydrochloride;
(b) about 65% (w/w) to about 85% (w/w) of lauroyl macrogol-32 glycerides;
(c) about 12% (w/w) to about 18% (w/w) a PEG;
(d) about 3% (w/w) to about 6% (w/w) of a copovidone; and
(e) about 0.08% (w/w) to about 0.12% (w/w) BHT.

In various embodiments, a pharmaceutical composition described herein comprises:
(a) about 2% (w/w) to about 15% (w/w) crystalline inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, a pharmaceutical composition described herein comprises:
(a) about 4% (w/w) to about 6% (w/w) crystalline inupadenant hydrochloride;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone; and
(e) about 0.08% (w/w) to about 0.12% (w/w) BHT.

In various embodiments, a pharmaceutical composition described herein comprises:
(a) about 2% (w/w) to about 15% (w/w) crystalline inupadenant hydrochloride hydrate; (b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, a pharmaceutical composition described herein comprises:
(a) about 4% (w/w) to about 6% (w/w) crystalline inupadenant hydrochloride hydrate; (b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone;
(e) about 0.08% (w/w) to about 0.12% (w/w) BHT.

In various embodiments, a pharmaceutical composition described herein comprises:
(a) about 2% (w/w) to about 15% (w/w) Form 1 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, a pharmaceutical composition described herein comprises:
(a) about 4% (w/w) to about 6% (w/w) Form 1 inupadenant hydrochloride;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone;
(e) about 0.08% (w/w) to about 0.12% (w/w) BHT.

In various embodiments, a pharmaceutical composition described herein comprises:
(a) about 2% (w/w) to about 15% (w/w) Form 2 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, a pharmaceutical composition described herein comprises:
(a) about 4% (w/w) to about 6% (w/w) Form 2 inupadenant hydrochloride;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone;
(e) about 0.08% (w/w) to about 0.12% (w/w) BHT.

In certain embodiments, the PEG present in a pharmaceutical composition described herein is selected from PEG 400 and PEG 1000. In certain embodiments, the PEG is PEG 400. In certain embodiments, the PEG is PEG 1000.

In another aspect, provided herein is a dosage form comprising a pharmaceutical composition described herein.

In certain embodiments, the dosage form is a solid dosage form. In certain embodiments, the dosage form is an oral dosage form. In certain embodiments, the dosage form is selected from the group consisting of a powder, a sachet, a stick pack, a capsule, a minitab, and a tablet.

In certain embodiments, the dosage form is a capsule. In certain embodiments, the capsule is a gel capsule. In certain embodiments, the gel capsule is a hard gel capsule. In certain embodiments, the size of the capsule is selected from the group consisting of 000, 00, 0, 1, 2, 3, 4, and 5.

In certain embodiments, the total weight of a pharmaceutical composition described herein in the capsule is about 200 mg to about 1000 mg. In certain embodiments, the total weight of a pharmaceutical composition described herein in the capsule is about 300 mg to about 500 mg. In certain embodiments, the total weight of a pharmaceutical composition described herein in the capsule is about 350 mg to 450 mg. In certain embodiments, the total weight of a pharmaceutical composition described herein in the capsule is about 700 mg to 900 mg. In certain embodiments, the total weight of a pharmaceutical composition described herein in the capsule is about 780 mg to 880 mg. In certain embodiments, the total weight of a pharmaceutical composition described herein in the capsule is about 150 mg to 300 mg. In certain embodiments, the total weight of a pharmaceutical composition described herein in the capsule is about 200 mg to 250 mg.

In various embodiments, provided herein is a capsule comprising a pharmaceutical composition, wherein the pharmaceutical composition comprises:
(a) about 2% (w/w) to about 15% (w/w) inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, the capsule comprises a pharmaceutical composition, wherein the pharmaceutical composition comprises:
(a) about 4% (w/w) to about 6% (w/w) inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, provided herein is a capsule comprising a pharmaceutical composition, wherein the pharmaceutical composition comprises:
(a) about 2% (w/w) to about 15% (w/w) crystalline inupadenant hydrochloride;
(b) about 50% (w/w) to about 90% (w/w) lauroyl macrogol-32 glycerides;
(c) about 10% (w/w) to about 20% (w/w) of a PEG,
(d) about 2% (w/w) to about 8% (w/w) of a copovidone; and
(e) about 0.05% (w/w) to about 0.2% (w/w) BHT.

In certain embodiments, the capsule comprises a pharmaceutical composition, wherein the pharmaceutical composition comprises:
(a) about 4% (w/w) to about 6% (w/w) crystalline inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, provided herein is a capsule comprising a pharmaceutical composition, wherein the pharmaceutical composition comprises:
(a) about 2% (w/w) to about 15% (w/w) crystalline inupadenant hydrochloride hydrate;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, the capsule comprises a pharmaceutical composition, wherein the pharmaceutical composition comprises:
(a) about 4% (w/w) to about 6% (w/w) crystalline inupadenant hydrochloride hydrate;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, provided herein is a capsule comprising a pharmaceutical composition, wherein the pharmaceutical composition comprises:
(a) about 2% (w/w) to about 15% (w/w) Form 1 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, the capsule comprises a pharmaceutical composition, wherein the pharmaceutical composition comprises:
(a) about 4% (w/w) to about 6% (w/w) Form 1 inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, provided herein is a capsule comprising a pharmaceutical composition, wherein the pharmaceutical composition comprises:
(a) about 2% (w/w) to about 15% (w/w) Form 2 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In certain embodiments, the capsule comprises a pharmaceutical composition, wherein the pharmaceutical composition comprises:
(a) about 4% (w/w) to about 6% (w/w) Form 2 inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In certain embodiments, the PEG is present in a capsule described herein is selected from PEG 400 and PEG 1000. In certain embodiments, the PEG is PEG 400. In certain embodiments, the PEG is PEG 1000.

In certain embodiments, the pharmaceutical composition is present in a capsule described herein as a solid composition or a semi-solid composition.

In certain embodiments, the inupadenant hydrochloride is present in a capsule described herein as amorphous inupadenant hydrochloride and/or crystalline inupadenant hydrochloride. In certain embodiments, the amorphous inupadenant hydrochloride is an amorphous inupadenant hydrochloride described herein. In certain embodiments, the inupadenant hydrochloride is a crystalline inupadenant hydrochloride described herein. In certain embodiments, the inupadenant hydrochloride is an inupadenant hydrochloride hydrate described herein. In certain embodiments, the inupadenant hydrochloride hydrate is the crystalline inupadenant hydrochloride hydrate described herein.

In another aspect, provided herein is a process for manufacturing a capsule comprising a pharmaceutical composition described herein, the process generally comprising the steps of:
(a) heating a lipid carrier and a polyethylene glycol to form a molten mixture;
(b) adding inupadenant hydrochloride, a copovidone, and BHT to the molten mixture to form an intermediate composition;
(c) homogenizing the intermediate composition;
(d) low shear mixing the intermediate composition to form a pharmaceutical composition; and
(e) filing the capsule with the pharmaceutical composition.

In this aspect, inupadenant hydrochloride is one or more of amorphous inupadenant hydrochloride, crystalline inupadenant hydrochloride, inupadenant hydrochloride hydrate, crystalline inupadenant hydrochloride hydrate, Form 1 inupadenant hydrochloride, and Form 2 inupadenant hydrochloride.

In another aspect, provided herein is a method for treating a cancer in a patient in need thereof, the method generally comprising administering to the patient a pharmaceutical composition described herein, a dosage form described herein, or a capsule described herein.

In another aspect, provided herein is a method for treating a cancer in a patient in need thereof, the method generally comprising administering to the patient an effective amount of an amorphous inupadenant hydrochloride described herein and/or a crystalline inupadenant hydrochloride described herein such as with, for example, Form 1 inupadenant hydrochloride and/or Form 2 inupadenant hydrochloride.

In certain embodiments, the cancer is metastatic. In certain embodiments, cancer is non-metastatic. In certain embodiments, the cancer is selected from breast, bladder, carcinoid, cervical, colorectal, endometrial, glioma, head and neck, liver, lung, melanoma, ovarian, parotid, pancreatic, prostate, metastatic castrate resistant prostate cancer, renal, gastric, sinus, nasal cavity, thyroid, renal transitional cell carcinoma (TCC), renal urothelial carcinoma (UC), and urothelial cancers.

In certain embodiments, the cancer is a non-small cell lung cancer (NSCLC). In certain embodiments, the NSCLC is nonsquamous cell carcinoma. In certain embodiments, the NSCLC is squamous cell carcinoma. In certain embodiments, the NSCLC is metastatic.

In another aspect, provided herein is a method for treating NSCLC in a patient in need thereof, the method comprising administering to the patient a pharmaceutical composition described herein, a dosage form described herein, or a capsule described herein, in combination with pemetrexed and carboplatin.

In another aspect, provided herein is a method for treating NSCLC in a patient in need thereof, the method comprising administering to the patient an effective amount of an amorphous inupadenant hydrochloride described herein and/or a crystalline inupadenant hydrochloride described herein such as, for example, Form 1 inupadenant hydrochloride and/or Form 2 inupadenant hydrochloride, in combination with pemetrexed and carboplatin.

In certain embodiments, the pharmaceutical composition, dosage form, or capsule is administered prior to carboplatin and pemetrexed. In certain embodiments, the effective amount of the amorphous inupadenant hydrochloride and/or the crystalline inupadenant hydrochloride such as, for example, Form 1 inupadenant hydrochloride and/or Form 2 inupadenant hydrochloride is administered prior to carboplatin and pemetrexed.

In certain embodiments, the method provides the patient with a daily dose of about 20 mg to about 1000 mg inupadenant. In certain embodiments, the method provides the patient with a daily dose between about 40 mg and about 640 mg inupadenant. In certain embodiments, the method provides the patient with a daily dose between about 40 mg and about 320 mg inupadenant. In certain embodiments, the method provides the patient with a daily dose of about 40 mg, about 80 mg, about 160 mg, about 320 mg, or about 640 mg inupadenant. In certain embodiments, the method provides the patient with a daily dose of about 40 mg inupadenant. In certain embodiments, the method provides the patient with a daily dose of about 80 mg inupadenant. In certain embodiments, the method provides the patient with a daily dose of about 160 mg inupadenant. In certain embodiments, the method provides the patient with a daily dose of about 320 mg inupadenant. In certain embodiments, the method provides the patient with a daily dose of about 640 mg inupadenant.

In certain embodiments, the pharmaceutical composition, dosage form, or capsule is administered once daily. In certain embodiments, the pharmaceutical composition, dosage form, or capsule is administered twice daily.

In certain embodiments, the method comprises administering to the patient twice daily (BID) a dose of about 10 mg to about 500 mg inupadenant. In certain embodiments, the method comprises administering to the patient twice daily (BID) a dose of about 20 mg to about 320 mg inupadenant. In certain embodiments, the method comprises administering to the patient twice daily (BID) a dose of about 20 mg to about 160 mg inupadenant. In certain embodiments, the method comprises administering to the patient twice daily (BID) a dose of about 20 mg, 20 mg, 80 mg, 160 mg, or 320 mg inupadenant. In certain embodiments, the method comprises administering to the patient twice daily (BID) a dose of about 20 mg inupadenant. In certain embodiments, the method comprises administering to the patient twice daily (BID) a dose of about 40 mg inupadenant. In certain embodiments, the method comprises administering to the patient twice daily (BID) a dose of about 80 mg inupadenant. In certain embodiments, the method comprises administering to the patient twice daily (BID) a dose of about 160 mg inupadenant. In certain embodiments, the method comprises administering to the patient twice daily (BID) a dose of about 320 mg inupadenant.

In certain embodiments, the method comprises administering carboplatin at the standard approved doses of platinum chemotherapy and a pemetrexed dose of 500 mg/m². In certain embodiments, the standard approved doses of platinum chemotherapy correspond to a carboplatin area under the curve of 5 mg/ml per min. In certain embodiments, the method comprises administering the platinum chemotherapy and the pemetrexed every 3 weeks [Q3W] for 4 cycles, followed by pemetrexed maintenance therapy.

In another aspect, provided herein is a method for treating nonsquamous NSCLC comprising administering to a patient in need thereof a pharmaceutical composition described herein, a dosage form described herein, or a capsule described herein.

In another aspect, provided herein is a method for treating nonsquamous NSCLC comprising administering to a patient in need thereof an effective amount of an amorphous inupadenant hydrochloride described herein and/or a crystalline inupadenant hydrochloride described herein such as, for example, Form 1 inupadenant hydrochloride and/or Form 2 inupadenant hydrochloride.

In certain embodiments, the NSCLC is metastatic. In certain embodiments, the NSCLC is stage 3. In certain embodiments, the NSCLC has relapsed or progressed after prior anti-programmed death (PD)-ligand (L)1 therapy.

In another aspect, provided herein is Form 1 inupadenant hydrochloride.

In certain embodiments, Form 1 inupadenant hydrochloride has an XRPD pattern comprising a peak at about 9.6° 2θ. In certain embodiments, the XRPD pattern further comprises one or more peaks selected from about 9.0°, about 15.7°, about 18.2°, about 25.6°, and about 27.0° 20. In certain embodiments, the XRPD pattern further comprises one or more peaks selected from about 16.2°, about 17.5°, about 17.8°, about 22.5°, about 22.7°, about 24.3°, about 24.8°, and about 25.2° 2θ. In certain embodiments, the XRPD pattern further comprises one or more peaks selected from about 15.10, about 19.4°, about 19.6°, about 21.5°, about 23.7°, and about 27.9° 2θ. In certain embodiments, the XRPD pattern further comprises one or more peaks selected from about 5.5°, about 7.6°, about 12.9°, about 13.5°, about 14.0°, and about 20.9° 2θ. In certain embodiments, the XRPD pattern further comprises one or more peaks selected from about 28.4°, about 29.5°, about 29.7°, about 30.8°, about 32.8°, and about 33.8° 2θ.

In certain embodiments, Form 1 inupadenant hydrochloride has an XRPD pattern substantially the same as shown in FIG. 12.

In certain embodiments, a weight loss of about 2.1% occurs upon heating Form 1 inupadenant hydrochloride from about 20° C. to about 80° C. In certain embodiments, Form 1 inupadenant hydrochloride has a TGA thermogram substantially the same as shown in FIG. 13.

In certain embodiments, Form 1 inupadenant hydrochloride has a DSC thermogram comprising one or more endotherms with peak onsets selected from about 60° C. and about 250° C. In certain embodiments, Form 1 inupadenant hydrochloride has a DSC thermogram substantially the same as shown in FIG. 13.

In certain embodiments, Form 1 inupadenant hydrochloride exhibits a change in mass of less than or equal to about 5% wt occurs when varying the relative humidity between 0% and about 95%. In certain embodiments, Form 1 inupadenant hydrochloride has a water sorption isotherm, when measured at 25° C., substantially the same as shown in FIG. 15.

In certain embodiments, the molar ratio of inupadenant to hydrochloride in Form 1 inupadenant hydrochloride is about 1:1. In certain embodiments, the molar ratio of inupadenant to hydrochloride in Form 1 inupadenant hydrochloride is 1:1.

In another aspect, provided herein is milled inupadenant hydrochloride.

In another aspect, provided herein is milled amorphous inupadenant hydrochloride.

In another aspect, provided herein is amorphous inupadenant hydrochloride prepared by milling crystalline inupadenant hydrochloride.

In certain embodiments, the amorphous inupadenant hydrochloride is an amorphous inupadenant hydrochloride described herein. In certain embodiments, the crystalline inupadenant hydrochloride is a crystalline inupadenant hydrochloride described herein.

In certain embodiments, the crystalline inupadenant hydrochloride is milled for between about 30 minutes and about 60 minutes.

### BRIEF DESCRIPTION OF THE DRAWINGS

FIG. 1 is a set of two exemplary X-ray powder diffraction (XRPD) pattern of amorphous inupadenant hydrochloride, prepared in accordance with Example 6.

FIGS. 2A-C represent the differential scanning calorimetry (DSC) analysis of amorphous inupadenant hydrochloride that is heated, cooled, and reheated, according to the method described in Example 1. FIG. 2A shows the DSC thermogram for the first heating cycle. FIG. 2B shows the DSC thermogram for the cooling cycle. FIG. 2C shows the DSC thermogram for the second heating cycle.

FIG. 3 is an overlay of DSC and thermogravimetric analysis (TGA) thermograms of amorphous inupadenant hydrochloride, prepared in accordance with Example 6.

FIG. 4 is an exemplary XRPD pattern of Form 2 inupadenant hydrochloride, prepared in accordance with Example 4.

FIG. 5 is an exemplary XRPD pattern of Form 2 inupadenant hydrochloride, prepared in accordance with Example 3.

FIG. 6 is an exemplary XRPD pattern of crystalline inupadenant hydrochloride heated to 100° C., as further described in Example 9.

FIG. 7 is an exemplary XRPD pattern of crystalline inupadenant hydrochloride heated to 200° C., as further described in Example 9.

FIG. 8 is an exemplary XRPD pattern of crystalline inupadenant hydrochloride at 2% relative humidity, as further described in Example 10.

FIG. 9 is an exemplary DSC thermogram of Form 2 inupadenant hydrochloride, prepared in accordance with Example 3.

FIG. 10A is an exemplary dynamic vapor sorption (DVS) isotherm of crystalline inupadenant hydrochloride, prepared in accordance with Example 3.

FIG. 10B is an exemplary DVS kinetic plot of crystalline inupadenant hydrochloride, prepared in accordance with Example 3.

FIG. 11A is an overlay of DSC and TGA thermograms, with initial weight stabilization, of Form 2 inupadenant hydrochloride, prepared in accordance with Example 3.

FIG. 11B is an overlay of DSC and TGA thermograms, without initial weight stabilization, of Form 2 inupadenant hydrochloride, prepared in accordance with Example 3.

FIG. 12 is an exemplary XRPD pattern for Form 1 inupadenant hydrochloride, prepared in accordance with Example 2.

FIG. 13 is an overlay of DSC and TGA thermograms of Form 1 inupadenant hydrochloride, prepared in accordance with Example 2.

FIG. 14 is an exemplary DSC thermogram of Form 1 inupadenant hydrochloride, prepared in accordance with Example 2.

FIG. 15 is an exemplary DVS isotherm of crystalline inupadenant hydrochloride, prepared in accordance with Example 2.

FIG. 16 is an exemplary XRPD pattern for inupadenant free base, prepared in accordance with Example 5.

FIG. 17 is an overlay of DSC and TGA thermograms of inupadenant free base, prepared in accordance with Example 5.

FIG. 18 is an exemplary DSC thermogram of inupadenant free base, prepared in accordance with Example 5.

FIG. 19 is an exemplary Fourier Transform Infrared (FT-IR) Spectrum for inupadenant free base, prepared in accordance with Example 5.

FIG. 20 is an exemplary FT-IR Spectrum for Form 2 inupadenant hydrochloride, prepared in accordance with Example 3.

FIG. 21 is a stack plot of the XRPD patterns for (i) Form 2 inupadenant hydrochloride; (ii) Form 2 inupadenant hydrochloride at 25° C.; (iii) Form 2 that was subject to heating and the resulting crystalline inupadenant hydrochloride at 100° C.; (iv) Form 2 that was subject to heating and the resulting crystalline inupadenant hydrochloride at 135° C.; (v) Form 2 that was subject to heating and the resulting crystalline inupadenant hydrochloride at 162° C.; (vi) Form 2 that was subject to heating and the resulting crystalline inupadenant hydrochloride at 200° C.; (vii) molten inupadenant hydrochloride at 260° C.; and (viii) Form 2 that was subject to heating to make molten and then cooled to 25° C., as described in Example 9.

FIG. 22 is a stack plot of the XRPD patterns for (i) Form 2 inupadenant hydrochloride at 25° C.; (ii) Form 2 that was subject to heating and the resulting crystalline inupadenant hydrochloride at 170° C. (first heating cycle); (iii) crystalline inupadenant hydrochloride at 170° C. that was subjected to cooling and the resulting crystalline inupadenant hydrochloride at 25° C. (first cooling cycle); (iv) crystalline inupadenant hydrochloride at 25° C. that was subject to heating and the resulting crystalline inupadenant hydrochloride at 170° C. (second heating cycle); and (v) crystalline inupadenant hydrochloride at 170° C. that was subject to cooling and the resulting crystalline inupadenant hydrochloride at 25° C. (second cooling cycle), as described in Example 9.

FIG. 23A is a stack plot of the XRPD patterns for (i) Form 2 inupadenant hydrochloride under ambient conditions; (ii) Form 2 that was subject to 40% relative humidity (RH) and the resulting crystalline inupadenant hydrochloride at 40% RH; (iii) crystalline inupadenant hydrochloride at 40% RH that was subjected to 30% RH and the resulting crystalline inupadenant hydrochloride at 30% RH; (iv) crystalline inupadenant hydrochloride at 30% RH that was subjected to 20% RH and the resulting crystalline inupadenant hydrochloride at 20% RH; (v) crystalline inupadenant hydrochloride at 20% RH that was subjected to 10% RH and the resulting crystalline inupadenant hydrochloride at 10% RH; and (vi) crystalline inupadenant hydrochloride at 10% RH that was subjected to 2% RH and the resulting crystalline inupadenant hydrochloride at 2% RH, as described in Example 10.

FIG. 23B is a stack plot of the XRPD patterns for (i) Form 2 inupadenant hydrochloride under ambient conditions; (ii) Form 2 that was subject to 10% relative humidity (RH) and the resulting crystalline inupadenant hydrochloride at 10% RH; (iii) crystalline inupadenant hydrochloride at 10% RH that was subjected to 20% RH and the resulting crystalline inupadenant hydrochloride at 20% RH; (iv) crystalline inupadenant hydrochloride at 20% RH that was subjected to 30% RH and the resulting crystalline inupadenant hydrochloride at 30% RH; (v) crystalline inupadenant hydrochloride at 30% RH that was subjected to 40% RH and the resulting crystalline inupadenant hydrochloride at 40% RH; and (vi) crystalline inupadenant hydrochloride at 40% RH that was subjected to 50% RH and the resulting crystalline inupadenant hydrochloride at 50% RH, as described in Example 10.

FIG. 23C is a stack plot of the XRPD patterns for (i) Form 2 inupadenant hydrochloride under ambient conditions; (ii) Form 2 that was subject to 60% relative humidity (RH) and the resulting crystalline inupadenant hydrochloride at 60% RH; (iii) crystalline inupadenant hydrochloride at 60% RH that was subjected to 70% RH and the resulting crystalline inupadenant hydrochloride at 70% RH; (iv) crystalline inupadenant hydrochloride at 70% RH that was subjected to 80% RH and the resulting crystalline inupadenant hydrochloride at 80% RH; (v) crystalline inupadenant hydrochloride at 80% RH that was subjected to 90% RH and the resulting crystalline inupadenant hydrochloride at 90% RH; and (vi) crystalline inupadenant hydrochloride at 90% RH that was subjected to 40% RH and the resulting crystalline inupadenant hydrochloride at 40% RH, as described in Example 10.

FIG. 24 is a dissolution plot of Form 2 inupadenant hydrochloride in pH 1.2 HCl/KCl buffer, as described in Example 15.

FIG. 25 represents the dissolution curves of formulations comprising inupadenant hydrochloride in various blends.

FIG. 26 represents the in-vitro dissolution curve for a formulation comprising inupadenant hydrochloride and copovidone (Kollidon®)

FIG. 27 is a schematic representation of the manufacturing process for an exemplary composition comprising inupadenant hydrochloride. Abbreviations: BHT=butylated hydroxytoluene; HCl=hydrochloride.

FIG. 28 represents the plasma concentration-time profile in a dog PK study for free base and hydrochloride salt formulations of inupadenant.

FIG. 29 represents an overlay of geometric mean plasma concentration data over time for inupadenant administered at 80 mg/dog for various inupadenant free base and Form 2 inupadenant hydrochloride formulations with or without pentagastrin, as described in Example 18.

FIG. 30 represents an overlay of geometric mean plasma concentration data over time for inupadenant administered at 80 mg/dog for various formulations of Form 2 inupadenant hydrochloride, with or without pentagastrin, as described in Example 18.

### DETAILED DESCRIPTION

As generally described herein, the present disclosure provides solid, including crystalline and amorphous, forms of inupadenant hydrochloride. The present disclosure also provides pharmaceutical compositions containing inupadenant hydrochloride (the free base of inupadenant may be represented as a compound of formula (Ia) or formula (Ib) below):

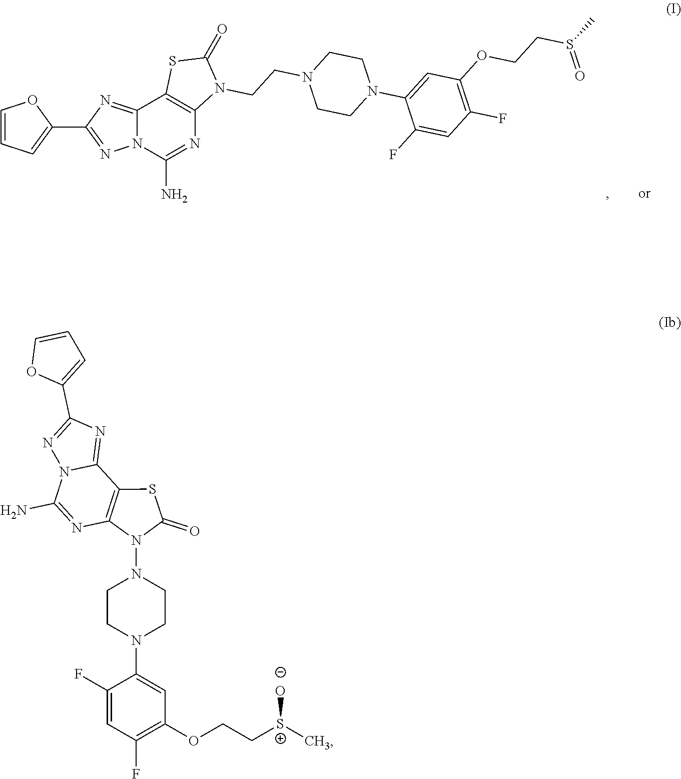 [SMILES: C[S@](=O)CCOc1cc(N2CCN(CCn3c(=O)sc4c3nc(N)n3nc(-c5ccco5)nc43)CC2)c(F)cc1F ; C[S@+]([O-])CCOc1cc(N2CCN(n3c(=O)sc4c3nc(N)n3nc(-c5ccco5)nc43)CC2)c(F)cc1F]

methods of making the pharmaceutical compositions disclosed herein, and methods of using the crystalline forms and pharmaceutical compositions in combination with carboplatin and pemetrexed to treat non-small cell lung cancer (NSCLC), including squamous and nonsquamous NSCLC.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. For example, the term “w/w” means “weight by weight”, that is the proportion of a particular substance within a mixture, as measured by weight. Thus, the term “about 12% (w/w)” means that a particular composition contains “about” 12% of a particular substance. The term “wt” means “weight. So, the term “12% wt” means “12% by weight”. When used in the context of weight percentages, the term “about” means±10% of the value modified by the term “about”. Further, if a variable is not accompanied by a definition, then the previous definition of the variable controls.

As used herein, “XRPD” refers to X-ray powder diffraction. An XRPD pattern is an x-y graph with 20 (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak are typically preceded with the term “about” or proceeded with an appropriate range defining the experimental variability. For purposes of data reported herein, that value is ±0.3° 2θ unless otherwise stated. This means that on a well-maintained instrument one would expect the variability in peak measurement to be ±0.3° 2θ. X-ray powder diffraction peaks cited herein are generally reported with this variability of ±0.3° 2θ unless stated otherwise and are intended to be reported with such a variability whenever disclosed herein whether the word “about” is present or not, unless context dictates otherwise.

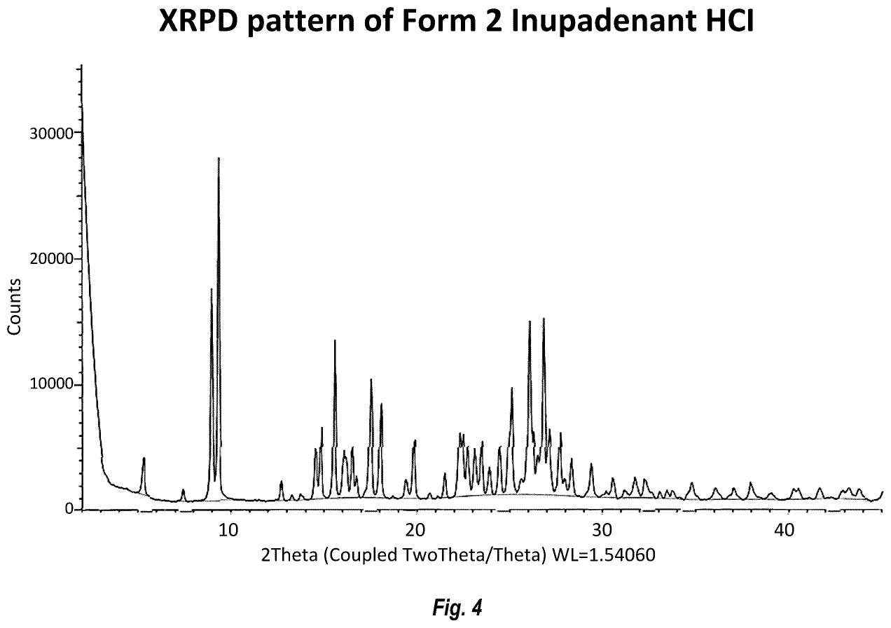

Crystalline forms, such as crystalline inupadenant hydrochloride, are readily analyzed by XRPD. The data from x-ray powder diffraction may be used in multiple ways to characterize crystalline forms. For example, the entire x-ray powder diffraction pattern output from a diffractometer may be used to characterize a crystalline form such as crystalline inupadenant hydrochloride. A smaller subset of such data, however, may also be suitable and used for characterizing such crystalline forms. Indeed, often even a single x-ray powder diffraction peak may be used to characterize such a crystalline form. With respect to crystalline inupadenant hydrochloride, any one or more of the peaks in the x-ray powder diffraction pattern of FIG. 4 may be used to characterize crystalline inupadenant hydrochloride.

There may be variability in the Fourier-transform infrared (FT-IR) data described herein. Due to such sources of variability, the values of each FT-IR peak are typically preceded with the term “about” or proceeded with an appropriate range defining the experimental variability. For purposes of data reported herein, that value is +4 cm⁻¹ unless otherwise stated. This means that on a well-maintained instrument one would expect the variability in peak measurement to be 4 cm⁻¹. FT-IR peaks cited herein are generally reported with this variability of ±4 cm⁻¹ unless stated otherwise and are intended to be reported with such a variability whenever disclosed herein whether the word “about” is present or not, unless context dictates otherwise.

As used herein, the term “one or more peaks” means that any combination of the peaks so given may be present in the corresponding analytical measurement, such as an XRPD pattern. It does not mean (in and of itself) that no other peak may be present in the corresponding analytical measurement, such as an XRPD pattern.

Because hydrates are known to often have greater variability with thermal measurements such as DSC, no explicit definition of “about” is provided herein with respect to thermal measurements. However, variability on the order of 10° C. is possible.

The term “substantially crystalline” refers to solid forms that may be at least a particular weight percent crystalline. Particular weight percentages may include 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, inupadenant hydrochloride can be a substantially crystalline sample of any of the crystalline solid forms described herein (e.g., a crystalline form of inupadenant hydrochloride with the XRPD pattern shown in FIG. 4 which is Form 2 inupadenant hydrochloride).

The term “substantially pure” relates to the composition of a specific crystalline solid form (e.g., a crystalline form of inupadenant hydrochloride) that may be at least a particular weight percent free of impurities and/or other solid forms. Particular weight percentages may include 70%, 75%, 80%, 85%, 90%, 95%, 99%, or any percentage between 70% and 100%. In certain embodiments, inupadenant hydrochloride can be a substantially pure sample of any of the crystalline solid forms described herein, (e.g., a crystalline form with the XRPD pattern shown in FIGS. 4, 5, 6, 7, 8, 12, 21, 22, 23A, 23B, and/or 23C). In certain embodiments, inupadenant hydrochloride can be a substantially pure crystalline form with the XRPD pattern shown in FIG. 4—which is an XRPD pattern of Form 2 inupadenant hydrochloride.

As used herein, “dissolution” refers to dissolution testing of a drug substance or drug product at multiple time points. For example, dissolution profiles for drug substances (e.g., inupadenant) or drug products (e.g., the pharmaceutical compositions described herein) may be performed for characterization and quality control to ensure the drug is released at a defined range of rates in a well-defined dissolution aqueous media that is at least sink conditions for that drug, or in biorelevant media such as simulated gastric or intestinal fluids representing either the fasted or fed states. In certain cases, but not others, dissolution testing may be predictive of or give insight into in vivo bioavailability of the drug substance. Dissolution testing may be performed using USP testing protocols and dissolution apparatus.

As used herein, “granulation” refers to a process of forming granules from a powdered or particulate material. As used herein, “dry granulation” refers to a process in which granules are formed without the presence of a liquid solution and may be useful in the preparation of granules of materials sensitive to heat, moisture, or solvents. For example, roller compaction is a dry granulation process. As used herein, “wet granulation” refers to the formation of granules wherein the particles are bound together using a binder or a liquid solution. Examples of wet granulation are high shear granulation and fluid bed granulation.

As used herein, “pharmaceutical composition” or “pharmaceutical formulation” refer to the combination of a therapeutically active agent with a pharmaceutically acceptable excipient, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or human, as appropriate; or means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

The pharmaceutical composition of the invention may optionally comprise one or more pharmaceutically acceptable carriers, diluents, excipients and/or adjuvants. As used herein, “pharmaceutically acceptable excipient” refers to a substance that aids the administration of an active agent to and/or absorption by a subject and can be included in the compositions of the present invention without causing a significant adverse toxicological effect on the patient. Non-limiting examples of pharmaceutically acceptable excipients include cosolvents, binders, antioxidants, surfactants, wetting agents, dissolution aids, emulsifying agents, buffering agents, pH modifying agents, preserving agents (or preservating agents), isotonifiers, stabilizing agents, granulating agents or binders, precipitation inhibitors, lubricants, disintegrants, glidants, diluents or fillers, adsorbents, dispersing agents, suspending agents, bulking agents, release agents, sweetening agents, flavoring agents, coatings, gelatins, carbohydrates such as lactose, amylose or starch, fatty acid esters, hydroxypropylmethylcellulose, polyvinyl pyrrolidine, and colors, and the like. For examples of excipients, see Martin, Remington's Pharmaceutical Sciences, 15th Ed., Mack Publ. Co., Easton, PA (1975) or Rowe, Shesky, and Quinn, Handbook of Pharmaceutical Excipients, 6^{th} Ed. Pharmaceutical Press, London, UK (2009).

The various classes of excipient examples are not rigid categories and often times excipients may be characterized in more than one category.

Examples of diluents or fillers include, but are not limited to, a sugar (e.g., mannitol, lactose, sorbitol, lactitol, erythritol, sucrose, fructose, glucose, agarose, maltose, isomalt, polydextrose, and combinations thereof), an inorganic material (e.g., dibasic calcium phosphate, hydroxyapatite, sodium carbonate, sodium bicarbonate, calcium carbonate, calcium sulfate, magnesium carbonate, magnesium oxide, bentonite, kaolin), calcium lactate, a starch (e.g., a pregelatinized starch), a microcrystalline cellulose, a silicified microcrystalline cellulose, a polysaccharide, a cellulose (e.g., a hydroxypropylcellulose, a hypromellose, a carboxymethylcellulose, a methylcellulose, a hydroxypropylmethylcellulose, a hydroxyethylcellulose), a dextrin, a maltodextrin, an alginate, a collagen, a polyvinylpyrrolidone, a polyvinylacrylate, polyethylene oxide, and polyethylene glycol. Sugar is defined herein to include sugar alcohols.

Examples of adsorbents include, but are not limited to silicon dioxide, purified aluminum silicate and the like.

Examples of disintegrants include, but are not limited to, alginic acid, an alginate, primogel, a cellulose (e.g., hydroxypropylcellulose), polacrillin potassium, sodium starch glycolate, sodium croscarmellose, a polyplasdone (e.g., a crospovidone), and a starch (e.g., corn starch, pregelatinized starch, hydroxypropyl starch, and carboxymethyl starch).

Examples of binders include, but are not limited to, a hydroxypropylcellulose, hydroxyethylcellulose, a hydroxypropylmethylcellulose (e.g., a low viscosity hydroxypropylmethylcellulose), a sugar, a polyvinylpyrrolidone, a polyvinyl alcohol, a polyvinyl acetate, a polydextrose, a chitosan, a carrageenan, carbophil, a microcrystalline cellulose, gum tragacanth, guar gum, gellan gum, gelatin, and a starch (e.g., corn starch).

Examples of wetting agents include, but are not limited to, a poloxamer (e.g., poloxamer 407), sodium dodecyl sulfate, sodium lauryl sulfate (SLS), sodium stearyl fumarate (SSF), a polydimethylsiloxane, a polysorbate (e.g., polyoxyethylene 20 sorbitan mono-oleate (Tween® 20)), sorbitan monooleate, sorbitan trioleate, sorbitan laurate, sorbitan stearate, sorbitan monopalmitate, lecithin, sodium taurocholate, ursodeoxycholate, polyethoxylated castor oil, cetyl trimethylammonium bromide, nonoxynol, 6-tocopherol polyethylene glycol 1000 succinate, and docusate sodium. In some embodiments, the wetting agent is sodium lauryl sulphate. Further examples of wetting and/or dissolution aids include certain cosolvents including, but not limited to caprylic acid, polyethylene glycol (PEG), propylene glycol, ethanol, dimethylsulfoxide, dimethylacetamide, dimethylisosorbide and mixtures thereof.

Examples of antioxidants include, but are not limited to butylated hydroxytoluene (BHT), butylated hydroxyanisole (BHA), citric acid, sodium metabisulfite, ascorbic acid, methionine and vitamin E. In some embodiments, the antioxidant is BHT.

Examples of surfactants include, but are not limited to polyethylene glycols, polyoxyethylene sorbitan fatty acid esters, sorbitan esters, sodium docusate, sodium lauryl sulfate, polysorbates (20, 80, etc.), poloxamers (188, 407 etc.), pluronic polyols, polyoxyethylene sorbitan monoethers (TWEEN®-20, TWEEN®-80, etc.), vitamin E TPGS (Vitamin E polyethylene glycol succinate), cremophor RH40 (polyoxyl 40 hydrogenated castor oil), cremophor EL (polyoxyl 35 hydrogenated castor oil), polyethylene glycol 660 12-monostearate, solutol HS15 (Polyoxyethylated 12-hydroxystearic acid), labrasol (caprylocaproyl polyoxyl-8 glycerides), labrafil M1944 (Oleoyl polyoxyl-6 glycerides).

Examples of lubricants and glidants include, but are not limited to, a wax, a glyceride, a light mineral oil, a polyethylene glycol, sodium stearyl fumarate, magnesium stearate, stearic acid, hydrogenated oil (e.g., hydrogenated vegetable oil), an alkyl sulfate, sodium benzoate, sodium acetate, glyceryl behenate, palmitic acid, and coconut oil.

Examples of glidants include, but are not limited to, colloidal silicon dioxide, talc, kaolin, bentonite, and activated carbon/charcoal.

Examples of emulsifying agents include, but are not limited to, carbomer, carrageenan, lanolin, lecithin, mineral oil, oleic acid, oleyl alcohol, pectin, poloxamer, polyoxyethylene sorbitan fatty acid esters, sorbitan esters, triethanolamine, propylene glycol monolaurate, propylene glycol dilaurate, propylene glycol monocaprylate. In some embodiments emulsifying agents are for example poloxamer, propylene glycol monolaurate, propylene glycol dilaurate, and propylene glycol monocaprylate.

Examples of buffering agents that are used to help to maintain the pH in the range that approximates physiological conditions include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Examples of pH modifiers include, but are not limited to sodium hydroxide, sodium bicarbonate, magnesium oxide, potassium hydroxide, meglumine, sodium carbonate, citric acid, tartaric acid, ascorbic acid, fumaric acid, succinic acid and malic acid.

Examples of preservatives agents added to retard microbial include, but are not limited to phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

Examples of isotonifiers (sometimes known as “stabilizers”) include, but are not limited to polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall or helps to inhibit the precipitation, particle growth or agglomeration of the active ingredient. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); monosaccharides, such as xylose, mannose, fructose, glucose; disaccharides such as lactose, maltose, sucrose and trisaccacharides such as raffinose; polysaccharides such as dextran; polyethylene glycol methyl ether-block-poly(D-L-lactide) copolymer; poly(butyl methacrylate-co-(2-dimethylaminoethyl) methacrylate-co-methyl methacrylate) 1:2:1. In some embodiments stabilizers are for example glycerol; polyethylene glycol; polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); polyethylene glycol methyl ether-block-poly(D-L-lactide) copolymer; and poly(butyl methacrylate-co-(2-dimethylaminoethyl) methacrylate-co-methyl methacrylate) 1:2:1.

Examples of granulating agent/binder(s) include, but are not limited to starch, gums (inclusive of natural, semisynthetic and synthetic), microcrystalline cellulose, ethyl cellulose, methylcellulose, hydroxypropylcellulose, liquid glucose polymers such as povidone, polyvinylpyrrolidone polyvinylacetate copolymer and the like. In some embodiments granulating agents are for example methylcellulose, hydroxypropylcellulose, povidone and polyvinylpyrrolidone polyvinylacetate copolymer.

Examples of precipitation inhibitors include, but are not limited to, water soluble derivatives of cellulose including hydroxypropylmethylcellulose and methylcellulose, and water soluble polymers such as polyvinylpyrrolidone or polyvinylpyrrolidone polyvinylacetate copolymer. In some embodiments the precipitation inhibitor is hydroxypropylmethylcellulose.

Examples of colorants include, but are not limited to, titanium dioxide, aluminum lakes, iron oxides and carbon black.

Examples of coatings include but are not limited to, a film forming polymer (e.g., a hypromellose, a methyl cellulose, an ethylcellulose, cellulose acetate, a hydroxypropylmethyl cellulose, a hydroxypropyl cellulose, hydroxypropylmethyl cellulose acetate succinate, cellulose acetate phthalate, a polyvinylpyrrolidone, polyvinyl alcohol, a Eudragit/acrylate) and a plasticizer (e.g., triacetin, polyethylene glycol, propylene glycol).

Pharmaceutical compositions for oral administration of inupadenant hydrochloride can take the form of bulk liquid solutions or suspensions or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pills, tablets, capsules or the like in the case of solid compositions and may be semisolid or liquid or solutions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, minitabs, sachets, stickpacks, reconstitutable powders, dry powder inhalers, lozenges, and chewables.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., anti-cancer agent, chemotherapeutic, or treatment for a neurodegenerative disease). Inupadenant, can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (e.g., “therapeutic treatment”).

In general, an “effective amount” of a compound refers to an amount sufficient to elicit the desired biological response, e.g., to treat a disease or disorder of the brain and/or central nervous system. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

### Solid Forms of Inupadenant Hydrochloride

The present invention provides inupadenant hydrochloride. Inupadenant may also be referred to herein as EOS-850, EOS100850, (S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one, or (+)-(S)-5-amino-3-(2-(4-(2,4-difluoro-5-(2-methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one. Inupadenant may also be represented as a compound of formula (Ia) or formula (Ib), whose structures are equivalent:

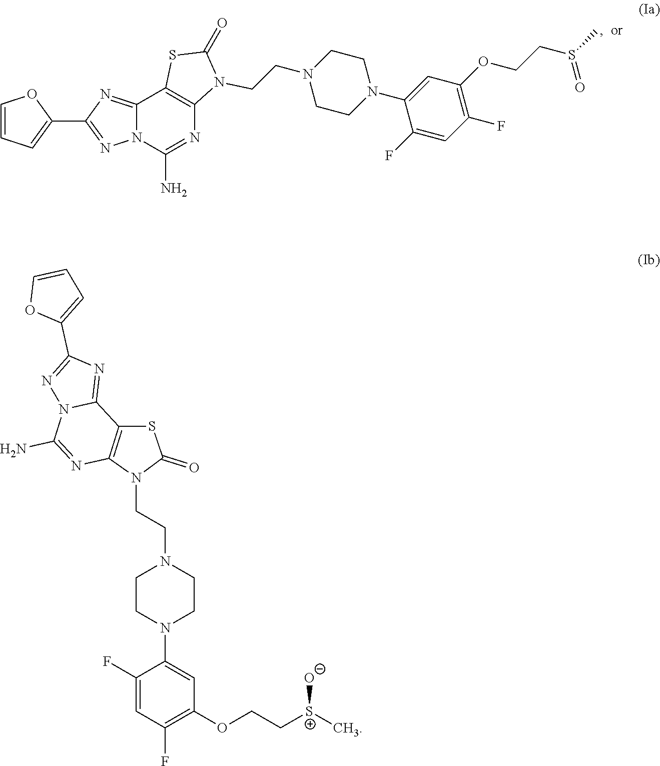 [SMILES: C[S@](=O)CCOc1cc(N2CCN(CCn3c(=O)sc4c3nc(N)n3nc(-c5ccco5)nc43)CC2)c(F)cc1F ; C[S@+]([O-])CCOc1cc(N2CCN(CCn3c(=O)sc4c3nc(N)n3nc(-c5ccco5)nc43)CC2)c(F)cc1F]

Methods for chemically synthesizing inupadenant have been described, for example, in PCT Application Publication No. WO2018178338, which is incorporated herein by reference in its entirety. A method for preparing inupadenant free base and inupadenant hydrochloride is depicted in Scheme 1.

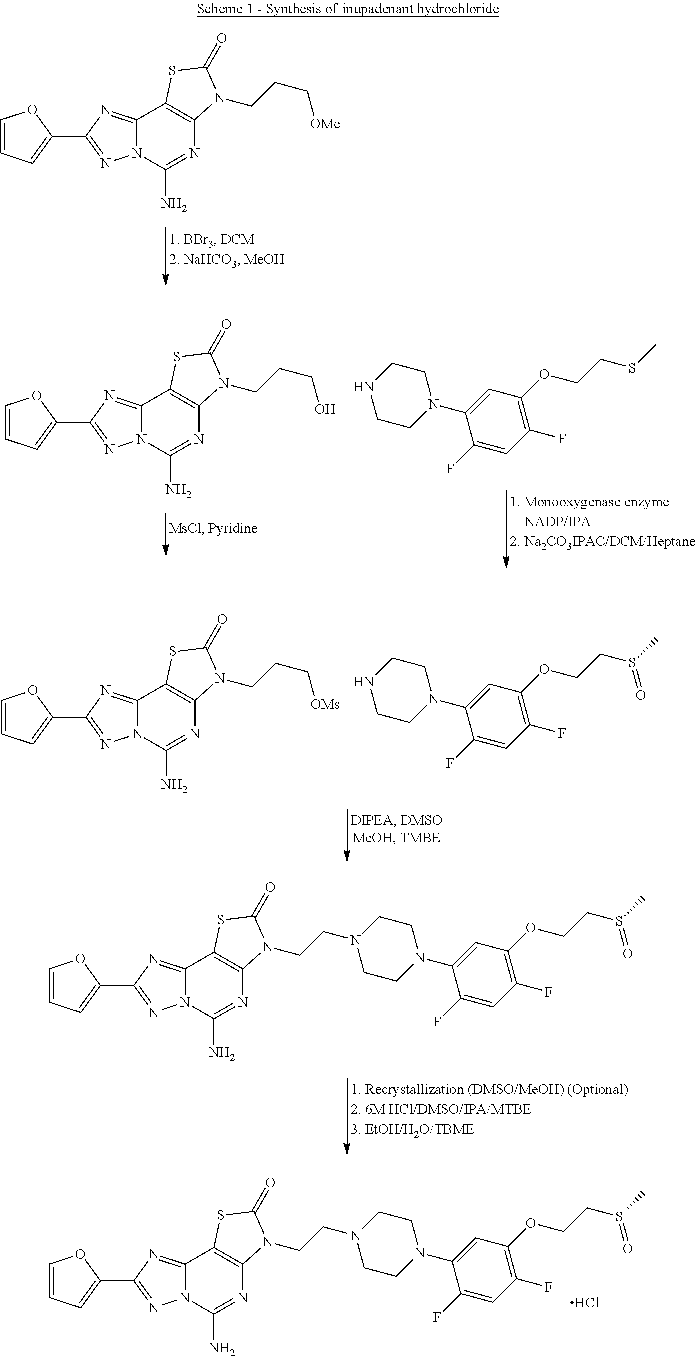

In certain embodiments, the hydrochloride salt of inupadenant is a crystalline hydrochloride salt of inupadenant. In some embodiments, the hydrochloride salt of inupadenant is a substantially crystalline hydrochloride salt of inupadenant, wherein the meaning of “substantially crystalline” is as defined herein.

In some embodiments, the crystalline inupadenant hydrochloride is a crystalline hydrate of inupadenant hydrochloride.

In some embodiments, the crystalline inupadenant hydrochloride is Form 1 inupadenant hydrochloride.

In some embodiments, the crystalline inupadenant hydrochloride is Form 2 inupadenant hydrochloride.

In certain embodiments, the hydrochloride salt of inupadenant is an amorphous hydrochloride salt of inupadenant.

In certain embodiments, crystalline inupadenant hydrochloride is a substantially pure crystalline form (e.g., a crystalline form with the XRPD pattern shown in FIG. 4), wherein the meaning of “substantially pure” is as defined herein. In certain embodiments, crystalline inupadenant hydrochloride comprises a mixture of two or more crystalline forms.

### (1) Crystalline Inupadenant Free Base

In one aspect provided herein is crystalline inupadenant free base.

In certain embodiments, crystalline inupadenant free base has an X-ray powder diffraction (XRPD) pattern comprising a peak at about 10° 2θ.

In certain embodiments, the XRPD pattern further comprises one or more peaks selected from about 20.2°, about 21.7°, about 22.4°, about 23°, and about 27.6° 2θ. In certain embodiments, the XRPD pattern further comprises one or more peaks selected from about 13.9°, about 15.3°, about 16.10, about 16.4°, about 17.3°, about 19.2°, and about 19.9° 2θ. In certain embodiments, the XRPD pattern further comprises one or more peaks selected from about 6.6°, about 7.6°, about 11.5°, about 12.2°, about 12.5°, about 13.1°, about 13.7°, about 16.6°, about 17.8°, about 18.4°, about 20.3°, about 21.2°, about 21.3°, about 21.4°, about 23.7°, about 24°, about 24.5°, about 25.2°, about 25.4°, about 25.9°, about 26°, about 26.7°, about 27°, about 27.10, and about 28° 2θ. In certain embodiments, the XRPD pattern further comprises one or more peaks selected from about 28.4°, about 28.6°, about 29°, about 29.4°, about 29.5°, about 29.9°, about 30.2°, about 30.5°, about 30.9°, about 31.6°, about 31.9°, about 32.3°, about 32.5°, about 32.9°, about 33.1°, about 33.4°, about 33.7°, about 33.9°, and about 34.2° 2θ.

In certain embodiments, crystalline inupadenant free base has an X-ray powder diffraction (XRPD) pattern comprising a peak at 10°±0.3° 2θ.

In certain embodiments, the XRPD pattern further comprises one or more peaks selected from 20.2°±0.3°, 21.7°±0.3°, 22.4°±0.3°, 23°±0.3°, and 27.6°±0.3° 2θ. In certain embodiments, the XRPD pattern further comprises one or more peaks selected from 13.9°±0.3°, 15.3°±0.3°, 16.1°±0.3°, 16.4°±0.3°, 17.3°±0.3°, 19.2°±0.3°, and 19.9°±0.3° 2θ. In certain embodiments, the XRPD pattern further comprises one or more peaks selected from 6.6°±0.3°, 7.6°±0.3°, 11.5°±0.3°, 12.2°±0.3°, 12.5°±0.3°, 13.1°±0.3°, 13.7°±0.3°, 16.6°±0.3°, 17.8°±0.3°, 18.4°±0.3°, 20.3°±0.3°, 21.2°±0.3°, 21.3°±0.3°, 21.4°±0.3°, 23.7°±0.3°, 24°±0.3°, 24.5°±0.3°, 25.2°±0.3°, 25.4°±0.3°, 25.9°±0.3°, 26°±0.3°, 26.7°±0.3°, 27°±0.3°, 27.1°±0.3°, and 28°±0.3° 2θ. In certain embodiments, the XRPD pattern further comprises one or more peaks selected from 28.4°±0.3°, 28.6°±0.3°, 29°±0.3°, 29.4°±0.3°, 29.5°±0.3°, 29.9°±0.3°, 30.2°±0.3°, 30.5°±0.3°, 30.9°±0.3°, 31.6°±0.3°, 31.9°±0.3°, 32.3°±0.3°, 32.5°±0.3°, 32.9°±0.3°, 33.1°±0.3°, 33.4°±0.3°, 33.7°±0.3°, 33.9°±0.3°, and 34.2°±0.3°.

In certain embodiments, crystalline inupadenant free base has an X-ray powder diffraction (XRPD) pattern comprising a peak at 10°±0.2°.

In certain embodiments, the XRPD pattern further comprises one or more peaks selected from 20.2°±0.2°, 21.7°±0.2°, 22.4°±0.2°, 23°±0.2°, and 27.6°±0.2° 2θ. In certain embodiments, the XRPD pattern further comprises one or more peaks selected from 13.9°±0.2°, 15.3°±0.2°, 16.1°±0.2°, 16.4°±0.2°, 17.3°±0.2°, 19.2°±0.2°, and 19.9°±0.2° 2θ. In certain embodiments, the XRPD pattern further comprises one or more peaks selected from 6.6°±0.2°, 7.6°±0.2°, 11.5°±0.2°, 12.2°±0.2°, 12.5°±0.2°, 13.1°±0.2°, 13.7°±0.2°, 16.6°±0.2°, 17.8°±0.2°, 18.4°±0.2°, 20.3°±0.2°, 21.2°±0.2°, 21.3°±0.2°, 21.4° °±0.2°, 23.7°±0.2, 24°±0.2°, 24.50°±0.20, 25.20°±0.20, 25.40°±0.20, 25.90°±0.20, 260°±0.20, 26.70°±0.20, 270°±0.20, 27.10° 0.2°, and 28°±0.2° 2θ. In certain embodiments, the XRPD pattern further comprises one or more peaks selected from 28.4°±0.2°, 28.6°±0.2°, 29°±0.2°, 29.4°±0.2°, 29.5°±0.2°, 29.9°±0.2°, 30.2°±0.2°, 30.5°±0.2°, 30.9°±0.2°, 31.6°±0.2°, 31.9°±0.2°, 32.3°±0.2°, 32.5°±0.2°, 32.9°±0.2°, 33.1°±0.2°, 33.4°±0.2°, 33.7°±0.2°, 33.9°±0.2°, and 34.2°±0.2°.

In certain embodiments, crystalline inupadenant free base has an X-ray powder diffraction (XRPD) pattern comprising a peak at 10°±0.1° 2θ.

In certain embodiments, the XRPD pattern further comprises one or more peaks selected from 20.2°±0.1°, 21.7°±0.1°, 22.4°±0.1°, 23°±0.1°, and 27.6°±0.1° 2θ. In certain embodiments, the XRPD pattern further comprises one or more peaks selected from 13.9°±0.1°, 15.3°±0.1°, 16.1°±0.1°, 16.4°±0.1°, 17.3°±0.1°, 19.2°±0.1°, and 19.9°±0.1° 2θ. In certain embodiments, the XRPD pattern further comprises one or more peaks selected from 6.6°±0.1°, 7.6°±0.1°, 11.5°±0.1°, 12.2°±0.1°, 12.5°±0.1°, 13.1°±0.1°, 13.7°±0.1°, 16.6°±0.1°, 17.8°±0.1°, 18.4°±0.1°, 20.3°±0.1°, 21.2°±0.1°, 21.3°±0.1°, 21.4°±0.1°, 23.7°±0.1°, 24°±0.1°, 24.5°±0.1°, 25.2°±0.1°, 25.4°±0.1°, 25.9°±0.1°, 26°±0.1°, 26.7°±0.1°, 27°±0.1°, 27.1°±0.1°, and 28°±0.1° 2θ. In certain embodiments, the XRPD pattern further comprises one or more peaks selected from 28.4°±0.1°, 28.6°±0.1°, 29°±0.1°, 29.4°±0.1°, 29.5°±0.1°, 29.9°±0.1°, 30.2°±0.1°, 30.5°±0.1°, 30.9°±0.1°, 31.6°±0.1°, 31.9°±0.1°, 32.3°±0.1°, 32.5°±0.1°, 32.9°±0.1°, 33.1°±0.1°, 33.4°±0.1°, 33.7°±0.1°, 33.9°±0.1°, and 34.2°±0.1°.

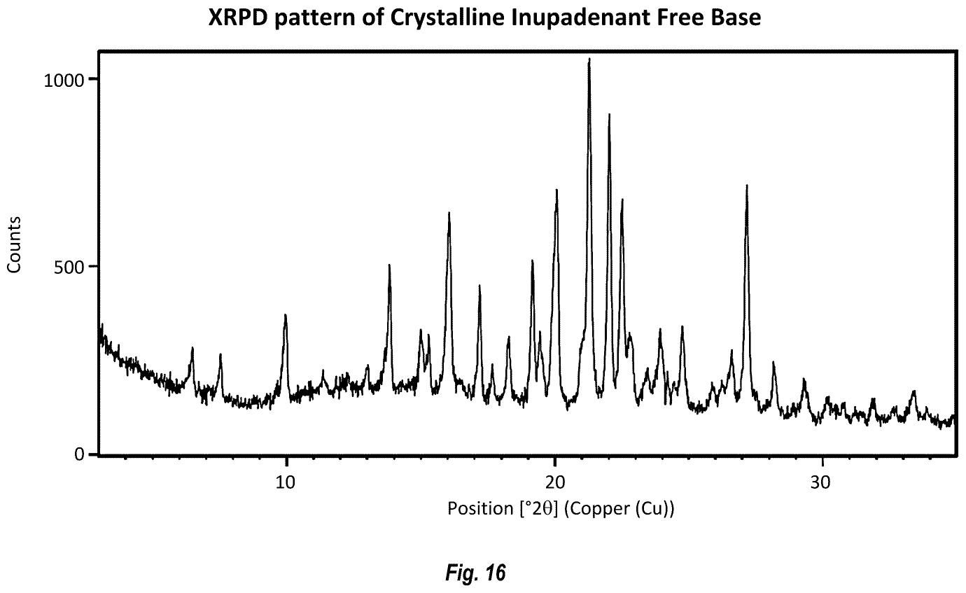

In certain embodiments, crystalline inupadenant free base has an XRPD pattern substantially the same as shown in FIG. 16.

In certain embodiments, crystalline inupadenant free base has an XRPD pattern comprising one or more diffraction peaks (20) disclosed in Table 4.

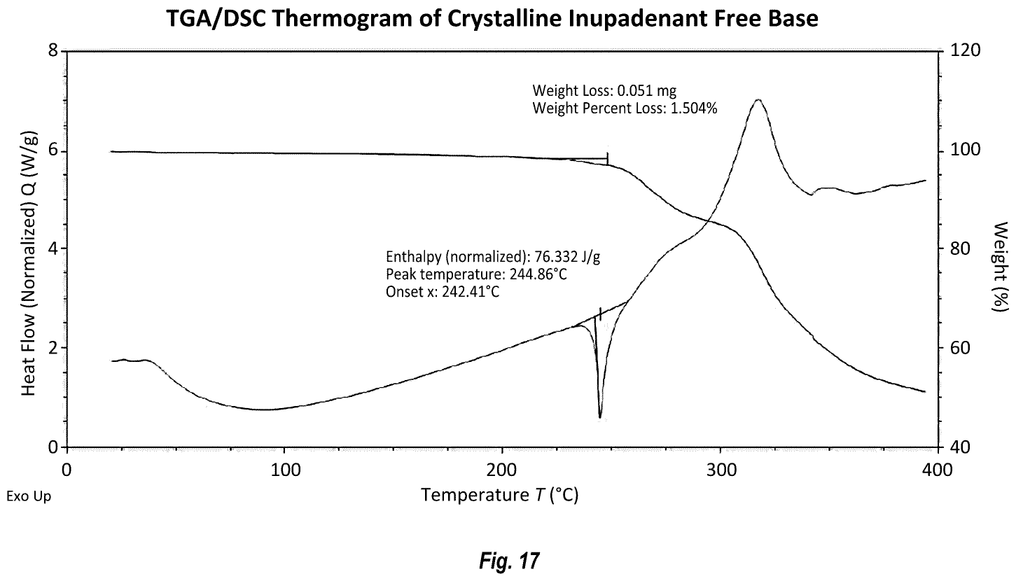

In certain embodiments, crystalline inupadenant hydrochloride has a thermogravimetric analysis (TGA) thermogram substantially the same as shown in FIG. 17.

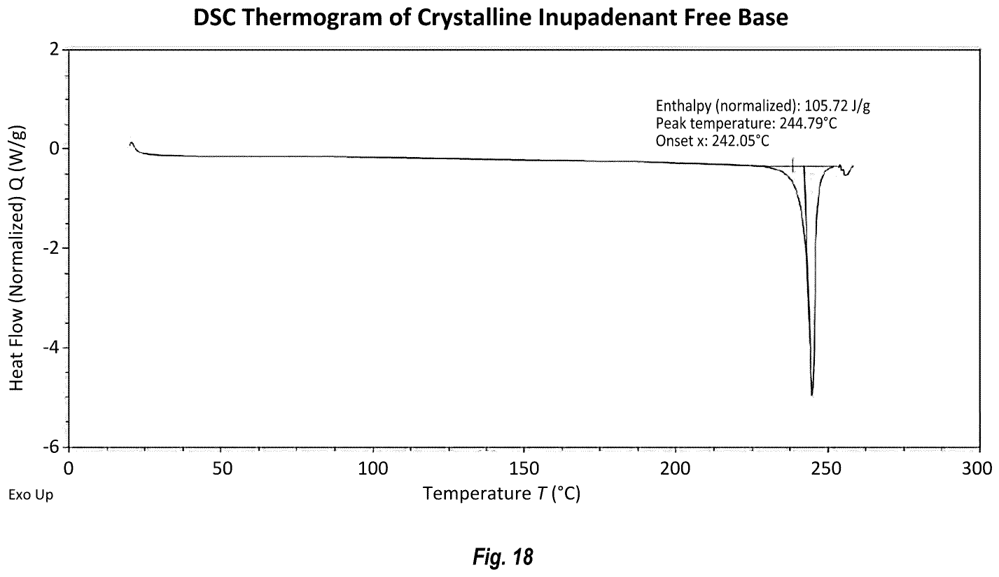

In certain embodiments, crystalline inupadenant free base has a differential scanning calorimetry (DSC) thermogram comprising an endotherm with a peak onset at about 242° C. In certain embodiments, crystalline inupadenant free base has a DSC thermogram comprising a melting peak onset at about 242° C. In certain embodiments, crystalline inupadenant free base has a DSC thermogram substantially the same as shown in FIG. 17. In certain embodiments, crystalline inupadenant free base has a DSC thermogram substantially the same as shown in FIG. 18.

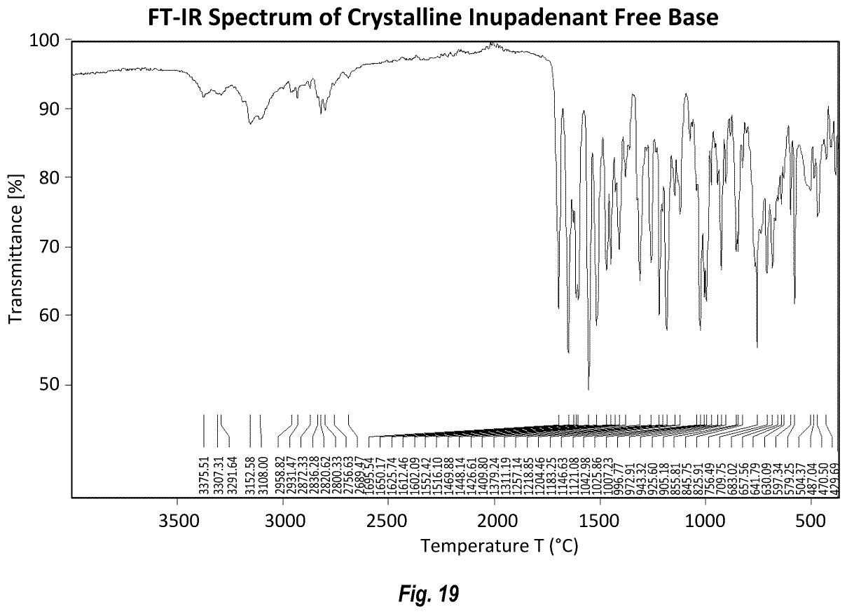

In certain embodiments, crystalline inupadenant free base has a Fourier Transform Infrared (FT-IR) spectrum comprising one or more peaks disclosed in Table 5. In certain embodiments, crystalline inupadenant free base has a FT-IR spectrum substantially the same as shown in FIG. 19.

### (2) Amorphous Inupadenant Hydrochloride

In one aspect provided herein is amorphous inupadenant hydrochloride.

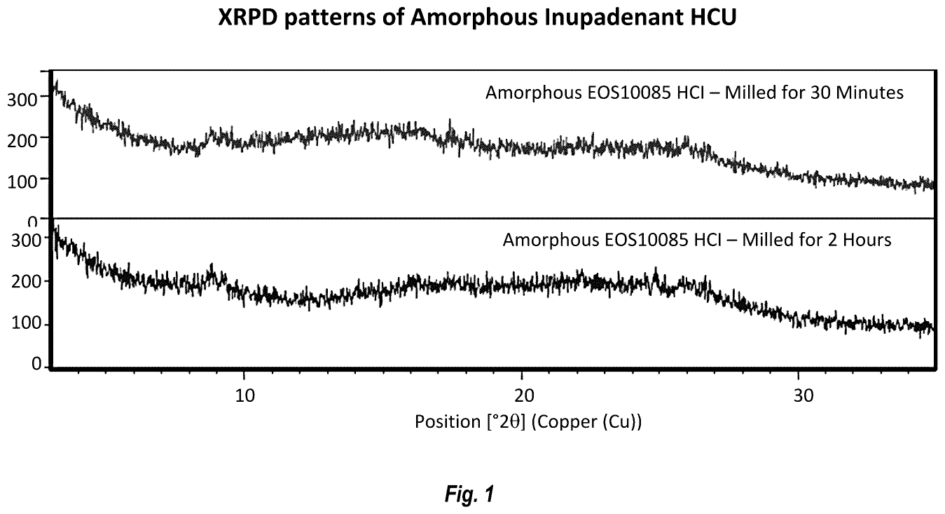

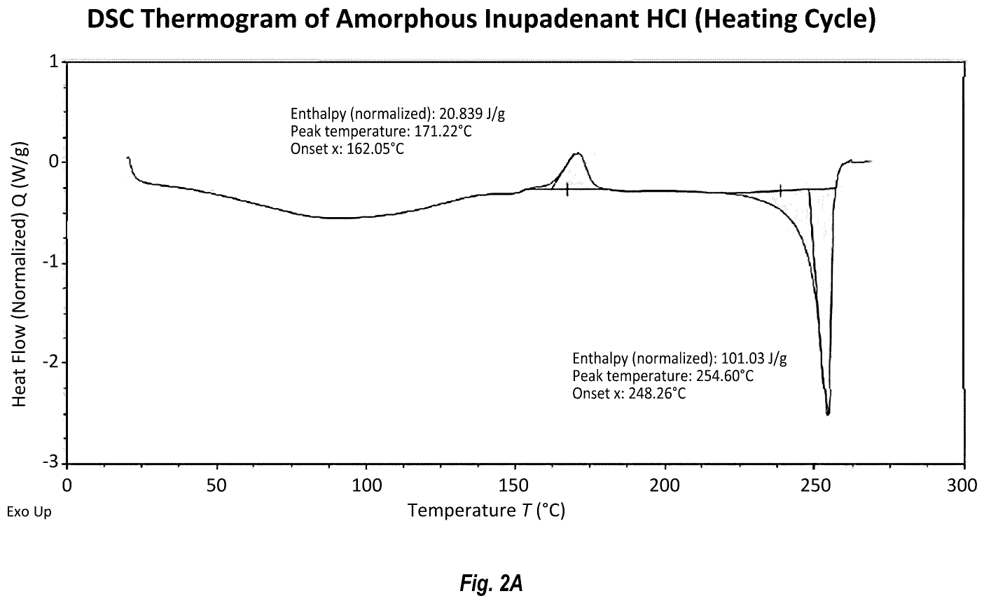

In certain embodiments, the amorphous inupadenant hydrochloride has an XRPD pattern substantially the same as either X-ray powder diffraction pattern shown in FIG. 1.

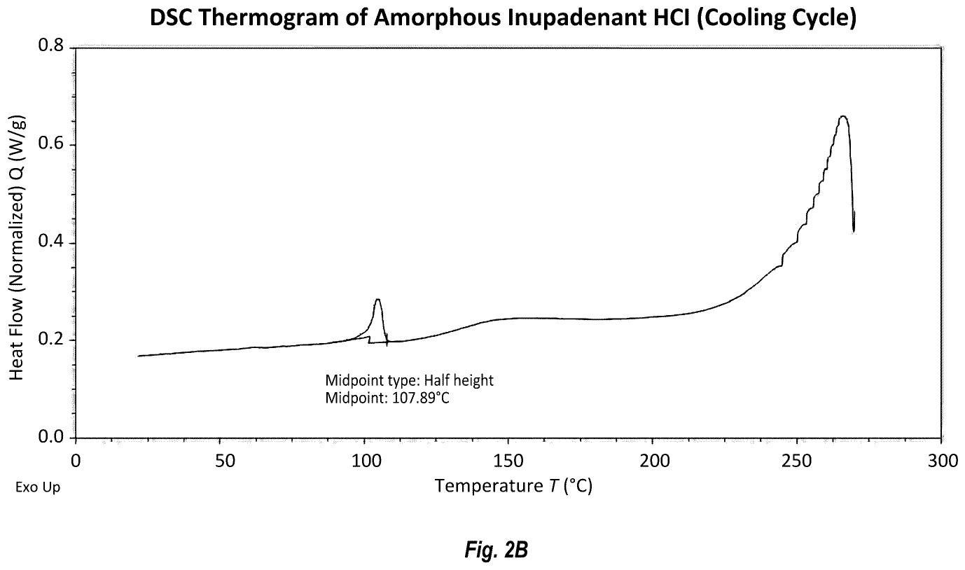

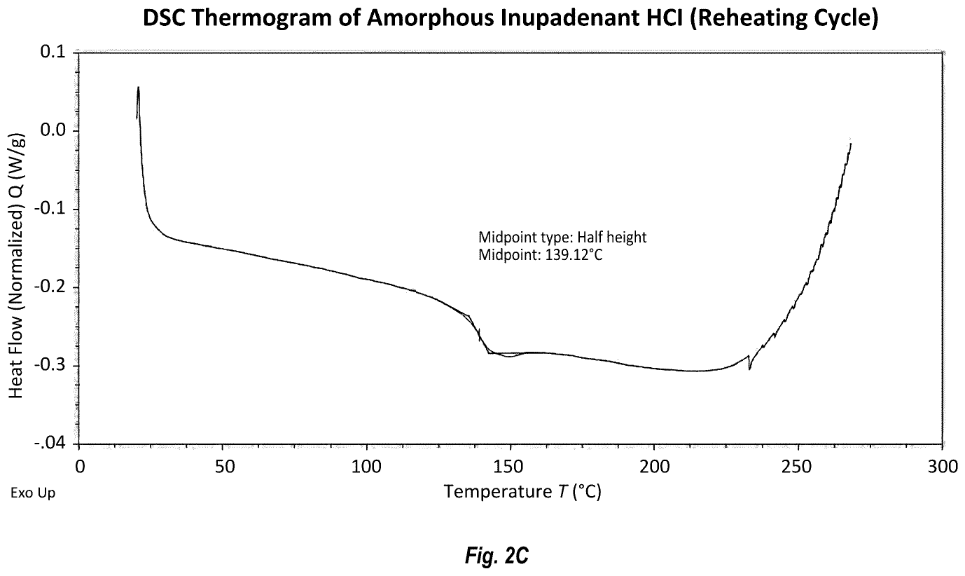

In certain embodiments, the amorphous inupadenant hydrochloride has a DSC thermogram comprising an exotherm with a peak onset temperature between about 155° C. and about 165° C. In certain embodiments, the amorphous inupadenant hydrochloride has a DSC thermogram comprising an endotherm with a peak onset between about 230° C. and about 250° C. In certain embodiments, the amorphous inupadenant hydrochloride has a DSC thermogram comprising an exotherm with a peak onset temperature between about 155° C. and about 165° C. and an endotherm with a peak onset between about 230° C. and about 250° C. In certain embodiments, the amorphous inupadenant hydrochloride is characterized by a DSC thermogram substantially the same as shown in FIG. 2A.

In certain embodiments, the amorphous inupadenant hydrochloride is characterized by a glass transition temperature with a midpoint at about 110° C. In certain embodiments, the amorphous inupadenant hydrochloride is characterized by a glass transition temperature with a midpoint at a temperature between about 140° C. and about 160° C. The glass transition temperature of amorphous inupadenant hydrochloride can be determined, for example, using DSC.

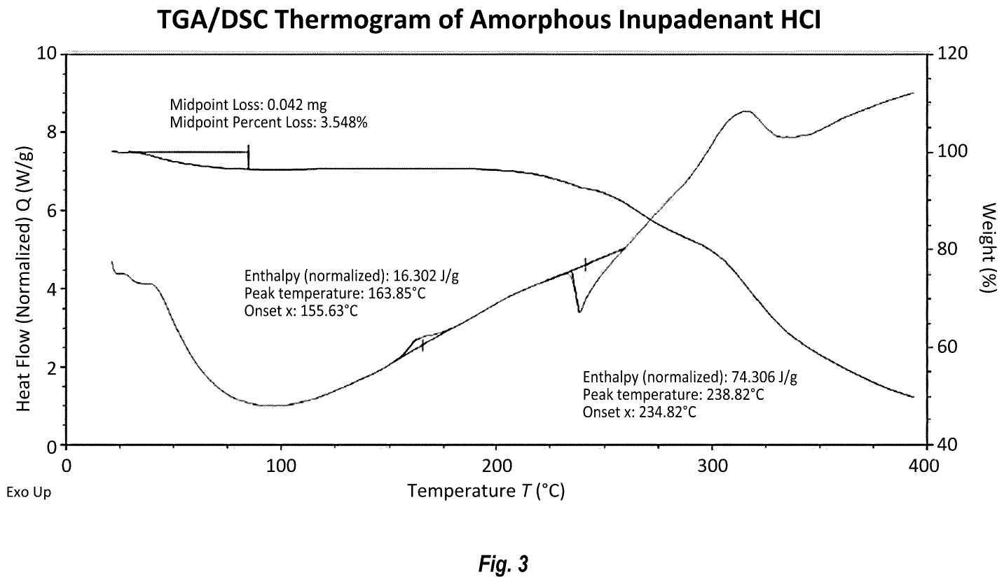

In certain embodiments, a weight loss of less than or equal to about 3.5% occurs upon heating the amorphous inupadenant hydrochloride from about 31° C. to about 83° C. The weight loss exhibited by amorphous inupadenant hydrochloride upon heating can be determined, for example, using TGA. In certain embodiments, the amorphous inupadenant hydrochloride has a TGA thermogram substantially the same as shown in FIG. 3.

In another aspect, provided herein is milled inupadenant hydrochloride.

In another aspect, provided herein is milled amorphous inupadenant hydrochloride. In certain embodiments, the milled amorphous inupadenant hydrochloride is an amorphous inupadenant hydrochloride described herein.

In another aspect provided herein is amorphous inupadenant hydrochloride prepared by milling crystalline inupadenant hydrochloride. In certain embodiments, the milled amorphous inupadenant hydrochloride is an amorphous inupadenant hydrochloride described herein. In certain embodiments, the crystalline inupadenant hydrochloride is a crystalline inupadenant hydrochloride described herein.

In certain embodiments, the crystalline inupadenant hydrochloride is milled for between about 10 minutes and about 60 minutes, about 20 minutes and about 60 minutes, about 30 minutes and about 60 minutes, about 40 minutes and about 60 minutes, about 50 minutes and about 60 minutes, about 10 minutes and about 50 minutes, about 10 minutes and about 40 minutes, about 10 minutes and about 30 minutes, about 10 minutes and about 20 minutes, about 20 minutes and about 50 minutes, about 20 minutes and about 40 minutes, about 20 minutes and about 30 minutes, about 30 minutes and about 50 minutes, about 30 minutes and about 40 minutes, or about 40 minutes and about 50 minutes. In certain embodiments, the crystalline inupadenant hydrochloride is milled for between about 30 minutes and about 60 minutes.

In certain embodiments, the crystalline inupadenant hydrochloride is milled for about 10 minutes, about 15 minutes, about 20 minutes, about 25 minutes, about 30 minutes, about 35 minutes, about 40 minutes, about 45 minutes, about 50 minutes, about 55 minutes, or about 60 minutes.

In certain embodiments, the amorphous inupadenant hydrochloride has a molar ratio of inupadenant to hydrochloride is about 1:1. In certain embodiments, the amorphous inupadenant hydrochloride has a molar ratio of inupadenant to hydrochloride is 1:1.

### (2) Crystalline Inupadenant Hydrochloride

In another aspect, provided herein is crystalline inupadenant hydrochloride.

In various embodiments, the crystalline inupadenant hydrochloride has an X-ray powder diffraction (XRPD) pattern comprising a peak at about 8.9° 2θ.

In various embodiments, the crystalline inupadenant hydrochloride has an XRPD pattern comprising a peak at about 9.3° 2θ.

In various embodiments, the crystalline inupadenant hydrochloride has an XRPD pattern comprising peaks at about 8.9° and about 9.3° 2θ.

In certain embodiments, the XRPD pattern further comprises one or more peaks selected from about 14.8° and about 26.7° 2θ. In certain embodiments, the XRPD pattern further comprises one or more peaks selected from about 5.3°, about 18.0°, and about 22.6° 2θ. In certain embodiments, the XRPD pattern further comprises a peak at about 32.2° 2θ.

In certain embodiments, the XRPD pattern further comprises one or more peaks selected from about 19.3°, about 23.4°, and about 27.5° 2θ. In certain embodiments, the XRPD pattern further comprises one or more peaks selected from about 28.2° and about 29.4° 2θ. In certain embodiments, the XRPD pattern further comprises one or more peaks selected from about 6.7°, about 12.6°, about 13.4°, about 15.4°, about 16.1°, about 16.5°, about 17.4°, about 19.8°, about 24.3°, and about 24.9° 2θ. In certain embodiments, the XRPD pattern further comprises one or more peaks selected from about 30.4°, about 31.0°, and about 31.6° 2θ. In certain embodiments, the XRPD pattern further comprises one or more peaks selected from about 14.5°, about 22.2°, about 23.10, about 25.4°, and about 25.9° 2θ. In certain embodiments, the XRPD pattern further comprises a peak at about 34.4° 2θ. In certain embodiments, the XRPD pattern further comprises one or more peaks selected from about 21.5°, about 22.8°, about 25.7°, and about 27.1° 2θ. In certain embodiments, the XRPD pattern further comprises one or more peaks selected from about 33.0° and about 33.9° 2θ. In certain embodiments, the XRPD pattern further comprises one or more peaks selected from about 25.0° and about 25.8° 2θ. In certain embodiments, the XRPD pattern further comprises one or more peaks selected from about 22.0° and about 25.2° 2θ. In certain embodiments, the XRPD pattern further comprises one or more peaks selected from about 9.4°, about 13.3°, about 15.5°, about 17.5°, about 23.5°, about 24.4°, and about 26.1° 2θ. In certain embodiments, the XRPD pattern further comprises one or more peaks selected from about 28.3°, about 30.5°, about 31.10, and about 33.7° 2θ. In certain embodiments, the XRPD pattern further comprises one or more peaks selected from about 12.7°, about 16.8°, about 19.9°, about 20.4°, about 22.10, about 25.5°, and about 27.6° 2θ. In certain embodiments, the XRPD pattern further comprises one or more peaks selected from about 14.6°, about 16.6°, about 22.4°, and about 24.0° 20. In certain embodiments, the XRPD pattern further comprises one or more peaks selected from about 31.7° and about 34.5° 2θ. In certain embodiments, the XRPD pattern further comprises one or more peaks selected from about 18.2° and about 18.8° 2θ. In certain embodiments, the XRPD pattern further comprises one or more peaks selected from about 9.5° and about 23.2° 2θ. In certain embodiments, the XRPD pattern further comprises a peak at about 18.9° 2θ. In certain embodiments, the XRPD pattern further comprises one or more peaks selected from about 13.5°, about 16.3°, about 16.7°, about 20.0°, about 21.7°, about 23.7°, about 24.10, about 25.10, about 26.0°, and about 27.7° 2θ. In certain embodiments, the XRPD pattern further comprises one or more peaks selected from about 28.6°, about 30.6°, and about 33.1° 2θ. In certain embodiments, the XRPD pattern further comprises one or more peaks selected from about 15.6° and about 27.0° 20. In certain embodiments, the XRPD pattern further comprises a peak at about 29.5° 2θ. In certain embodiments, the XRPD pattern further comprises a peak at about 19.4° 2θ. In certain embodiments, the XRPD pattern further comprises one or more peaks selected from about 12.9°, about 15.3°, about 16.0°, about 18.4°, and about 27.4° 2θ. In certain embodiments, the XRPD pattern further comprises a peak at about 29.9° 2θ. In certain embodiments, the XRPD pattern further comprises one or more peaks selected from about 3.3°, about 6.5°, about 22.3°, about 23.9°, about 24.8°, and about 26.8° 2θ. In certain embodiments, the XRPD pattern further comprises one or more peaks selected from about 26.8°, about 29.10, about 29.3°, and about 32.9° 2θ.

In various embodiments, the crystalline inupadenant hydrochloride has an X-ray powder diffraction (XRPD) pattern comprising a peak at 8.9°±0.3° 2θ.

In various embodiments, the crystalline inupadenant hydrochloride has an XRPD pattern comprising a peak at 9.3°±0.3° 2θ.

In various embodiments, the crystalline inupadenant hydrochloride has an XRPD pattern comprising peaks at 8.9°±0.3° and 9.3°±0.3° 2θ.

In certain embodiments, the XRPD pattern further comprises one or more peaks selected from 14.8°±0.3° and 26.7°±0.3° 2θ. In certain embodiments, the XRPD pattern further comprises one or more peaks selected from 5.3°±0.3°, 18.0°±0.3°, and 22.6°±0.3° 2θ. In certain embodiments, the XRPD pattern further comprises a peak at 32.2°±0.3° 2θ.

In certain embodiments, the XRPD pattern further comprises one or more peaks selected from 19.3°±0.3°, 23.4°±0.3°, and 27.5°±0.3° 2θ. In certain embodiments, the XRPD pattern further comprises one or more peaks selected from 28.2°±0.3° and 29.4°±0.3° 2θ. In certain embodiments, the XRPD pattern further comprises one or more peaks selected from 6.7°±0.3°, 12.6°±0.3°, 13.4°±0.3°, 15.4°±0.3°, 16.1°±0.3°, 16.5°±0.3°, 17.4°±0.3°, 19.8°±0.3°, 24.3°±0.3°, and 24.9°±0.3° 2θ. In certain embodiments, the XRPD pattern further comprises one or more peaks selected from 30.4°±0.3°, 31.0°±0.3°, and 31.6°±0.3° 2θ. In certain embodiments, the XRPD pattern further comprises one or more peaks selected from 14.5°±0.3°, 22.2°±0.3°, 23.1°±0.3°, 25.4°±0.3°, and 25.9°±0.3° 2θ. In certain embodiments, the XRPD pattern further comprises a peak at 34.4°±0.3° 2θ. In certain embodiments, the XRPD pattern further comprises one or more peaks selected from 21.5°±0.3°, 22.8°±0.3°, 25.7°±0.3°, and 27.1° °±0.3° 2θ. In certain embodiments, the XRPD pattern further comprises one or more peaks selected from 33.0°±0.3° and 33.9°±0.3° 2θ. In certain embodiments, the XRPD pattern further comprises one or more peaks selected from 25.0°±0.3° and 25.8°±0.3° 2θ. In certain embodiments, the XRPD pattern further comprises one or more peaks selected from 22.0°±0.3° and 25.2°±0.3° 2θ. In certain embodiments, the XRPD pattern further comprises one or more peaks selected from 9.4° 0.3°, 13.3°±0.3°, 15.5°±0.3°, 17.5°±0.3°, 23.5°±0.3°, 24.4°±0.3°, and 26.1°±0.3° 2θ. In certain embodiments, the XRPD pattern further comprises one or more peaks selected from 28.3°±0.3°, 30.5°±0.3°, 31.1°±0.3°, and 33.7°±0.3° 2θ. In certain embodiments, the XRPD pattern further comprises one or more peaks selected from 12.7°±0.3°, 16.8°±0.3°, 19.9°±0.3°, 20.4°±0.3°, 22.1°±0.3°, 25.5°±0.3°, and 27.6°±0.3° 2θ. In certain embodiments, the XRPD pattern further comprises one or more peaks selected from 14.6°±0.3°, 16.6°±0.3°, 22.4°±0.3°, and 24.0°±0.3° 2θ. In certain embodiments, the XRPD pattern further comprises one or more peaks selected from 31.7°±0.3° and 34.5°±0.3° 2θ. In certain embodiments, the XRPD pattern further comprises one or more peaks selected from 18.2°±0.3° and 18.8°±0.3° 2θ. In certain embodiments, the XRPD pattern further comprises one or more peaks selected from 9.5°±0.3° and 23.2°±0.3° 2θ. In certain embodiments, the XRPD pattern further comprises a peak at 18.9°±0.3° 2θ. In certain embodiments, the XRPD pattern further comprises one or more peaks selected from 13.5°±0.3°, 16.3°±0.3°, 16.7°±0.3°, 20.0°±0.3°, 21.7°±0.3°, 23.7°±0.3°, 24.1°±0.3°, 25.1°±0.3°, 26.0°±0.3°, and 27.7°±0.3° 2θ. In certain embodiments, the XRPD pattern further comprises one or more peaks selected from 28.6°±0.3°, 30.6°±0.3°, and 33.1°±0.3° 2θ. In certain embodiments, the XRPD pattern further comprises one or more peaks selected from 15.6°±0.3° and 27.0°±0.3° 2θ. In certain embodiments, the XRPD pattern further comprises a peak at 29.5°±0.3° 2θ. In certain embodiments, the XRPD pattern further comprises a peak at 19.4°±0.3° 2θ. In certain embodiments, the XRPD pattern further comprises one or more peaks selected from 12.9°±0.3°, 15.3°±0.3°, 16.0°±0.3°, 18.4°±0.3°, and 27.4°±0.3° 2θ. In certain embodiments, the XRPD pattern further comprises a peak at 29.9°±0.3° 2θ. In certain embodiments, the XRPD pattern further comprises one or more peaks selected from 3.3°±0.3°, 6.5°±0.3°, 22.3°±0.3°, 23.9°±0.3°, 24.8°±0.3°, and 26.8°±0.3° 2θ. In certain embodiments, the XRPD pattern further comprises one or more peaks selected from 26.8°±0.3°, 29.1° °±0.3°, 29.3°±0.30, and 32.9°±0.30 20.

In various embodiments, the crystalline inupadenant hydrochloride has an XRPD pattern comprising a peak at 8.9°±0.2°.

In various embodiments, the crystalline inupadenant hydrochloride has an XRPD pattern comprising a peak at 9.3°±0.2°.

In various embodiments, the crystalline inupadenant hydrochloride has an XRPD pattern comprising peaks at 8.9°±0.2° and 9.3°±0.2° 2θ.

In certain embodiments, the XRPD pattern further comprises one or more peaks selected from 14.8°±0.2° and 26.7°±0.2° 2θ. In certain embodiments, the XRPD pattern further comprises one or more peaks selected from 5.3°±0.2°, 18.0°±0.2°, and 22.6°±0.2° 2θ. In certain embodiments, the XRPD pattern further comprises a peak at 32.2°±0.2° 2θ.

In certain embodiments, the XRPD pattern further comprises one or more peaks selected from 19.3°±0.2°, 23.4°±0.2°, and 27.5°±0.2° 2θ. In certain embodiments, the XRPD pattern further comprises one or more peaks selected from 28.2°±0.2° and 29.4°±0.2° 2θ. In certain embodiments, the XRPD pattern further comprises one or more peaks selected from 6.7°±0.2°, 12.6°±0.2°, 13.4°±0.2°, 15.4°±0.2°, 16.1°±0.2°, 16.5°±0.2°, 17.4°±0.2°, 19.8°±0.2°, 24.3°±0.2°, and 24.9°±0.2° 2θ. In certain embodiments, the XRPD pattern further comprises one or more peaks selected from 30.4°±0.2°, 31.0°±0.2°, and 31.6°±0.2° 2θ. In certain embodiments, the XRPD pattern further comprises one or more peaks selected from 14.5°±0.2°, 22.2°±0.2°, 23.1°±0.2°, 25.4°±0.2°, and 25.9°±0.2° 2θ. In certain embodiments, the XRPD pattern further comprises a peak at 34.4°±0.2° 2θ. In certain embodiments, the XRPD pattern further comprises one or more peaks selected from 21.5°±0.2°, 22.8°±0.2°, 25.7°±0.2°, and 27.1°±0.2° 2θ. In certain embodiments, the XRPD pattern further comprises one or more peaks selected from 33.0°±0.2° and 33.9°±0.2° 2θ. In certain embodiments, the XRPD pattern further comprises one or more peaks selected from 25.0°±0.2° and 25.8°±0.2° 2θ. In certain embodiments, the XRPD pattern further comprises one or more peaks selected from 22.0°±0.2° and 25.2°±0.2° 2θ. In certain embodiments, the XRPD pattern further comprises one or more peaks selected from 9.4°±0.2°, 13.3°±0.2°, 15.5°±0.2°, 17.5°±0.2°, 23.5°±0.2°, 24.4°±0.2°, and 26.1°±0.2° 2θ. In certain embodiments, the XRPD pattern further comprises one or more peaks selected from 28.3°±0.2°, 30.5°±0.2°, 31.1°±0.2°, and 33.7°±0.2° 2θ. In certain embodiments, the XRPD pattern further comprises one or more peaks selected from 12.7°±0.2°, 16.8°±0.2°, 19.9°±0.2°, 20.4°±0.2°, 22.1°±0.2°, 25.5°±0.2°, and 27.6°±0.2° 2θ. In certain embodiments, the XRPD pattern further comprises one or more peaks selected from 14.6°±0.2°, 16.6°±0.2°, 22.4°±0.2°, and 24.0°±0.2° 2θ. In certain embodiments, the XRPD pattern further comprises one or more peaks selected from 31.7°±0.2° and 34.5°±0.2° 2θ. In certain embodiments, the XRPD pattern further comprises one or more peaks selected from 18.2°±0.2° and 18.8°±0.2° 2θ. In certain embodiments, the XRPD pattern further comprises one or more peaks selected from 9.5°±0.2° and 23.2°±0.2° 2θ. In certain embodiments, the XRPD pattern further comprises a peak at 18.9°±0.2° 2θ. In certain embodiments, the XRPD pattern further comprises one or more peaks selected from 13.5°±0.2°, 16.3°±0.2°, 16.7°±0.2°, 20.0°±0.2°, 21.7°±0.2°, 23.7°±0.2°, 24.1°±0.2°, 25.1°±0.2°, 26.0°±0.2°, and 27.7°±0.2° 2θ. In certain embodiments, the XRPD pattern further comprises one or more peaks selected from 28.6°±0.2°, 30.6°±0.2°, and 33.1°±0.2° 2θ. In certain embodiments, the XRPD pattern further comprises one or more peaks selected from 15.6°±0.2° and 27.0°±0.2° 2θ. In certain embodiments, the XRPD pattern further comprises a peak at 29.5°±0.2° 2θ. In certain embodiments, the XRPD pattern further comprises a peak at 19.4°±0.2° 2θ. In certain embodiments, the XRPD pattern further comprises one or more peaks selected from 12.9°±0.2°, 15.3°±0.2°, 16.0°±0.2°, 18.4°±0.2°, and 27.4°±0.2° 2θ. In certain embodiments, the XRPD pattern further comprises a peak at 29.9°±0.2° 2θ. In certain embodiments, the XRPD pattern further comprises one or more peaks selected from 3.3°±0.2°, 6.5°±0.2°, 22.3°±0.2°, 23.9°±0.2°, 24.8°±0.2°, and 26.8°±0.2° 2θ. In certain embodiments, the XRPD pattern further comprises one or more peaks selected from 26.8°±0.2°, 29.1°±0.2°, 29.3°±0.2°, and 32.9°±0.2°.

In various embodiments, the crystalline inupadenant hydrochloride has an XRPD pattern comprising a peak at 8.9°±0.1°.

In various embodiments, the crystalline inupadenant hydrochloride has an XRPD pattern comprising a peak at 9.3°±0.1°.

In various embodiments, the crystalline inupadenant hydrochloride has an XRPD pattern comprising peaks at 8.9°±0.1° and 9.3°±0.1°.

In certain embodiments, the XRPD pattern further comprises one or more peaks selected from 14.8°±0.1° and 26.7°±0.1° 2θ. In certain embodiments, the XRPD pattern further comprises one or more peaks selected from 5.3°±0.1°, 18.0°±0.1°, and 22.6°±0.1° 2θ. In certain embodiments, the XRPD pattern further comprises a peak at 32.2°±0.1°.

In certain embodiments, the XRPD pattern further comprises one or more peaks selected from 19.3°±0.1°, 23.4°±0.1°, and 27.5°±0.1° 2θ. In certain embodiments, the XRPD pattern further comprises one or more peaks selected from 28.2°±0.1° and 29.4°±0.1° 2θ. In certain embodiments, the XRPD pattern further comprises one or more peaks selected from 6.7°±0.1°, 12.6°±0.1°, 13.4°±0.1°, 15.4°±0.1°, 16.1°±0.1°, 16.5°±0.1°, 17.4°±0.1°, 19.8°±0.1°, 24.3° ±0.10, and 24.9°±0.10 20. In certain embodiments, the XRPD pattern further comprises one or more peaks selected from 30.4°±0.1°, 31.0°±0.1°, and 31.6°±0.1° 2θ. In certain embodiments, the XRPD pattern further comprises one or more peaks selected from 14.5°±0.10, 22.2°±0.1°, 23.1°±0.1°, 25.4°±0.1°, and 25.9°±0.1° 2θ. In certain embodiments, the XRPD pattern further comprises a peak at 34.4°±0.1° 2θ. In certain embodiments, the XRPD pattern further comprises one or more peaks selected from 21.5°±0.1°, 22.8°±0.1°, 25.7°±0.1°, and 27.1°±0.1° 2θ. In certain embodiments, the XRPD pattern further comprises one or more peaks selected from 33.0°±0.1° and 33.9°±0.1° 2θ. In certain embodiments, the XRPD pattern further comprises one or more peaks selected from 25.0°±0.1° and 25.8°±0.1° 2θ. In certain embodiments, the XRPD pattern further comprises one or more peaks selected from 22.0°±0.1° and 25.2°±0.1° 2θ. In certain embodiments, the XRPD pattern further comprises one or more peaks selected from 9.4°±0.1°, 13.3°±0.1°, 15.5°±0.1°, 17.5°±0.1°, 23.5°±0.1°, 24.4°±0.1°, and 26.1°±0.1° 2θ. In certain embodiments, the XRPD pattern further comprises one or more peaks selected from 28.3°±0.1°, 30.5°±0.1°, 31.1°±0.1°, and 33.7°±0.1° 2θ. In certain embodiments, the XRPD pattern further comprises one or more peaks selected from 12.7°±0.1°, 16.8°±0.1°, 19.9°±0.1°, 20.4°±0.1°, 22.1°±0.1°, 25.5°±0.1°, and 27.6°±0.1° 2θ. In certain embodiments, the XRPD pattern further comprises one or more peaks selected from 14.6°±0.1°, 16.6°±0.1°, 22.4°±0.1°, and 24.0°±0.1° 2θ. In certain embodiments, the XRPD pattern further comprises one or more peaks selected from 31.7°±0.1° and 34.5°±0.1° 2θ. In certain embodiments, the XRPD pattern further comprises one or more peaks selected from 18.2°±0.1° and 18.8°±0.1° 2θ. In certain embodiments, the XRPD pattern further comprises one or more peaks selected from 9.5°±0.1° and 23.2°±0.1° 2θ. In certain embodiments, the XRPD pattern further comprises a peak at 18.9°±0.1° 2θ. In certain embodiments, the XRPD pattern further comprises one or more peaks selected from 13.5°±0.1°, 16.3°±0.1°, 16.7°±0.1°, 20.0°±0.1°, 21.7°±0.1°, 23.7°±0.1°, 24.1°±0.1°, 25.1°±0.1°, 26.0°±0.1°, and 27.7°±0.1° 2θ. In certain embodiments, the XRPD pattern further comprises one or more peaks selected from 28.6°±0.1°, 30.6°±0.1°, and 33.1°±0.1° 2θ. In certain embodiments, the XRPD pattern further comprises one or more peaks selected from 15.6°±0.1° and 27.0°±0.1° 2θ. In certain embodiments, the XRPD pattern further comprises a peak at 29.5°±0.1° 2θ. In certain embodiments, the XRPD pattern further comprises a peak at 19.4°±0.1° 2θ. In certain embodiments, the XRPD pattern further comprises one or more peaks selected from 12.9°±0.1°, 15.3°±0.1°, 16.0° °±0.1°, 18.4°±0.1°, and 27.4°±0.1° 2θ. In certain embodiments, the XRPD pattern further comprises a peak at 29.9°±0.1° 2θ. In certain embodiments, the XRPD pattern further comprises one or more peaks selected from 3.3°±0.10, 6.5°±0.1°, 22.3°±0.1°, 23.9°±0.1°, 24.8°±0.1°, and 26.8°±0.1° 2θ. In certain embodiments, the XRPD pattern further comprises one or more peaks selected from 26.8°±0.1°, 29.1°±0.1°, 29.30±0.10, and 32.90±0.10 20.

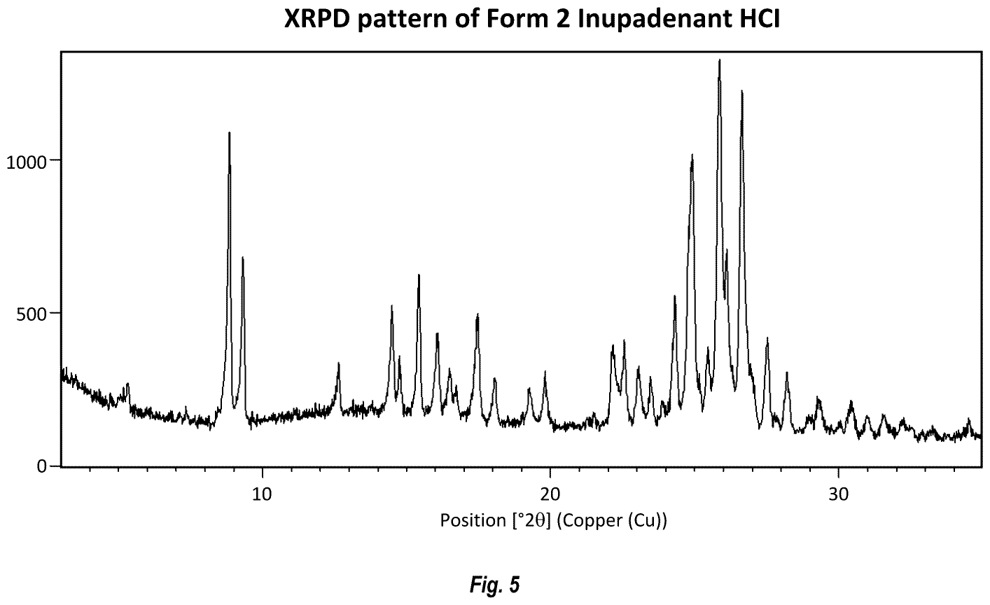

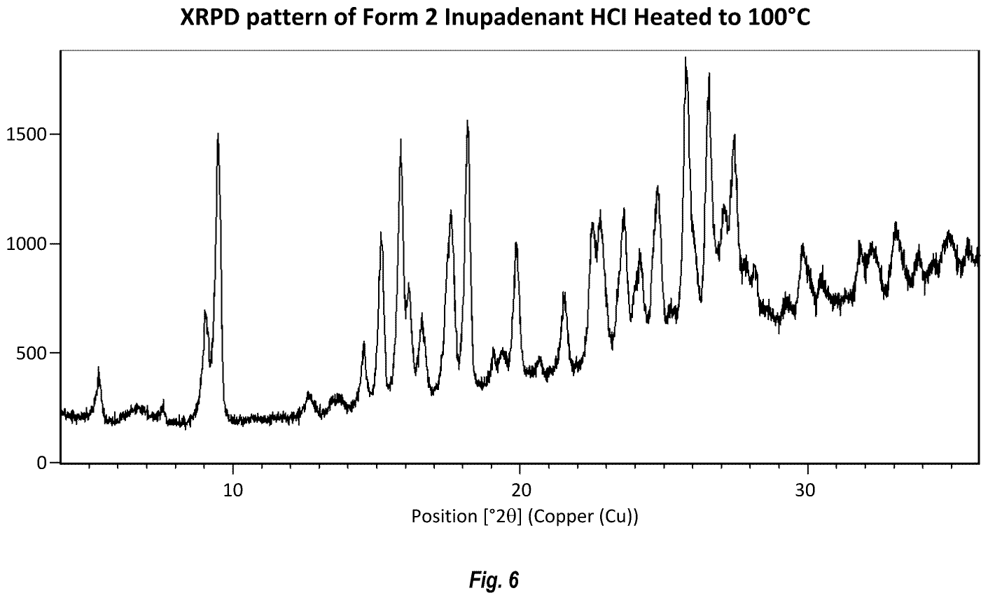

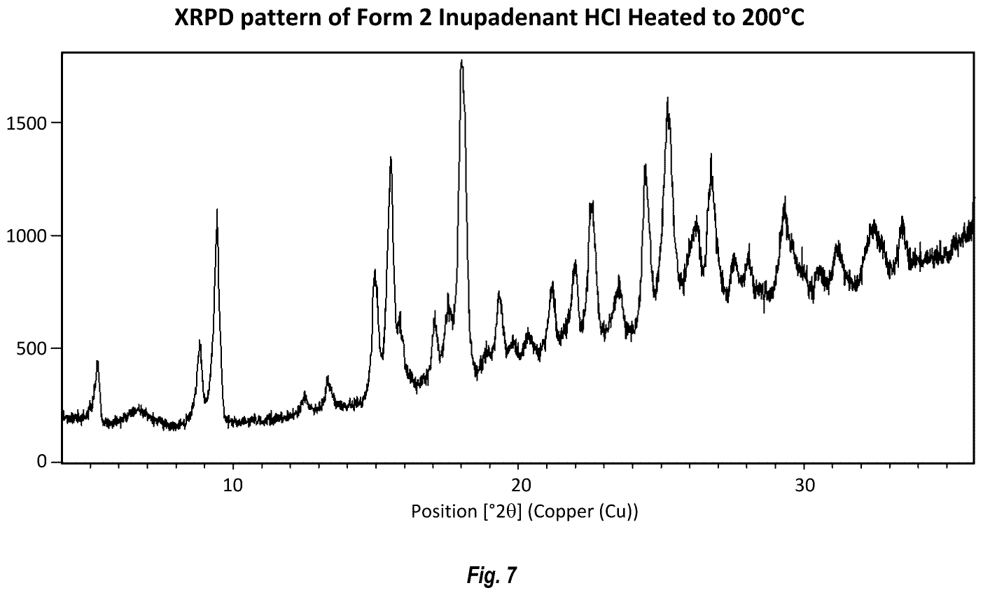

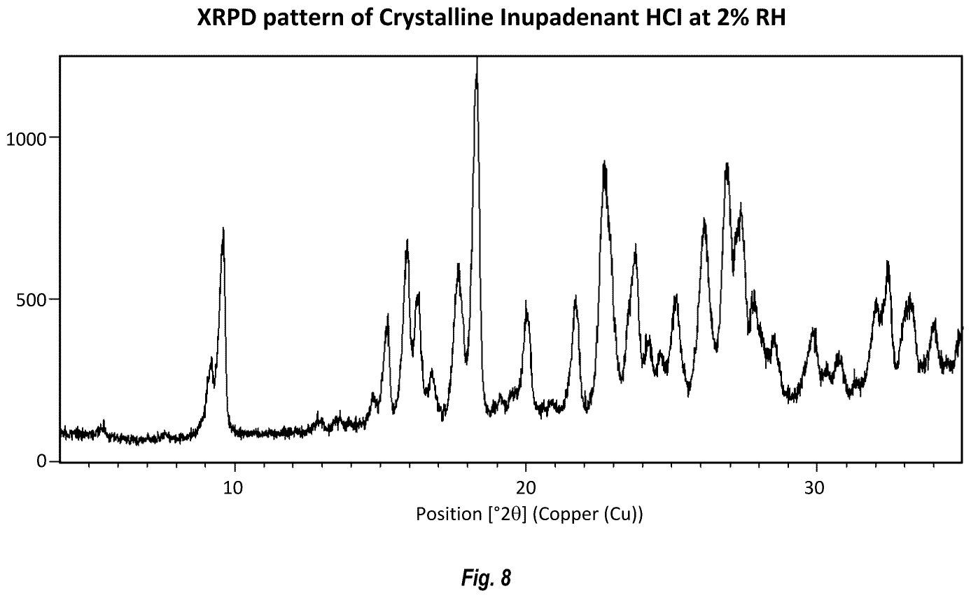

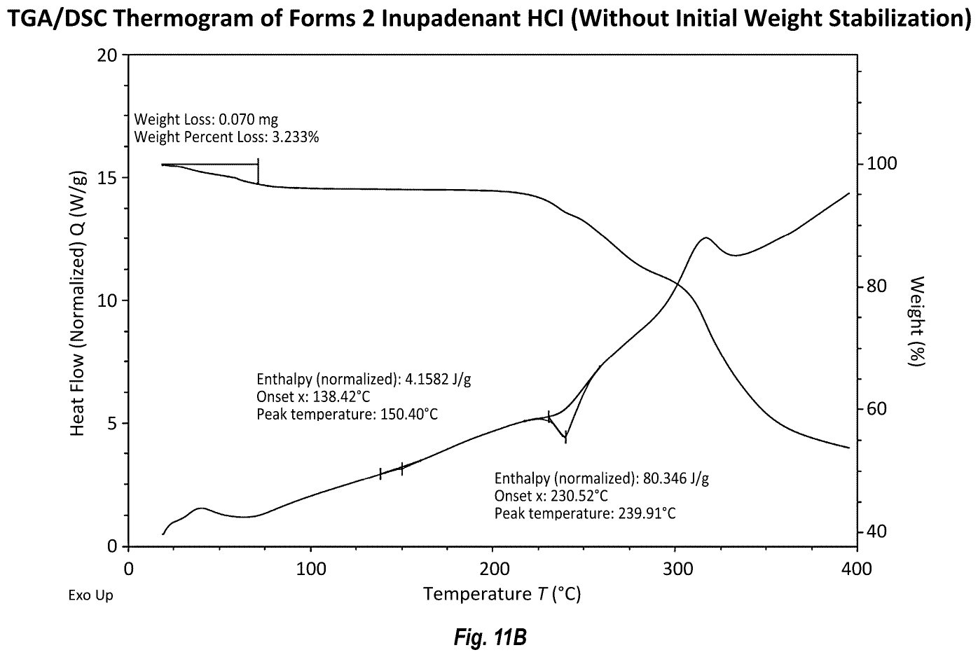

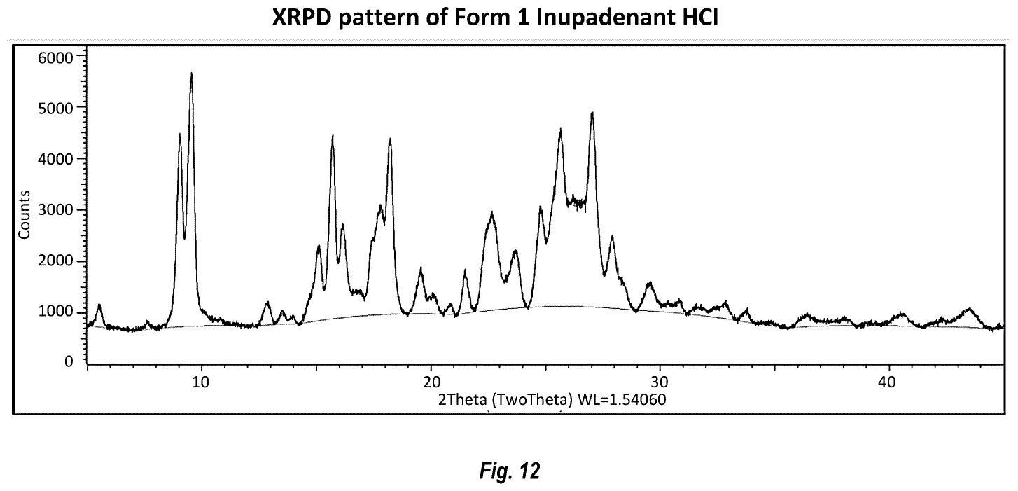

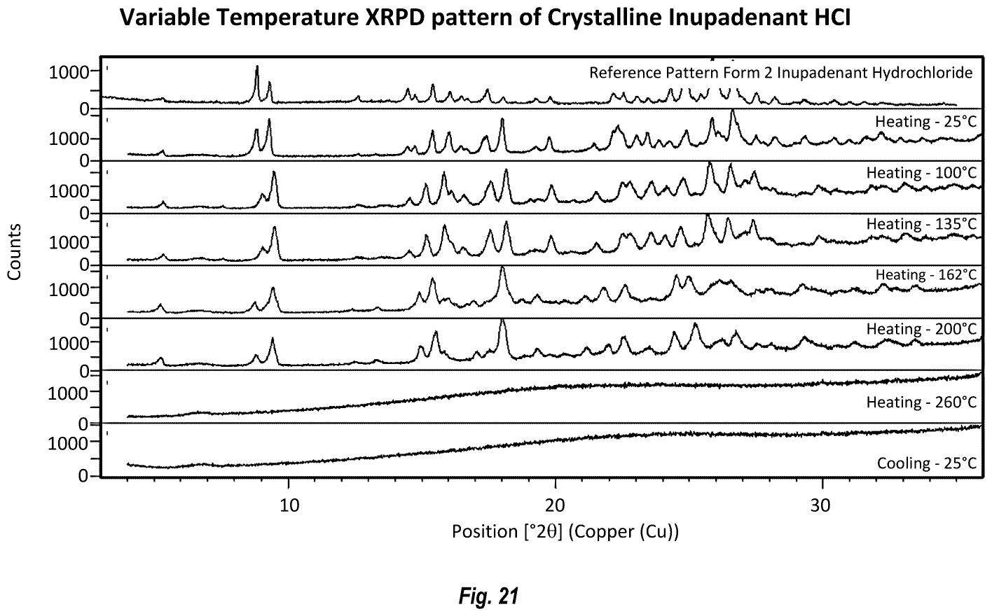

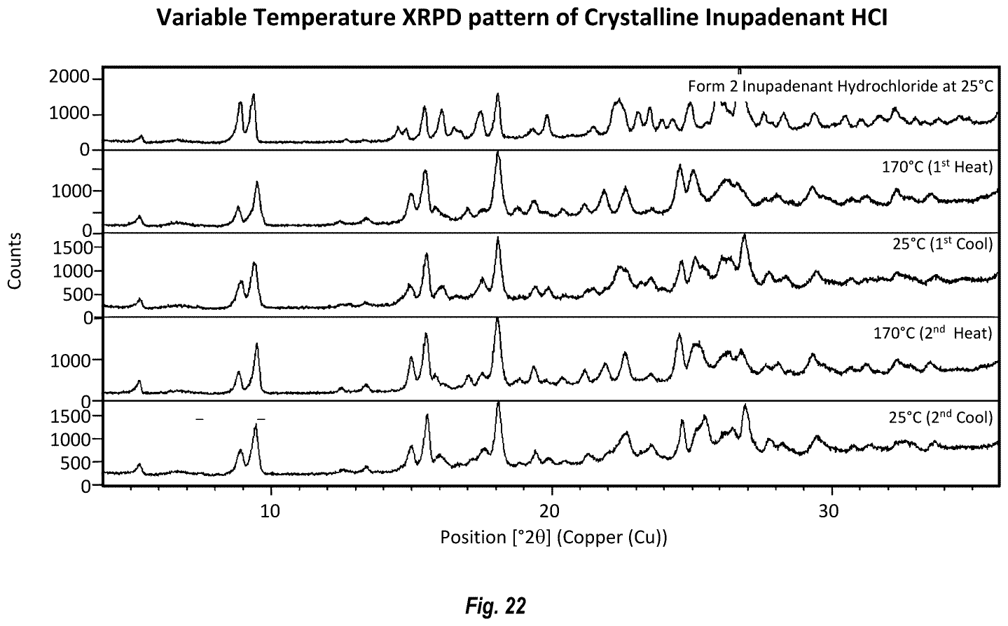

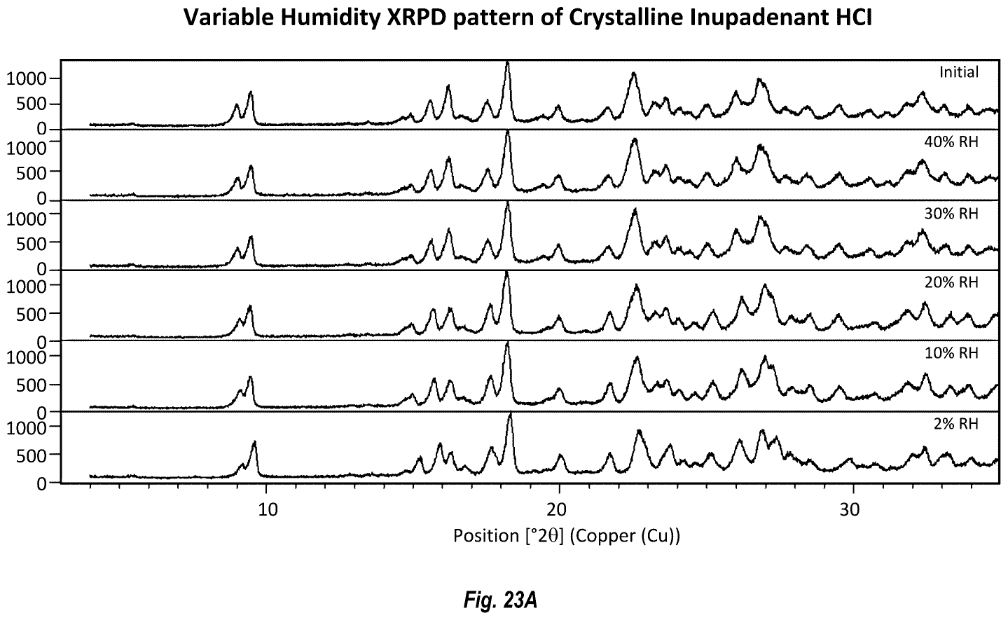

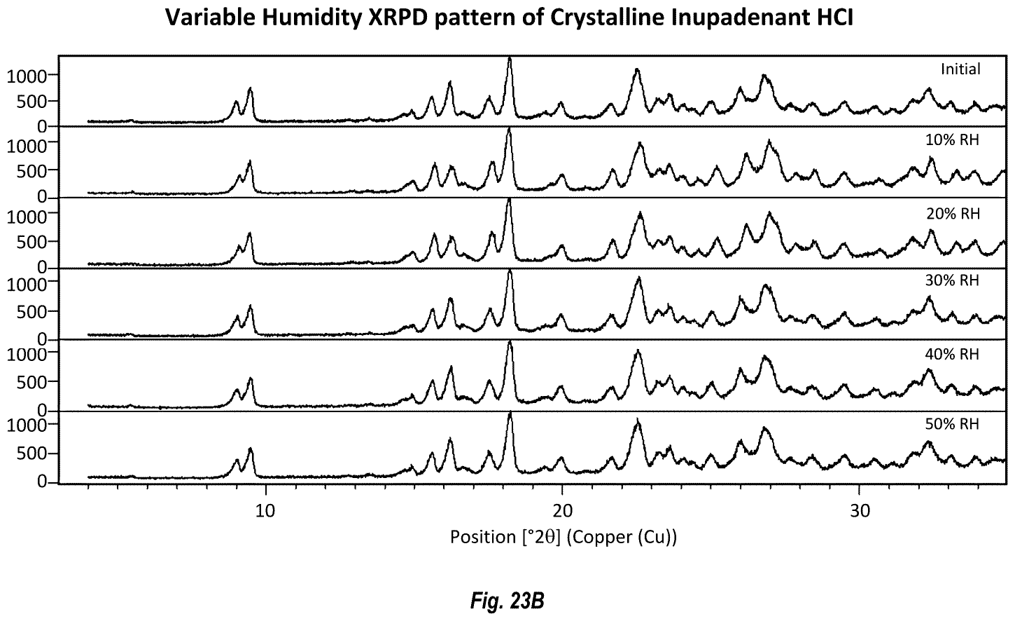

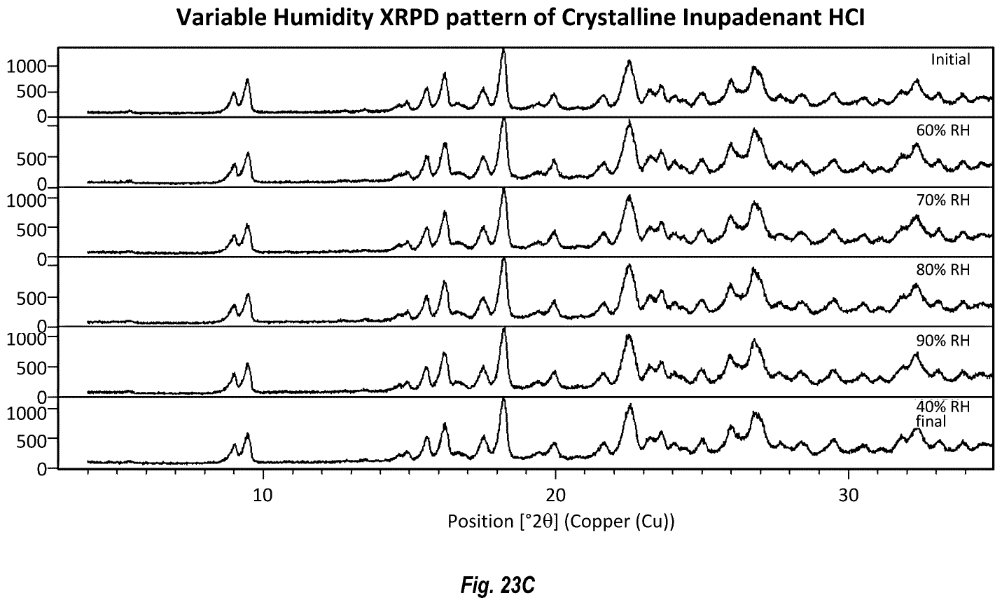

In certain embodiments, the crystalline inupadenant hydrochloride has an XRPD pattern substantially corresponding to the XRPD pattern shown in FIG. 4. In certain embodiments, the crystalline inupadenant hydrochloride has an XRPD pattern substantially corresponding to the XRPD pattern shown in FIG. 5. In certain embodiments, the crystalline inupadenant hydrochloride has an XRPD pattern substantially corresponding to the XRPD pattern shown in FIG. 6. In certain embodiments, the crystalline inupadenant hydrochloride has an XRPD pattern substantially corresponding to the XRPD pattern shown in FIG. 7. In certain embodiments, the crystalline inupadenant hydrochloride has an XRPD pattern substantially corresponding to the XRPD pattern shown in FIG. 8. In certain embodiments, the crystalline inupadenant hydrochloride has an XRPD pattern substantially corresponding to the XRPD pattern shown in FIG. 12. In certain embodiments, the crystalline inupadenant hydrochloride has an XRPD pattern substantially corresponding to any one of the XRPD patterns shown in FIG. 21. In certain embodiments, the crystalline inupadenant hydrochloride has an XRPD pattern substantially corresponding to any one of the XRPD patterns shown in FIG. 22. In certain embodiments, the crystalline inupadenant hydrochloride has an XRPD pattern substantially corresponding to the XRPD pattern shown in FIGS. 23A, 23B, and/or 23C.

In certain embodiments, Form 1 inupadenant hydrochloride has an XRPD pattern comprising one or more diffraction peaks (20) disclosed in Table 6.

In certain embodiments, Form 2 inupadenant hydrochloride has an XRPD pattern comprising one or more diffraction peaks (20) disclosed in Table 7. In certain embodiments, Form 2 inupadenant hydrochloride has a Fourier Transform Infrared (FTIR) spectrum comprising one or more peaks disclosed in Table 8. In certain embodiments, Form 2 inupadenant hydrochloride has an XRPD pattern comprising one or more diffraction peaks (20) disclosed in Table 9. In certain embodiments, Form 2 inupadenant hydrochloride has an X-ray powder diffraction (XRPD) pattern comprising one or more diffraction peaks (20) disclosed in Table 14.

In certain embodiments, crystalline inupadenant hydrochloride has an XRPD pattern comprising one or more diffraction peaks (20) disclosed in Table 10. In certain embodiments, crystalline inupadenant hydrochloride has an XRPD pattern comprising one or more diffraction peaks (20) disclosed in Table 11. In certain embodiments, crystalline inupadenant hydrochloride has an XRPD pattern comprising one or more diffraction peaks (20) disclosed in Table 12. In certain embodiments, crystalline inupadenant hydrochloride has an XRPD pattern comprising one or more diffraction peaks (20) disclosed in Table 13. In certain embodiments, crystalline inupadenant hydrochloride has an X-ray powder diffraction (XRPD) pattern comprising one or more diffraction peaks (20) disclosed in Table 15.

In certain embodiments, crystalline inupadenant hydrochloride has an X-ray powder diffraction (XRPD) pattern comprising one or more diffraction peaks (20) disclosed in Table 16.

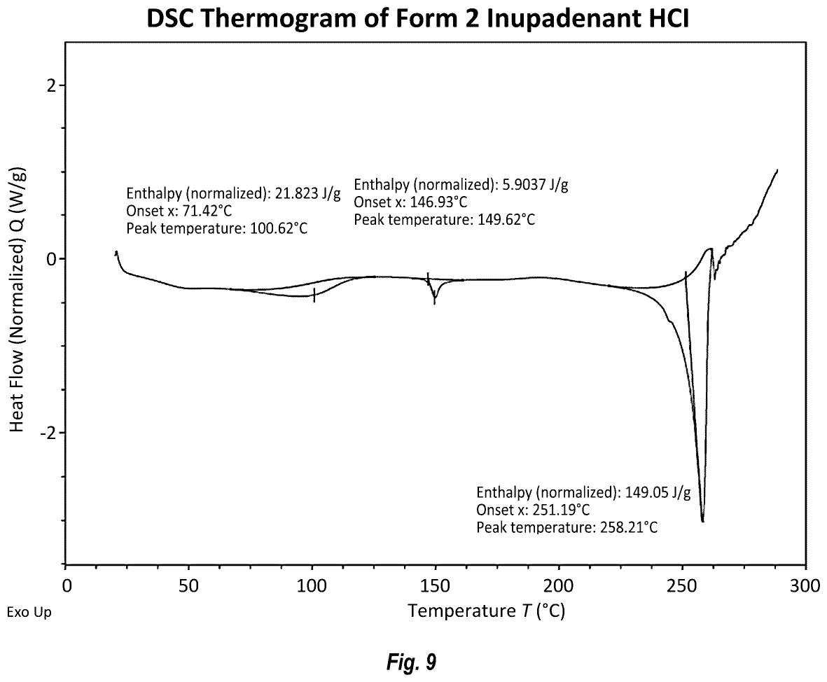

In certain embodiments, the crystalline inupadenant hydrochloride has a DSC thermogram comprising an endotherm with a peak onset at about 70° C. In certain embodiments, the crystalline inupadenant hydrochloride has a DSC thermogram comprising an endotherm with a peak onset at about 140° C. In certain embodiments, the crystalline inupadenant hydrochloride has a DSC thermogram comprising an endotherm with a peak onset at about 240° C. In certain embodiments, the crystalline inupadenant hydrochloride has a DSC thermogram comprising one or more endotherms with peak onsets selected from about 70° C., about 140° C., and about 240° C. In certain embodiments, the crystalline inupadenant hydrochloride has a DSC thermogram substantially the same as shown in FIG. 9.

In various embodiments, the crystalline inupadenant hydrochloride is anhydrous crystalline inupadenant hydrochloride.

In various embodiments, the crystalline inupadenant hydrochloride is crystalline inupadenant hydrochloride hydrate.

In various embodiments, the crystalline inupadenant hydrochloride is Form 1 inupadenant hydrochloride.

In various embodiments, the crystalline inupadenant hydrochloride is Form 2 inupadenant hydrochloride.

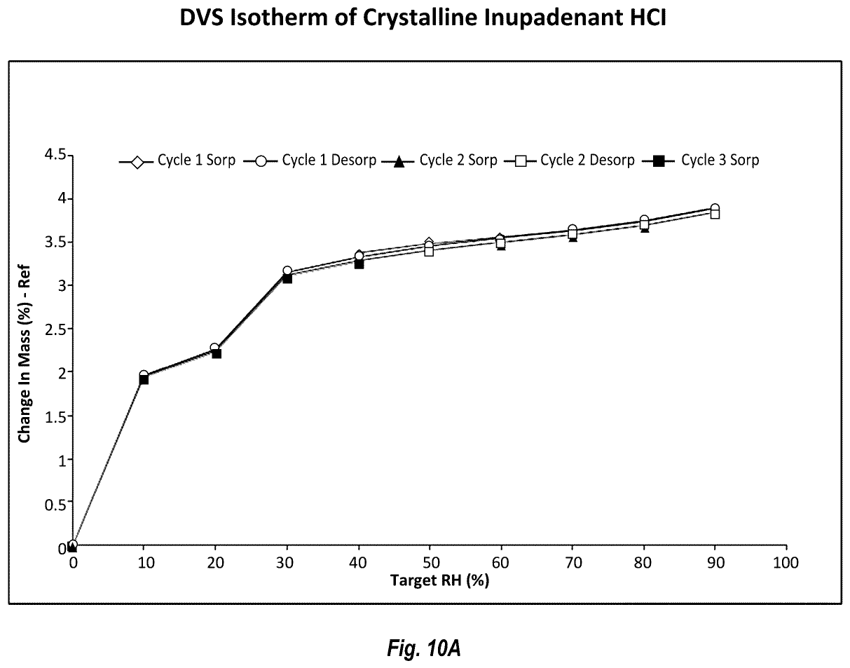

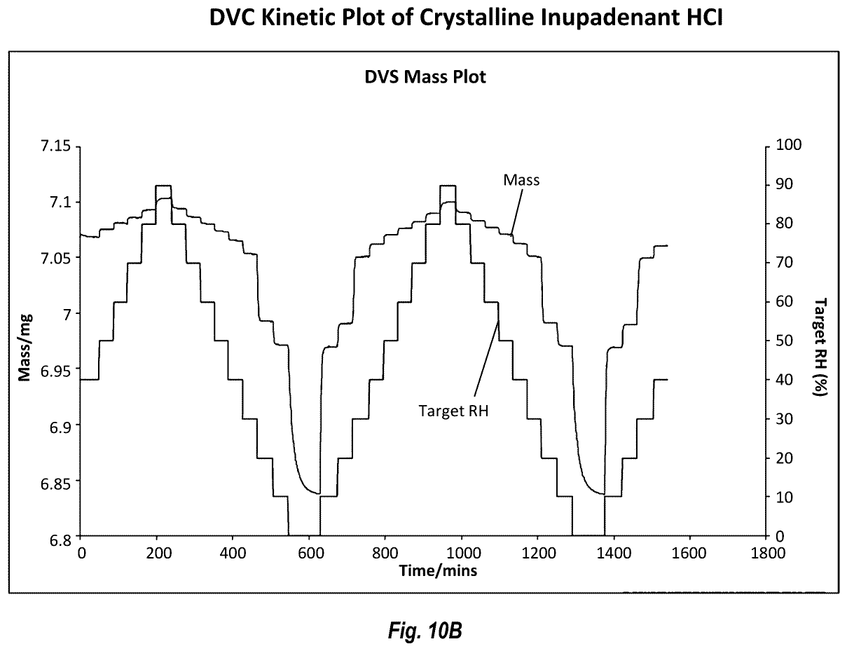

In certain embodiments, the crystalline inupadenant hydrochloride exhibits a change in mass of less than or equal to about 5% wt occurs when varying the relative humidity between 0% and about 95%, when measured at 25° C. The mass change exhibited by crystalline inupadenant hydrochloride as a function of humidity can be determined, for example, using Dynamic Vapor Sorption (DVS). In certain embodiments, the crystalline inupadenant hydrochloride has a water sorption isotherm, when measured at 25° C., substantially the same as shown in FIG. 10A.

In certain embodiments, the crystalline inupadenant hydrochloride has a molar ratio of inupadenant to water of about 1:1 to about 1:1.5, about 1:1.1 to about 1:1.5, about 1:1.2 to about 1:1.5, about 1:1.3 to about 1:1.5, about 1:1.4 to about 1:1.5, about 1:1 to about 1:1.4, about 1:1 to about 1:1.3, about 1:1 to about 1:1.2, about 1:1 to about 1:1.1, about 1:1.1 to about 1:1.4, about 1:1.1 to about 1:1.3, about 1:1.1 to about 1:1.2, about 1:1.2 to about 1:1.4, about 1:1.2 to about 1:1.3, or about 1:1.3 to about 1:1.4. In certain embodiments, the crystalline inupadenant hydrochloride has a molar ratio of inupadenant to water of about 1:1 to about 1:1.5.

In certain embodiments, the crystalline inupadenant hydrochloride has a molar ratio of inupadenant to water of about 1:1, about 1:1.05, about 1:1.1, about 1:1.15, about 1:1.2, about 1:1.25, about 1:1.3, about 1:1.35, about 1:1.4, about 1:1.45, or about 1:1.5.

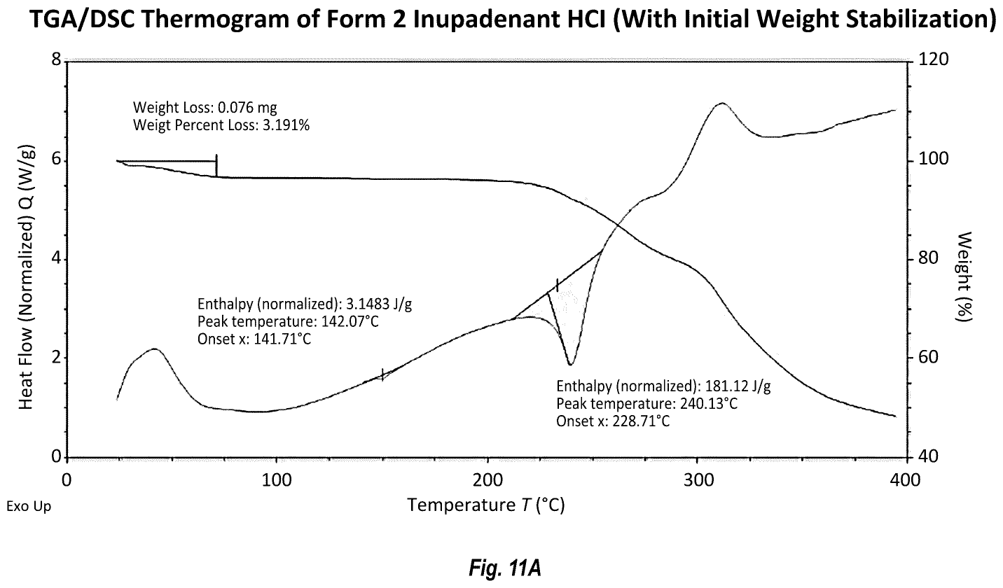

In certain embodiments, a weight loss of between about 1% wt and about 3.2% wt occurs upon heating the crystalline inupadenant hydrochloride from about 31° C. to about 83° C. The weight loss exhibited by crystalline inupadenant hydrochloride upon heating can be determined, for example, using TGA. In certain embodiments, the crystalline inupadenant hydrochloride has a TGA thermogram substantially the same as shown in FIG. 11A. In certain embodiments, the crystalline inupadenant hydrochloride has a TGA thermogram substantially the same as shown in FIG. 11B.

In certain embodiments, the crystalline inupadenant hydrochloride has a water content of about 2.5% wt to about 4.5% wt, about 2.7% wt to about 4.5% wt, about 2.9% wt to about 4.5% wt, about 3.1% wt to about 4.5% wt, about 3.3% wt to about 4.5% wt, about 3.5% wt to about 4.5% wt, about 3.7% wt to about 4.5% wt, about 3.9% wt to about 4.5% wt, about 4.1% wt to about 4.5% wt, about 4.3% wt to about 4.5% wt, about 2.5% wt to about 4.3% wt, about 2.5% wt to about 4.1% wt, about 2.5% wt to about 3.9% wt, about 2.5% wt to about 3.7% wt, about 2.5% wt to about 3.5% wt, about 2.5% wt to about 3.3% wt, about 2.5% wt to about 3.1% wt, about 2.5% wt to about 2.9% wt, about 2.5% wt to about 2.7% wt, about 2.7% wt to about 4.3% wt, about 2.7% wt to about 4.1% wt, about 2.7% wt to about 3.9% wt, about 2.7% wt to about 3.7% wt, about 2.7% wt to about 3.5% wt, about 2.7% wt to about 3.3% wt, about 2.7% wt to about 3.1% wt, about 2.7% wt to about 2.9% wt, about 2.9% wt to about 4.3% wt, about 2.9% wt to about 4.1% wt, about 2.9% wt to about 3.9% wt, about 2.9% wt to about 3.7% wt, about 2.9% wt to about 3.5% wt, about 2.9% wt to about 3.3% wt, about 2.9% wt to about 3.1% wt, about 3.1% wt to about 4.3% wt, about 3.1% wt to about 4.1% wt, about 3.1% wt to about 3.9% wt, about 3.1% wt to about 3.7% wt, about 3.1% wt to about 3.5% wt, about 3.1% wt to about 3.3% wt, about 3.3% wt to about 4.3% wt, about 3.3% wt to about 4.1% wt, about 3.3% wt to about 3.9% wt, about 3.3% wt to about 3.7% wt, about 3.3% wt to about 3.5% wt, about 3.5% wt to about 4.3% wt, about 3.5% wt to about 4.1% wt, about 3.5% wt to about 3.9% wt, about 3.5% wt to about 3.7% wt, about 3.7% wt to about 4.3% wt, about 3.7% wt to about 4.1% wt, about 3.7% wt to about 3.9% wt, about 3.9% wt to about 4.3% wt, about 3.9% wt to about 4.1% wt, or about 4.1% wt to about 4.3% wt. In certain embodiments, the crystalline inupadenant hydrochloride has a water content of about 2.7% wt to about 3.5% wt.

In certain embodiments, the crystalline inupadenant hydrochloride has a water content of 0% wt, about 0.1% wt, about 0.2% wt, about 0.3% wt, about 0.4% wt, about 0.5% wt, about 0.6% wt, about 0.7% wt, about 0.8% wt, about 0.9% wt, about 1.0% wt, about 1.1% wt, about 1.2% wt, about 1.3% wt, about 1.4% wt, about 1.5% wt, about 1.7% wt, about 1.9% wt, about 2.1% wt, about 2.3% wt, about 2.5% wt, about 2.7% wt, about 2.9% wt, about 3.1% wt, about 3.3% wt, about 3.5% wt, about 3.7% wt, about 3.9% wt, about 4.1% wt, about 4.3% wt, about 4.5% wt, or about 5.0% wt.

In certain embodiments, the crystalline inupadenant hydrochloride has a molar ratio of inupadenant to hydrochloride of about 1:1. In certain embodiments, the crystalline inupadenant hydrochloride has a molar ratio of inupadenant to hydrochloride of 1:1.

### (a) Crystalline Inupadenant Hydrate

In one aspect, provided herein is inupadenant hydrochloride hydrate.

In another aspect, provided herein is crystalline inupadenant hydrochloride hydrate.

In various embodiments, the crystalline inupadenant hydrochloride hydrate has an X-ray powder diffraction (XRPD) pattern comprising a peak at about 8.9° 2θ.

In various embodiments, the crystalline inupadenant hydrochloride hydrate has an XRPD pattern comprising a peak at about 9.3° 2θ.

In various embodiments, the crystalline inupadenant hydrochloride hydrate has an XRPD pattern comprising peaks at about 8.9° and about 9.3° 2θ.

In certain embodiments, the XRPD pattern further comprises one or more peaks selected from about 14.8° and about 26.7° 2θ. In certain embodiments, the XRPD pattern further comprises one or more peaks selected from about 5.3°, about 18.0°, and about 22.6° 2θ. In certain embodiments, the XRPD pattern further comprises a peak at about 32.2° 2θ.

In certain embodiments, the XRPD pattern further comprises one or more peaks selected from about 12.6°, about 14.5°, about 15.4°, about 16.1°, about 16.5°, about 17.5°, about 19.3°, about 19.8°, about 22.1°, about 23.1°, about 23.5°, about 24.3°, about 24.9°, about 25.4°, about 25.9°, and about 27.5° 2θ. In certain embodiments, the XRPD pattern further comprises one or more peaks selected from about 3.3°, about 24.8°, and about 26.1° 2θ. In certain embodiments, the XRPD pattern further comprises one or more peaks selected from about 6.5°, about 13.3°, about 16.7°, about 21.5°, about 22.3°, about 23.9°, about 24.10, and about 26.8° 2θ. In certain embodiments, the XRPD pattern further comprises one or more peaks selected from about 28.2°, about 29.3°, about 30.5°, about 31.0°, about 31.6°, and about 34.5° 2θ. In certain embodiments, the XRPD pattern further comprises a peak at about 29.1° 2θ. In certain embodiments, the XRPD pattern further comprises one or more peaks selected from about 32.9° and about 33.7° 2θ. In certain embodiments, the XRPD pattern further comprises one or more peaks selected from about 3.3°, about 6.5°, about 22.3°, about 23.9°, about 24.8°, and about 26.8° 2θ. In certain embodiments, the XRPD pattern further comprises one or more peaks selected from about 26.8°, about 29.10, about 29.3°, and about 32.9° 2θ.

In various embodiments, the crystalline inupadenant hydrochloride hydrate has an X-ray powder diffraction (XRPD) pattern comprising a peak at 8.9°±0.3° 2θ.

In various embodiments, the crystalline inupadenant hydrochloride hydrate has an XRPD pattern comprising a peak at 9.3°±0.3° 2θ.

In various embodiments, the crystalline inupadenant hydrochloride hydrate has an XRPD pattern comprising peaks at 8.9°±0.3° and 9.3°±0.3° 2θ.

In certain embodiments, the XRPD pattern further comprises one or more peaks selected from 14.8°±0.3° and 26.7°±0.3° 2θ. In certain embodiments, the XRPD pattern further comprises one or more peaks selected from 5.3°±0.3°, 18.0°±0.3°, and 22.6°±0.3° 2θ. In certain embodiments, the XRPD pattern further comprises a peak at 32.2°±0.3° 2θ.

In certain embodiments, the XRPD pattern further comprises one or more peaks selected from 12.6°±0.3°, 14.5°±0.3°, 15.4°±0.3°, 16.1°±0.3°, 16.5°±0.3°, 17.5°±0.3°, 19.3°±0.3°, 19.8°±0.3°, 22.1°±0.3°, 23.1°±0.3°, 23.5°±0.3°, 24.3°±0.3°, 24.9°±0.3°, 25.4°±0.3°, 25.9°±0.3°, and 27.5°±0.3° 2θ. In certain embodiments, the XRPD pattern further comprises one or more peaks selected from 3.3°±0.3°, 24.8°±0.3°, and 26.1°±0.3° 2θ. In certain embodiments, the XRPD pattern further comprises one or more peaks selected from 6.5°±0.3°, 13.3°±0.3°, 16.7°±0.3°, 21.5°±0.3°, 22.3°±0.3°, 23.9°±0.3°, 24.1°±0.3°, and 26.8°±0.3° 2θ. In certain embodiments, the XRPD pattern further comprises one or more peaks selected from 28.2°±0.3°, 29.3°±0.3°, 30.5°±0.3°, 31.0°±0.3°, 31.6°±0.3°, and 34.5°±0.3° 2θ. In certain embodiments, the XRPD pattern further comprises a peak at 29.1°±0.3° 2θ. In certain embodiments, the XRPD pattern further comprises one or more peaks selected from 32.9°±0.3° and 33.7°±0.3° 2θ. In certain embodiments, the XRPD pattern further comprises one or more peaks selected from 3.3°±0.3°, 6.5°±0.3°, 22.3°±0.3°, 23.9°±0.3°, 24.8°±0.3°, and 26.8°±0.3° 2θ. In certain embodiments, the XRPD pattern further comprises one or more peaks selected from 26.8°±0.3°, 29.1°±0.30, 29.3°±0.30, and 32.9°±0.30 20.

In various embodiments, the crystalline inupadenant hydrochloride hydrate has an X-ray powder diffraction (XRPD) pattern comprising a peak at 8.9°±0.2°.

In various embodiments, the crystalline inupadenant hydrochloride hydrate has an XRPD pattern comprising a peak at 9.3°±0.2°.

In various embodiments, the crystalline inupadenant hydrochloride hydrate has an XRPD pattern comprising peaks at 8.9°±0.2° and 9.3°±0.2°.

In certain embodiments, the XRPD pattern further comprises one or more peaks selected from 14.8°±0.2° and 26.7°±0.2° 2θ. In certain embodiments, the XRPD pattern further comprises one or more peaks selected from 5.3°±0.2°, 18.0°±0.2°, and 22.6°±0.2° 2θ. In certain embodiments, the XRPD pattern further comprises a peak at 32.2°±0.2°.

In certain embodiments, the XRPD pattern further comprises one or more peaks selected from 12.6°±0.2°, 14.5°±0.2°, 15.4°±0.2°, 16.1°±0.2°, 16.5°±0.2°, 17.5°±0.2°, 19.3°±0.2°, 19.8°±0.2°, 22.1°±0.2°, 23.1°±0.2°, 23.5°±0.2°, 24.3°±0.2°, 24.9°±0.2°, 25.4°±0.2°, 25.9°±0.2°, and 27.5°±0.2° 2θ. In certain embodiments, the XRPD pattern further comprises one or more peaks selected from 3.3°±0.2°, 24.8°±0.2°, and 26.1°±0.2° 2θ. In certain embodiments, the XRPD pattern further comprises one or more peaks selected from 6.5°±0.2°, 13.3°±0.2°, 16.7°±0.2°, 21.5°±0.2°, 22.3°±0.2°, 23.9°±0.2°, 24.1°±0.2°, and 26.8°±0.2° 2θ. In certain embodiments, the XRPD pattern further comprises one or more peaks selected from 28.2°±0.2°, 29.3°±0.2°, 30.5°±0.2°, 31.0°±0.2°, 31.6°±0.2°, and 34.5°±0.2° 2θ. In certain embodiments, the XRPD pattern further comprises a peak at 29.1°±0.2° 2θ. In certain embodiments, the XRPD pattern further comprises one or more peaks selected from 32.9°±0.2° and 33.7°±0.2° 2θ. In certain embodiments, the XRPD pattern further comprises one or more peaks selected from 3.3°±0.2°, 6.5°±0.2°, 22.3°±0.2°, 23.9°±0.2°, 24.8°±0.2°, and 26.8°±0.2° 2θ. In certain embodiments, the XRPD pattern further comprises one or more peaks selected from 26.8°±0.2°, 29.1°±0.2°, 29.3°±0.2°, and 32.9°±0.2°.

In various embodiments, the crystalline inupadenant hydrochloride hydrate has an X-ray powder diffraction (XRPD) pattern comprising a peak at 8.9°±0.1°.

In various embodiments, the crystalline inupadenant hydrochloride hydrate has an XRPD pattern comprising a peak at 9.3°±0.1°.

In various embodiments, the crystalline inupadenant hydrochloride hydrate has an XRPD pattern comprising peaks at 8.9°±0.1° and 9.3°±0.1°.

In certain embodiments, the XRPD pattern further comprises one or more peaks selected from 14.8°±0.1° and 26.7°±0.1° 2θ. In certain embodiments, the XRPD pattern further comprises one or more peaks selected from 5.3°±0.1°, 18.0°±0.1°, and 22.6°±0.1° 2θ. In certain embodiments, the XRPD pattern further comprises a peak at 32.2°±0.1°.

In certain embodiments, the XRPD pattern further comprises one or more peaks selected from 12.6°±0.1°, 14.5°±0.1°, 15.4°±0.1°, 16.1°±0.1°, 16.5°±0.1°, 17.5°±0.1°, 19.3°±0.1°, 19.8°±0.1°, 22.1°±0.1°, 23.1°±0.1°, 23.5°±0.1°, 24.3°±0.1°, 24.9°±0.1°, 25.4°±0.1°, 25.9°±0.1°, and 27.5°±0.1° 2θ. In certain embodiments, the XRPD pattern further comprises one or more peaks selected from 3.3°±0.1°, 24.8°±0.1°, and 26.1°±0.1° 2θ. In certain embodiments, the XRPD pattern further comprises one or more peaks selected from 6.5°±0.1°, 13.3°±0.1°, 16.7°±0.1°, 21.5°±0.1°, 22.3°±0.1°, 23.9°±0.1°, 24.1°±0.1°, and 26.8°±0.1° 2θ. In certain embodiments, the XRPD pattern further comprises one or more peaks selected from 28.2°±0.1°, 29.3°±0.1°, 30.5°±0.1°, 31.0°±0.1°, 31.6°±0.1°, and 34.5°±0.1° 2θ. In certain embodiments, the XRPD pattern further comprises a peak at 29.1°±0.1° 2θ. In certain embodiments, the XRPD pattern further comprises one or more peaks selected from 32.9°±0.1° and 33.7°±0.1° 2θ. In certain embodiments, the XRPD pattern further comprises one or more peaks selected from 3.3° 0.1°, 6.5°±0.1°, 22.3°±0.1°, 23.9°±0.1°, 24.8°±0.1°, and 26.8° °±0.1° 2θ. In certain embodiments, the XRPD pattern further comprises one or more peaks selected from 26.8°±0.1°, 29.1°±0.1°, 29.3°±0.1°, and 32.9°±0.10 2°.

In certain embodiments, the crystalline inupadenant hydrochloride hydrate has an XRPD pattern substantially corresponding to the XRPD pattern shown in FIG. 4. In certain embodiments, the crystalline inupadenant hydrochloride hydrate has an XRPD pattern substantially corresponding to the XRPD pattern shown in FIG. 5. In certain embodiments, the crystalline inupadenant hydrochloride hydrate has an XRPD pattern substantially corresponding to the XRPD pattern shown in FIG. 12.

In certain embodiments, crystalline inupadenant hydrochloride hydrate has an XRPD pattern comprising one or more diffraction peaks (20) disclosed in Table 6. In certain embodiments, crystalline inupadenant hydrochloride hydrate has an XRPD pattern comprising one or more diffraction peaks (20) disclosed in Table 7. In certain embodiments, crystalline inupadenant hydrochloride hydrate has an XRPD pattern comprising one or more diffraction peaks (20) disclosed in Table 9. In certain embodiments, crystalline inupadenant hydrochloride hydrate has an XRPD pattern comprising one or more diffraction peaks (20) disclosed in Table 14.

In certain embodiments, the crystalline inupadenant hydrochloride hydrate has a DSC thermogram comprising an endotherm with a peak onset at about 70° C. In certain embodiments, the crystalline inupadenant hydrochloride hydrate has a DSC thermogram comprising an endotherm with a peak onset at about 140° C. In certain embodiments, the crystalline inupadenant hydrochloride hydrate has a DSC thermogram comprising an endotherm with a peak onset at about 240° C. In certain embodiments, the crystalline inupadenant hydrochloride hydrate has a DSC thermogram comprising one or more endotherms with peak onsets selected from about 70° C., about 140° C., and about 240° C. In certain embodiments, the crystalline inupadenant hydrochloride hydrate has a DSC thermogram substantially the same as shown in FIG. 9.

In various embodiments, the crystalline inupadenant hydrochloride is Form 1 inupadenant hydrochloride.

In various embodiments, the crystalline inupadenant hydrochloride hydrate is Form 2 inupadenant hydrochloride.

In certain embodiments, the crystalline inupadenant hydrochloride hydrate exhibits a change in mass of less than or equal to about 5% wt occurs when varying the relative humidity between 0% and about 95%, when measured at 25° C. The mass change exhibited by crystalline inupadenant hydrochloride hydrate as a function of humidity can be determined, for example, using DVS. In certain embodiments, the crystalline inupadenant hydrochloride hydrate has a water sorption isotherm, when measured at 25° C., substantially the same as shown in FIG. 10.

In certain embodiments, the crystalline inupadenant hydrochloride hydrate has a molar ratio of inupadenant to water of about 1:1 to about 1:1.5, about 1:1.1 to about 1:1.5, about 1:1.2 to about 1:1.5, about 1:1.3 to about 1:1.5, about 1:1.4 to about 1:1.5, about 1:1 to about 1:1.4, about 1:1 to about 1:1.3, about 1:1 to about 1:1.2, about 1:1 to about 1:1.1, about 1:1.1 to about 1:1.4, about 1:1.1 to about 1:1.3, about 1:1.1 to about 1:1.2, about 1:1.2 to about 1:1.4, about 1:1.2 to about 1:1.3, or about 1:1.3 to about 1:1.4. In certain embodiments, the crystalline inupadenant hydrochloride hydrate has a molar ratio of inupadenant to water of about 1:1 to about 1:1.5.

In certain embodiments, the crystalline inupadenant hydrochloride hydrate has a molar ratio of inupadenant to water of about 1:1, about 1:1.05, about 1:1.1, about 1:1.15, about 1:1.2, about 1:1.25, about 1:1.3, about 1:1.35, about 1:1.4, about 1:1.45, or about 1:1.5.

In certain embodiments, a weight loss of between about 1% wt and about 3.2% wt occurs upon heating the crystalline inupadenant hydrochloride hydrate from about 31° C. to about 83° C. The weight loss exhibited by crystalline inupadenant hydrochloride hydrate upon heating can be determined, for example, using TGA. In certain embodiments, the crystalline inupadenant hydrochloride hydrate has a TGA thermogram substantially the same as shown in FIG. 11A. In certain embodiments, the crystalline inupadenant hydrochloride hydrate has a TGA thermogram substantially the same as shown in FIG. 111B.

In certain embodiments, the crystalline inupadenant hydrochloride hydrate has a water content of about 2.5% wt to about 4.5% wt, about 2.7% wt to about 4.5% wt, about 2.9% wt to about 4.5% wt, about 3.1% wt to about 4.5% wt, about 3.3% wt to about 4.5% wt, about 3.5% wt to about 4.5% wt, about 3.7% wt to about 4.5% wt, about 3.9% wt to about 4.5% wt, about 4.1% wt to about 4.5% wt, about 4.3% wt to about 4.5% wt, about 2.5% wt to about 4.3% wt, about 2.5% wt to about 4.1% wt, about 2.5% wt to about 3.9% wt, about 2.5% wt to about 3.7% wt, about 2.5% wt to about 3.5% wt, about 2.5% wt to about 3.3% wt, about 2.5% wt to about 3.1% wt, about 2.5% wt to about 2.9% wt, about 2.5% wt to about 2.7% wt, about 2.7% wt to about ⁴0.3% wt, about 2.7% wt to about 4.1% wt, about 2.7% wt to about 3.9% wt, about 2.7% wt to about 3.7% wt, about 2.7% wt to about 3.5% wt, about 2.7% wt to about 3.3% wt, about 2.7% wt to about 3.1% wt, about 2.7% wt to about 2.9% wt, about 2.9% wt to about 4.3% wt, about 2.9% wt to about 4.1% wt, about 2.9% wt to about 3.9% wt, about 2.9% wt to about 3.7% wt, about 2.9% wt to about 3.5% wt, about 2.9% wt to about 3.3% wt, about 2.9% wt to about 3.1% wt, about 3.1% wt to about 4.3% wt, about 3.1% wt to about 4.1% wt, about 3.1% wt to about 3.9% wt, about 3.1% wt to about 3.7% wt, about 3.1% wt to about 3.5% wt, about 3.1% wt to about 3.3% wt, about 3.3% wt to about 4.3% wt, about 3.3% wt to about 4.1% wt, about 3.3% wt to about 3.9% wt, about 3.3% wt to about 3.7% wt, about 3.3% wt to about 3.5% wt, about 3.5% wt to about 4.3% wt, about 3.5% wt to about 4.1% wt, about 3.5% wt to about 3.9% wt, about 3.5% wt to about 3.7% wt, about 3.7% wt to about 4.3% wt, about 3.7% wt to about 4.1% wt, about 3.7% wt to about 3.9% wt, about 3.9% wt to about 4.3% wt, about 3.9% wt to about 4.1% wt, or about 4.1% wt to about 4.3% wt. In certain embodiments, the crystalline inupadenant hydrochloride hydrate has a water content of about 2.7% wt to about 3.5% wt.

In certain embodiments, the crystalline inupadenant hydrochloride hydrate has a water content of about 2.5% wt, about 2.7% wt, about 2.9% wt, about 3.1% wt, about 3.3% wt, about 3.5% wt, about 3.7% wt, about 3.9% wt, about 4.1% wt, about 4.3% wt, about 4.5% wt, or about 5.0% wt.

In certain embodiments, the crystalline inupadenant hydrochloride hydrate has a molar ratio of inupadenant to hydrochloride of about 1:1. In certain embodiments, the crystalline inupadenant hydrochloride hydrate has a molar ratio of inupadenant to hydrochloride of 1:1.

### (b) Form 1 Inupadenant Hydrochloride

In one aspect, provided herein is Form 1 inupadenant hydrochloride.

In certain embodiments, Form 1 inupadenant hydrochloride has an X-ray powder diffraction (XRPD) pattern comprising a peak at about 9.6° 2θ.

In certain embodiments, the XRPD pattern further comprises one or more peaks selected from about 9.0°, about 15.7°, about 18.2°, about 25.6°, and about 27.0° 2θ. In certain embodiments, the XRPD pattern further comprises one or more peaks selected from about 16.2°, about 17.5°, about 17.8°, about 22.5°, about 22.7°, about 24.3°, about 24.8°, and about 25.2° 2θ. In certain embodiments, the XRPD pattern further comprises one or more peaks selected from about 15.1°, about 19.4°, about 19.6°, about 21.5°, about 23.7°, and about 27.9° 2θ. In certain embodiments, the XRPD pattern further comprises one or more peaks selected from about 5.5°, about 7.6°, about 12.9°, about 13.5°, about 14.0°, and about 20.9° 2θ. In certain embodiments, the XRPD pattern further comprises one or more peaks selected from about 28.4°, about 29.5°, about 29.7°, about 30.8°, about 32.8°, and about 33.8° 2θ.

In certain embodiments, Form 1 inupadenant hydrochloride has an X-ray powder diffraction (XRPD) pattern comprising a peak at 9.6°±0.3°.

In certain embodiments, the XRPD pattern further comprises one or more peaks selected from 9.0°±0.3°, 15.7°±0.3°, 18.2°±0.3°, 25.6°±0.3°, and 27.0°±0.3° 2θ. In certain embodiments, the XRPD pattern further comprises one or more peaks selected from 16.2°±0.3°, 17.5°±0.3°, 17.8°±0.3°, 22.5°±0.3°, 22.7°±0.3°, 24.3°±0.3°, 24.8°±0.3°, and 25.2°±0.3°±2θ. In certain embodiments, the XRPD pattern further comprises one or more peaks selected from 15.1°±0.3°, 19.4°±0.3°, 19.6°±0.3°, 21.5°±0.3°, 23.7°±0.3°, and 27.9°±0.3° 2θ. In certain embodiments, the XRPD pattern further comprises one or more peaks selected from 5.5°±0.3°, 7.6°±0.3°, 12.9°±0.3°, 13.5°±0.3°, 14.0°±0.3°, and 20.9°±0.3° 2θ. In certain embodiments, the XRPD pattern further comprises one or more peaks selected from 28.4°±0.3°, 29.5°±0.3°, 29.7°±0.3°, 30.8°±0.3°, 32.8°±0.3°, and 33.8°±0.3°.

In certain embodiments, Form 1 inupadenant hydrochloride has an X-ray powder diffraction (XRPD) pattern comprising a peak at 9.6°±0.2°.

In certain embodiments, the XRPD pattern further comprises one or more peaks selected from 9.0°±0.2°, 15.7°±0.2°, 18.2°±0.2°, 25.6°±0.2°, and 27.0°±0.2° 2θ. In certain embodiments, the XRPD pattern further comprises one or more peaks selected from 16.2°±0.2°, 17.5°±0.2°, 17.8°±0.2°, 22.5°±0.2°, 22.7°±0.2°, 24.3°±0.2°, 24.8°±0.2°, and 25.2°±0.2°±2θ. In certain embodiments, the XRPD pattern further comprises one or more peaks selected from 15.1°±0.2°, 19.4°±0.2°, 19.6°±0.2°, 21.5°±0.2°, 23.7°±0.2°, and 27.9°±0.2° 2θ. In certain embodiments, the XRPD pattern further comprises one or more peaks selected from 5.5°±0.2°, 7.6°±0.2°, 12.9°±0.2°, 13.5°±0.2°, 14.0°±0.2°, and 20.9°±0.2° 2θ. In certain embodiments, the XRPD pattern further comprises one or more peaks selected from 28.4°±0.2°, 29.5°±0.2°, 29.7°±0.2°, 30.8°±0.2°, 32.8°±0.2°, and 33.8°±0.2°.

In certain embodiments, Form 1 inupadenant hydrochloride has an X-ray powder diffraction (XRPD) pattern comprising a peak at 9.6°±0.1°.

In certain embodiments, the XRPD pattern further comprises one or more peaks selected from 9.0°±0.1°, 15.7°±0.1°, 18.2°±0.1°, 25.6°±0.1°, and 27.0°±0.1° 2θ. In certain embodiments, the XRPD pattern further comprises one or more peaks selected from 16.2°±0.1°, 17.5°±0.1°, 17.8°±0.1°, 22.5°±0.1°, 22.7°±0.1°, 24.3°±0.1°, 24.8°±0.1°, and 25.2°±0.1° 20. In certain embodiments, the XRPD pattern further comprises one or more peaks selected from 15.1°±0.1°, 19.4°±0.1°, 19.6°±0.1°, 21.5°±0.1°, 23.7°±0.1°, and 27.9°±0.1° 2θ. In certain embodiments, the XRPD pattern further comprises one or more peaks selected from 5.5°±0.1°, 7.6°±0.1°, 12.9°±0.1°, 13.5°±0.1°, 14.0°±0.1°, and 20.9°±0.1° 2θ. In certain embodiments, the XRPD pattern further comprises one or more peaks selected from 28.4°±0.1°, 29.5°±0.1°, 29.7°±0.1°, 30.8°±0.1°, 32.8°±0.1°, and 33.8°±0.1°.

In certain embodiments, Form 1 inupadenant hydrochloride has an XRPD pattern substantially the same as shown in FIG. 12.

In certain embodiments, Form 1 inupadenant hydrochloride has an X-ray powder diffraction (XRPD) pattern comprising one or more diffraction peaks (20) disclosed in Table 6.

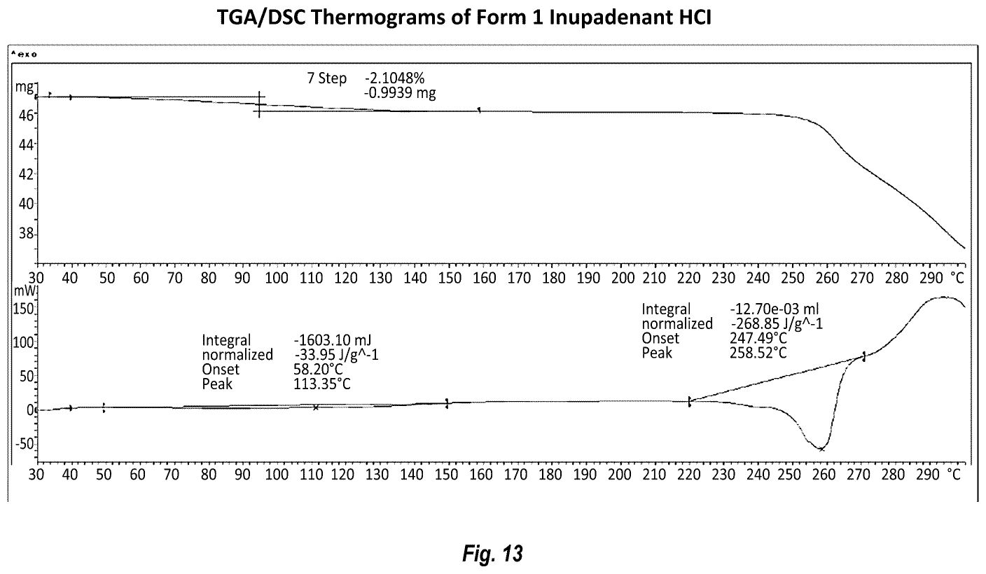

In certain embodiments, a weight loss of about 2.1% occurs upon heating Form 1 inupadenant hydrochloride from about 20° C. to about 80° C. The weight loss exhibited by Form 1 inupadenant hydrochloride upon heating can be determined, for example, using TGA. In certain embodiments, Form 1 inupadenant hydrochloride has a TGA thermogram substantially the same as shown in FIG. 13.

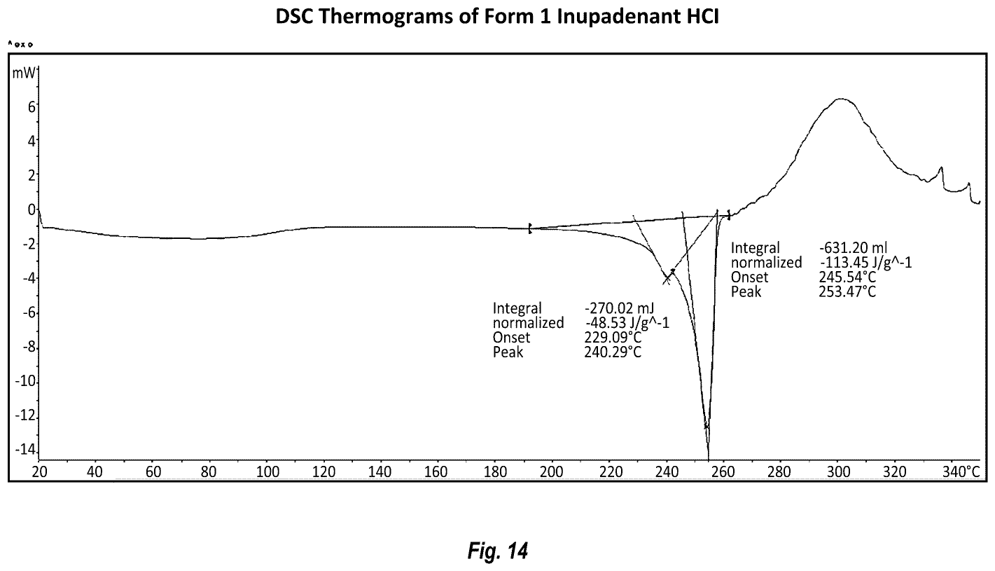

In certain embodiments, Form 1 inupadenant hydrochloride has a DSC thermogram comprising an endotherm with a peak onset at about 60° C. In certain embodiments, Form 1 inupadenant hydrochloride has a DSC thermogram comprising an endotherm with a peak onset at about 250° C. In certain embodiments, Form 1 inupadenant hydrochloride has a DSC thermogram comprising one or more endotherms with peak onsets selected from about 60° C. and about 250° C. In certain embodiments, Form 1 inupadenant hydrochloride has a DSC thermogram substantially the same as shown in FIG. 13. In certain embodiments, Form 1 inupadenant hydrochloride has a DSC thermogram substantially the same as shown in FIG. 14.

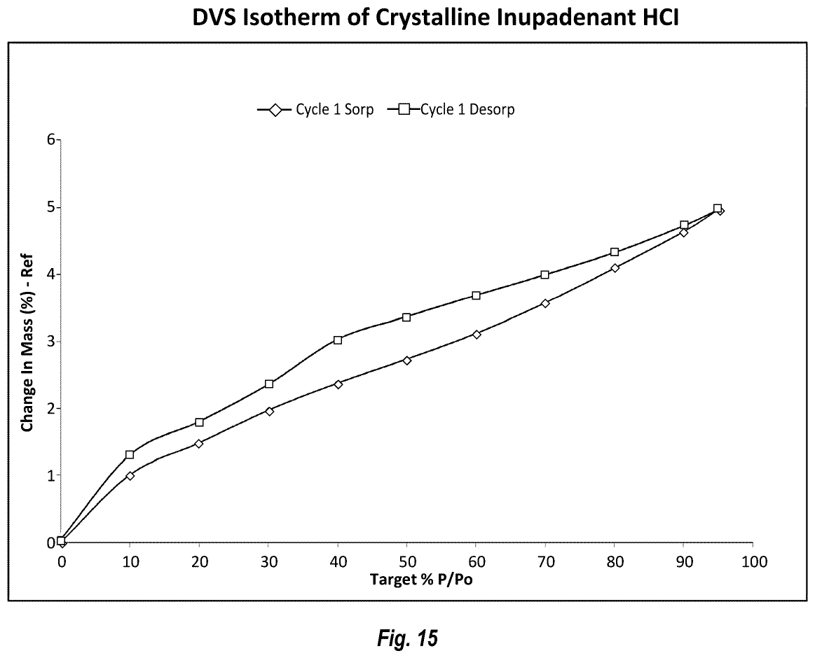

In certain embodiments, Form 1 inupadenant hydrochloride exhibits a change in mass of less than or equal to about 5% wt occurs when varying the relative humidity between 0% and about 95%. The mass change exhibited by Form 1 inupadenant hydrochloride as a function of humidity can be determined, for example, using DVS. In certain embodiments, Form 1 inupadenant hydrochloride has a water sorption isotherm, when measured at 25° C., substantially the same as shown in FIG. 15.

In certain embodiments, Form 1 inupadenant hydrochloride has a molar ratio of inupadenant to hydrochloride of about 1:1. In certain embodiments, Form 1 inupadenant hydrochloride has a molar ratio of inupadenant to hydrochloride of 1:1.

### (c) Form 2 Inupadenant Hydrochloride

In one aspect, Form 2 inupadenant hydrochloride.

In various embodiments, Form 2 inupadenant hydrochloride has an X-ray powder diffraction (XRPD) pattern comprising a peak at about 8.9° 2θ.

In various embodiments, Form 2 inupadenant hydrochloride has an XRPD pattern comprising a peak at about 9.3° 2θ.

In various embodiments, Form 2 inupadenant hydrochloride has an XRPD pattern comprising peaks at about 8.9° and about 9.3° 2θ.

In certain embodiments, the XRPD pattern further comprises one or more peaks selected from about 14.8° and about 26.7° 2θ. In certain embodiments, the XRPD pattern further comprises one or more peaks selected from about 5.3°, about 18.0°, and about 22.6° 2θ. In certain embodiments, the XRPD pattern further comprises a peak at about 32.2° 2θ.

In certain embodiments, the XRPD pattern further comprises one or more peaks selected from about 12.6°, about 14.5°, about 15.4°, about 16.1°, about 16.5°, about 17.5°, about 19.3°, about 19.8°, about 22.1°, about 23.1°, about 23.5°, about 24.3°, about 24.9°, about 25.4°, about 25.9°, and about 27.5° 2θ. In certain embodiments, the XRPD pattern further comprises one or more peaks selected from about 3.3°, about 24.8°, and about 26.1θ 2θ. In certain embodiments, the XRPD pattern further comprises one or more peaks selected from about 6.5°, about 13.3°, about 16.7°, about 21.5°, about 22.3°, about 23.9°, about 24.10, and about 26.8° 2θ. In certain embodiments, the XRPD pattern further comprises one or more peaks selected from about 28.2°, about 29.3°, about 30.5°, about 31.0°, about 31.6°, and about 34.5° 2θ. In certain embodiments, the XRPD pattern further comprises a peak at about 29.1° 2θ. In certain embodiments, the XRPD pattern further comprises one or more peaks selected from about 32.9° and about 33.7° 2θ. In certain embodiments, the XRPD pattern further comprises one or more peaks selected from about 3.3°, about 6.5°, about 22.3°, about 23.9°, about 24.8°, and about 26.8° 2θ. In certain embodiments, the XRPD pattern further comprises one or more peaks selected from about 26.8°, about 29.10, about 29.3°, and about 32.9° 2θ.

In certain embodiments, the XRPD pattern of Form 2 inupadenant hydrochloride does not comprise a discernable peak at about 7.6° 2θ.

In various embodiments, Form 2 inupadenant hydrochloride has an X-ray powder diffraction (XRPD) pattern comprising a peak at 8.9°±0.3° 2θ.

In various embodiments, Form 2 inupadenant hydrochloride has an XRPD pattern comprising a peak at 9.3°±0.3° 2θ.

In various embodiments, Form 2 inupadenant hydrochloride has an XRPD pattern comprising peaks at 8.9°±0.3° and 9.3°±0.3° 2θ.

In certain embodiments, the XRPD pattern further comprises one or more peaks selected from 14.8°±0.3° and 26.7°±0.3° 2θ. In certain embodiments, the XRPD pattern further comprises one or more peaks selected from 5.3°±0.3°, 18.0°±0.3°, and 22.6°±0.3° 2θ. In certain embodiments, the XRPD pattern further comprises a peak at 32.2°±0.3° 2θ.

In certain embodiments, the XRPD pattern further comprises one or more peaks selected from 12.6°±0.3°, 14.5°±0.3°, 15.4°±0.3°, 16.1°±0.3°, 16.5°±0.3°, 17.5°±0.3°, 19.3°±0.3°, 19.8°±0.3°, 22.1°±0.3°, 23.1°±0.3°, 23.5°±0.3°, 24.3°±0.3°, 24.9°±0.3°, 25.4°±0.3°, 25.9°±0.3°, and 27.5°±0.3° 2θ. In certain embodiments, the XRPD pattern further comprises one or more peaks selected from 3.3°±0.3°, 24.8°±0.3°, and 26.1°±0.3° 2θ. In certain embodiments, the XRPD pattern further comprises one or more peaks selected from 6.5°±0.3°, 13.3°±0.3°, 16.7°±0.3°, 21.5°±0.3°, 22.3°±0.3°, 23.9°±0.3°, 24.1°±0.3°, and 26.8°±0.3° 2θ. In certain embodiments, the XRPD pattern further comprises one or more peaks selected from 28.2°±0.3°, 29.3°±0.3°, 30.5°±0.3°, 31.0°±0.3°, 31.6°±0.3°, and 34.5°±0.3° 2θ. In certain embodiments, the XRPD pattern further comprises a peak at 29.1°±0.3° 2θ. In certain embodiments, the XRPD pattern further comprises one or more peaks selected from 32.9°±0.3° and 33.7°±0.3° 2θ. In certain embodiments, the XRPD pattern further comprises one or more peaks selected from 3.3° 0.3°, 6.5°±0.3°, 22.3°±0.3°, 23.9°±0.3°, 24.8°±0.3°, and 26.8°±0.3° 2θ. In certain embodiments, the XRPD pattern further comprises one or more peaks selected from 26.8°±0.3°, 29.1°±0.30, 29.3°±0.30, and 32.9°±0.30 20.

In certain embodiments, the XRPD pattern of Form 2 inupadenant hydrochloride does not comprise a discernable peak at 7.6°±0.3°.

In various embodiments, Form 2 inupadenant hydrochloride has an X-ray powder diffraction (XRPD) pattern comprising a peak at 8.9° °±0.2°.

In various embodiments, Form 2 inupadenant hydrochloride has an XRPD pattern comprising a peak at 9.3°±0.2° 2θ.

In various embodiments, Form 2 inupadenant hydrochloride has an XRPD pattern comprising peaks at 8.9°±0.2° and 9.3°±0.2° 2θ.

In certain embodiments, the XRPD pattern further comprises one or more peaks selected from 14.8°±0.2° and 26.7°±0.2° 2θ. In certain embodiments, the XRPD pattern further comprises one or more peaks selected from 5.3°±0.2°, 18.0°±0.2°, and 22.6°±0.2° 2θ. In certain embodiments, the XRPD pattern further comprises a peak at 32.2°±0.2° 2θ.

In certain embodiments, the XRPD pattern further comprises one or more peaks selected from 12.6°±0.2°, 14.5°±0.2°, 15.4°±0.2°, 16.1°±0.2°, 16.5°±0.2°, 17.5°±0.2°, 19.3°±0.2°, 19.8°±0.2°, 22.1°±0.2°, 23.1°±0.2°, 23.5°±0.2°, 24.3°±0.2°, 24.9°±0.2°, 25.4°±0.2°, 25.9° °±0.2°, and 27.5°±0.2° 2θ. In certain embodiments, the XRPD pattern further comprises one or more peaks selected from 3.3°±0.2°, 24.8°±0.2°, and 26.1°±0.2° 2θ. In certain embodiments, the XRPD pattern further comprises one or more peaks selected from 6.5°±0.2°, 13.3°±0.2°, 16.7°±0.2°, 21.5°±0.2°, 22.3°±0.2°, 23.9°±0.2°, 24.1°±0.2°, and 26.8°±0.2° 2θ. In certain embodiments, the XRPD pattern further comprises one or more peaks selected from 28.2°±0.2°, 29.3°±0.2°, 30.5°±0.2°, 31.0°±0.2°, 31.6°±0.2°, and 34.5°±0.2° 2θ. In certain embodiments, the XRPD pattern further comprises a peak at 29.1°±0.2° 2θ. In certain embodiments, the XRPD pattern further comprises one or more peaks selected from 32.9°±0.2° and 33.7°±0.2° 2θ. In certain embodiments, the XRPD pattern further comprises one or more peaks selected from 3.3° 0.2°, 6.5°±0.2°, 22.3°±0.2°, 23.9°±0.2°, 24.8°±0.2°, and 26.8°±0.2° 2θ. In certain embodiments, the XRPD pattern further comprises one or more peaks selected from 26.8°±0.2°, 29.1°±0.2°, 29.3°±0.2°, and 32.9°±0.2°.

In certain embodiments, the XRPD pattern of Form 2 inupadenant hydrochloride does not comprise a discernable peak at 7.6°±0.2°.

In various embodiments, Form 2 inupadenant hydrochloride has an X-ray powder diffraction (XRPD) pattern comprising a peak at 8.9°±0.1°.

In various embodiments, Form 2 inupadenant hydrochloride has an XRPD pattern comprising a peak at 9.3°±0.1°.

In various embodiments, Form 2 inupadenant hydrochloride has an XRPD pattern comprising peaks at 8.9°±0.1° and 9.3°±0.1°.

In certain embodiments, the XRPD pattern further comprises one or more peaks selected from 14.8°±0.1° and 26.7°±0.1° 2θ. In certain embodiments, the XRPD pattern further comprises one or more peaks selected from 5.3°±0.1°, 18.0°±0.1°, and 22.6°±0.1° 2θ. In certain embodiments, the XRPD pattern further comprises a peak at 32.2°±0.1° 2θ.

In certain embodiments, the XRPD pattern further comprises one or more peaks selected from 12.6°±0.1°, 14.5°±0.1°, 15.4°±0.1°, 16.1°±0.1°, 16.5°±0.1°, 17.5°±0.1°, 19.3°±0.1°, 19.8°±0.1°, 22.1°±0.1°, 23.1°±0.1°, 23.5°±0.1°, 24.3°±0.1°, 24.9°±0.1°, 25.4°±0.1°, 25.9° °±0.1°, and 27.5°±0.1° 2θ. In certain embodiments, the XRPD pattern further comprises one or more peaks selected from 3.3°±0.1°, 24.8°±0.1°, and 26.1°±0.1° 2θ. In certain embodiments, the XRPD pattern further comprises one or more peaks selected from 6.5°±0.1°, 13.3°±0.1°, 16.7°±0.1°, 21.5°±0.1°, 22.3°±0.1°, 23.9°±0.1°, 24.1°±0.1°, and 26.8°±0.1° 2θ. In certain embodiments, the XRPD pattern further comprises one or more peaks selected from 28.2°±0.1°, 29.3°±0.1°, 30.5°±0.1°, 31.0°±0.1°, 31.6°±0.1°, and 34.5°±0.1° 2θ. In certain embodiments, the XRPD pattern further comprises a peak at 29.1°±0.1° 2θ. In certain embodiments, the XRPD pattern further comprises one or more peaks selected from 32.9°±0.1° and 33.7°±0.1° 2θ. In certain embodiments, the XRPD pattern further comprises one or more peaks selected from 3.3° 0.1°, 6.5°±0.1°, 22.3°±0.1°, 23.9°±0.1°, 24.8°±0.1°, and 26.8°±0.1° 2θ. In certain embodiments, the XRPD pattern further comprises one or more peaks selected from 26.8°±0.1°, 29.10°±0.10, 29.3°±0.10, and 32.9°±0.10 20.

In certain embodiments, the XRPD pattern of Form 2 inupadenant hydrochloride does not comprise a discernable peak at 7.6°±0.1°.

In certain embodiments, Form 2 inupadenant hydrochloride has an XRPD pattern substantially corresponding to the XRPD pattern shown in FIG. 4. In certain embodiments, Form 2 inupadenant hydrochloride has an XRPD pattern substantially corresponding to the XRPD pattern shown in FIG. 5.

In certain embodiments, Form 2 inupadenant hydrochloride has an X-ray powder diffraction (XRPD) pattern comprising one or more diffraction peaks (20) disclosed in Table 7. In certain embodiments, Form 2 inupadenant hydrochloride has an X-ray powder diffraction (XRPD) pattern comprising one or more diffraction peaks (20) disclosed in Table 9. In certain embodiments, Form 2 inupadenant hydrochloride has an X-ray powder diffraction (XRPD) pattern comprising one or more diffraction peaks (20) disclosed in Table 14.

In certain embodiments, Form 2 inupadenant hydrochloride has a DSC thermogram comprising an endotherm with a peak onset at about 70° C. In certain embodiments, Form 2 inupadenant hydrochloride has a DSC thermogram comprising an endotherm with a peak onset at about 140° C. In certain embodiments, Form 2 inupadenant hydrochloride has a DSC thermogram comprising an endotherm with a peak onset at about 240° C. In certain embodiments, Form 2 inupadenant hydrochloride has a DSC thermogram comprising one or more endotherms with peak onsets selected from about 70° C., about 140° C., and about 240° C. In certain embodiments, the crystalline inupadenant hydrochloride hydrate has a DSC thermogram substantially the same as shown in FIG. 9.

In certain embodiments, Form 2 inupadenant hydrochloride exhibits a change in mass of less than or equal to about 5% wt occurs when varying the relative humidity between 0% and about 95%, when measured at 25° C. The mass change exhibited by crystalline inupadenant hydrochloride hydrate as a function of humidity can be determined, for example, using DVS. In certain embodiments, Form 2 inupadenant hydrochloride has a water sorption isotherm, when measured at 25° C., substantially the same as shown in FIG. 10A.

In certain embodiments, Form 2 inupadenant hydrochloride has a molar ratio of inupadenant to water of about 1:1 to about 1:1.5, about 1:1.1 to about 1:1.5, about 1:1.2 to about 1:1.5, about 1:1.3 to about 1:1.5, about 1:1.4 to about 1:1.5, about 1:1 to about 1:1.4, about 1:1 to about 1:1.3, about 1:1 to about 1:1.2, about 1:1 to about 1:1.1, about 1:1.1 to about 1:1.4, about 1:1.1 to about 1:1.3, about 1:1.1 to about 1:1.2, about 1:1.2 to about 1:1.4, about 1:1.2 to about 1:1.3, or about 1:1.3 to about 1:1.4. In certain embodiments, Form 2 inupadenant hydrochloride has a molar ratio of inupadenant to water of about 1:1 to about 1:1.5.

In certain embodiments, Form 2 inupadenant hydrochloride has a molar ratio of inupadenant to water of about 1:1, about 1:1.05, about 1:1.1, about 1:1.15, about 1:1.2, about 1:1.25, about 1:1.3, about 1:1.35, about 1:1.4, about 1:1.45, or about 1:1.5.

In certain embodiments, a weight loss of between about 1% wt and about 3.2% wt occurs upon heating Form 2 inupadenant hydrochloride from about 31° C. to about 83° C. The weight loss exhibited by Form 2 inupadenant hydrochloride upon heating can be determined, for example, using TGA. In certain embodiments, Form 2 inupadenant hydrochloride has a TGA thermogram substantially the same as shown in FIG. 11A. In certain embodiments, Form 2 inupadenant hydrochloride has a TGA thermogram substantially the same as shown in FIG. 111B.

In certain embodiments, Form 2 inupadenant hydrochloride has a water content of about 2.5% wt to about 4.5% wt, about 2.7% wt to about 4.5% wt, about 2.9% wt to about 4.5% wt, about 3.1% wt to about 4.5% wt, about 3.3% wt to about 4.5% wt, about 3.5% wt to about 4.5% wt, about 3.7% wt to about 4.5% wt, about 3.9% wt to about 4.5% wt, about 4.1% wt to about 4.5% wt, about 4.3% wt to about 4.5% wt, about 2.5% wt to about 4.3% wt, about 2.5% wt to about 4.1% wt, about 2.5% wt to about 3.9% wt, about 2.5% wt to about 3.7% wt, about 2.5% wt to about 3.5% wt, about 2.5% wt to about 3.3% wt, about 2.5% wt to about 3.1% wt, about 2.5% wt to about 2.9% wt, about 2.5% wt to about 2.7% wt, about 2.7% wt to about 4.3% wt, about 2.7% wt to about 4.1% wt, about 2.7% wt to about 3.9% wt, about 2.7% wt to about 3.7% wt, about 2.7% wt to about 3.5% wt, about 2.7% wt to about 3.3% wt, about 2.7% wt to about 3.1% wt, about 2.7% wt to about 2.9% wt, about 2.9% wt to about 4.3% wt, about 2.9% wt to about 4.1% wt, about 2.9% wt to about 3.9% wt, about 2.9% wt to about 3.7% wt, about 2.9% wt to about 3.5% wt, about 2.9% wt to about 3.3% wt, about 2.9% wt to about 3.1% wt, about 3.1% wt to about 4.3% wt, about 3.1% wt to about 4.1% wt, about 3.1% wt to about 3.9% wt, about 3.1% wt to about 3.7% wt, about 3.1% wt to about 3.5% wt, about 3.1% wt to about 3.3% wt, about 3.3% wt to about 4.3% wt, about 3.3% wt to about 4.1% wt, about 3.3% wt to about 3.9% wt, about 3.3% wt to about 3.7% wt, about 3.3% wt to about 3.5% wt, about 3.5% wt to about 4.3% wt, about 3.5% wt to about 4.1% wt, about 3.5% wt to about 3.9% wt, about 3.5% wt to about 3.7% wt, about 3.7% wt to about 4.3% wt, about 3.7% wt to about 4.1% wt, about 3.7% wt to about 3.9% wt, about 3.9% wt to about 4.3% wt, about 3.9% wt to about 4.1% wt, or about 4.1% wt to about 4.3% wt. In certain embodiments, Form 2 inupadenant hydrochloride has a water content of about 2.7% wt to about 3.5% wt.

In certain embodiments, Form 2 inupadenant hydrochloride has a water content of about 2.5% wt, about 2.7% wt, about 2.9% wt, about 3.1% wt, about 3.3% wt, about 3.5% wt, about 3.7% wt, about 3.9% wt, about 4.1% wt, about 4.3% wt, about 4.5% wt, or about 5.0% wt.

In certain embodiments, Form 2 inupadenant hydrochloride has a molar ratio of inupadenant to hydrochloride of about 1:1. In certain embodiments, Form 2 inupadenant hydrochloride has a molar ratio of inupadenant to hydrochloride of 1:1.

### (3) Pharmacokinetics of Inupadenant

Inupadenant in free base form has a solubility in fasted simulated intestinal fluid (FaSSIF) of approximately 1 μg/ml and is considered practically insoluble in aqueous media.

In dog PK studies, the highest exposure for the free base formulation was achieved when the stomach pH was controlled below pH of 3.

In human clinical studies (Example 19) evaluating formulations containing inupadenant as a free base, PK data from 5 cohorts demonstrate high intra and inter-participant variability in exposure level upon BID administration, despite the protocol requiring administration with an acidic beverage and prohibiting acid-reducing medications such as proton pump inhibitors. Possible reasons for this could be the variability in stomach pH, even in fasted state. This pH variability could have led to the precipitation of inupadenant, affecting transintestinal absorption. Additionally, exposure does not increase when the dose of inupadenant free base is increased from 80 mg BID to 160 mg BID.

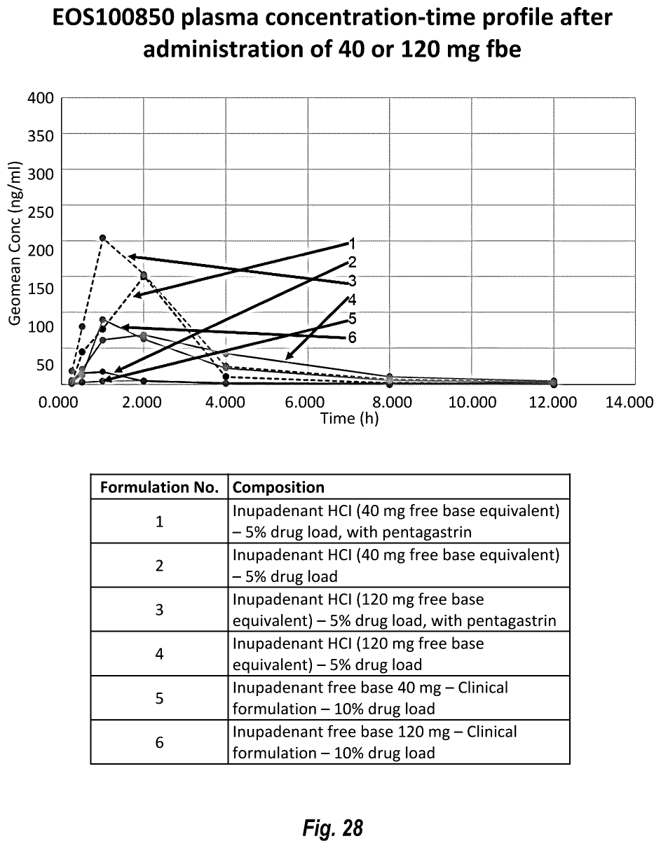

In-vivo testing in dogs of a hydrochloride salt of inupadenant resulted in an approximately 2-fold increase in plasma exposure compared to a corresponding free base formulation. (see Example 18, FIG. 28).

### Pharmaceutical Compositions

The disclosure relates to pharmaceutical compositions generally comprising, as the pharmaceutically active ingredient, inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), and at least one pharmaceutically acceptable excipient.

Also provided herein are pharmaceutical compositions generally comprising, as the pharmaceutically active ingredient, inupadenant free base (e.g., crystalline inupadenant free base) and at least one pharmaceutically acceptable excipient. In various embodiments, a pharmaceutical composition described herein comprises crystalline inupadenant free base and at least one pharmaceutically acceptable excipient.

In various embodiments, a pharmaceutical composition described herein comprises amorphous inupadenant hydrochloride; and at least one pharmaceutically acceptable excipient.

In various embodiments, a pharmaceutical composition described herein comprises amorphous inupadenant hydrochloride and/or crystalline inupadenant hydrochloride such as, for example, Form 1 inupadenant hydrochloride and/or Form 2 inupadenant hydrochloride; and at least one pharmaceutically acceptable excipient.

In various embodiments, a pharmaceutical composition described herein comprises crystalline inupadenant hydrochloride such as, for example, Form 1 inupadenant hydrochloride and/or Form 2 inupadenant hydrochloride; and at least one pharmaceutically acceptable excipient.

In certain embodiments, the at least one pharmaceutically acceptable excipient comprises a lipid carrier.

In various embodiments, a pharmaceutical composition described herein comprises: inupadenant hydrochloride; and a lipid carrier.

In various embodiments, a pharmaceutical composition described herein comprises: amorphous inupadenant hydrochloride; and a lipid carrier.

In one embodiment, the invention thus provides a pharmaceutical composition comprising: (a) inupadenant hydrochloride; (b) a lipid carrier; and (c) optionally one or more other pharmaceutically acceptable excipients.

In one embodiment, the invention thus provides a pharmaceutical composition comprising: (a) amorphous inupadenant hydrochloride; (b) a lipid carrier; and (c) optionally one or more other pharmaceutically acceptable excipients.

In one embodiment, the invention thus provides a pharmaceutical composition comprising: (a) crystalline inupadenant hydrochloride such as, for example, Form 1 inupadenant hydrochloride and/or Form 2 inupadenant hydrochloride; (b) a lipid carrier; and (c) optionally one or more other pharmaceutically acceptable excipients.

In various embodiments, a pharmaceutical composition described herein comprises: crystalline inupadenant hydrochloride such as, for example, Form 1 inupadenant hydrochloride and/or Form 2 inupadenant hydrochloride; and a lipid carrier. In certain embodiments, a pharmaceutical composition described herein further comprises a copovidone.

In various embodiments, a pharmaceutical composition described herein comprises: (a) inupadenant hydrochloride; (b) a lipid carrier; and (c) a copovidone.

In certain embodiments, a pharmaceutical composition described herein further comprises a polyethylene glycol (PEG). In certain embodiments, a pharmaceutical composition described herein further comprises an antioxidant.

In various embodiments, a pharmaceutical composition described herein comprises: (a) inupadenant hydrochloride; (b) a lipid carrier; (c) a copovidone; a polyethylene glycol; and (e) an antioxidant.

In various embodiments, a pharmaceutical composition described herein comprises: (a) amorphous inupadenant hydrochloride; (b) a lipid carrier; (c) a copovidone; a polyethylene glycol; and (e) an antioxidant.

In various embodiments, a pharmaceutical composition described herein comprises: (a) crystalline inupadenant hydrochloride such as, for example, Form 1 inupadenant hydrochloride and/or Form 2 inupadenant hydrochloride; (b) a lipid carrier; (c) a copovidone; a polyethylene glycol; and (e) an antioxidant.

### (1) Inupadenant Hydrochloride

In certain embodiments, the amorphous inupadenant hydrochloride is an amorphous inupadenant hydrochloride described herein.

In certain embodiments, the inupadenant hydrochloride is a crystalline inupadenant hydrochloride described herein such as, for example, Form 1 inupadenant hydrochloride and/or Form 2 inupadenant hydrochloride. In certain embodiments, the inupadenant hydrochloride is an inupadenant hydrochloride hydrate described herein. In certain embodiments, the inupadenant hydrochloride is a crystalline inupadenant hydrochloride hydrate described herein such as, for example, Form 1 inupadenant hydrochloride and/or Form 2 inupadenant hydrochloride.

In certain embodiments, the amount of inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride) in the pharmaceutical composition is about 0.4% (w/w) to about 60% (w/w), about 5% (w/w) to about 60% (w/w), about 10% (w/w) to about 60% (w/w), about 15% (w/w) to about 60% (w/w), about 20% (w/w) to about 60% (w/w), about 30% (w/w) to about 60% (w/w), about 40% (w/w) to about 60% (w/w), about 50% (w/w) to about 60% (w/w), about 0.4% (w/w) to about 50% (w/w), about 0.4% (w/w) to about 40% (w/w), about 0.4% (w/w) to about 30% (w/w), about 0.4% (w/w) to about 20% (w/w), about 0.4% (w/w) to about 15% (w/w), about 0.4% (w/w) to about 10% (w/w), about 0.4% (w/w) to about 5% (w/w), about 5% (w/w) to about 50% (w/w), about 5% (w/w) to about 40% (w/w), about 5% (w/w) to about 30% (w/w), about 5% (w/w) to about 20% (w/w), about 5% (w/w) to about 15% (w/w), about 5% (w/w) to about 10% (w/w), about 10% (w/w) to about 50% (w/w), about 10% (w/w) to about 40% (w/w), about 10% (w/w) to about 30% (w/w), about 10% (w/w) to about 20% (w/w), about 10% (w/w) to about 15% (w/w), about 15% (w/w) to about 50% (w/w), about 15% (w/w) to about 40% (w/w), about 15% (w/w) to about 30% (w/w), about 15% (w/w) to about 20% (w/w), about 20% (w/w) to about 50% (w/w), about 20% (w/w) to about 40% (w/w), about 20% (w/w) to about 30% (w/w), about 30% (w/w) to about 50% (w/w), about 30% (w/w) to about 40% (w/w), or about 40% (w/w) to about 50% (w/w). In certain embodiments, the amount of inupadenant hydrochloride in the pharmaceutical composition is about 4% (w/w) to about 6% (w/w).

In certain embodiments, the amount of inupadenant hydrochloride in the pharmaceutical composition is about 4.5% (w/w) to about 5.5% (w/w). In certain embodiments, the amount of inupadenant hydrochloride in the pharmaceutical composition is about 4.8% (w/w) to about 5.2% (w/w).

In certain embodiments, the amount of inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride) in the pharmaceutical composition is about 0.4% (w/w), about 0.5% (w/w), about 0.6% (w/w), about 0.7% (w/w), about 0.8% (w/w), about 0.9% (w/w), about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 310% (w/w), about 32% (w/w), about 33% (w/w), about 34% (w/w), about 35% (w/w), about 36% (w/w), about 37% (w/w), about 388% (w/w), about 399% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), about 50% (w/w), about 510% (w/w), about 52% (w/w), about 53% (w/w), about 54% (w/w), about 5 5% (w/w), about 56% (w/w), about 57% (w/w), about 58% (w/w), about 59% (w/w), or about 60% (w/w). In certain embodiments, the amount of inupadenant hydrochloride in the pharmaceutical composition is about 5% (w/w). In certain embodiments, the amount of inupadenant hydrochloride in the pharmaceutical composition is about 5.1% (w/w).

In certain embodiments, the amount of inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride) in the pharmaceutical composition is about 0.1 mg to about 100 mg, about 1 mg to about 100 mg, about 5 mg to about 100 mg, about 10 mg to about 100 mg, about 15 mg to about 100 mg, about 20 mg to about 100 mg, about 30 mg to about 100 mg, about 40 mg to about 100 mg, about 50 mg to about 100 mg, about 60 mg to about 100 mg, about 70 mg to about 100 mg, about 80 mg to about 100 mg, about 90 mg to about 100 mg, about 0.1 mg to about 90 mg, about 0.1 mg to about 80 mg, about 0.1 mg to about 70 mg, about 0.1 mg to about 60 mg, about 0.1 mg to about 50 mg, about 0.1 mg to about 40 mg, about 0.1 mg to about 30 mg, about 0.1 mg to about 20 mg, about 0.1 mg to about 15 mg, about 0.1 mg to about 10 mg, about 0.1 mg to about 5 mg, about 0.1 mg to about 1 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 5 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 40 mg, about 10 mg to about 30 mg, about 10 mg to about 20 mg, about 10 mg to about 15 mg, about 15 mg to about 90 mg, about 15 mg to about 80 mg, about 15 mg to about 70 mg, about 15 mg to about 60 mg, about 15 mg to about 50 mg, about 15 mg to about 40 mg, about 15 mg to about 30 mg, about 15 mg to about 20 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg. In certain embodiments, the amount of inupadenant hydrochloride in the pharmaceutical composition is about 5 mg to about 60 mg. In certain embodiments, the amount of inupadenant hydrochloride in the pharmaceutical composition is about 5 mg to about 15 mg. In certain embodiments, the amount of inupadenant hydrochloride in the pharmaceutical composition is about 15 mg to about 25 mg. In certain embodiments, the amount of inupadenant hydrochloride in the pharmaceutical composition is about 35 mg to about 45 mg.

In certain embodiments, the amount of inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride) in the pharmaceutical composition is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg. In certain embodiments, the amount of inupadenant hydrochloride in the pharmaceutical composition is about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 35 mg, about 36 mg, about 37 mg, about 38 mg, about 39 mg, about 40 mg, about 41 mg, about 42 mg, about 43 mg, about 44 mg or about 45 mg. In certain embodiments, the amount of inupadenant hydrochloride in the pharmaceutical composition is about 9.6 mg. In certain embodiments, the amount of inupadenant hydrochloride in the pharmaceutical composition is about 9.8 mg. In certain embodiments, the amount of inupadenant hydrochloride in the pharmaceutical composition is about 10 mg. In certain embodiments, the amount of inupadenant hydrochloride in the pharmaceutical composition is about 10.2 mg. In certain embodiments, the amount of inupadenant hydrochloride in the pharmaceutical composition is about 10.4 mg. In certain embodiments, the amount of inupadenant hydrochloride in the pharmaceutical composition is about 10.5 mg. In certain embodiments, the amount of inupadenant hydrochloride in the pharmaceutical composition is about 10.6 mg. In certain embodiments, the amount of inupadenant hydrochloride in the pharmaceutical composition is about 10.8 mg. In certain embodiments, the amount of inupadenant hydrochloride in the pharmaceutical composition is about 11 mg. In certain embodiments, the amount of inupadenant hydrochloride in the pharmaceutical composition is about 11.2 mg. In certain embodiments, the amount of inupadenant hydrochloride in the pharmaceutical composition is about 11.4 mg. In certain embodiments, the amount of inupadenant hydrochloride in the pharmaceutical composition is about 11.6 mg. In certain embodiments, the amount of inupadenant hydrochloride in the pharmaceutical composition is about 20 mg. In certain embodiments, the amount of inupadenant hydrochloride in the pharmaceutical composition is about 20.2 mg. In certain embodiments, the amount of inupadenant hydrochloride in the pharmaceutical composition is about 20.4 mg. In certain embodiments, the amount of inupadenant hydrochloride in the pharmaceutical composition is about 20.6 mg. In certain embodiments, the amount of inupadenant hydrochloride in the pharmaceutical composition is about 20.8 mg. In certain embodiments, the amount of inupadenant hydrochloride in the pharmaceutical composition is about 21 mg. In certain embodiments, the amount of inupadenant hydrochloride in the pharmaceutical composition is about 21.2 mg. In certain embodiments, the amount of inupadenant hydrochloride in the pharmaceutical composition is about 21.4 mg. In certain embodiments, the amount of inupadenant hydrochloride in the pharmaceutical composition is about 21.5 mg. In certain embodiments, the amount of inupadenant hydrochloride in the pharmaceutical composition is about 21.6 mg. In certain embodiments, the amount of inupadenant hydrochloride in the pharmaceutical composition is about 21.8 mg. In certain embodiments, the amount of inupadenant hydrochloride in the pharmaceutical composition is about 22 mg. In certain embodiments, the amount of inupadenant hydrochloride in the pharmaceutical composition is about 23 mg. In certain embodiments, the amount of inupadenant hydrochloride in the pharmaceutical composition is about 24 mg. In certain embodiments, the amount of inupadenant hydrochloride in the pharmaceutical composition is about 25 mg. In certain embodiments, the amount of inupadenant hydrochloride in the pharmaceutical composition is about 30 mg. In certain embodiments, the amount of inupadenant hydrochloride in the pharmaceutical composition is about 40 mg. In certain embodiments, the amount of inupadenant hydrochloride in the pharmaceutical composition is about 41 mg. In certain embodiments, the amount of inupadenant hydrochloride in the pharmaceutical composition is about 41.2 mg. In certain embodiments, the amount of inupadenant hydrochloride in the pharmaceutical composition is about 41.4 mg. In certain embodiments, the amount of inupadenant hydrochloride in the pharmaceutical composition is about 41.6 mg. In certain embodiments, the amount of inupadenant hydrochloride in the pharmaceutical composition is about 41.8 mg. In certain embodiments, the amount of inupadenant hydrochloride in the pharmaceutical composition is about 42 mg. In certain embodiments, the amount of inupadenant hydrochloride in the pharmaceutical composition is about 42.2 mg. In certain embodiments, the amount of inupadenant hydrochloride in the pharmaceutical composition is about 42.4 mg. In certain embodiments, the amount of inupadenant hydrochloride in the pharmaceutical composition is about 42.6 mg. In certain embodiments, the amount of inupadenant hydrochloride in the pharmaceutical composition is about 42.8 mg. In certain embodiments, the amount of inupadenant hydrochloride in the pharmaceutical composition is about 43 mg. In certain embodiments, the amount of inupadenant hydrochloride in the pharmaceutical composition is about 44 mg. In certain embodiments, the amount of inupadenant hydrochloride in the pharmaceutical composition is about 45 mg. In certain embodiments, the amount of inupadenant hydrochloride in the pharmaceutical composition is about 46 mg. In certain embodiments, the amount of inupadenant hydrochloride in the pharmaceutical composition is about 47 mg. In certain embodiments, the amount of inupadenant hydrochloride in the pharmaceutical composition is about 50 mg. In certain embodiments, the amount of inupadenant hydrochloride in the pharmaceutical composition is about 60 mg.

In certain embodiments, the amount of crystalline inupadenant hydrochloride hydrate such as, for example, Form 1 inupadenant hydrochloride and/or Form 2 inupadenant hydrochloride, in the pharmaceutical composition is about 0.4% (w/w) to about 60% (w/w), about 5% (w/w) to about 60% (w/w), about 10% (w/w) to about 60% (w/w), about 15% (w/w) to about 60% (w/w), about 20% (w/w) to about 60% (w/w), about 30% (w/w) to about 60% (w/w), about 40% (w/w) to about 60% (w/w), about 50% (w/w) to about 60% (w/w), about 0.4% (w/w) to about 50% (w/w), about 0.4% (w/w) to about 40% (w/w), about 0.4% (w/w) to about 30% (w/w), about 0.4% (w/w) to about 20% (w/w), about 0.4% (w/w) to about 15% (w/w), about 0.4% (w/w) to about 10% (w/w), about 0.4% (w/w) to about 5% (w/w), about 5% (w/w) to about 50% (w/w), about 5% (w/w) to about 40% (w/w), about 5% (w/w) to about 30% (w/w), about 5% (w/w) to about 20% (w/w), about 5% (w/w) to about 15% (w/w), about 5% (w/w) to about 10% (w/w), about 10% (w/w) to about 50% (w/w), about 10% (w/w) to about 40% (w/w), about 10% (w/w) to about 30% (w/w), about 10% (w/w) to about 20% (w/w), about 10% (w/w) to about 15% (w/w), about 15% (w/w) to about 50% (w/w), about 15% (w/w) to about 40% (w/w), about 15% (w/w) to about 30% (w/w), about 15% (w/w) to about 20% (w/w), about 20% (w/w) to about 50% (w/w), about 20% (w/w) to about 40% (w/w), about 20% (w/w) to about 30% (w/w), about 30% (w/w) to about 50% (w/w), about 30% (w/w) to about 40% (w/w), or about 40% (w/w) to about 50% (w/w). In certain embodiments, the amount of crystalline inupadenant hydrochloride hydrate such as, for example, Form 1 inupadenant hydrochloride and/or Form 2 inupadenant hydrochloride, in the pharmaceutical composition is about 4% (w/w) to about 6% (w/w). In certain embodiments, the amount of crystalline inupadenant hydrochloride hydrate such as, for example, Form 1 inupadenant hydrochloride and/or Form 2 inupadenant hydrochloride in the pharmaceutical composition is about 4.5% (w/w) to about 5.5% (w/w).

In certain embodiments, the amount of crystalline inupadenant hydrochloride hydrate such as, for example, Form 1 inupadenant hydrochloride and/or Form 2 inupadenant hydrochloride, in the pharmaceutical composition is about 0.4% (w/w), about 0.5% (w/w), about 0.6% (w/w), about 0.7% (w/w), about 0.8% (w/w), about 0.9% (w/w), about 1% (w/w), about 2% (w/w), about 3% (w/w), about 4% (w/w), about 5% (w/w), about 6% (w/w), about 7% (w/w), about 8% (w/w), about 9% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 21% (w/w), about 22% (w/w), about 23% (w/w), about 24% (w/w), about 25% (w/w), about 26% (w/w), about 27% (w/w), about 28% (w/w), about 29% (w/w), about 30% (w/w), about 310% (w/w), about 32% (w/w), about 33% (w/w), about 34% (w/w), about 35% (w/w), about 36% (w/w), about 37% (w/w), about 388% (w/w), about 39% (w/w), about 40% (w/w), about 41% (w/w), about 42% (w/w), about 43% (w/w), about 44% (w/w), about 45% (w/w), about 46% (w/w), about 47% (w/w), about 48% (w/w), about 49% (w/w), about 50% (w/w), about 51% (w/w), about 52% (w/w), about 53% (w/w), about 54% (w/w), about 55% (w/w), about 56% (w/w), about 57% (w/w), about 58% (w/w), about 59% (w/w), or about 60% (w/w).

In certain embodiments, the amount of crystalline inupadenant hydrochloride hydrate such as, for example, Form 1 inupadenant hydrochloride and/or Form 2 inupadenant hydrochloride, in the pharmaceutical composition is about 0.1 mg to about 100 mg, about 1 mg to about 100 mg, about 5 mg to about 100 mg, about 10 mg to about 100 mg, about 15 mg to about 100 mg, about 20 mg to about 100 mg, about 30 mg to about 100 mg, about 40 mg to about 100 mg, about 50 mg to about 100 mg, about 60 mg to about 100 mg, about 70 mg to about 100 mg, about 80 mg to about 100 mg, about 90 mg to about 100 mg, about 0.1 mg to about 90 mg, about 0.1 mg to about 80 mg, about 0.1 mg to about 70 mg, about 0.1 mg to about 60 mg, about 0.1 mg to about 50 mg, about 0.1 mg to about 40 mg, about 0.1 mg to about 30 mg, about 0.1 mg to about 20 mg, about 0.1 mg to about 15 mg, about 0.1 mg to about 10 mg, about 0.1 mg to about 5 mg, about 0.1 mg to about 1 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 15 mg, about 1 mg to about 10 mg, about 1 mg to about 5 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 15 mg, about 5 mg to about 10 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 40 mg, about 10 mg to about 30 mg, about 10 mg to about 20 mg, about 10 mg to about 15 mg, about 15 mg to about 90 mg, about 15 mg to about 80 mg, about 15 mg to about 70 mg, about 15 mg to about 60 mg, about 15 mg to about 50 mg, about 15 mg to about 40 mg, about 15 mg to about 30 mg, about 15 mg to about 20 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 30 mg to about 90 mg, about 30 mg to about 80 mg, about 30 mg to about 70 mg, about 30 mg to about 60 mg, about 30 mg to about 50 mg, about 30 mg to about 40 mg, about 40 mg to about 90 mg, about 40 mg to about 80 mg, about 40 mg to about 70 mg, about 40 mg to about 60 mg, about 40 mg to about 50 mg, about 50 mg to about 90 mg, about 50 mg to about 80 mg, about 50 mg to about 70 mg, about 50 mg to about 60 mg, about 60 mg to about 90 mg, about 60 mg to about 80 mg, about 60 mg to about 70 mg, about 70 mg to about 90 mg, about 70 mg to about 80 mg, or about 80 mg to about 90 mg. In certain embodiments, the amount of crystalline inupadenant hydrochloride hydrate in the pharmaceutical composition is about 5 mg to about 60 mg. In certain embodiments, the amount of crystalline inupadenant hydrochloride hydrate in the pharmaceutical composition is about 5 mg to about 15 mg. In certain embodiments, the amount of crystalline inupadenant hydrochloride hydrate in the pharmaceutical composition is about 15 mg to about 25 mg. In certain embodiments, the amount of crystalline inupadenant hydrochloride hydrate in the pharmaceutical composition is about 35 mg to about 45 mg.

In certain embodiments, the amount of crystalline inupadenant hydrochloride hydrate such as, for example, Form 1 inupadenant hydrochloride and/or Form 2 inupadenant hydrochloride, in the pharmaceutical composition is about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg. In certain embodiments, the amount of crystalline inupadenant hydrochloride hydrate such as, for example, Form 1 inupadenant hydrochloride and/or Form 2 inupadenant hydrochloride, in the pharmaceutical composition is about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 35 mg, about 36 mg, about 37 mg, about 38 mg, about 39 mg, about 40 mg, about 41 mg, about 42 mg, about 43 mg, about 44 mg or about 45 mg.

### (2) Lipid Carriers

In these and other embodiments, the amount of the lipid carrier in the pharmaceutical composition is 0% (w/w) to about 90% (w/w), about 10% (w/w) to about 90% (w/w), about 20% (w/w) to about 90% (w/w), about 30% (w/w) to about 90% (w/w), about 40% (w/w) to about 90% (w/w), about 50% (w/w) to about 90% (w/w), about 60% (w/w) to about 90% (w/w), about 70% (w/w) to about 90% (w/w), about 75% (w/w) to about 90% (w/w), about 80% (w/w) to about 90% (w/w), about 85% (w/w) to about 90% (w/w), 0% (w/w) to about 85% (w/w), 0% (w/w) to about 80% (w/w), 0% (w/w) to about 75% (w/w), 0% (w/w) to about 70% (w/w), 0% (w/w) to about 60% (w/w), 0% (w/w) to about 50% (w/w), 0% (w/w) to about 40% (w/w), 0% (w/w) to about 30% (w/w), 0% (w/w) to about 20% (w/w), 0% (w/w) to about 10% (w/w), about 10% (w/w) to about 85% (w/w), about 10% (w/w) to about 80% (w/w), about 10% (w/w) to about 75% (w/w), about 10% (w/w) to about 70% (w/w), about 10% (w/w) to about 60% (w/w), about 10% (w/w) to about 50% (w/w), about 10% (w/w) to about 40% (w/w), about 10% (w/w) to about 30% (w/w), about 10% (w/w) to about 20% (w/w), about 20% (w/w) to about 85% (w/w), about 20% (w/w) to about 80% (w/w), about 20% (w/w) to about 75% (w/w), about 20% (w/w) to about 70% (w/w), about 20% (w/w) to about 60% (w/w), about 20% (w/w) to about 50% (w/w), about 20% (w/w) to about 40% (w/w), about 20% (w/w) to about 30% (w/w), about 30% (w/w) to about 85% (w/w), about 30% (w/w) to about 80% (w/w), about 30% (w/w) to about 75% (w/w), about 30% (w/w) to about 70% (w/w), about 30% (w/w) to about 60% (w/w), about 30% (w/w) to about 50% (w/w), about 30% (w/w) to about 40% (w/w), about 40% (w/w) to about 80% (w/w), about 40% (w/w) to about 70% (w/w), about 40% (w/w) to about 60% (w/w), about 40% (w/w) to about 50% (w/w), about 50% (w/w) to about 85% (w/w), about 50% (w/w) to about 80% (w/w), about 50% (w/w) to about 75% (w/w), about 50% (w/w) to about 70% (w/w), about 50% (w/w) to about 60% (w/w), about 60% (w/w) to about 85% (w/w), about 60% (w/w) to about 80% (w/w), about 60% (w/w) to about 75% (w/w), about 60% (w/w) to about 70% (w/w), about 70% (w/w) to about 85% (w/w), about 70% (w/w) to about 80% (w/w), about 70% (w/w) to about 75% (w/w), about 75% (w/w) to about 85% (w/w), about 75% (w/w) to about 80% (w/w), or about 80% (w/w) to about 85% (w/w). In certain embodiments, the amount of the lipid carrier in the pharmaceutical composition is about 70% (w/w) to about 90% (w/w). In certain embodiments, the amount of the lipid carrier in the pharmaceutical composition is about 70% (w/w) to about 85% (w/w). In certain embodiments, the amount of the lipid carrier in the pharmaceutical composition is about 75% (w/w) to about 85% (w/w). In certain embodiments, the amount of the lipid carrier in the pharmaceutical composition is about 70% (w/w) to about 80% (w/w). In certain embodiments, the amount of the lipid carrier in the pharmaceutical composition is about 72% (w/w) to about 76% (w/w). In certain embodiments, the amount of the lipid carrier in the pharmaceutical composition is about 80% (w/w) to about 85% (w/w).

In these and other embodiments, the amount of the lipid carrier in the pharmaceutical composition is about 70% (w/w) to about 82% (w/w), about 71% (w/w) to about 82% (w/w), about 72% (w/w) to about 82% (w/w), about 73% (w/w) to about 82% (w/w), about 74% (w/w) to about 82% (w/w), about 75% (w/w) to about 82% (w/w), about 70% (w/w) to about 80% (w/w), about 71% (w/w) to about 80% (w/w), about 72% (w/w) to about 80% (w/w), about 73% (w/w) to about 80% (w/w), about 74% (w/w) to about 80% (w/w), about 70% (w/w) to about 79% (w/w), about 70% (w/w) to about 78% (w/w), about 70% (w/w) to about 77% (w/w), about 70% (w/w) to about 76% (w/w), about 70% (w/w) to about 75% (w/w), about 71% (w/w) to about 79% (w/w), about 72% (w/w) to about 78% (w/w), about 73% (w/w) to about 77% (w/w), about 74% (w/w) to about 76% (w/w), or about 74% (w/w) to about 75% (w/w).

In these and other embodiments, the amount of the lipid carrier in the pharmaceutical composition is about 70% (w/w), about 71% (w/w), about 72% (w/w), about 73% (w/w), about 73.4% (w/w), about 73.5% (w/w), about 73.6% (w/w), about 74% (w/w), about 75% (w/w), about 76% (w/w), about 77% (w/w), about 78% (w/w), about 79% (w/w), about 80% (w/w), about 81% (w/w), about 82% (w/w), about 83% (w/w), about 84% (w/w), or about 85% (w/w).

In these and other embodiments, the amount of the lipid carrier in the pharmaceutical composition is less than 90% (w/w).

In these and other embodiments, the amount of the lipid carrier in the pharmaceutical composition is about 100 mg to about 800 mg, about 150 mg to about 800 mg, about 175 mg to about 800 mg, about 200 mg to about 800 mg, about 225 mg to about 800 mg, about 250 mg to about 800 mg, about 275 mg to about 800 mg, about 300 mg to about 800 mg, about 325 mg to about 800 mg, about 350 mg to about 800 mg, about 375 mg to about 800 mg, about 400 mg to about 800 mg, about 425 mg to about 800 mg, about 450 mg to about 800 mg, about 475 mg to about 800 mg, about 100 mg to about 700 mg, about 100 mg to about 600 mg, about 100 mg to about 500 mg, about 100 mg to about 400 mg, about 100 mg to about 350 mg, about 100 mg to about 300 mg, about 150 mg to about 700 mg, about 150 mg to about 600 mg, about 150 mg to about 500 mg, about 150 mg to about 400 mg, about 150 mg to about 350 mg, about 150 mg to about 300 mg, about 175 mg to about 700 mg, about 175 mg to about 600 mg, about 175 mg to about 500 mg, about 175 mg to about 400 mg, about 175 mg to about 350 mg, about 175 mg to about 300 mg, about 200 mg to about 775 mg, about 200 mg to about 750 mg, about 200 mg to about 725 mg, about 200 mg to about 700 mg, about 200 mg to about 675 mg, about 200 mg to about 650 mg, about 200 mg to about 625 mg, about 200 mg to about 600 mg, about 200 mg to about 575 mg, about 200 mg to about 550 mg, about 200 mg to about 525 mg, about 225 mg to about 575 mg, about 225 mg to about 550 mg, about 225 mg to about 525 mg, about 225 mg to about 500 mg, about 225 mg to about 475 mg, about 225 mg to about 450 mg, about 225 mg to about 425 mg, about 225 mg to about 400 mg, about 225 mg to about 375 mg, about 225 mg to about 350 mg, about 230 mg to about 350 mg, about 235 mg to about 350 mg, about 240 mg to about 350 mg, about 245 mg to about 350 mg, about 250 mg to about 350 mg, about 500 mg to about 800 mg, about 510 mg to about 800 mg, about 520 mg to about 800 mg, about 530 mg to about 800 mg, about 530 mg to about 800 mg, about 540 mg to about 800 mg, about 550 mg to about 800 mg, about 560 mg to about 800 mg, about 570 mg to about 800 mg, about 580 mg to about 800 mg, about 590 mg to about 800 mg, about 600 mg to about 800 mg, about 520 mg to about 780 mg, about 540 mg to about 780 mg, about 560 mg to about 780 mg, about 580 mg to about 780 mg, about 600 mg to about 780 mg, about 540 mg to about 760 mg, about 560 mg to about 760 mg, about 580 mg to about 760 mg, about 600 mg to about 760 mg, about 540 mg to about 740 mg, about 540 mg to about 720 mg, about 540 mg to about 700 mg, about 540 mg to about 680 mg, about 540 mg to about 660 mg, about 540 mg to about 660 mg, about 540 mg to about 740 mg, or about 540 mg to about 620 mg. In certain embodiments, the amount of the lipid carrier in the pharmaceutical composition is about 150 mg to about 800 mg. In certain embodiments, the amount of the lipid carrier in the pharmaceutical composition is about 150 mg to about 250 mg. In certain embodiments, the amount of the lipid carrier in the pharmaceutical composition is about 300 mg to about 400 mg. In certain embodiments, the amount of the lipid carrier in the pharmaceutical composition is about 600 mg to about 700 mg of the lipid carrier.

In these and other embodiments, the amount of the lipid carrier in the pharmaceutical composition is about 100 mg, about 125 mg, about 150 mg, about 175 mg, about 200 mg, about 225 mg, about 250 mg, about 275 mg, about 300 mg, about 305 mg, about 310 mg, about 315 mg, about 320 mg, about 325 mg, about 350 mg, about 375 mg, about 400 mg, about 425 mg, about 450 mg, about 475 mg, about 525 mg, about 550 mg, about 575 mg, about 600 mg, about 605 mg, about 610 mg, about 615 mg, about 620 mg, about 625 mg, about 650 mg, about 675 mg, about 700 mg, about 725 mg, about 750 mg, about 775 mg, or about 800 mg. In certain embodiments, the amount of the lipid carrier in the pharmaceutical composition is about 175 mg. In certain embodiments, the amount of the lipid carrier in the pharmaceutical composition is about 305 mg. In certain embodiments, the amount of the lipid carrier in the pharmaceutical composition is about 350 mg. In certain embodiments, the amount of the lipid carrier in the pharmaceutical composition is about 610 mg. In certain embodiments, the amount of the lipid carrier in the pharmaceutical composition is about 700 mg.

In some embodiments, the lipid carrier is lauroyl polyoxyl-32 glycerides.

One example of such a lipid carrier is to Gelucire® 44/14 manufactured by Gattefosse (Saint-Priest—France). This lipid carrier is also known under the following references:
lauroyl polyoxyl-32 glycerides NF/USP (NF: National Formulary; USP: US Pharmacopeia);
lauroyl macrogol-32 glycerides EP (European Pharmacopeia);
hydrogenated coconut PEG-32 esters (INCI); and
CAS number 57107-95-6.

Gelucire® 44/14 corresponds to a well-defined multi-constituent substance constituted of mono-, di- and triglycerides and PEG-32 mono- and diesters of lauric acid (C12). Gelucire® 44/14 has a melting point ranging from 42.5° C. to 47.5° C. (with a mean at 44° C.) and a hydrophilic/lipophilic balance (HLB) value of 14.

Gelucire® 44/14 is used in order to enhance wetting, dissolution, solubility and bioavailability of the active ingredient.

In these and other embodiments, the amount of the lauroyl polyoxyl-32 glycerides in the pharmaceutical composition is 0% (w/w) to about 90% (w/w), about 10% (w/w) to about 90% (w/w), about 20% (w/w) to about 90% (w/w), about 30% (w/w) to about 90% (w/w), about 40% (w/w) to about 90% (w/w), about 50% (w/w) to about 90% (w/w), about 60% (w/w) to about 90% (w/w), about 70% (w/w) to about 90% (w/w), about 75% (w/w) to about 90% (w/w), about 80% (w/w) to about 90% (w/w), about 85% (w/w) to about 90% (w/w), 0% (w/w) to about 85% (w/w), 0% (w/w) to about 80% (w/w), 0% (w/w) to about 75% (w/w), 0% (w/w) to about 70% (w/w), 0% (w/w) to about 60% (w/w), 0% (w/w) to about 50% (w/w), 0% (w/w) to about 40% (w/w), 0% (w/w) to about 30% (w/w), 0% (w/w) to about 20% (w/w), 0% (w/w) to about 10% (w/w), about 10% (w/w) to about 85% (w/w), about 10% (w/w) to about 80% (w/w), about 10% (w/w) to about 75% (w/w), about 10% (w/w) to about 70% (w/w), about 10% (w/w) to about 60% (w/w), about 10% (w/w) to about 50% (w/w), about 10% (w/w) to about 40% (w/w), about 10% (w/w) to about 30% (w/w), about 10% (w/w) to about 20% (w/w), about 20% (w/w) to about 85% (w/w), about 20% (w/w) to about 80% (w/w), about 20% (w/w) to about 75% (w/w), about 20% (w/w) to about 70% (w/w), about 20% (w/w) to about 60% (w/w), about 20% (w/w) to about 50% (w/w), about 20% (w/w) to about 40% (w/w), about 20% (w/w) to about 30% (w/w), about 30% (w/w) to about 85% (w/w), about 30% (w/w) to about 80% (w/w), about 30% (w/w) to about 75% (w/w), about 30% (w/w) to about 70% (w/w), about 30% (w/w) to about 60% (w/w), about 30% (w/w) to about 50% (w/w), about 30% (w/w) to about 40% (w/w), about 40% (w/w) to about 80% (w/w), about 40% (w/w) to about 70% (w/w), about 40% (w/w) to about 60% (w/w), about 40% (w/w) to about 50% (w/w), about 50% (w/w) to about 85% (w/w), about 50% (w/w) to about 80% (w/w), about 50% (w/w) to about 75% (w/w), about 50% (w/w) to about 70% (w/w), about 50% (w/w) to about 60% (w/w), about 60% (w/w) to about 85% (w/w), about 60% (w/w) to about 80% (w/w), about 60% (w/w) to about 75% (w/w), about 60% (w/w) to about 70% (w/w), about 70% (w/w) to about 85% (w/w), about 70% (w/w) to about 80% (w/w), about 70% (w/w) to about 75% (w/w), about 75% (w/w) to about 85% (w/w), about 75% (w/w) to about 80% (w/w), or about 80% (w/w) to about 85% (w/w). In certain embodiments, the amount of the lauroyl polyoxyl-32 glycerides in the pharmaceutical composition is about 70% (w/w) to about 90% (w/w). In certain embodiments, the amount of the lauroyl polyoxyl-32 glycerides in the pharmaceutical composition is about 70% (w/w) to about 85% (w/w). In certain embodiments, the amount of the lauroyl polyoxyl-32 glycerides in the pharmaceutical composition is about 75% (w/w) to about 85% (w/w). In certain embodiments, the amount of the lauroyl polyoxyl-32 glycerides in the pharmaceutical composition is about 70% (w/w) to about 80% (w/w). In certain embodiments, the amount of the lauroyl polyoxyl-32 glycerides in the pharmaceutical composition is about 72% (w/w) to about 76% (w/w). In certain embodiments, the amount of the lauroyl polyoxyl-32 glycerides in the pharmaceutical composition is about 80% (w/w) to about 85% (w/w).

In these and other embodiments, the amount of the lauroyl polyoxyl-32 glycerides in the pharmaceutical composition is about 70% (w/w) to about 82% (w/w), about 71% (w/w) to about 82% (w/w), about 72% (w/w) to about 82% (w/w), about 73% (w/w) to about 82% (w/w), about 74% (w/w) to about 82% (w/w), about 75% (w/w) to about 82% (w/w), about 70% (w/w) to about 80% (w/w), about 71% (w/w) to about 80% (w/w), about 72% (w/w) to about 80% (w/w), about 73% (w/w) to about 80% (w/w), about 74% (w/w) to about 80% (w/w), about 70% (w/w) to about 79% (w/w), about 70% (w/w) to about 78% (w/w), about 70% (w/w) to about 77% (w/w), about 70% (w/w) to about 76% (w/w), about 70% (w/w) to about 75% (w/w), about 71% (w/w) to about 79% (w/w), about 72% (w/w) to about 78% (w/w), about 73% (w/w) to about 77% (w/w), about 74% (w/w) to about 76% (w/w), or about 74% (w/w) to about 75% (w/w).

In these and other embodiments, the amount of the lauroyl polyoxyl-32 glycerides in the pharmaceutical composition is about 70% (w/w), about 71% (w/w), about 72% (w/w), about 73% (w/w), about 73.4% (w/w), about 73.5% (w/w), about 73.6% (w/w), about 74% (w/w), about 75% (w/w), about 76% (w/w), about 77% (w/w), about 78% (w/w), about 79% (w/w), about 80% (w/w), about 81% (w/w), about 82% (w/w), about 83% (w/w), about 84% (w/w), or about 85% (w/w).

In these and other embodiments, the amount of the lauroyl polyoxyl-32 glycerides in the pharmaceutical composition is less than 90% (w/w).

In these and other embodiments, the amount of the lauroyl polyoxyl-32 glycerides in the pharmaceutical composition is about 100 mg to about 800 mg, about 150 mg to about 800 mg, about 175 mg to about 800 mg, about 200 mg to about 800 mg, about 225 mg to about 800 mg, about 250 mg to about 800 mg, about 275 mg to about 800 mg, about 300 mg to about 800 mg, about 325 mg to about 800 mg, about 350 mg to about 800 mg, about 375 mg to about 800 mg, about 400 mg to about 800 mg, about 425 mg to about 800 mg, about 450 mg to about 800 mg, about 475 mg to about 800 mg, about 100 mg to about 700 mg, about 100 mg to about 600 mg, about 100 mg to about 500 mg, about 100 mg to about 400 mg, about 100 mg to about 350 mg, about 100 mg to about 300 mg, about 150 mg to about 700 mg, about 150 mg to about 600 mg, about 150 mg to about 500 mg, about 150 mg to about 400 mg, about 150 mg to about 350 mg, about 150 mg to about 300 mg, about 175 mg to about 700 mg, about 175 mg to about 600 mg, about 175 mg to about 500 mg, about 175 mg to about 400 mg, about 175 mg to about 350 mg, about 175 mg to about 300 mg, about 200 mg to about 775 mg, about 200 mg to about 750 mg, about 200 mg to about 725 mg, about 200 mg to about 700 mg, about 200 mg to about 675 mg, about 200 mg to about 650 mg, about 200 mg to about 625 mg, about 200 mg to about 600 mg, about 200 mg to about 575 mg, about 200 mg to about 550 mg, about 200 mg to about 525 mg, about 225 mg to about 575 mg, about 225 mg to about 550 mg, about 225 mg to about 525 mg, about 225 mg to about 500 mg, about 225 mg to about 475 mg, about 225 mg to about 450 mg, about 225 mg to about 425 mg, about 225 mg to about 400 mg, about 225 mg to about 375 mg, about 225 mg to about 350 mg, about 230 mg to about 350 mg, about 235 mg to about 350 mg, about 240 mg to about 350 mg, about 245 mg to about 350 mg, about 250 mg to about 350 mg, about 500 mg to about 800 mg, about 510 mg to about 800 mg, about 520 mg to about 800 mg, about 530 mg to about 800 mg, about 530 mg to about 800 mg, about 540 mg to about 800 mg, about 550 mg to about 800 mg, about 560 mg to about 800 mg, about 570 mg to about 800 mg, about 580 mg to about 800 mg, about 590 mg to about 800 mg, about 600 mg to about 800 mg, about 520 mg to about 780 mg, about 540 mg to about 780 mg, about 560 mg to about 780 mg, about 580 mg to about 780 mg, about 600 mg to about 780 mg, about 540 mg to about 760 mg, about 560 mg to about 760 mg, about 580 mg to about 760 mg, about 600 mg to about 760 mg, about 540 mg to about 740 mg, about 540 mg to about 720 mg, about 540 mg to about 700 mg, about 540 mg to about 680 mg, about 540 mg to about 660 mg, about 540 mg to about 660 mg, about 540 mg to about 740 mg, or about 540 mg to about 620 mg. In certain embodiments, the amount of the lauroyl polyoxyl-32 glycerides in the pharmaceutical composition is about 150 mg to about 800 mg. In certain embodiments, the amount of the lauroyl polyoxyl-32 glycerides in the pharmaceutical composition is about 150 mg to about 250 mg. In certain embodiments, the amount of the lauroyl polyoxyl-32 glycerides in the pharmaceutical composition is about 300 mg to about 400 mg. In certain embodiments, the amount of the lauroyl polyoxyl-32 glycerides in the pharmaceutical composition is about 600 mg to about 700 mg of the lauroyl polyoxyl-32 glycerides.

In these and other embodiments, the amount of the lauroyl polyoxyl-32 glycerides in the pharmaceutical composition is about 100 mg, about 125 mg, about 150 mg, about 175 mg, about 200 mg, about 225 mg, about 250 mg, about 275 mg, about 300 mg, about 305 mg, about 310 mg, about 315 mg, about 320 mg, about 325 mg, about 350 mg, about 375 mg, about 400 mg, about 425 mg, about 450 mg, about 475 mg, about 525 mg, about 550 mg, about 575 mg, about 600 mg, about 605 mg, about 610 mg, about 615 mg, about 620 mg, about 625 mg, about 650 mg, about 675 mg, about 700 mg, about 725 mg, about 750 mg, about 775 mg, or about 800 mg. In certain embodiments, the amount of the lauroyl polyoxyl-32 glycerides in the pharmaceutical composition is about 175 mg. In certain embodiments, the amount of the lauroyl polyoxyl-32 glycerides in the pharmaceutical composition is about 305 mg. In certain embodiments, the amount of the lauroyl polyoxyl-32 glycerides in the pharmaceutical composition is about 350 mg. In certain embodiments, the amount of the lauroyl polyoxyl-32 glycerides in the pharmaceutical composition is about 610 mg. In certain embodiments, the amount of the lauroyl polyoxyl-32 glycerides in the pharmaceutical composition is about 700 mg.

### (3) Copovidone

In these and other embodiments, a pharmaceutical composition described herein comprises a polyvinylpyrrolidone. In certain embodiments, a pharmaceutical composition described herein comprises a copovidone. In some embodiments, the copovidone is Kollidon® VA 64. In certain embodiments, the molar ratio of N-vinylpyrrolidone monomers to vinyl acetate monomers in the copovidone is about 6:4. In certain embodiments, the weight average molecular weight (Mw) of the copovidone is about 45000 to about 70000.

In these and other embodiments, the amount of the copovidone in the pharmaceutical composition is about 0.10% (w/w) to about 10% (w/w), about 0.10% (w/w) to about 8% (w/w), about 0.10% (w/w) to about 7.0% (w/w), about 0.10% (w/w) to about⁶0.5% (w/w), about 0.10% (w/w) to about 6.0% (w/w), about 0.10% (w/w) to about 5.50% (w/w), about 0.10% (w/w) to about 5.0% (w/w), about 0.10% (w/w) to about 4.0% (w/w), about 0.10% (w/w) to about 3.0% (w/w), about 0.10% (w/w) to about 2.0% (w/w), about 0.10% (w/w) to about 1.50% (w/w), about 0.10% (w/w) to about 1.250% (w/w), about 0.50% (w/w) to about 5.0% (w/w), about 0.50% (w/w) to about 4.0% (w/w), about 0.50% (w/w) to about 3.0% (w/w), about 0.50% (w/w) to about 2.0% (w/w), about 0.500% (w/w) to about 1.50% (w/w), about 0.500% (w/w) to about 1.25% (w/w), about 0.75% (w/w) to about 5.0% (w/w), about 0.75% (w/w) to about 4.0% (w/w), about 0.75% (w/w) to about 3.0% (w/w), about 0.75% (w/w) to about 2.0% (w/w), about 0.75% (w/w) to about 1.5% (w/w), about 0.75% (w/w) to about 1.25% (w/w), about 1.0% (w/w) to about 5.0% (w/w), about 1.0% (w/w) to about 4.0% (w/w), about 1.0% (w/w) to about 3.0% (w/w), about 1.0% (w/w) to about 2.0% (w/w), about 1.0% (w/w) to about 1.5% (w/w), about 1.0% (w/w) to about 1.25% (w/w), 1.25% (w/w) to about 5.0% (w/w), about 1.5% (w/w) to about 5.0% (w/w), about 1.75% (w/w) to about 5.0% (w/w), about 2.0% (w/w) to about 5.0% (w/w), about 2.50% (w/w) to about 5.0% (w/w), about 3.0% (w/w) to about 5.0% (w/w), about 3.5% (w/w) to about 5.0% (w/w), about 4.0% (w/w) to about 5.0% (w/w), about 4.1% (w/w) to about 4.9% (w/w), about 4.2% (w/w) to about 4.8% (w/w), about 4.3% (w/w) to about 4.7% (w/w), about 4.4% (w/w) to about 4.7% (w/w), about 4.4% (w/w) to about 4.8% (w/w), or about 4.5% (w/w) to about 4.7% (w/w). In certain embodiments, the amount of the copovidone in the pharmaceutical composition is about 0.5% (w/w) to about 2.0% (w/w). In certain embodiments, the amount of the copovidone in the pharmaceutical composition is about 1.3% (w/w) to about 1.8% (w/w). In certain embodiments, the amount of the copovidone in the pharmaceutical composition is about 1.0% (w/w) to about 1.5% (w/w). In certain embodiments, the amount of the copovidone in the pharmaceutical composition is about 0.1% (w/w) to about 1.0% (w/w).

In these and other embodiments, the amount of the copovidone in the pharmaceutical composition is about 0.10% (w/w) to about 1.5% (w/w), about 0.15% (w/w) to about 1.45% (w/w), about 0.20% (w/w) to about 1.4% (w/w), about 0.25% (w/w) to about 1.35% (w/w), about 0.30% (w/w) to about 1.3% (w/w), about 0.35% (w/w) to about 1.25% (w/w), about 0.40% (w/w) to about 1.2% (w/w), about 0.45% (w/w) to about 1.15% (w/w), about 0.50% (w/w) to about 1.10% (w/w), about 0.55% (w/w) to about 1.05% (w/w), about 0.60% (w/w) to about 1.00% (w/w), about 0.65% (w/w) to about 0.95% (w/w), or about 0.70% (w/w) to about 0.90% (w/w).

In these and other embodiments, the amount of the copovidone in the pharmaceutical composition is about 0.10% (w/w) to about 9.0% (w/w), about 0.5% (w/w) to about 8.5% (w/w), about 1.0% (w/w) to about 8.0% (w/w), about 1.5% (w/w) to about 7.5% (w/w), about 2.0% (w/w) to about 7.0% (w/w), about 2.5% (w/w) to about 6.5% (w/w), about 3.0% (w/w) to about 6.0% (w/w), about 3.5% (w/w) to about 5.5% (w/w), about 3.6% (w/w) to about 5.4% (w/w), about 3.7% (w/w) to about 5.3% (w/w), about 3.8% (w/w) to about 5.2% (w/w), about 3.9% (w/w) to about 5.1% (w/w), about 4.0% (w/w) to about 5.0% (w/w), about 4.1% (w/w) to about 5.0% (w/w), about 4.2% (w/w) to about 5.0% (w/w), about 4.3% (w/w) to about 4.9% (w/w), about 4.4% (w/w) to about 4.8% (w/w), about 4.45% (w/w) to about 4.75% (w/w), about 4.5% (w/w) to about 4.7% (w/w), or about 4.55% (w/w) to about 4.65% (w/w).

In these and other embodiments, the amount of the copovidone in the pharmaceutical composition is about 4.3% (w/w) to about 4.9% (w/w), about 4.4% (w/w) to about 4.8% (w/w), about 4.45% (w/w) to about 4.75% (w/w), about 4.5% (w/w) to about 4.7% (w/w), or about 4.55% (w/w) to about 4.65% (w/w). In certain embodiments, the amount of the copovidone in the pharmaceutical composition is about 4.3% (w/w) to about 4.9% (w/w). In certain embodiments, the amount of the copovidone in the pharmaceutical composition is about 4.4% (w/w) to about 4.8% (w/w). In certain embodiments, the amount of the copovidone in the pharmaceutical composition is about 4.45% (w/w) to about 4.75% (w/w). In certain embodiments, the amount of the copovidone in the pharmaceutical composition is about 4.5% (w/w) to about 4.7% (w/w). In certain embodiments, the amount of the copovidone in the pharmaceutical composition about 4.55% (w/w) to about 4.65% (w/w).

In these and other embodiments, the amount of the copovidone in the pharmaceutical composition is about 0.10% (w/w), about 0.20% (w/w), about 0.30% (w/w), about 0.40% (w/w), about 0.50% (w/w), about 0.60% (w/w), about 0.70% (w/w), about 0.80% (w/w), about 0.90% (w/w), about 1.0% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2.0% (w/w), about 2.1% (w/w), about 2.2% (w/w), about 2.3% (w/w), about 2.4% (w/w), about 2.5% (w/w), about 2.6% (w/w), about 2.7% (w/w), about 2.8% (w/w), about 2.9% (w/w), about 3.0% (w/w), about 3.1% (w/w), about 3.2% (w/w), about 3.3% (w/w), about 3.4% (w/w), about 3.5% (w/w), about 3.6% (w/w), about 3.7% (w/w), about 3.8% (w/w), about 3.9% (w/w), about 4.0% (w/w), about 4.1% (w/w), about 4.2% (w/w), about 4.3% (w/w), about 4.4% (w/w), about 4.5% (w/w), about 4.6% (w/w), about 4.7% (w/w), about 4.8% (w/w), about 4.9% (w/w), or about 5.0% (w/w).

In these and other embodiments, the amount of the copovidone in the pharmaceutical composition is about 2 mg to about 500 mg, about 2 mg to about 250 mg, about 2 mg to about 100 mg, about 2 mg to about 90 mg, about 2 mg to about 80 mg, about 2 mg to about 70 mg, about 2 mg to about 60 mg, about 2 mg to about 50 mg, about 2 mg to about 40 mg, about 2 mg to about 30 mg, about 2 mg to about 20 mg, about 2 mg to about 10 mg, about 10 mg to about 500 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, or about 1 mg to about 40 mg. In certain embodiments, the amount of the copovidone in the pharmaceutical composition is about 2 mg to about 20 mg.

In these and other embodiments, the amount of the copovidone in the pharmaceutical composition is about 5 mg to about 35 mg, about 9 mg to about 29 mg, about 10 mg to about 28 mg, about 11 mg to about 27 mg, about 12 mg to about 26 mg, about 12 mg to about 26 mg, about 13 mg to about 25 mg, about 14 mg to about 24 mg, about 15 mg to about 23 mg, about 16 mg to about 22 mg, about 17 mg to about 21 mg, or about 18 mg to about 20 mg. In some embodiments, the amount of the copovidone in the pharmaceutical composition is about 28 mg to about 48 mg, about 29 mg to about 27 mg, about 30 mg to about 46 mg, about 31 mg to about 45 mg, about 32 mg to about 44 mg, about 33 mg to about 43 mg, about 34 mg to about 42 mg, about 35 mg to about 41 mg, about 36 mg to about 40 mg, or about 37 mg to about 39 mg.

In these and other embodiments, the amount of the copovidone in the pharmaceutical composition is about 1 mg, about 1.5 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg. In certain embodiments, the amount of the copovidone in the pharmaceutical composition is about 2.5 mg. In certain embodiments, the amount of the copovidone in the pharmaceutical composition is about 3 mg. In certain embodiments, the amount of the copovidone in the pharmaceutical composition is about 4 mg. In certain embodiments, the amount of the copovidone in the pharmaceutical composition is about 5 mg. In certain embodiments, the amount of the copovidone in the pharmaceutical composition is about 6 mg. In certain embodiments, the amount of the copovidone in the pharmaceutical composition is about 7 mg. In certain embodiments, the amount of the copovidone in the pharmaceutical composition is about 8 mg. In certain embodiments, the amount of the copovidone in the pharmaceutical composition is about 9 mg. In certain embodiments, the amount of the copovidone in the pharmaceutical composition is about 10 mg. In certain embodiments, the amount of the copovidone in the pharmaceutical composition is about 11 mg. In certain embodiments, the amount of the copovidone in the pharmaceutical composition is about 12 mg. In certain embodiments, the amount of the copovidone in the pharmaceutical composition is about 13 mg. In certain embodiments, the amount of the copovidone in the pharmaceutical composition is about 14 mg. In certain embodiments, the amount of the copovidone in the pharmaceutical composition is about 15 mg. In certain embodiments, the amount of the copovidone in the pharmaceutical composition is about 16 mg. In certain embodiments, the amount of the copovidone in the pharmaceutical composition is about 17 mg. In certain embodiments, the amount of the copovidone in the pharmaceutical composition is about 18 mg. In certain embodiments, the amount of the copovidone in the pharmaceutical composition is about 19 mg. In certain embodiments, the amount of the copovidone in the pharmaceutical composition is about 20 mg. In certain embodiments, the amount of the copovidone in the pharmaceutical composition is about 21 mg. In certain embodiments, the amount of the copovidone in the pharmaceutical composition is about 22 mg. In certain embodiments, the amount of the copovidone in the pharmaceutical composition is about 23 mg. In certain embodiments, the amount of the copovidone in the pharmaceutical composition is about 24 mg. In certain embodiments, the amount of the copovidone in the pharmaceutical composition is about 25 mg. In certain embodiments, the amount of the copovidone in the pharmaceutical composition is about 26 mg. In certain embodiments, the amount of the copovidone in the pharmaceutical composition is about 27 mg. In certain embodiments, the amount of the copovidone in the pharmaceutical composition is about 28 mg. In certain embodiments, the amount of the copovidone in the pharmaceutical composition is about 29 mg. In certain embodiments, the amount of the copovidone in the pharmaceutical composition is about 30 mg. In certain embodiments, the amount of the copovidone in the pharmaceutical composition is about 31 mg. In certain embodiments, the amount of the copovidone in the pharmaceutical composition is about 32 mg. In certain embodiments, the amount of the copovidone in the pharmaceutical composition is about 33 mg. In certain embodiments, the amount of the copovidone in the pharmaceutical composition is about 34 mg. In certain embodiments, the amount of the copovidone in the pharmaceutical composition is about 35 mg. In certain embodiments, the amount of the copovidone in the pharmaceutical composition is about 36 mg. In certain embodiments, the amount of the copovidone in the pharmaceutical composition is about 37 mg. In certain embodiments, the amount of the copovidone in the pharmaceutical composition is about 38 mg. In certain embodiments, the amount of the copovidone in the pharmaceutical composition is about 39 mg. In certain embodiments, the amount of the copovidone in the pharmaceutical composition is about 40 mg. In certain embodiments, the amount of the copovidone in the pharmaceutical composition is about 41 mg. In certain embodiments, the amount of the copovidone in the pharmaceutical composition is about 42 mg. In certain embodiments, the amount of the copovidone in the pharmaceutical composition is about 43 mg. In certain embodiments, the amount of the copovidone in the pharmaceutical composition is about 44 mg. In certain embodiments, the amount of the copovidone in the pharmaceutical composition is about 45 mg. In certain embodiments, the amount of the copovidone in the pharmaceutical composition is about 46 mg. In certain embodiments, the amount of the copovidone in the pharmaceutical composition is about 47 mg. In certain embodiments, the amount of the copovidone in the pharmaceutical composition is about 48 mg. In certain embodiments, the amount of the copovidone in the pharmaceutical composition is about 49 mg. In certain embodiments, the amount of the copovidone in the pharmaceutical composition is about 50 mg.

### (4) Polyethylene Glycol (PEG)

In these and other embodiments, a pharmaceutical composition described herein comprises a PEG. PEGs suitable for use in a pharmaceutical composition described herein include, but are not limited to, PEG 200, PEG 300, PEG 400, PEG 500, PEG 600, PEG 700, PEG 800, PEG 900, PEG 1000, PEG 1500, PEG 2000, PEG 3000, PEG 4000, PEG 5000, PEG 6000, PEG 7000, PEG 8000, PEG 9000, and PEG 10000. In certain embodiments, the PEG is selected from PEG 400 and PEG 1000. In certain embodiments, the PEG is PEG 400. In certain embodiments, the PEG is PEG 1000. In certain embodiments, the PEG has an average molecular weight of about 100 to about 10000, about 200 to about 10000, about 300 to about 10000, about 400 to about 10000, about 500 to about 10000, about 600 to about 10000, about 700 to about 10000, about 800 to about 10000, about 900 to about 10000, about 1000 to about 10000, about 100 to about 1000, about 100 to about 900, about 100 to about 800, about 100 to about 700, about 100 to about 600, about 100 to about 500, about 100 to about 400, about 100 to about 300, about 100 to about 200, about 200 to about 1000, about 200 to about 900, about 200 to about 800, about 200 to about 700, about 200 to about 600, about 200 to about 500, about 200 to about 400, about 200 to about 300, about 300 to about 1000, about 300 to about 900, about 300 to about 800, about 300 to about 700, about 300 to about 600, about 300 to about 500, about 300 to about 400, about 400 to about 1000, about 400 to about 900, about 400 to about 800, about 400 to about 700, about 400 to about 600, about 400 to about 500, about 500 to about 1500, about 600 to about 1400, about 700 to about 1300, and about 800 to about 1200. In certain embodiments, the PEG has an average molecular weight of about 380 to about 420. In certain embodiments, the PEG has an average molecular weight of about 570 to about 630. In certain embodiments, the PEG has an average molecular weight of about 950 to about 1000.

In these and other embodiments, the amount of the PEG in the pharmaceutical composition is about 1% (w/w) to about 40% (w/w), about 1% (w/w) to about 30% (w/w), about 1% (w/w) to about 20% (w/w), about 1% (w/w) to about 10% (w/w), about 10% (w/w) to about 40% (w/w), about 10% (w/w) to about 30% (w/w), about 10% (w/w) to about 20% (w/w), about 10% (w/w) to about 15% (w/w), about 11% (w/w) to about 19% (w/w), about 11% (w/w) to about 17% (w/w), about 11% (w/w) to about 15% (w/w), about 11% (w/w) to about 13% (w/w), about 12% (w/w) to about 18% (w/w), about 12% (w/w) to about 16% (w/w), about 12% (w/w) to about 14% (w/w), about 13% (w/w) to about 18% (w/w), about 14% (w/w) to about 18% (w/w), about 15% (w/w) to about 18% (w/w), or about 16% (w/w) to about 17% (w/w). In certain embodiments, the amount of PEG in the pharmaceutical composition is about 10% (w/w) to about 20% (w/w). In certain embodiments, the amount of the PEG in the pharmaceutical composition is about 11% (w/w) to about 20% (w/w). In certain embodiments, the amount of the PEG in the pharmaceutical composition is about 11% (w/w) to about 17% (w/w). In certain embodiments, the amount of the PEG in the pharmaceutical composition is about 11% (w/w) to about 15% (w/w). In certain embodiments, the amount of the PEG in the pharmaceutical composition is about 11% (w/w) to about 13% (w/w). In certain embodiments, the amount of the PEG in the pharmaceutical composition is about 12% (w/w) to about 20% (w/w). In certain embodiments, the amount of the PEG in the pharmaceutical composition is about 12% (w/w) to about 18% (w/w). In certain embodiments, the amount of the PEG in the pharmaceutical composition is about 12% (w/w) to about 16% (w/w). In certain embodiments, the amount of the PEG in the pharmaceutical composition is about 12% (w/w) to about 14% (w/w). In certain embodiments, the amount of the PEG in the pharmaceutical composition is about 13% (w/w) to about 20% (w/w). In certain embodiments, the amount of the PEG in the pharmaceutical composition is about 14% (w/w) to about 20% (w/w). In certain embodiments, the amount of the PEG in the pharmaceutical composition is about 14.5% (w/w) to about 19.5% (w/w). In certain embodiments, the amount of the PEG in the pharmaceutical composition is about 15% (w/w) to about 19% (w/w). In certain embodiments, the amount of the PEG in the pharmaceutical composition is about 15.5% (w/w) to about 18.5% (w/w). In certain embodiments, the amount of the PEG in the pharmaceutical composition is about 16% (w/w) to about 18% (w/w). In certain embodiments, the amount of the PEG in the pharmaceutical composition is about 16.5% (w/w) to about 17.5% (w/w).

In these and other embodiments, the amount of the PEG in the pharmaceutical composition is about 10% (w/w), about 10.5% (w/w), about 11% (w/w), about 11.5% (w/w), about 12% (w/w), about 12.5% (w/w), about 13% (w/w), about 13.5% (w/w), about 14% (w/w), about 14.5% (w/w), about 15% (w/w), about 15.5% (w/w), about 16% (w/w), about 16.8% (w/w), about 17% (w/w), about 17.5% (w/w), about 18% (w/w), about 18.5% (w/w), about 19% (w/w), about 19.5% (w/w), about 20% (w/w), about 20.5% (w/w), about 210% (w/w), about 21.5% (w/w), or about 22% (w/w). In certain embodiments, the amount of the PEG in the pharmaceutical composition is about 10% (w/w). In certain embodiments, the amount of the PEG in the pharmaceutical composition is about 11% (w/w). In certain embodiments, the amount of the PEG in the pharmaceutical composition is about 12% (w/w). In certain embodiments, the amount of the PEG in the pharmaceutical composition is about 12.5% (w/w). In certain embodiments, the amount of the PEG in the pharmaceutical composition is about 14% (w/w). In certain embodiments, the amount of the PEG in the pharmaceutical composition is about 15% (w/w). In certain embodiments, the amount of the PEG in the pharmaceutical composition is about 15.5% (w/w). In certain embodiments, the amount of the PEG in the pharmaceutical composition is about 16% (w/w). In certain embodiments, the amount of the PEG in the pharmaceutical composition is about 16.8% (w/w). In certain embodiments, the amount of the PEG in the pharmaceutical composition is about 17% (w/w). In certain embodiments, the amount of the PEG in the pharmaceutical composition is about 17.5% (w/w). In certain embodiments, the amount of the PEG in the pharmaceutical composition is about 18% (w/w). In certain embodiments, the amount of the PEG in the pharmaceutical composition is about 18.5% (w/w). In certain embodiments, the amount of the PEG in the pharmaceutical composition is about 19% (w/w).

In these and other embodiments, the amount of the PEG in the pharmaceutical composition is about 20 mg to about 1000 mg, about 20 mg to about 900 mg, about 20 mg to about 800 mg, about 20 mg to about 700 mg, about 20 mg to about 600 mg, about 20 mg to about 500 mg, about 20 mg to about 400 mg, about 20 mg to about 300 mg, about 20 mg to about 250 mg, about 20 mg to about 200 mg, about 20 mg to about 150 mg, about 20 mg to about 100 mg, about 20 mg to about 75 mg, about 20 mg to about 50 mg, about 20 mg to about 25 mg, about 25 mg to about 1000 mg, about 25 mg to about 900 mg, about 25 mg to about 800 mg, about 25 mg to about 700 mg, about 25 mg to about 600 mg, about 25 mg to about 500 mg, about 25 mg to about 400 mg, about 25 mg to about 300 mg, about 25 mg to about 250 mg, about 25 mg to about 200 mg, about 25 mg to about 150 mg, about 25 mg to about 100 mg, about 25 mg to about 75 mg, about 25 mg to about 50 mg, about 50 mg to about 900 mg, about 50 mg to about 800 mg, about 50 mg to about 700 mg, about 50 mg to about 600 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 50 mg to about 75 mg, about 75 mg to about 900 mg, about 75 mg to about 800 mg, about 75 mg to about 700 mg, about 75 mg to about 600 mg, about 75 mg to about 500 mg, about 75 mg to about 400 mg, about 75 mg to about 300 mg, or about 75 mg to about 200 mg. In certain embodiments, the amount of the PEG in the pharmaceutical composition is about 20 mg to about 200 mg, about 20 mg to about 150 mg, about 20 mg to about 120 mg, about 20 mg to about 100 mg, about 20 mg to about 75 mg, about 20 mg to about 50 mg, about 50 mg to about 75 mg, or about 75 mg to about 100 mg. In certain embodiments, the amount of the PEG in the pharmaceutical composition is about 90 mg to about 190 mg, about 100 mg to about 180 mg, about 110 mg to about 170 mg, about 120 mg to about 160 mg, about 125 mg to about 155 mg, about 130 mg to about 150 mg, about 135 mg to about 145 mg, about 136 mg to about 144 mg, about 137 mg to about 143 mg, about 138 mg to about 142 mg, or about 139 mg to about 141 mg. In certain embodiments, the amount of the PEG in the pharmaceutical composition is about 40 mg to about 100 mg, about 50 mg to about 90 mg, about 55 mg to about 85 mg, about 60 mg to about 80 mg, about 65 mg to about 75 mg, about 66 mg to about 74 mg, about 67 mg to about 73 mg, about 68 mg to about 72 mg, or about 69 mg to about 71 mg. In certain embodiments, the amount of the PEG in the pharmaceutical composition is about 20 mg to about 200 mg. In certain embodiments, the amount of the PEG in the pharmaceutical composition is about 20 mg to about 150 mg. In certain embodiments, the amount of the PEG in the pharmaceutical composition is about 50 mg to about 200 mg. In certain embodiments, the amount of the PEG in the pharmaceutical composition is about 100 mg to about 150 mg.

In these and other embodiments, the amount of the PEG in the pharmaceutical composition is about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, 101 mg, 102 mg, 103 mg, 104 mg, 105 mg, 106 mg, 107 mg, 108 mg, 109 mg, 110 mg, about 111 mg, about 112 mg, about 113 mg, about 114 mg, about 115 mg, about 116 mg, about 117 mg, about 118 mg, about 119 mg, 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, or about 160 mg. In certain embodiments, the amount of the PEG in the pharmaceutical composition is about 24 mg. In certain embodiments, the amount of the PEG in the pharmaceutical composition is about 25 mg. In certain embodiments, the amount of the PEG in the pharmaceutical composition is about 26 mg. In certain embodiments, the amount of the PEG in the pharmaceutical composition is about 27 mg. In certain embodiments, the amount of the PEG in the pharmaceutical composition is about 28 mg. In certain embodiments, the amount of the PEG in the pharmaceutical composition is about 51 mg. In certain embodiments, the amount of the PEG in the pharmaceutical composition is about 52 mg. In certain embodiments, the amount of the PEG in the pharmaceutical composition is about 53 mg. In certain embodiments, the amount of the PEG in the pharmaceutical composition is about 54 mg. In certain embodiments, the amount of the PEG in the pharmaceutical composition is about 55 mg. In certain embodiments, the amount of the PEG in the pharmaceutical composition is about 68 mg. In certain embodiments, the amount of the PEG in the pharmaceutical composition is about 69 mg. In certain embodiments, the amount of the PEG in the pharmaceutical composition is about 70 mg. In certain embodiments, the amount of the PEG in the pharmaceutical composition is about 71 mg. In certain embodiments, the amount of the PEG in the pharmaceutical composition is about 72 mg. In certain embodiments, the amount of the PEG in the pharmaceutical composition is about 73 mg. In certain embodiments, the amount of the PEG in the pharmaceutical composition is about 77 mg. In certain embodiments, the amount of the PEG in the pharmaceutical composition is about 78 mg. In certain embodiments, the amount of the PEG in the pharmaceutical composition is about 79 mg. In certain embodiments, the amount of the PEG in the pharmaceutical composition is about 80 mg. In certain embodiments, the amount of the PEG in the pharmaceutical composition is about 81 mg. In certain embodiments, the amount of the PEG in the pharmaceutical composition is about 82 mg. In certain embodiments, the amount of the PEG in the pharmaceutical composition is about 104 mg. In certain embodiments, the amount of the PEG in the pharmaceutical composition is about 105 mg. In certain embodiments, the amount of the PEG in the pharmaceutical composition is about 106 mg. In certain embodiments, the amount of the PEG in the pharmaceutical composition is about 107 mg. In certain embodiments, the amount of the PEG in the pharmaceutical composition is about 108 mg. In certain embodiments, the amount of the PEG in the pharmaceutical composition is about 109 mg. In certain embodiments, the amount of the PEG in the pharmaceutical composition is about 110 mg. In certain embodiments, the amount of the PEG in the pharmaceutical composition is about 138 mg. In certain embodiments, the amount of the PEG in the pharmaceutical composition is about 139 mg. In certain embodiments, the amount of the PEG in the pharmaceutical composition is about 140 mg. In certain embodiments, the amount of the PEG in the pharmaceutical composition is about 141 mg. In certain embodiments, the amount of the PEG in the pharmaceutical composition is about 142 mg.

### (5) Antioxidants

In these and other embodiments, a pharmaceutical composition described herein comprises an antioxidant. In certain embodiments, the antioxidant is butylated hydroxytoluene (BHT). In certain embodiments, a pharmaceutical composition described herein comprises BHT.

In these and other embodiments, the amount of BHT in the pharmaceutical composition is about 0.01% (w/w) to about 5% (w/w), 0.01% (w/w) to about 4% (w/w), 0.01% (w/w) to about 3% (w/w), 0.01% (w/w) to about 2% (w/w), 0.01% (w/w) to about 1% (w/w), 0.01% (w/w) to about 0.9% (w/w), 0.01% (w/w) to about 0.8% (w/w), 0.01% (w/w) to about 0.7% (w/w), 0.01% (w/w) to about 0.6% (w/w), 0.01% (w/w) to about 0.5% (w/w), 0.01% (w/w) to about 0.4% (w/w), 0.01% (w/w) to about 0.3% (w/w), 0.01% (w/w) to about 0.2% (w/w), 0.02% (w/w) to about 0.2% (w/w), 0.03% (w/w) to about 0.2% (w/w), 0.04% (w/w) to about 0.2% (w/w), 0.05% (w/w) to about 0.2% (w/w), 0.06% (w/w) to about 0.2% (w/w), 0.07% (w/w) to about 0.2% (w/w), 0.08% (w/w) to about 0.2% (w/w), 0.09% (w/w) to about 0.2% (w/w), 0.05% (w/w) to about 0.15% (w/w), 0.06% (w/w) to about 0.14% (w/w), 0.07% (w/w) to about 0.13% (w/w), 0.08% (w/w) to about 0.12% (w/w), or 0.09% (w/w) to about 0.11% (w/w). In certain embodiments, the amount of BHT in the pharmaceutical composition is about 0.05% (w/w) to about 0.15% (w/w). In certain embodiments, the amount of BHT in the pharmaceutical composition is about 0.06% (w/w) to about 0.14% (w/w). In certain embodiments, the amount of BHT in the pharmaceutical composition is about 0.07% (w/w) to about 0.13% (w/w). In certain embodiments, the amount of BHT in the pharmaceutical composition is about 0.08% (w/w) to about 0.12% (w/w). In certain embodiments, the amount of BHT in the pharmaceutical composition is about 0.09% (w/w) to about 0.11% (w/w).

In these and other embodiments, the amount of BHT in the pharmaceutical composition is about 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.11% (w/w), 0.12% (w/w), 0.13% (w/w), 0.14% (w/w), 0.15% (w/w), 0.16% (w/w), 0.17% (w/w), 0.18% (w/w), 0.19% (w/w), or 0.20% (w/w). In certain embodiments, the amount of BHT in the pharmaceutical composition is about 0.07% (w/w). In certain embodiments, the amount of BHT in the pharmaceutical composition is about 0.08% (w/w). In certain embodiments, the amount of BHT in the pharmaceutical composition is about 0.09% (w/w). In certain embodiments, the amount of BHT in the pharmaceutical composition is about 0.1% (w/w). In certain embodiments, the amount of BHT in the pharmaceutical composition is about 0.11% (w/w). In certain embodiments, the amount of BHT in the pharmaceutical composition is about 0.12% (w/w). In certain embodiments, the amount of BHT in the pharmaceutical composition is about 0.13% (w/w).

In these and other embodiments, the amount of BHT in the pharmaceutical composition is about 0.20 mg to about 10 mg, about 0.20 mg to about 9.0 mg, about 0.20 mg to about 8.0 mg, about 0.20 mg to about 7.0 mg, about 0.20 mg to about 6.0 mg, about 0.20 mg to about 5.0 mg, about 0.20 mg to about 4.0 mg, about 0.20 mg to about 3.0 mg, about 0.20 mg to about 2.5 mg, about 0.20 mg to about 2.0 mg, about 0.20 mg to about 1.5 mg, about 0.20 mg to about 1.0 mg, about 0.20 mg to about 0.5 mg, about 0.25 mg to about 10 mg, about 0.25 mg to about 9.0 mg, about 0.25 mg to about 8.0 mg, about 0.25 mg to about 7.0 mg, about 0.25 mg to about 6.0 mg, about 0.25 mg to about 5.0 mg, about 0.25 mg to about 4.0 mg, about 0.25 mg to about 3.0 mg, about 0.25 mg to about 2.5 mg, about 0.25 mg to about 2.0 mg, about 0.25 mg to about 1.5 mg, about 0.25 mg to about 1.00 mg, about 0.50 mg to about 9.0 mg, about 0.50 mg to about 8.0 mg, about 0.50 mg to about 7.0 mg, about 0.50 mg to about 6.0 mg, about 0.50 mg to about 5.0 mg, about 0.50 mg to about 4.0 mg, about 0.50 mg to about 3.0 mg, about 0.50 mg to about 2.50 mg, about 0.50 mg to about 2.0 mg, about 0.50 mg to about 1.50 mg, or about 0.50 mg to about 1.00 mg.

In these and other embodiments, the amount of BHT in the pharmaceutical composition is about 0.20 mg to about 0.50 mg, about 0.30 mg to about 0.50 mg, about 0.40 mg to about 0.50 mg, about 0.20 mg to about 0.40 mg, about 0.20 mg to about 0.30 mg, or about 0.30 mg to about 0.40 mg.

In these and other embodiments, the amount of BHT in the pharmaceutical composition is about 0.32 mg to about 0.52 mg, about 0.33 mg to about 0.51 mg, about 0.34 mg to about 0.50 mg, about 0.35 mg to about 0.49 mg, about 0.36 mg to about 0.48 mg, about 0.37 mg to about 0.47 mg, about 0.38 mg to about 0.46 mg, about 0.39 mg to about 0.45 mg, about 0.40 mg to about 0.44 mg, or about 0.41 mg to about 0.43 mg.

In these and other embodiments, the amount of BHT in the pharmaceutical composition is about 0.73 mg to about 0.93 mg, about 0.74 mg to about 0.92 mg, about 0.75 mg to about 0.91 mg, about 0.76 mg to about 0.90 mg, about 0.77 mg to about 0.89 mg, about 0.78 mg to about 0.88 mg, about 0.79 mg to about 0.87 mg, about 0.80 mg to about 0.86 mg, about 0.81 mg to about 0.85 mg, or about 0.82 mg to about 0.84 mg.

In these and other embodiments, the amount of BHT in the pharmaceutical composition is about 0.01 mg, about 0.02 mg, about 0.03 mg, about 0.04 mg, about 0.05 mg, about 0.06 mg, about 0.07 mg, about 0.08 mg, about 0.09 mg, about 0.10 mg, about 0.11 mg, about 0.12 mg, about 0.13 mg, about 0.14 mg, about 0.15 mg, about 0.16 mg, about 0.17 mg, about 0.18 mg, about 0.19 mg, about 0.20 mg, about 0.21 mg, about 0.22 mg, about 0.23 mg, about 0.24 mg, about 0.25 mg, about 0.26 mg, about 0.27 mg, about 0.28 mg, about 0.29 mg, or about 0.30 mg, about 0.31 mg, about 0.32 mg, about 0.33 mg, about 0.34 mg, about 0.35 mg, about 0.36 mg, about 0.37 mg, about 0.38 mg, about 0.39 mg, about 0.40 mg, about 0.41 mg, about 0.42 mg, about 0.43 mg, about 0.44 mg, about 0.45 mg, about 0.46 mg, about 0.47 mg, about 0.48 mg, about 0.49 mg, about 0.50 mg, about 0.51 mg, about 0.52 mg, about 0.53 mg, about 0.54 mg, about 0.55 mg, about 0.56 mg, about 0.57 mg, about 0.58 mg, about 0.59 mg, about 0.60 mg, about 0.61 mg, about 0.62 mg, about 0.63 mg, about 0.64 mg, about 0.65 mg, about 0.66 mg, about 0.67 mg, about 0.68 mg, about 0.69 mg, about 0.70 mg, about 0.71 mg, about 0.72 mg, about 0.73 mg, about 0.74 mg, about 0.75 mg, about 0.76 mg, about 0.77 mg, about 0.78 mg, about 0.79 mg, about 0.80 mg, about 0.81 mg, about 0.82 mg, about 0.83 mg, about 0.84 mg, about 0.85 mg, about 0.86 mg, about 0.87 mg, about 0.88 mg, about 0.89 mg, about 0.90 mg, about 0.91 mg, about 0.92 mg, about 0.93 mg, about 0.94 mg, about 0.95 mg, about 0.96 mg, about 0.97 mg, about 0.98 mg, about 0.99 mg, or about 1.0 mg.

### (6) One or More Additional Pharmaceutically Acceptable Excipients

In these and other embodiments, a pharmaceutical composition described herein comprises one or more additional pharmaceutically acceptable excipients. In certain embodiments, the one or more additional pharmaceutical excipients is a pharmaceutically acceptable excipient as described in the “Definitions” section. In some embodiments, one or more additional pharmaceutical excipients is a dissolution aid.

### (7) Exemplary Pharmaceutical Compositions

In various embodiments, a pharmaceutical composition comprises:
(a) about 2% (w/w) to about 15% (w/w) inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a pharmaceutical composition comprises:
(a) about 4% (w/w) to about 6% (w/w) inupadenant hydrochloride;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone; and
(e) about 0.08% (w/w) to about 0.12% (w/w) BHT.

In various embodiments, a pharmaceutical composition comprises:
(a) about 2% (w/w) to about 15% (w/w) crystalline inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a pharmaceutical composition comprises:
(a) about 4% (w/w) to about 6% (w/w) crystalline inupadenant hydrochloride;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone; and
(e) about 0.08% (w/w) to about 0.12% (w/w) BHT.

In various embodiments, a pharmaceutical composition comprises:
(a) about 2% (w/w) to about 15% (w/w) inupadenant hydrochloride hydrate;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a pharmaceutical composition comprises:
(a) about 4% (w/w) to about 6% (w/w) inupadenant hydrochloride hydrate;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone; and
(e) about 0.08% (w/w) to about 0.12% (w/w) BHT.

In various embodiments, a pharmaceutical composition comprises:
(a) about 2% (w/w) to about 15% (w/w) crystalline inupadenant hydrochloride hydrate;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a pharmaceutical composition comprises:
(a) about 4% (w/w) to about 6% (w/w) crystalline inupadenant hydrochloride hydrate;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone; and
(e) about 0.08% (w/w) to about 0.12% (w/w) BHT.

In various embodiments, a pharmaceutical composition comprises:
(a) about 2% (w/w) to about 15% (w/w) Form 1 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a pharmaceutical composition comprises:
(a) about 4% (w/w) to about 6% (w/w) Form 1 inupadenant hydrochloride;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone; and
(e) about 0.08% (w/w) to about 0.12% (w/w) BHT.

In various embodiments, a pharmaceutical composition comprises:
(a) about 2% (w/w) to about 15% (w/w) Form 2 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a pharmaceutical composition comprises:
(a) about 4% (w/w) to about 6% (w/w) Form 2 inupadenant hydrochloride;
(b) about 65% (w/w) to about 85% (w/w) of lauroyl macrogol-32 glycerides;
(c) about 12% (w/w) to about 18% (w/w) of a PEG;
(d) about 3% (w/w) to about 6% (w/w) of a copovidone; and
(e) about 0.08% (w/w) to about 0.12% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 2% (w/w) and 15% (w/w) inupadenant, between 50% (w/w) and 90% (w/w) of lauroyl macrogol-32 glycerides, between 10% (w/w) and 20% (w/w) a PEG (e.g., PEG 400 or PEG 1000), between 2% (w/w) and 8% (w/w) of a copovidone and between 0.05% (w/w) and 0.2% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 2% (w/w) and 15% (w/w) inupadenant, between 50% (w/w) and 90% (w/w) of lauroyl macrogol-32 glycerides, between 10% (w/w) and 20% (w/w) PEG 400, between 2% (w/w) and 8% (w/w) of a copovidone, and between 0.05% (w/w) and 0.2% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 2% (w/w) and 15% (w/w) inupadenant, between 50% (w/w) and 90% (w/w) of lauroyl macrogol-32 glycerides, between 10% (w/w) and 20% (w/w) PEG 1000, between 2% (w/w) and 8% (w/w) of a copovidone, and between 0.05% (w/w) and 0.2% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 4% (w/w) and 6% w/w inupadenant, between 60% (w/w) and 80% (w/w) of lauroyl macrogol-32 glycerides, between 14% (w/w) and 18% (w/w) of a PEG (e.g., PEG 400 or PEG 1000), between 3% (w/w) and 6% (w/w) of a copovidone, and between 0.08% (w/w) and 0.12% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride, wherein the pharmaceutical composition comprises between 4% (w/w) and 6% (w/w) inupadenant, between 65% (w/w) and 85% (w/w) of lauroyl macrogol-32 glycerides, between 12% (w/w) and 18% (w/w) of a PEG (e.g., PEG 400 or PEG 1000), between 1% (w/w) and 6% (w/w) of a copovidone, and between 0.08% (w/w) and 0.12% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 4% (w/w) and 6% (w/w) inupadenant, between 65% (w/w) and 85% (w/w) of lauroyl macrogol-32 glycerides, between 12% (w/w) and 18% (w/w) PEG 400, between 1% (w/w) and 6% (w/w) of a copovidone, and between 0.08% (w/w) and 0.12% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 4% (w/w) and 6% (w/w) inupadenant, between 65% (w/w) and 85% (w/w) of lauroyl macrogol-32 glycerides, between 12% (w/w) and 18% (w/w) PEG 1000, between 1% (w/w) and 6% (w/w) of a copovidone, and between 0.08% (w/w) and 0.12% (w/w) BHT.

In various embodiments, provided herein is a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises about 5.1% (w/w) inupadenant, about 73.4% (w/w) of lauroyl macrogol-32 glycerides, about 16.8% (w/w) of a PEG (e.g., PEG 400 or PEG 1000), about 4.6% (w/w) of a copovidone, and about 0.1% (w/w) BHT.

### Dosage Forms

In one aspect, provided herein are dosage forms comprising a pharmaceutical composition described herein. In certain embodiments, the dosage form is a solid dosage form. In certain embodiments, the dosage form is an oral dosage form.

In another aspect, provided herein are dosage forms intended for oral administration comprising a pharmaceutical composition described herein.

In certain embodiments, the dosage form is selected from the group consisting of a powder, a sachet, a stickpack, a capsule, a minitab, and a tablet.

In certain embodiments, the dosage form is a capsule. In certain embodiments, the capsule is a gel capsule. In certain embodiments, the capsule is a hard gel capsule. In certain embodiments, the size of the capsule is selected from the group consisting of 000, 00, 0, 1, 2, 3, 4, and 5.

In certain embodiments, the total weight of the pharmaceutical composition in the capsule is about 25 mg to about 1000 mg, about 50 mg to about 1000 mg, about 75 mg to about 1000 mg, about 100 mg to about 1000 mg, about 150 mg to about 1000 mg, about 200 mg to about 1000 mg, about 250 mg to about 1000 mg, about 300 mg to about 1000 mg, about 400 mg to about 1000 mg, about 500 mg to about 1000 mg, about 600 mg to about 1000 mg, about 700 mg to about 1000 mg, about 800 mg to about 1000 mg, about 900 mg to about 1000 mg, about 25 mg to about 900 mg, about 25 mg to about 800 mg, about 25 mg to about 700 mg, about 25 mg to about 600 mg, about 25 mg to about 500 mg, about 25 mg to about 400 mg, about 25 mg to about 300 mg, about 25 mg to about 250 mg, about 25 mg to about 200 mg, about 25 mg to about 150 mg, about 25 mg to about 100 mg, about 25 mg to about 75 mg, about 25 mg to about 50 mg, about 50 mg to about 900 mg, about 50 mg to about 800 mg, about 50 mg to about 700 mg, about 50 mg to about 600 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 50 mg to about 75 mg, about 75 mg to about 900 mg, about 75 mg to about 800 mg, about 75 mg to about 700 mg, about 75 mg to about 600 mg, about 75 mg to about 500 mg, about 75 mg to about 400 mg, about 75 mg to about 300 mg, about 75 mg to about 200 mg, about 75 mg to about 100 mg, about 100 mg to about 900 mg, about 100 mg to about 800 mg, about 100 mg to about 700 mg, about 100 mg to about 600 mg, about 100 mg to about 500 mg, about 100 mg to about 400 mg, about 100 mg to about 300 mg, about 100 mg to about 250 mg, about 100 mg to about 200 mg, about 100 mg to about 150 mg, about 150 mg to about 900 mg, about 150 mg to about 800 mg, about 150 mg to about 700 mg, about 150 mg to about 600 mg, about 150 mg to about 500 mg, about 150 mg to about 400 mg, about 150 mg to about 300 mg, about 150 mg to about 250 mg, about 150 mg to about 200 mg, about 200 mg to about 900 mg, about 200 mg to about 800 mg, about 200 mg to about 700 mg, about 200 mg to about 600 mg, about 200 mg to about 500 mg, about 200 mg to about 400 mg, about 200 mg to about 300 mg, about 200 mg to about 250 mg, about 250 mg to about 900 mg, about 250 mg to about 800 mg, about 250 mg to about 700 mg, about 250 mg to about 600 mg, about 250 mg to about 500 mg, about 250 mg to about 400 mg, about 250 mg to about 300 mg, about 300 mg to about 900 mg, about 300 mg to about 800 mg, about 300 mg to about 700 mg, about 300 mg to about 600 mg, about 300 mg to about 500 mg, about 300 mg to about 400 mg, about 400 mg to about 900 mg, about 400 mg to about 800 mg, about 400 mg to about 700 mg, about 400 mg to about 600 mg, about 400 mg to about 500 mg, about 500 mg to about 900 mg, about 500 mg to about 800 mg, about 500 mg to about 700 mg, about 500 mg to about 600 mg, about 600 mg to about 900 mg, about 600 mg to about 800 mg, about 600 mg to about 700 mg, about 700 mg to about 900 mg, about 700 mg to about 800 mg, or about 800 mg to about 900 mg. In certain embodiments, the total weight of the pharmaceutical composition in the capsule is about 200 mg to about 1000 mg. In certain embodiments, the total weight of the pharmaceutical composition in the capsule is about 300 mg to about 500 mg. In certain embodiments, the total weight of the pharmaceutical composition in the capsule is about 350 mg to 450 mg. In certain embodiments, the total weight of the pharmaceutical composition in the capsule is about 700 mg to 900 mg. In certain embodiments, the total weight of the pharmaceutical composition in the capsule is about 780 mg to 880 mg. In certain embodiments, the total weight of the pharmaceutical composition in the capsule is about 150 mg to 300 mg. In certain embodiments, the total weight of the pharmaceutical composition in the capsule is about 200 mg to 250 mg.

In certain embodiments, the total weight of the pharmaceutical composition in the capsule is about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 240 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg or about 1090 mg. In certain embodiments, the total weight of the pharmaceutical composition in the capsule is about 200 mg. In certain embodiments, the total weight of the pharmaceutical composition in the capsule is about 205 mg. In certain embodiments, the total weight of the pharmaceutical composition in the capsule is about 210 mg. In certain embodiments, the total weight of the pharmaceutical composition in the capsule is about 215 mg. In certain embodiments, the total weight of the pharmaceutical composition in the capsule is about 220 mg. In certain embodiments, the total weight of the pharmaceutical composition in the capsule is about 225 mg. In certain embodiments, the total weight of the pharmaceutical composition in the capsule is about 230 mg. In certain embodiments, the total weight of the pharmaceutical composition in the capsule is about 400 mg. In certain embodiments, the total weight of the pharmaceutical composition in the capsule is about 405 mg. In certain embodiments, the total weight of the pharmaceutical composition in the capsule is about 410 mg. In certain embodiments, the total weight of the pharmaceutical composition in the capsule is about 415 mg. In certain embodiments, the total weight of the pharmaceutical composition in the capsule is about 420 mg. In certain embodiments, the total weight of the pharmaceutical composition in the capsule is about 425 mg. In certain embodiments, the total weight of the pharmaceutical composition in the capsule is about 820 mg. In certain embodiments, the total weight of the pharmaceutical composition in the capsule is about 825 mg. In certain embodiments, the total weight of the pharmaceutical composition in the capsule is about 830 mg. In certain embodiments, the total weight of the pharmaceutical composition in the capsule is about 835 mg. In certain embodiments, the total weight of the pharmaceutical composition in the capsule is about 840 mg. In certain embodiments, the total weight of the pharmaceutical composition in the capsule is about 845 mg. In certain embodiments, the total weight of the pharmaceutical composition in the capsule is about 831 mg.

In certain embodiments, the weight of pharmaceutical composition in the capsule is about 207.82 mg. In certain embodiments, the weight of pharmaceutical composition in the capsule is about 415.63 mg. In certain embodiments, the weight of pharmaceutical composition in the capsule is about 831.25 mg.

In certain embodiments, the pharmaceutical composition is present in the capsule as a solid composition or a semi-solid composition. In certain embodiments, the inupadenant hydrochloride is present in the pharmaceutical composition as amorphous inupadenant hydrochloride and/or crystalline inupadenant hydrochloride. In certain embodiments, the amorphous inupadenant hydrochloride is an amorphous inupadenant hydrochloride described herein. In certain embodiments, the inupadenant hydrochloride is a crystalline inupadenant hydrochloride described herein. In certain embodiments, the inupadenant hydrochloride is an inupadenant hydrochloride hydrate. In certain embodiments, the inupadenant hydrochloride hydrate is a crystalline inupadenant hydrochloride hydrate described herein.

### Exemplary Dosage Forms

In various embodiments, a capsule comprising a pharmaceutical composition, wherein the pharmaceutical composition comprises:
(a) about 2% (w/w) to about 15% (w/w) inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a capsule comprising a pharmaceutical composition, wherein the pharmaceutical composition comprises:
(a) about 4% (w/w) to about 6% (w/w) inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, a capsule comprising a pharmaceutical composition, wherein the pharmaceutical composition comprises:
(a) about 2% (w/w) to about 15% (w/w) crystalline inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a capsule comprising a pharmaceutical composition, wherein the pharmaceutical composition comprises:
(a) about 4% (w/w) to about 6% (w/w) crystalline inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, a capsule comprising a pharmaceutical composition, wherein the pharmaceutical composition comprises:
(a) about 2% (w/w) to about 15% (w/w) crystalline inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a capsule comprising a pharmaceutical composition, wherein the pharmaceutical composition comprises:
(a) about 4% (w/w) to about 6% (w/w) crystalline inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, a capsule comprising a pharmaceutical composition, wherein the pharmaceutical composition comprises:
(a) about 2% (w/w) to about 15% (w/w) crystalline inupadenant hydrochloride hydrate;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a capsule comprising a pharmaceutical composition, wherein the pharmaceutical composition comprises:
(a) about 4% (w/w) to about 6% (w/w) crystalline inupadenant hydrochloride hydrate;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, a capsule comprising a pharmaceutical composition, wherein the pharmaceutical composition comprises:
(a) about 2% (w/w) to about 15% (w/w) Form 1 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a capsule comprising a pharmaceutical composition, wherein the pharmaceutical composition comprises:
(a) about 4% (w/w) to about 6% (w/w) Form 1 inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, a capsule comprising a pharmaceutical composition, wherein the pharmaceutical composition comprises:
(a) about 2% (w/w) to about 15% (w/w) Form 2 inupadenant hydrochloride;
(b) about 50% (w/w) to about 90% (w/w) lauroyl macrogol-32 glycerides;
(c) about 10% (w/w) to about 20% (w/w) of a PEG;
(d) about 2% (w/w) to about 8% (w/w) of a copovidone; and
(e) about 0.05% (w/w) to about 0.2% (w/w) BHT.

In various embodiments, a capsule comprising a pharmaceutical composition, wherein the pharmaceutical composition comprises:
(a) about 4% (w/w) to about 6% (w/w) Form 1 inupadenant hydrochloride;
(b) about 65% (w/w) to about 85% (w/w) lauroyl macrogol-32 glycerides;
(c) about 12% (w/w) to about 18% (w/w) of a PEG;
(d) about 1% (w/w) to about 6% (w/w) of a copovidone; and
(e) about 0.08% (w/w) to about 0.12% (w/w) BHT.

In various embodiments, provided herein is a capsule dosage form comprising a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 2% (w/w) and 15% (w/w) inupadenant, between 50% (w/w) and 90% (w/w) of lauroyl macrogol-32 glycerides, between 10% (w/w) and 20% (w/w) of a PEG (e.g., PEG 400 or PEG 1000), between 2% (w/w) and 8% (w/w) of a copovidone, and between 0.05% (w/w) and 0.2% (w/w) BHT.

In various embodiments, provided herein is a capsule dosage form comprising a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 2% (w/w) and 15% (w/w) inupadenant, between 50% (w/w) and 90% (w/w) of lauroyl macrogol-32 glycerides, between 10% (w/w) and 20% (w/w) PEG 400, between 2% (w/w) and 8% (w/w) of a copovidone, and between 0.05% (w/w) and 0.2% (w/w) BHT.

In various embodiments, provided herein is a capsule dosage form comprising a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 4% (w/w) and 6% (w/w) inupadenant, between 60% (w/w) and 80% (w/w) of lauroyl macrogol-32 glycerides, between 14% (w/w) and 18% (w/w) of a PEG (e.g., PEG 400 or PEG 1000), between 3% (w/w) and 6% (w/w) of a copovidone, and between 0.08% (w/w) and 0.12% (w/w) BHT.

In various embodiments, provided herein is a capsule dosage form comprising a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 4% (w/w) and 6% (w/w) inupadenant, between 65% (w/w) and 85% (w/w) of lauroyl macrogol-32 glycerides, between 12% (w/w) and 18% (w/w) of a PEG (e.g., PEG 400 or PEG 1000), between 1% (w/w) and 6% (w/w) of a copovidone, and between 0.08% (w/w) and 0.12% (w/w) BHT.

In various embodiments, provided herein is a capsule dosage form comprising a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 4% (w/w) and 6% (w/w) inupadenant, between 65% (w/w) and 85% (w/w) of lauroyl macrogol-32 glycerides, between 12% (w/w) and 18% (w/w) PEG 400, between 1% (w/w) and 6% (w/w) of a copovidone, and between 0.08% (w/w) and 0.12% (w/w) BHT.

In various embodiments, provided herein is a capsule dosage form comprising a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises between 4% (w/w) and 6% (w/w) inupadenant, between 65% (w/w) and 85% (w/w) of lauroyl macrogol-32 glycerides, between 12% (w/w) and 18% (w/w) PEG 1000, between 1% (w/w) and 6% (w/w) of a copovidone, and between 0.08% (w/w) and 0.12% (w/w) BHT.

In various embodiments, provided herein is a capsule dosage form comprising a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), wherein the pharmaceutical composition comprises about 5.1% (w/w) inupadenant, about 73.4% (w/w) of lauroyl macrogol-32 glycerides, about 16.8% (w/w) of a PEG (e.g., PEG 400 or PEG 1000), about 4.6% (w/w) of a copovidone, and about 0.1% (w/w) of BHT.

### Methods of Making

The pharmaceutical composition of the invention may be manufactured by methods well known by one skilled in the art.

In one embodiment, the pharmaceutical composition of the invention is under solid or semi-solid form.

In one embodiment, the pharmaceutical composition of the invention is under the form of capsules, preferably hard gelatin capsules. In such case, the capsules may be manufactured from a common blend using conventional mixing and capsule filling processes according to Good Manufacturing Practice.

The pharmaceutical composition described herein may be manufactured by methods well known by one skilled in the art.

In one aspect, provided herein is a process for manufacturing a capsule comprising a pharmaceutical composition described herein, the process generally comprising the steps of:
(i) melting a lipid carrier (e.g., lauroyl polyoxyl-32 glycerides) at a temperature not less than 50° C. but not exceeding 80° C.;
(ii) adding a PEG (e.g., PEG 400 or PEG 1000) to the lipid carrier (e.g., lauroyl polyoxyl-32 glycerides) and mixing together;
(iii) adding copovidone and an antioxidant (e.g., BHT), with continued mixing;
(iv) adding the inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride) gradually under continuous mixing using a suitable mixer to produce a visually uniform distribution of the drug substance with no observable lumps or agglomerates;
(v) continuing mixing for at least 30 minutes to ensure that the drug substance is homogeneously distributed as determined visually;
(vi) maintaining the melt in the molten state with continued mixing and is filing into appropriately sized gelatin capsule shells to the target capsule fill weight; and
(vii) optionally, banding the capsules with banding solution.

In various embodiment, the process for manufacturing a capsule comprising a pharmaceutical composition described herein comprises:
(i) melting a lipid carrier (e.g., lauroyl polyoxyl-32 glycerides) at a temperature not less than 50° C. but not exceeding 80° C.;
(ii) adding a PEG (e.g., PEG 400 or PEG 1000) to the lipid carrier (e.g., lauroyl polyoxyl-32 glycerides) and mixing together;
(iii) adding copovidone and an antioxidant (e.g., BHT), with continued mixing;
(iv) adding Form 1 inupadenant hydrochloride gradually under continuous mixing using a suitable mixer to produce a visually uniform distribution of the drug substance with no observable lumps or agglomerates;
(v) continuing mixing for at least 30 minutes to ensure that the drug substance is homogeneously distributed as determined visually;
(vi) maintaining the melt in the molten state with continued mixing and is filing into appropriately sized gelatin capsule shells to the target capsule fill weight; and
(vii) optionally, banding the capsules with banding solution.

In various embodiment, the process for manufacturing a capsule comprising a pharmaceutical composition described herein comprises:
(i) melting a lipid carrier (e.g., lauroyl polyoxyl-32 glycerides) at a temperature not less than 50° C. but not exceeding 80° C.;
(ii) adding a PEG (e.g., PEG 400 or PEG 1000) to the lipid carrier (e.g., lauroyl polyoxyl-32 glycerides) and mixing together;
(iii) adding copovidone and an antioxidant (e.g., BHT), with continued mixing;
(iv) adding Form 1 inupadenant hydrochloride gradually under continuous mixing using a suitable mixer to produce a visually uniform distribution of the drug substance with no observable lumps or agglomerates;
(v) continuing mixing for at least 30 minutes to ensure that the drug substance is homogeneously distributed as determined visually;
(vi) maintaining the melt in the molten state with continued mixing and is filing into appropriately sized gelatin capsule shells to the target capsule fill weight; and
(vii) optionally, banding the capsules with banding solution.

In another aspect, provided herein is a process for manufacturing a capsule comprising a pharmaceutical composition described herein, the process comprising the steps of:
(a) heating a lipid carrier (e.g., lauroyl polyoxyl-32 glycerides) and a PEG (e.g., PEG 400 or PEG 1000) to form a molten mixture;
(b) adding inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), a copovidone, and BHT to the molten mixture to form an intermediate composition;
(c) homogenizing the intermediate composition;
(d) low shear mixing the intermediate composition to form the pharmaceutical composition; and
(e) filing the capsule with the pharmaceutical composition.

In certain embodiments, in step (b), the inupadenant hydrochloride is amorphous inupadenant hydrochloride and/or crystalline inupadenant hydrochloride. In certain embodiments, the amorphous inupadenant hydrochloride is an amorphous inupadenant hydrochloride described herein. In certain embodiments, the inupadenant hydrochloride is a crystalline inupadenant hydrochloride described herein (e.g., Form 1 inupadenant hydrochloride and/or Form 2 inupadenant hydrochloride). In certain embodiments, the inupadenant hydrochloride is an inupadenant hydrochloride hydrate (e.g., Form 1 inupadenant hydrochloride and/or Form 2 inupadenant hydrochloride). In certain embodiments, the inupadenant hydrochloride hydrate is a crystalline inupadenant hydrochloride hydrate described herein (e.g., Form 1 inupadenant hydrochloride and/or Form 2 inupadenant hydrochloride).

In various embodiment, the process for manufacturing a capsule comprising a pharmaceutical composition described herein comprises:
(a) heating a lipid carrier (e.g., lauroyl polyoxyl-32 glycerides) and a PEG (e.g., PEG 400 or PEG 1000) to form a molten mixture;
(b) adding Form 1 inupadenant hydrochloride, a copovidone, and BHT to the molten mixture to form an intermediate composition;
(c) homogenizing the intermediate composition;
(d) low shear mixing the intermediate composition to form the pharmaceutical composition; and
(e) filing the capsule with the pharmaceutical composition.

In various embodiment, the process for manufacturing a capsule comprising a pharmaceutical composition described herein comprises:
(a) heating a lipid carrier (e.g., lauroyl polyoxyl-32 glycerides) and a PEG (e.g., PEG 400 or PEG 1000) to form a molten mixture;
(b) adding Form 2 inupadenant hydrochloride, a copovidone, and BHT to the molten mixture to form an intermediate composition;
(c) homogenizing the intermediate composition;
(d) low shear mixing the intermediate composition to form the pharmaceutical composition; and
(e) filing the capsule with the pharmaceutical composition.

In certain embodiments, in step (a), the lipid carrier and the PEG are heated to a temperature not less than 50° C. but not exceeding 80° C. In certain embodiments, in step (a), the lipid carrier and the PEG are heated to a temperature of about 50° C., about 55° C., about 60° C., about 65° C., or about 70° C.

In certain embodiments, in step (a), the lipid carrier is lauroyl polyoxyl-32 glycerides. In certain embodiments, in step (a), the PEG is PEG 1000.

In certain embodiments, in step (a), the process further comprises maintaining the molten mixture at a temperature not less than 50° C. but not exceeding 80° C. In certain embodiments, the molten mixture is maintained at a temperature of about 50° C., about 55° C., about 60° C., about 65° C., or about 70° C.

In certain embodiments, in step (b), addition of the inupadenant hydrochloride, the copovidone, and BHT to the molten mixture is conducted at a temperature not less than 50° C. but not exceeding 80° C. In certain embodiments, in step (b), addition of the inupadenant hydrochloride, the copovidone, and BHT to the molten mixture is conducted at a temperature of about 50° C., about 55° C., about 60° C., about 65° C., or about 70° C.

In certain embodiments, in step (b), the process further comprises mixing the intermediate composition until fully wetted. In certain embodiments, in step (b), the process further comprises maintaining the intermediate composition at a temperature not less than 50° C. but not exceeding 80° C. In certain embodiments, the intermediate composition is maintained at a temperature of about 50° C., about 55° C., about 60° C., about 65° C., or about 70° C.

In certain embodiments, in step (c), homogenization of the intermediate composition is conducted at a temperature not less than 50° C. but not exceeding 80° C. In certain embodiments, homogenization of the intermediate composition is conducted at a temperature of about 50° C., about 55° C., about 60° C., about 65° C., or about 70° C.

In certain embodiments, in step (c), homogenization of the intermediate composition is conducted for about 5 mins, about 10 mins, about 15 mins, about 20 mins, about 25 mins, about 30 mins, about 35 mins, about 40 mins, about 45 mins, about 50 mins, about 55 mins, or about 60 mins.

In certain embodiments, in step (d), low shear mixing of the intermediate composition is conducted at a temperature not less than 50° C. but not exceeding 80° C. In certain embodiments, low shear mixing of the intermediate composition is conducted at a temperature of about 50° C., about 55° C., about 60° C., about 65° C., or about 70° C.

In certain embodiments, in step (d), low shear mixing of the intermediate composition is conducted for about 5 mins, about 10 mins, about 15 mins, about 20 mins, about 25 mins, about 30 mins, about 35 mins, about 40 mins, about 45 mins, about 50 mins, about 55 mins, or about 60 mins.

In certain embodiments, in step (e), the temperature of the intermediate composition is maintained at a temperature not less than 50° C. but not exceeding 80° C. while filling the capsule.

### Methods of Use and Treatment

In one aspect, provided herein is a method for treating a cancer comprising administering to a patient in need thereof a therapeutically effective amount of a pharmaceutical composition described herein, for example, a pharmaceutical composition comprising inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride) or a pharmaceutical composition comprising inupadenant free base (e.g., crystalline inupadenant free base).

In another aspect, provided herein is a medicament comprising a pharmaceutical composition described herein.

In certain embodiments, the disclosure relates to the use of the pharmaceutical composition described herein for increasing immune recognition and destruction of the cancer cells.

A pharmaceutical composition described herein is therefore useful for the prevention and/or treatment of cancer, especially useful for the treatment of cancer.

The disclosure further relates to a method for treatment of cancer, which comprises administering to a mammalian species in need thereof a therapeutically effective amount of a pharmaceutical composition described herein.

The disclosure further provides the use of a pharmaceutical composition described herein for the manufacture of a medicament for treating and/or preventing cancer.

The disclosure also provides for a method for delaying in patient the onset of cancer comprising the administration of a therapeutically effective amount of a pharmaceutical composition described herein to a patient in need thereof.

Preferably, the patient is a warm-blooded animal, more preferably a human.

Various cancers are known in the art. The cancer may be metastatic or non-metastatic. The cancer may be familial or sporadic. In some embodiments, the cancer is selected from the group consisting of leukemia and multiple myeloma. Additional cancers that can be treated using the methods of the invention include, for example, benign and malignant solid tumors and benign and malignant non-solid tumors. In a specific embodiment, the cancer is selected from breast, bladder, carcinoid, cervical, colorectal, endometrial, glioma, head and neck, liver, lung, melanoma, ovarian, parotid, pancreatic, prostate, metastatic castrate resistant prostate cancer, renal, gastric, sinus, nasal cavity, thyroid, renal transitional cell carcinoma (TCC), renal urothelial carcinoma (UC) and urothelial cancers. In a specific embodiment, the cancer is breast cancer. In a specific embodiment, the cancer is carcinoid cancer. In a specific embodiment, the cancer is cervical cancer. In a specific embodiment, the cancer is colorectal cancer. In a specific embodiment, the cancer is endometrial cancer. In a specific embodiment, the cancer is glioma. In a specific embodiment, the cancer is head and neck cancer. In a specific embodiment, the cancer is liver cancer. In a specific embodiment, the cancer is lung cancer. In a specific embodiment, the cancer is melanoma. In a specific embodiment, the cancer is ovarian cancer. In a specific embodiment, the cancer is pancreatic cancer. In a specific embodiment, the cancer is prostate cancer. In a specific embodiment, the cancer is renal cancer. In a specific embodiment, the cancer is gastric cancer. In a specific embodiment, the cancer is thyroid cancer. In a specific embodiment, the cancer is urothelial cancer.

In a specific embodiment the cancer is non-small cell lung cancer (NSCLC). In a further embodiment, the NSCLC is nonsquamous cell carcinoma. In another embodiment, the NSCLC is squamous cell carcinoma.

In a further embodiment, the cancer is metastatic NSCLC including cell pathology of nonsquamous origin. In a still further embodiment, the cancer is Stage III NSCLC such as locally advanced, unresectable Stage III NSCLC, for example. In these and other embodiments, the NSCLC has relapsed or progressed after prior anti-programmed death (PD)-ligand (L)1 therapy

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

The disclosure further relates to the use of a pharmaceutical composition described herein for the prevention and/or treatment of radiation-induced fibrosis, connective tissue diseases (such as for example Sjogrën syndrome, i.e., scleroderma), chronic bacterial infection (such as for example Helicobacter Pylori), abnormal scarring (keloids), and polymicrobial sepsis.

The disclosure further relates to a method for treatment or prevention of radiation-induced fibrosis, connective tissue diseases (such as for example Sjogrën syndrome, i.e., scleroderma), chronic bacterial infection (such as for example Helicobacter Pylori), abnormal scarring (keloids), and polymicrobial sepsis, which comprises administering to a mammalian species in need thereof a therapeutically effective amount of a pharmaceutical composition described herein.

The disclosure further provides the use of a pharmaceutical composition described herein for the manufacture of a medicament for treating and/or preventing radiation-induced fibrosis, connective tissue diseases (such as for example Sjogrën syndrome, i.e., scleroderma), chronic bacterial infection (such as for example Helicobacter Pylori), abnormal scarring (keloids), and polymicrobial sepsis.

The disclosure also provides for a method for delaying in patient the onset of radiation-induced fibrosis, connective tissue diseases (such as for example Sjogrën syndrome, i.e., scleroderma), chronic bacterial infection (such as for example Helicobacter Pylori), abnormal scarring (keloids), and polymicrobial sepsis, comprising the administration of a therapeutically effective amount of a pharmaceutical composition described herein to a patient in need thereof.

In yet another aspect, provided herein is a combination of inupadenant or inupadenant hydrochloride (e.g., crystalline inupadenant hydrochloride described herein, such as, for example, Form 1 inupadenant hydrochloride and/or Form 2 inupadenant hydrochloride; or amorphous inupadenant hydrochloride), and another pharmacologically active agent (e.g., an anti-cancer therapy as described herein). Inupadenant or inupadenant hydrochloride, can be administered as the sole active agent or they can be administered in combination with other agents. Administration in combination can proceed by any technique apparent to those of skill in the art including, for example, separate, sequential, concurrent and alternating administration.

In one embodiment, provided herein is a method for treating non-small cell lung cancer (NSCLC) (e.g., squamous NSCLC or nonsquamous NSCLC) by administering to a patient in need thereof a pharmaceutical composition comprising inupadenant hydrochloride (e.g., a pharmaceutical composition described herein) in combination with pemetrexed and carboplatin.

In various embodiments, provided herein is a method for treating a cancer in a patient in need thereof, the method comprising administering to the patient a pharmaceutical composition described herein, a dosage form described herein, or a capsule described herein.

In various embodiments, provided herein is a method for treating a cancer in a patient in need thereof, the method comprising administering to the patient an effective amount of amorphous inupadenant hydrochloride and/or crystalline inupadenant hydrochloride (e.g., Form 1 inupadenant hydrochloride and/or Form 2 inupadenant hydrochloride).

In certain embodiments, the cancer is metastatic. In certain embodiments, the cancer is non-metastatic. In certain embodiments, the cancer is selected from breast, bladder, carcinoid, cervical, colorectal, endometrial, glioma, head and neck, liver, lung, melanoma, ovarian, parotid, pancreatic, prostate, metastatic castrate resistant prostate cancer, renal, gastric, sinus, nasal cavity, thyroid, renal transitional cell carcinoma (TCC), renal urothelial carcinoma (UC), and urothelial cancers. In certain embodiments, the cancer is a NSCLC. In certain embodiments, the NSCLC is nonsquamous cell carcinoma. In certain embodiments, the NSCLC is squamous cell carcinoma. In certain embodiments, the NSCLC is metastatic.

In various embodiments, provided herein is a method for treating a NSCLC in a patient in need thereof, the method comprising administering to the patient the pharmaceutical composition described herein, a dosage form described herein, or a capsule described herein, in combination with pemetrexed and carboplatin.

In various embodiments, provided herein is a method for treating a NSCLC in a patient in need thereof, the method comprising administering to the patient an effective amount of amorphous inupadenant hydrochloride and/or crystalline inupadenant hydrochloride, in combination with pemetrexed and carboplatin.

In various embodiments, provided herein is a method for treating nonsquamous NSCLC comprising administering to a patient in need thereof a pharmaceutical composition described herein, a dosage form described herein, or a capsule described herein.

In various embodiments, provided herein is a method for treating nonsquamous NSCLC comprising administering to a patient in need thereof an effective amount of amorphous inupadenant hydrochloride and/or crystalline inupadenant hydrochloride.

In certain embodiments, the amorphous inupadenant hydrochloride is an amorphous inupadenant hydrochloride described herein. In certain embodiments, the inupadenant hydrochloride is a crystalline inupadenant hydrochloride described herein (e.g., Form 1 inupadenant hydrochloride and/or Form 2 inupadenant hydrochloride). In certain embodiments, the inupadenant hydrochloride is an inupadenant hydrochloride hydrate described herein (e.g., Form 1 inupadenant hydrochloride and/or Form 2 inupadenant hydrochloride). In certain embodiments, the inupadenant hydrochloride hydrate is a crystalline inupadenant hydrochloride hydrate described herein (e.g., Form 1 inupadenant hydrochloride and/or Form 2 inupadenant hydrochloride).

In various embodiments, provided herein is a method for treating a NSCLC in a patient in need thereof, the method comprising administering to the patient an effective amount of Form 1 inupadenant hydrochloride, in combination with pemetrexed and carboplatin.

In various embodiments, provided herein is a method for treating a NSCLC in a patient in need thereof, the method comprising administering to the patient an effective amount of Form 2 inupadenant hydrochloride, in combination with pemetrexed and carboplatin.

In various embodiments, provided herein is a method for treating nonsquamous NSCLC comprising administering to a patient in need thereof an effective amount of Form 1 inupadenant hydrochloride.

In various embodiments, provided herein is a method for treating nonsquamous NSCLC comprising administering to a patient in need thereof an effective amount of Form 2 inupadenant hydrochloride.

In certain embodiments, a pharmaceutical composition described herein (e.g., comprising inupadenant hydrochloride) is administered prior to one or more of carboplatin and pemetrexed.

In certain embodiments, an amorphous inupadenant hydrochloride and/or crystalline inupadenant hydrochloride described herein is administered prior to one or more of carboplatin and pemetrexed.

The dose ranges and recitations below refer to the equivalent dose of inupadenant free base contained in the pharmaceutical composition containing inupadenant hydrochloride.

In certain embodiments, the method comprises administering to a patient a daily dose between about 20 mg and about 1000 mg inupadenant. In one embodiment, the method comprises administering to a patient a daily dose between about 20 mg and about 900 mg inupadenant. In one embodiment, the method comprises administering to a patient a daily dose between about 20 mg and about 800 mg inupadenant. In one embodiment, the method comprises administering to a patient a daily dose between about 20 mg and about 700 mg inupadenant. In one embodiment, the method comprises administering to a patient a daily dose between about 20 mg and about 660 mg inupadenant. In one embodiment, the method comprises administering to a patient a daily dose between about 40 mg and about 640 mg inupadenant. In one embodiment, the method comprises administering to a patient a daily dose between about 20 mg and about 500 mg inupadenant. In one embodiment, the method comprises administering to a patient a daily dose between about 20 mg and about 400 mg inupadenant. In one embodiment, the method comprises administering to a patient a daily dose between about 40 mg and about 320 mg inupadenant. In one embodiment, the method comprises administering to a patient a daily dose of inupadenant between about 10 mg and about 70 mg, about 15 mg and about 65 mg, about 20 mg and about 60 mg, about 25 mg and about 55 mg, about 30 mg and about 50 mg, about 31 mg and about 49 mg, about 32 mg and about 48 mg, about 33 mg and about 47 mg, about 34 mg and about 46 mg, about 35 mg and about 45 mg, about 36 mg and about 44 mg, about 37 mg and about 43 mg, about 38 mg and about 42 mg, or about 39 mg and about 41 mg. In one embodiment, the method comprises administering to a patient a daily dose of inupadenant between about 37 mg and about 43 mg. In one embodiment, the method comprises administering to a patient a daily dose of inupadenant between about 38 mg and about 42 mg. In one embodiment, the method comprises administering to a patient a daily dose of inupadenant is between about 39 mg and about 41 mg.

In one embodiment, the method comprises administering to a patient a daily dose of inupadenant between about 130 mg and about 190 mg, about 135 mg and about 185 mg, about 140 mg and about 180 mg, about 145 mg and about 175 mg, about 150 mg and about 170 mg, about 155 mg and about 165 mg, about 156 mg and about 164 mg, about 157 mg and about 163 mg, about 158 mg and about 162 mg, or about 159 mg and about 161 mg. In one embodiment, the method comprises administering to a patient a daily dose of inupadenant between about 157 mg and about 163 mg. In one embodiment, the method comprises administering to a patient a daily dose of inupadenant is between about 158 mg and about 162 mg. In one embodiment, the method comprises administering to a patient a daily dose of inupadenant between about 159 mg and about 161 mg.

In some embodiments, the method comprises administering to a patient a daily dose of inupadenant between about 290 mg and about 350 mg, about 295 mg and about 345 mg, about 300 mg and about 340 mg, about 305 mg and about 335 mg, about 310 mg and about 330 mg, about 315 mg and about 325 mg, about 316 mg and about 324 mg, about 317 mg and about 323 mg, about 318 mg and about 322 mg, or about 319 mg and about 321 mg. In one embodiment, the method comprises administering to a patient a daily dose of inupadenant between about 317 mg and about 323 mg. In one embodiment, the method comprises administering to a patient a daily dose of inupadenant between about 318 mg and about 322 mg. In one embodiment, the method comprises administering to a patient a daily dose of inupadenant between about 319 mg and about 321 mg.

In some embodiments, the method comprises administering to a patient a daily dose of inupadenant of about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about or 60 mg. In some embodiments, the method comprises administering to a patient a daily dose of inupadenant of about 38 mg. In some embodiments, the method comprises administering to a patient a daily dose of inupadenant is about 39 mg. In some embodiments, the method comprises administering to a patient a daily dose of inupadenant of about 40 mg. In some embodiments, the method comprises administering to a patient a daily dose of inupadenant of about 41 mg. In some embodiments, the method comprises administering to a patient a daily dose of inupadenant is about 42 mg.

In some embodiments, the method comprises administering to a patient a daily dose of inupadenant of about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, or about 180 mg. In some embodiments, the method comprises administering to a patient a daily dose of inupadenant of about 158 mg. In some embodiments, the method comprises administering to a patient a daily dose of inupadenant of about 159 mg. In some embodiments, the method comprises administering to a patient a daily dose of inupadenant of about 160 mg. In some embodiments, the method comprises administering to a patient a daily dose of inupadenant of about 161 mg. In some embodiments, the method comprises administering to a patient a daily dose of inupadenant of about 162 mg.

In some embodiments, the method comprises administering to a patient a daily dose of inupadenant of about 300 mg, about 301 mg, about 302 mg, about 303 mg, about 304 mg, about 305 mg, about 306 mg, about 307 mg, about 308 mg, about 309 mg, about 310 mg, about 311 mg, about 312 mg, about 313 mg, about 314 mg, about 315 mg, about 316 mg, about 317 mg, about 318 mg, about 319 mg, about 320 mg, about 321 mg, about 322 mg, about 323 mg, about 324 mg, about 325 mg, about 326 mg, about 327 mg, about 328 mg, about 329 mg, about 330 mg, about 331 mg, about 332 mg, about 333 mg, about 334 mg, about 335 mg, about 336 mg, about 337 mg, about 338 mg, about 339 mg, or about 340 mg. In some embodiments, the method comprises administering to a patient a daily dose of inupadenant of about 318 mg. In some embodiments, the method comprises administering to a patient a daily dose of inupadenant of about 319 mg. In some embodiments, the method comprises administering to a patient a daily dose of inupadenant of about 320 mg. In some embodiments, the method comprises administering to a patient a daily dose of inupadenant of about 321 mg. In some embodiments, the method comprises administering to a patient a daily dose of inupadenant of about 322 mg.

In certain embodiments, the method provides the patient with a daily dose of about 40 mg, about 80 mg, about 160 mg, about 320 mg, or about 640 mg inupadenant. In certain embodiments, the method provides the patient with a daily dose of about 40 mg inupadenant. In certain embodiments, the method provides the patient with a daily dose of about 80 mg inupadenant. In certain embodiments, the method provides the patient with a daily dose of about 160 mg inupadenant. In certain embodiments, the method provides the patient with a daily dose of about 320 mg inupadenant. In certain embodiments, the method provides the patient with a daily dose of about 640 mg inupadenant.

In certain embodiments, a pharmaceutical composition, dosage form, or capsule described herein is administered 1, 2, 3, 4, 5, 6, 7, 8, 9, or 10 times daily. In certain embodiments, a pharmaceutical composition, dosage form, or capsule described herein is administered once daily. In certain embodiments, a pharmaceutical composition, dosage form, or capsule described herein is administered twice daily.

In some embodiments, the method comprises administering twice daily (BID) to a patient a dose of inupadenant between about 10 mg and about 500 mg. In some embodiments, the method comprises administering twice daily (BID) to a patient a dose of inupadenant between about 1 mg and about 400 mg. In some embodiments, the method comprises administering twice daily (BID) to a patient a dose of inupadenant between about 5 mg and about 375 mg. In some embodiments, the method comprises administering twice daily (BID) to a patient a dose of inupadenant between about 10 mg and about 350 mg. In some embodiments, the method comprises administering twice daily (BID) to a patient a dose of inupadenant between about 20 mg and about 320 mg. In some embodiments, the method comprises administering twice daily (BID) to a patient a dose of inupadenant between about 20 mg and about 160 mg.

In some embodiments, the method comprises administering twice daily (BID) to a patient a dose of inupadenant between about 1 mg and about 40 mg, 3 mg and about 38 mg, 5 mg and about 35 mg, 10 mg and about 30 mg, 11 mg and about 29 mg, 12 mg and about 28 mg, 13 mg and about 37 mg, 14 mg and about 16 mg, 15 mg and about 25 mg, 16 mg and about 24 mg, 17 mg and about 23 mg, 18 mg and about 22 mg, or 19 mg and about 21 mg. In some embodiments, the method comprises administering twice daily (BID) to a patient a dose of inupadenant between about 15 mg and about 25 mg. In some embodiments, the method comprises administering twice daily (BID) to a patient a dose of inupadenant between about 18 mg and about 22 mg. In some embodiments, the method comprises administering twice daily (BID) to a patient a dose of inupadenant between about 19 mg and about 21 mg.

In some embodiments, the method comprises administering twice daily (BID) to a patient a dose of inupadenant between about 50 mg and about 110 mg, 55 mg and about 105 mg, 60 mg and about 100 mg, 65 mg and about 95 mg, 70 mg and about 90 mg, 75 mg and about 85 mg, 76 mg and about 84 mg, 77 mg and about 83 mg, 78 mg and about 82 mg, or 79 mg and about 81 mg. In some embodiments, the method comprises administering twice daily (BID) to a patient a dose of inupadenant between about 75 mg and about 85 mg. In some embodiments, the method comprises administering twice daily (BID) to a patient a dose of inupadenant between about 77 mg and about 83 mg. In some embodiments, the method comprises administering twice daily (BID) to a patient a dose of inupadenant between about 79 mg and about 81 mg.

In some embodiments, the method comprises administering twice daily (BID) to a patient a dose of inupadenant between about 130 mg and about 190 mg, about 135 mg and about 185 mg, about 140 mg and about 180 mg, about 145 mg and about 175 mg, about 150 mg and about 170 mg, about 155 mg and about 165 mg, about 156 mg and about 164 mg, about 157 mg and about 163 mg, about 158 mg and about 162 mg, or about 159 mg and about 161 mg. In some embodiments, the method comprises administering twice daily (BID) to a patient a dose of inupadenant between about 157 mg and about 163 mg. In some embodiments, the method comprises administering twice daily (BID) to a patient a dose of inupadenant between about 158 mg and about 162 mg. In some embodiments, the method comprises administering twice daily (BID) to a patient a dose of inupadenant between about 159 mg and about 161 mg.

In some embodiments, the method comprises administering twice daily (BID) to a patient a dose of inupadenant of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg. In some embodiments, the method comprises administering twice daily (BID) to a patient a dose of inupadenant of about 5 mg. In some embodiments, the method comprises administering twice daily (BID) to a patient a dose of inupadenant of about 6 mg. In some embodiments, the method comprises administering twice daily (BID) to a patient a dose of inupadenant of about 7 mg. In some embodiments, the method comprises administering twice daily (BID) to a patient a dose of inupadenant of about 8 mg. In some embodiments, the method comprises administering twice daily (BID) to a patient a dose of inupadenant of about 9 mg. In some embodiments, the method comprises administering twice daily (BID) to a patient a dose of inupadenant of about 10 mg. In some embodiments, the method comprises administering twice daily (BID) to a patient a dose of inupadenant of about 11 mg. In some embodiments, the method comprises administering twice daily (BID) to a patient a dose of inupadenant of about 12 mg. In some embodiments, the method comprises administering twice daily (BID) to a patient a dose of inupadenant of about 13 mg. In some embodiments, the method comprises administering twice daily (BID) to a patient a dose of inupadenant of about 14 mg. In some embodiments, the method comprises administering twice daily (BID) to a patient a dose of inupadenant of about 15 mg. In some embodiments, the method comprises administering twice daily (BID) to a patient a dose of inupadenant of about 16 mg. In some embodiments, the method comprises administering twice daily (BID) to a patient a dose of inupadenant of about 17 mg. In some embodiments, the method comprises administering twice daily (BID) to a patient a dose of inupadenant of about 18 mg. In some embodiments, the method comprises administering twice daily (BID) to a patient a dose of inupadenant of about 19 mg. In some embodiments, the method comprises administering twice daily (BID) to a patient a dose of inupadenant of about 20 mg. In some embodiments, the method comprises administering twice daily (BID) to a patient a dose of inupadenant of about 21 mg. In some embodiments, the method comprises administering twice daily (BID) to a patient a dose of inupadenant of about 22 mg. In some embodiments, the method comprises administering twice daily (BID) to a patient a dose of inupadenant of about 23 mg. In some embodiments, the method comprises administering twice daily (BID) to a patient a dose of inupadenant of about 24 mg. In some embodiments, the method comprises administering twice daily (BID) to a patient a dose of inupadenant of about 25 mg. In some embodiments, the method comprises administering twice daily (BID) to a patient a dose of inupadenant of about 26 mg. In some embodiments, the method comprises administering twice daily (BID) to a patient a dose of inupadenant of about 27 mg. In some embodiments, the method comprises administering twice daily (BID) to a patient a dose of inupadenant of about 28 mg. In some embodiments, the method comprises administering twice daily (BID) to a patient a dose of inupadenant of about 29 mg. In some embodiments, the method comprises administering twice daily (BID) to a patient a dose of inupadenant of about 30 mg. In some embodiments, the method comprises administering twice daily (BID) to a patient a dose of inupadenant of about 31 mg. In some embodiments, the method comprises administering twice daily (BID) to a patient a dose of inupadenant of about 32 mg. In some embodiments, the method comprises administering twice daily (BID) to a patient a dose of inupadenant of about 33 mg. In some embodiments, the method comprises administering twice daily (BID) to a patient a dose of inupadenant of about 34 mg. In some embodiments, the method comprises administering twice daily (BID) to a patient a dose of inupadenant of about 35 mg. In some embodiments, the method comprises administering twice daily (BID) to a patient a dose of inupadenant of about 36 mg. In some embodiments, the method comprises administering twice daily (BID) to a patient a dose of inupadenant of about 37 mg. In some embodiments, the method comprises administering twice daily (BID) to a patient a dose of inupadenant of about 38 mg. In some embodiments, the method comprises administering twice daily (BID) to a patient a dose of inupadenant of about 39 mg. In some embodiments, the method comprises administering twice daily (BID) to a patient a dose of inupadenant of about 40 mg.

In some embodiments, the method comprises administering twice daily (BID) to a patient a dose of inupadenant of about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg. In some embodiments, the method comprises administering twice daily (BID) to a patient a dose of inupadenant of about 70 mg. In some embodiments, the method comprises administering twice daily (BID) to a patient a dose of inupadenant of about 71 mg. In some embodiments, the method comprises administering twice daily (BID) to a patient a dose of inupadenant of about 72 mg. In some embodiments, the method comprises administering twice daily (BID) to a patient a dose of inupadenant of about 73 mg. In some embodiments, the method comprises administering twice daily (BID) to a patient a dose of inupadenant of about 74 mg. In some embodiments, the method comprises administering twice daily (BID) to a patient a dose of inupadenant of about 75 mg. In some embodiments, the method comprises administering twice daily (BID) to a patient a dose of inupadenant of about 76 mg. In some embodiments, the method comprises administering twice daily (BID) to a patient a dose of inupadenant of about 77 mg. In some embodiments, the method comprises administering twice daily (BID) to a patient a dose of inupadenant of about 78 mg. In some embodiments, the method comprises administering twice daily (BID) to a patient a dose of inupadenant of about 79 mg. In some embodiments, the method comprises administering twice daily (BID) to a patient a dose of inupadenant of about 80 mg. In some embodiments, the method comprises administering twice daily (BID) to a patient a dose of inupadenant of about 81 mg. In some embodiments, the method comprises administering twice daily (BID) to a patient a dose of inupadenant of about 82 mg. In some embodiments, the method comprises administering twice daily (BID) to a patient a dose of inupadenant of about 83 mg. In some embodiments, the method comprises administering twice daily (BID) to a patient a dose of inupadenant of about 84 mg. In some embodiments, the method comprises administering twice daily (BID) to a patient a dose of inupadenant of about 85 mg. In some embodiments, the method comprises administering twice daily (BID) to a patient a dose of inupadenant of about 86 mg. In some embodiments, the method comprises administering twice daily (BID) to a patient a dose of inupadenant of about 87 mg. In some embodiments, the method comprises administering twice daily (BID) to a patient a dose of inupadenant of about 88 mg. In some embodiments, the method comprises administering twice daily (BID) to a patient a dose of inupadenant of about 89 mg. In some embodiments, the method comprises administering twice daily (BID) to a patient a dose of inupadenant of about 90 mg.

In some embodiments, the method comprises administering twice daily (BID) to a patient a dose of inupadenant of about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, or about 180 mg. In some embodiments, the method comprises administering twice daily (BID) to a patient a dose of inupadenant of about 150 mg. In some embodiments, the method comprises administering twice daily (BID) to a patient a dose of inupadenant of about 151 mg. In some embodiments, the method comprises administering twice daily (BID) to a patient a dose of inupadenant of about 152 mg. In some embodiments, the method comprises administering twice daily (BID) to a patient a dose of inupadenant of about 153 mg. In some embodiments, the method comprises administering twice daily (BID) to a patient a dose of inupadenant of about 154 mg. In some embodiments, the method comprises administering twice daily (BID) to a patient a dose of inupadenant of about 155 mg. In some embodiments, the method comprises administering twice daily (BID) to a patient a dose of inupadenant of about 156 mg. In some embodiments, the method comprises administering twice daily (BID) to a patient a dose of inupadenant of about 157 mg. In some embodiments, the method comprises administering twice daily (BID) to a patient a dose of inupadenant of about 158 mg. In some embodiments, the method comprises administering twice daily (BID) to a patient a dose of inupadenant of about 159 mg. In some embodiments, the method comprises administering twice daily (BID) to a patient a dose of inupadenant of about 160 mg. In some embodiments, the method comprises administering twice daily (BID) to a patient a dose of inupadenant of about 161 mg. In some embodiments, the method comprises administering twice daily (BID) to a patient a dose of inupadenant of about 162 mg. In some embodiments, the method comprises administering twice daily (BID) to a patient a dose of inupadenant of about 163 mg. In some embodiments, the method comprises administering twice daily (BID) to a patient a dose of inupadenant of about 164 mg. In some embodiments, the method comprises administering twice daily (BID) to a patient a dose of inupadenant of about 165 mg. In some embodiments, the method comprises administering twice daily (BID) to a patient a dose of inupadenant of about 166 mg. In some embodiments, the method comprises administering twice daily (BID) to a patient a dose of inupadenant of about 167 mg. In some embodiments, the method comprises administering twice daily (BID) to a patient a dose of inupadenant of about 168 mg. In some embodiments, the method comprises administering twice daily (BID) to a patient a dose of inupadenant of about 169 mg. In some embodiments, the method comprises administering twice daily (BID) to a patient a dose of inupadenant of about 170 mg. In some embodiments, the method comprises administering carboplatin at the standard approved doses of platinum chemotherapy (carboplatin area under the curve 5 mg/ml per min [AUC5] and pemetrexed 500 mg/m². In some embodiments, the method comprises administering the platinum chemotherapy and the pemetrexed every 3 weeks [Q3W] for 4 cycles, followed by pemetrexed maintenance therapy. Each cycle will cover 3 weeks. In some embodiments, the dose of carboplatin is adjusted to 95%, 90%, 85%, 80%, 75%, 70%, 65%, 60%, 50%, 45%, 40%, 35%, 30%, or 25% of the standard dose of platinum chemotherapy. In some embodiments, the dose of pemetrexed is adjusted to 95%, 90%, 85%, 80%, 75%, 70%, 65%, 60%, 50%, 45%, 40%, 35%, 30%, or 25% of the standard dose of pemetrexed (500 mg/m2).

In certain embodiments, the method comprises administering to the patient twice daily (BID) a dose of about 20 mg, 20 mg, 80 mg, 160 mg, or 320 mg inupadenant. In certain embodiments, the method comprises administering to the patient twice daily (BID) a dose of about 20 mg inupadenant. In certain embodiments, the method comprises administering to the patient twice daily (BID) a dose of about 40 mg inupadenant. In certain embodiments, the method comprises administering to the patient twice daily (BID) a dose of about 80 mg inupadenant. In certain embodiments, the method comprises administering to the patient twice daily (BID) a dose of about 160 mg inupadenant. In certain embodiments, the method comprises administering to the patient twice daily (BID) a dose of about 320 mg inupadenant.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

Abbreviations and Definitions

| | |
|---|---|
| API | active pharmaceutical ingredient |
| AUC | area under the curve |
| BA | bioavailability |
| BU | blend uniformity |
| Cₘₐₓ | maximum observed plasma concentration |
| CU | content uniformity |
| DP | drug product |
| DS | drug substance |
| DSC | differential scanning calorimetry |
| DVS | dynamic vapor sorption |
| FaSSIF | fasted state simulated intestinal fluid |
| FeSSIF | fed state simulated intestinal fluid |
| FRI | Flow Rate Index |
| GMP | Good Manufacturing Practice |
| HDPE | high density polyethylene |
| HPLC | high performance liquid chromatography |
| Kp | kilopond(s) |
| LOD | limit of detection |
| Max | maximum |
| Min | minimum |
| NA | not applicable |
| ND | not detected (or <0.05% area on HPLC analysis) |
| PVA | Polyvinyl alcohol |
| RSD | relative standard deviation |
| RT | room temperature or real time |
| SD | Standard Deviation |
| SDS | sodium dodecyl sulfate |
| SLS | sodium lauryl sulfate |
| USP | United States Pharmacopoeia |
| w/w | weight-to-weight |

### Example 1. Analytical Methods for the Characterization of Amorphous Inupadenant Hydrochloride, Crystalline Inupadenant Hydrochloride (e.g., Crystalline Inupadenant Hydrochloride Hydrate, Form 1 Inupadenant Hydrate, and Form 2 Inupadenant Hydrate), and Pharmaceutical Compositions Comprising Inupadenant Hydrochloride

### (1) X-Ray Powder Diffraction (XRPD)

### Method A

XRPD analysis was carried out on a PANalytical X'pert pro with PIXcel detector (128 channels), scanning the samples between 3° and 35° 2θ. The material was gently ground (where required) to release any agglomerates and loaded onto a multi-well plate with Mylar polymer film to support the sample. The multi-well plate was then placed into the diffractometer and analyzed using Cu K radiation (α₁λ=1.54060 Å; α₂=1.54443 Å; β=1.39225 Å; α₁:α₂ ratio=0.5) running in transmission mode (step size 0.0130° 2θ, step time 18.87 s) using 40 kV/40 mA generator settings. Data were visualized and images generated using the HighScore Plus 4.7 desktop application (PANalytical, 2017).

### Method B: Variable Temperature XRPD Analysis (VT-XRPD)

VT-XRPD analysis was carried out on a Philips X'Pert Pro Multipurpose diffractometer equipped with a temperature chamber. The samples were scanned between 4° and 35.99° 2θ using Cu K radiation (α₁λ=1.54060 Å; α₂=1.54443 Å; β=1.39225 Å; α₁:α₂ ratio=0.5) running in Bragg-Brentano geometry (step size 0.008° 2θ) using 40 kV/40 mA generator settings. XRPD measurements were performed between 25-260° C.

### Method C. Variable Humidity XRPD Analysis (VH-XRPD)

VH-XRPD analysis was carried out on a Philips X'Pert Pro Multipurpose diffractometer equipped with a humidity chamber. The samples were scanned between 4° and 35.99° 2θ using Cu K radiation (α₁λ=1.54060 Å; α₂=1.54443 Å; β=1.39225 Å; α₁:α₂ ratio=0.5) running in Bragg-Brentano geometry (step size 0.008° 2θ) using 40 kV/40 mA generator settings. XRPD measurements were performed between 2-90% RH.

### Method D

XRPD analysis was carried out on a Brucker D8 Advance Eco XRPD under the following conditions:
Generator Setting: Cu 40 kV, 25 mA
Divergence slit: 0.3° (0.6 mm)
Axial Soller slits (Primary and secondary): 2.5° and 2.5°
Anti-scatter-slit: 0.3° (0.6 mm)
Secondary monochromator anti-scatter slit: 1 mm
Detector slit: 0.1 mm
Linear detector LYNXEYE: 3° detector opening
Angles scanned: 2 to 45° 2θ.

### Method E

XRPD data were collected using a Bruker AXS D2 PHASER in Bragg-Brentano configuration, equipment #1549. A Cu anode at 30 kV, 10 mA, sample stage standard rotating (5/min) with beam stop and monochromatization by a Kβ-filter (0.5% Ni) was used. The slits used were fixed divergence slits 1.0 mm (=0.61°), a primary axial Soller slit 2.5°, and a secondary axial Soller slit 2.5°. The detector was a linear detector LYNXEYE with receiving slit 5° detector opening. The standard sample holder (0.1 mm cavity in (510) silicon wafer) had a minimal contribution to the background signal. The measurement conditions: scan range 5-45° 2θ, sample rotation 5 rpm, 0.5 s/step, 0.010°/step, and 8.0 mm detector slit. The software used for data collection was Diffrac.Measurement Centre v4.6. Data analysis was performed using Diffrac.Eva V4.1.1 evaluation software. No background correction or smoothing was applied to the XRPD patterns.

As system suitability check the XRPD pattern of a corundum standard was checked for peak position, peak shape, intensity, and linearity.

### (2) Thermogravimetric Analysis/Differential Scanning Calorimetry (TGA/DSC)

### Method A

5-10 mg of material was added into a pre-tared open aluminum pan and loaded into a TA Instruments Discovery SDT 650 Auto—Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### Method B

TGA/DSC thermograms were collected using a Mettler Toledo TGA/DSC-01/03 STARe System with a 34-position auto sampler, equipment #1547/#3119. The samples were made using Al crucibles (40 μl; pierced). Typically, 5-10 mg of sample was loaded into a pre-weighed Al crucible and was kept at 20° C. for 5 minutes, after which it was heated at 10° C./min from 20° C. to 350° C. A nitrogen purge of 40 ml/min is maintained over the sample. The software used for data collection and evaluation was STARe Software v15.00 build 8668. No corrections were applied to the collected thermograms.

As system suitability check indium and zinc were measured. For calibration of the instrument benzophenone, indium, lead, tin and zinc were used as references.

### (3) Differential Scanning Calorimetry (DSC)

### Method A

1-5 mg of material was weighed into an aluminum DSC pan and sealed non-hermetically with an aluminum lid. The sample pan was then loaded into a TA Instruments Discovery DSC 2500 differential scanning calorimeter equipped with a RC90 cooler. The sample and reference were heated to ca. 260-290° C. (sample dependent) at a scan rate of 10° C./min and the resulting heat flow response monitored. The sample was cooled to −80° C. and then reheated again to the 300° C. all at 10° C./min. Nitrogen was used as the purge gas, at a flow rate of 50 cm³/min.

### Method B

DSC thermograms were collected using a Mettler Toledo DSC1 STARe System, equipment #1564. The samples were made using Al crucibles (40 μl; pierced). Typically, 1-8 mg of sample was loaded onto a pre-weighed Al crucible and was kept at 20° C. for 5 minutes, after which it was heated at 10° C./min from 20° C. to 350° C., and then kept at 350° C. for 1 minute. A nitrogen purge of 40 ml/min was maintained over the sample. The software used for data collection and evaluation was STARe Software v15.00 build 8668. No corrections were applied to the collected thermograms.

As system suitability check indium and zinc were measured. For calibration indium, lead and zinc were used as references.

### (4) Karl Fischer Coulometric Titration (KF)

15-20 mg of solid was weighed into a 10 mL glass vial and tightly sealed with a screw cap. Samples were analyzed using a Mettler Toledo C30SX and an InMotion KFOven Autosampler at 170° C. Samples were analyzed in duplicate and an average moisture content reported. See Table 1 below for further details.

TABLE 1
Experimental Parameters for KF

| | | |
|---|---|---|
| Blank | Oven Temperature/° C. | 170 |
| | Source for Drift | Determination |
| | Max. Start Drift/μg/min | 10 |
| | Carrier Gas Flow Rate/ | 80 |
| | mL/min | |
| | Transfer Tube Heating | No |
| | Mix Time/s | 60 |
| | Stir Speed/% | 45 |
| | Drift Termination/s | 10 (Delay Time) |
| | Max. Titration Time/s | 600 |
| Sample | Oven Temperature/° C. | 170 |
| | Source for Drift | Determination |
| | Max. Start Drift/μg/min | 10 |
| | Carrier Gas Flow Rate/ | 80 |
| | mL/min | |
| | Mix Time/s | 60 |
| | Stir Speed/% | 45 |
| | Drift Termination/s | 10 (Delay Time) |
| | Max. Titration Time/s | 600 |

### (5) Fourier-Transform Infrared Spectroscopy (FT-IR)

Infrared spectroscopy was carried out on a Bruker ALPHA P spectrometer. Sufficient material was placed onto the center of the plate of the spectrometer and the spectra were obtained using the following parameters:
Resolution: 4 cm⁻¹
Background Scan Time: 16 scans
Sample Scan Time: 16 scans
Data Collection: 4000 to 400 cm-1
Result Spectrum: Transmittance
Software: OPUS version 6

### (6) Dynamic Vapor Sorption (DVS)

### Method A

10-20 mg of sample was placed into a mesh vapor sorption balance pan and loaded into a DVS Intrinsic or Advantage dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis (XRPD Method A) was then carried out on any solid retained.

### Method B

DVS isotherms were collected using a Surface Measurement Systems Ltd. DVS-1 No Video, equipment #2126. The sample was loaded into balance pan, typically 10-30 mg, and equilibrated at 0% RH. After the material has dried the RH was increased with 10% per step for 1 hour per increment, ending at 95% RH. After completion of the sorption cycle, the sample was dried using the same method. The software used for data collection is DVSWin v3.01 No Video. Data analysis was performed using DVS Standard Analysis Suite v6.3.0 (Standard).

### (7) High Performance Liquid Chromatography (HPLC)

### Method A: High Performance Liquid Chromatography-Ultraviolet Detection (HPLC-UV) Purity Method

Experimental parameters and conditions:
Instrument: Dionex Ultimate 3000
Column: Waters X-Select CSH C18 150×4.6 mm, 3.5 μm
Column Temperature: 40° C.
Autosampler Temperature: Ambient
UV wavelength: 274 nm UV
Injection Volume: 6 mL
Flow Rate: 1.0 mL/min
Mobile Phase A: 0.05% Formic Acid in Water
Mobile Phase B: 0.05% Formic Acid in acetonitrile
Diluent: Acetonitrile:Water (50:50% v/v)
Needle Wash: Acetonitrile:Water (50:50% v/v)

### Method B: High Performance Liquid Chromatography—Charged Aerosol Detection (HPLC-CAD)

### Experimental Parameters and Conditions:

Instrument: Dionex Ultimate 3000 with Dionex Corona Ultra CAD
Column: In order: Phenomenex Aeris Peptide XB-C18 100 Å, 150×4.6 mm, 3.6 μm (as guard column) and Dionex Acclaim Trinity P2, 50×2.1 mm, 3 μm
Column Temperature: 30° C.
Injection Volume: 4 μL
Flow Rate: 0.45 mL/min
Mobile Phase A: Water
Mobile Phase B: 100 mM ammonium formate pH 3.65
Detection: CAD; Nebulizer Temperature: 30° C.; Filter: Corona

TABLE 2
Gradient Program for HPLC-CAD Method

| Time (minutes) | Solvent B [%] |
|---|---|
| 0.0 | 20 |
| 1.7 | 20 |
| 7.0 | 95 |
| 11.7 | 95 |
| 12.3 | 20 |
| 23.0 | 20 |

### Method C. HPLC—Concentration Method

### Experimental Parameters and Conditions:

Instrument: Dionex Ultimate 3000
Column: Waters X-Select CSH C18 150×4.6 mm, 3.5 μm
Column Temperature: 40° C.
Autosampler Temperature: Ambient
UV wavelength: 274 nm UV
Injection Volume: 6 mL
Flow Rate: 2.0 mL/min
Mobile Phase A: 0.05% Formic Acid in Water
Mobile Phase B: 0.05% Formic Acid in acetonitrile
Diluent: Acetonitrile:Water (50:50% v/v)
Needle Wash: Acetonitrile:Water (50:50% v/v)

TABLE 3
Gradient Program for HPLC-Concentration Method

| Time (min) | Solvent A (%) | Solvent B (%) |
|---|---|---|
| 0.0 | 85 | 15 |
| 1.5 | 85 | 15 |
| 12.5 | 5 | 95 |
| 12.6 | 85 | 15 |
| 15.0 | 85 | 15 |

### (8) Thermogravimetric Analysis—Fourier Transform Infrared Spectroscopy (TGA-IR)

The off-gassing materials were directed through a transfer line to a gas cell, where the infrared light interacts with the gases. The temperature ramp and first derivative weight loss information from the TGA is shown as a Gram-Schmidt (GS) profile; the GS profile essentially shows the total change in the IR signal relative to the initial state. In most cases, the GS and the derivative weight loss are similar in shape, although the intensity of the two may differ. For this experiment two devices were coupled to each other. The TGA studies were performed using a Mettler Toledo TGA/DSC1 STARe System with a 34-position auto sampler, equipment #1547. The samples were made using Al crucibles (100 μl; pierced). Typically, 20-50 mg of sample was loaded into a pre-weighed Al crucible and is kept at 30° C. for 5 minutes, after which it is heated at 10° C./min from 30° C. to 350° C. A nitrogen purge of 40 ml/min is maintained over the sample. The TGA-IR module of a Nicolet iS50 was coupled to the TGA/DSC1. The IR studies were performed using a Thermo Scientific Nicolet iS50, equipment #2357.

Experiment setup of the collected series, the profile Gram-Schmidt was used number of scans 10 with a resolution of 4. The software OMNIC version 9.2 was used for data collection and evaluation.

### (9) Single Crystal X-Ray Diffraction

A suitable crystal of inupadenant free base was selected and mounted in a nylon loop while protected in a protective layer of paratone oil. Data were collected using a Bruker D8Venture diffractometer equipped with a Photon III detector operating in shutterless mode at 100(2) K with Cu-Kα radiation (1.54178 Å). The structure was solved in the Olex2 software package with the ShelXT (intrinsic phasing) structure solution program and refined with the ShelXL3 refinement package using Least Squares minimization. Data were collected, solved and refined in the Triclinic space-group P1.

All non-hydrogen atoms were located in the Fourier map and their positions refined prior to describing the thermal movement of all non-hydrogen atoms anisotropically. Within the structure, two complete EOS100850 formula units were refined. All hydrogen atoms were placed in calculated positions using a riding model with fixed Uiso at 1.2 times for all CH, CH₂ and NH₂ groups and at 1.5 times for all CH₃ groups.

The highest residual Fourier peak was found to be 0.41 e.Å⁻³ approx. 0.91 Å from S36′ and the deepest Fourier hole was found to be −0.55 e.Å⁻³ approx. 0.71 Å from S36′.

### Example 2. Process for Preparing Form 1 Inupadenant Hydrochloride

At room temperature, charged 86.9 mL (104.3 g) 37% HCl to a flask. Then charged 86.9 mL (86.9 g) deionized water to the flask and stirred at room temperature. The temperature was maintained at room temperature over the course of the reaction. The process provided 173.8 mL (191.2 g) 6N HCl.

Into a 20 L reactor (reactor comprises a digital thermometer, digital stirring agitator, and N₂ inlet) was charged 600 g inupadenant free base (which may be made in accordance with the process described in PCT/EP2018/058301 or in PCT/CN2021/127308), which was stirred at 80-90 rpm with nitrogen bubbling. 2.7 L 4.5 V DMSO was then charged to the reactor. The resulting slurry mixture was then stirred at room temperature for 15 minutes. The slurry mixture was then slowly heated to 40° C. and the 6N HCl was then slowly added (20-30 minutes) to the reactor. The reaction mixture was then stirred at 40° C. for 15-20 minutes. The reaction mixture was then slowly heated to 50-60° C. (target temperature 55° C.) and maintained at this temperature for 30 minutes.

The reaction mixture was then polish filtered using 9F filter paper. The reactor was then washed with 0.5 V DMSO and rinsed. The filtered reaction mixture is then added back to the cleaned reactor and slowly cooled to 40-50° C. (target temperature 45° C.). 2 V IPA was then slowly added to the reaction mixture (added 1200 mL over 1 hour). After the IPA addition was complete, the reaction mixture was then stirred at 40° C. for 4 hours.

4.8 L 8V IPA was then slowly added to the reaction mixture at 40° C. over 1-2 hours. The reaction mixture was then stirred at 40° C. for 4-6 hours. Thereafter reaction mixture was cooled to 20-25° C. and then stirred for a minimum of 2 hours at this temperature.

The resulting solids were then filtered over 8 hours using polypad filter paper. The reactor was rinsed with 2V IPA. The filtered solids were then washed with 2×2V MTBE and then dried at 40-45° C. to provide Form 1 inupadenant hydrochloride. The yield of product was 94%. The HPLC purity was 99.2%, with a chiral purity of 99.9%.

### Example 3. A Process for Preparing Form 2 Inupadenant Hydrochloride

560 g Form 1 inupadenant hydrochloride (prepared in accordance with Example 2), 8 V (4.4 L) ethanol (200 proof), and 2 V (1.12 L) water was charged to a clean reactor. The mixture was then stirred at room temperature for 10-15 minutes. The mixture is then slowly heated to 55° C. 1 V ethanol was then added and the mixture was then stirrer for 5-7 hours at 55° C. The mixture was then cooled to 25° C. over a period of 2 hours and then stirred at 25° C. for ˜16 hours. The resulting solids was filtered and washed with 2×2 V ethanol and 2×2 V MTBE. The solids were then dried at 45° for over 20 hours to provide Form 2 inupadenant hydrochloride. The yield of the product was 95%. The HPLC purity was 99.6%, with a chiral purity of 99.6%.

### Example 4. A Process for Preparing Form 2 Inupadenant Hydrochloride

Inupadenant free base (which may be made in accordance with PCT/EP2018/058301) is charged to a reactor along with dimethyl sulfoxide. Hydrochloric acid (6M) is added, and the mixture is heated to 40-60° C. until dissolved. The reaction mixture is filtered, and isopropanol is added. The mixture is stirred for 4-6 hours at 35-45° C., additional isopropanol is added, and the reaction mixture stirred for a further 4-6 hours. The slurry is cooled to 20-25° C. and stirred for at least 4 hours. The crude HCl salt is filtered, washed with a mixture of isopropanol and MTBE. The crude HCl salt is dried to constant weight, then re-slurried with stirring in an ethanol/water mixture (4:1) for 4-6 hours at 50-60° C. The suspension is cooled to 20-30° C. and stirred for at least 16 hours. The product—crystalline inupadenant hydrochloride is filtered, washed successively with ethanol and methyl tert-butyl ether and dried at 45° C. An XRPD pattern for the product was collected (Example 1, XRPD Method D) and is shown in FIG. 4. The XRPD pattern is consistent with the known XRPD pattern of Form 2 inupadenant hydrochloride.

### Example 5. Characterization of Crystalline Inupadenant Free Base

Crystalline inupadenant free base may be prepared in accordance with the method detailed in Scheme 1.

An XRPD pattern of crystalline inupadenant free base was collected (Example 1, XRPD Method A) and is shown in FIG. 16. Tabulated characteristics of a simulated XRPD pattern calculated from single crystal X-ray diffraction data are provided below in Table 4, which lists diffraction angle (2θ) and relative intensity (expressed as a percentage with respect to the most intense peak).

TABLE 4
Simulated XRPD Data of Crystalline Inupadenant Free Base

| Diffraction Angle [°2θ] | Relative Intensity [%] |
|---|---|
| 6.6 | 14.5 |
| 7.6 | 9.5 |
| 10 | 20.8 |
| 11.5 | 7.4 |
| 12.2 | 2.2 |
| 12.5 | 2.7 |
| 13.1 | 6.1 |
| 13.7 | 5 |
| 13.9 | 34.7 |
| 15.3 | 25.9 |
| 16.1 | 28.9 |
| 16.4 | 30.8 |
| 16.6 | 2.9 |
| 17.3 | 30.7 |
| 17.8 | 5.9 |
| 18.4 | 18.7 |
| 19.2 | 45 |
| 19.9 | 25.9 |
| 20.2 | 74.6 |
| 20.3 | 26.6 |
| 21.2 | 10.4 |
| 21.3 | 15.9 |
| 21.4 | 18.9 |
| 21.7 | 100 |
| 22.4 | 98.5 |
| 23 | 70.6 |
| 23.7 | 10.4 |
| 24 | 10.7 |
| 24.5 | 18.6 |
| 25.2 | 18.8 |
| 25.4 | 10.3 |
| 25.9 | 5.6 |
| 26 | 3.6 |
| 26.7 | 17 |
| 27 | 5.1 |
| 27.1 | 6 |
| 27.6 | 84.8 |
| 28.0 | 3.2 |
| 28.4 | 18.5 |
| 28.6 | 4.3 |
| 29.0 | 3.6 |
| 29.4 | 1 |
| 29.5 | 1.9 |
| 29.9 | 12.2 |
| 30.2 | 3 |
| 30.5 | 5.1 |
| 30.9 | 9 |
| 31.6 | 0.6 |
| 31.9 | 4.7 |
| 32.3 | 5.2 |
| 32.5 | 3.7 |
| 32.9 | 5.6 |
| 33.1 | 1.3 |
| 33.4 | 2.6 |
| 33.7 | 7.8 |
| 33.9 | 12.6 |
| 34.2 | 2.3 |

TGA/DSC analysis (Example 1, TGA/DSC Method A) showed Form 1 inupadenant free base to be likely anhydrous. No weight losses were observed, except for a 1.5% weight loss which was observed during the material melt, followed by degradation (FIG. 17). DSC showed a single endothermic event with a peak onset of about 242° C. (peak maximum at about 245° C.), which corresponded to the melting of Form 1 inupadenant free base (FIG. 17).

DSC analysis (Example 1, DSCMethodA) of Form 1 inupadenant hydrochloride showed an endothermic event with a peak onset at about 242° C. and a peak maximum at about 245° C. (FIG. 18). This endotherm was attributed to the melting of Form 1 inupadenant hydrochloride. The melting temperature was found to be in agreement with the melting temperature determined by the TGA/DSC analysis described above.

An FT-IR spectrum of Form 1 inupadenant free base was collected (Example 1) and is shown in FIG. 19. Tabulated characteristics of the FT-IR spectrum in FIG. 19 are provided below in Table 5, which lists peak wavenumber (cm⁻¹) and transmittance (%). The following stretches were observed: NH₂ stretches ˜3300 cm⁻¹, aromatic C—H stretches ˜3000 cm⁻¹, C═O stretches 1700-1600 cm⁻¹, and aromatic C═C stretches ˜1660 cm⁻¹.

TABLE 5
FT-IR Data of Form 1 Inupadenant Free Base

| | Transmittance |
|---|---|
| Wavenumber (cm⁻¹) | (%) |
| 3375.51 | 91.70 |
| 3307.31 | 92.10 |
| 3291.64 | 92.00 |
| 3152.58 | 87.80 |
| 3108.00 | 88.40 |
| 2958.82 | 92.30 |
| 2931.47 | 91.40 |
| 2872.33 | 92.90 |
| 2836.28 | 91.60 |
| 2836.28 | 91.60 |
| 2820.62 | 89.10 |
| 2800.33 | 89.70 |
| 2756.63 | 93.30 |
| 2689.47 | 94.30 |
| 1695.54 | 60.90 |
| 1650.17 | 54.60 |
| 1625.74 | 73.30 |
| 1612.46 | 62.50 |
| 1602.09 | 62.20 |
| 1552.42 | 49.20 |
| 1516.10 | 58.50 |
| 1469.88 | 66.60 |
| 1448.14 | 67.20 |
| 1426.61 | 77.80 |
| 1409.80 | 69.60 |
| 1379.24 | 80.00 |
| 1311.19 | 65.00 |
| 1257.14 | 67.60 |
| 1218.85 | 59.80 |
| 1204.46 | 75.00 |
| 1183.25 | 57.70 |
| 1146.63 | 77.30 |
| 1121.08 | 74.60 |
| 1042.98 | 78.10 |
| 1025.86 | 57.80 |
| 1007.23 | 62.60 |
| 996.77 | 62.00 |
| 972.91 | 78.90 |
| 943.32 | 79.00 |
| 925.60 | 66.50 |
| 905.18 | 79.10 |
| 855.81 | 69.30 |
| 845.75 | 69.30 |
| 825.91 | 81.50 |
| 756.49 | 55.30 |
| 709.75 | 66.20 |
| 683.02 | 66.90 |
| 657.56 | 76.60 |
| 641.79 | 76.00 |
| 630.09 | 79.90 |
| 597.34 | 74.40 |
| 579.25 | 61.60 |
| 504.37 | 78.10 |
| 487.04 | 79.90 |
| 470.50 | 74.30 |
| 429.69 | 82.50 |

HPLC purity analysis showed Form 1 inupadenant free base to have a solid purity of 98.73% (by relative area).

### Example 6. Preparation and Characterization of Amorphous Inupadenant Hydrochloride

Amorphous inupadenant hydrochloride was prepared by bead milling Form 2 inupadenant hydrochloride (as prepared in Example 3) for between 30 minutes and 2 hours.

An XRPD pattern of milled Form 2 inupadenant hydrochloride was collected (Example 1, XRPD Method A) and is shown in FIG. 1. The XRPD shows that the milled sample is predominantly amorphous.

TGA/DSC analysis (Example 1, TGA/DSC Method A) of amorphous inupadenant hydrochloride showed a 3.5% weight loss upon heating amorphous inupadenant hydrochloride from 31° C. to 83° C. (1.2 equiv. water theoretically) (FIG. 3). DSC showed an exothermic crystallization event with a peak onset of about 156° C. (peak maximum at about 164° C.). This was followed by an endothermic melting event with a peak onset of about 235° C. (peak maximum at 239° C.) (FIG. 3).

DSC analysis (Example 1, DSCMethodA) of amorphous inupadenant hydrochloride was performed by heating a sample to between 260-290° C., then cooling the sample to −80° C., and finally reheating the sample to 300° C. The results showed that, on the first heating cycle, an exothermic crystallization event from the amorphous form with a peak onset of about 162° C. (peak maximum at about 171° C.). This was followed by an endothermic melting event with a peak onset of about 248° C. (peak maximum at about 254° C.) (FIG. 2A). Upon cooling a potential glass transition with enthalpic recovery was observed with a midpoint of about 108° C. by DSC (FIG. 2B). Finally, upon reheating a potential glass transition with a midpoint of 139° C. was observed (FIG. 2C).

### Example 7. Characterization of Form 1 Inupadenant Hydrochloride

Form 1 inupadenant hydrochloride was prepared according to the method described in Example 2.

An XRPD pattern of Form 1 inupadenant hydrochloride was collected (Example 1, XRPD Method E) and is shown in FIG. 12. Tabulated characteristics of the XRPD pattern in FIG. 12 are provided below in Table 6, which lists diffraction angle (20) and relative intensity (expressed as a percentage with respect to the most intense peak).

TABLE 6
XRPD Data of Form 1 Inupadenant Hydrochloride

| Diffraction Angle [°2θ] | Relative Intensity [%] |
|---|---|
| 5.5 | 8.2 |
| 7.6 | 3.4 |
| 9 | 76.2 |
| 9.6 | 100 |
| 12.9 | 8.3 |
| 13.5 | 5.7 |
| 14 | 3.1 |
| 15.1 | 26.7 |
| 15.7 | 72.6 |
| 16.2 | 36.4 |
| 17.5 | 30.7 |
| 17.8 | 43 |
| 18.2 | 69.3 |
| 19.4 | 11.2 |
| 19.6 | 18.2 |
| 20.9 | 3.8 |
| 21.5 | 17.2 |
| 22.5 | 32.1 |
| 22.7 | 37 |
| 23.7 | 22.7 |
| 24.3 | 38.6 |
| 24.8 | 39.4 |
| 25.2 | 36.4 |
| 25.6 | 68.4 |
| 27 | 76.5 |
| 27.9 | 28.7 |
| 28.4 | 11.3 |
| 29.5 | 10.9 |
| 29.7 | 9.5 |
| 30.8 | 4.9 |
| 32.8 | 6.2 |
| 33.8 | 5.3 |
| 42.3 | 1.9 |
| 43.3 | 6.9 |

TGA/DSC analysis of Form 1 inupadenant hydrochloride (Example 1, TGA/DSCMethod B) showed a 2.1% weight loss from 20 to 80° C. (FIG. 13). DSC analysis showed a small endothermic event with a peak onset of about 58° C. (peak maximum at about 113° C.). An endothermic event (e.g., melting and/or decomposition) was observed with a peak onset of about 247° C. (peak maximum at about 258° C.) (FIG. 13). TGA-IR analysis (Example 1) determined that the weight loss was due to loss of water from the crystal lattice.

DSC analysis (Example 1, DSC Method B) of Form 1 inupadenant hydrochloride showed an endothermic event with a peak onset at about 229° C. (peak maximum at about 240° C.). This was followed by a larger endothermic event with a peak onset at about 246° C. (peak maximum at about 253° C.) (FIG. 14).

DVS analysis (Example 1, DVS Method B) of Form 1 inupadenant hydrochloride showed a total mass uptake of 5 wt. % (stepwise) at 95% RH (FIG. 15).

### Example 8. Characterization of Form 2 Inupadenant Hydrochloride

Form 2 inupadenant hydrochloride was prepared according to the method described in Example 3.

An XRPD pattern of Form 2 inupadenant hydrochloride was collected (Example 1, XRPD Method A) and is shown in FIG. 5. Tabulated characteristics of the XRPD pattern in FIG. 5 are provided below in Table 7, which lists diffraction angle (20) and relative intensity (expressed as a percentage with respect to the most intense peak).

TABLE 7
XRPD Data of Form 2 Inupadenant Hydrochloride

| Diffraction Angle [°2θ] | Relative Intensity [%] |
|---|---|
| 3.3 | 11 |
| 5.3 | 9.9 |
| 8.9 | 80 |
| 9.3 | 44.2 |
| 12.6 | 13.8 |
| 14.5 | 30.9 |
| 14.8 | 15.7 |
| 15.4 | 40.9 |
| 16.1 | 20.8 |
| 16.5 | 13.7 |
| 17.5 | 30.1 |
| 18.1 | 12.2 |
| 19.3 | 10 |
| 19.8 | 14.1 |
| 22.1 | 20.6 |
| 22.6 | 22.6 |
| 23.1 | 15.5 |
| 23.5 | 12.5 |
| 24.3 | 36 |
| 24.8 | 49.6 |
| 24.9 | 72.6 |
| 25.4 | 21.7 |
| 25.9 | 100 |
| 26.1 | 50.2 |
| 26.7 | 94.3 |
| 27.5 | 24.2 |
| 28.2 | 15.8 |
| 29.1 | 3.5 |
| 29.3 | 8.6 |
| 30.5 | 7.1 |
| 31 | 5.2 |
| 31.6 | 5.5 |
| 32.2 | 3.6 |
| 34.5 | 3.9 |

TGA/DSC analysis (with initial weight stabilization) of Form 2 inupadenant hydrochloride (Example 1, TGA/DSCMethod A) showed a 3.2% weight loss from 25 to 70° C. (1.1 mole equiv. water theoretically) (FIG. 11A). DSC analysis showed a small endothermic event with a peak onset of 141.7° C. (peak maximum at 142.1° C.). An endothermic/decomposition event was observed with a peak onset of about 229° C. (peak maximum at about 240° C.) (FIG. 11A).

TGA/DSC analysis (without initial weight stabilization) of Form 2 inupadenant hydrochloride (Example 1, TGA/DSC Method A) showed a 3.2% weight loss from 25 to 71° C. (1.1 mole equiv. water theoretically) (FIG. 11B). DSC analysis showed a small endothermic event with a peak onset of about 138° C. (peak maximum at about 150° C.). An endothermic/decomposition event was observed with a peak onset of 231° C. (peak maximum at 240° C.) (FIG. 11B).

DSC analysis (Example 1, DSCMethodA) of Form 2 inupadenant hydrochloride showed a broad endothermic event with a peak onset at about 71° C. (peak maximum at about 101° C.), a small endothermic event with a peak onset at about 147° C. (peak maximum at about 150° C.). This was followed by a larger endothermic event with a peak onset at about 251° C. (peak maximum at about 258° C.) (FIG. 9).

DVS analysis (Example 1, DVS Method A) of Form 2 inupadenant hydrochloride showed a total mass increase of 3.8 wt. % at 90% RH which corresponded to 1.4 mole equiv. water (FIG. 10A). Two stepwise mass increases/decreases were observed: (1) between 0 and 10% RH, a 1.94% wt. (0.7 mole equiv. water) mass loss/increase was observed, which was found to be reversible. It should be noted that at 0% RH, complete equilibration was not observed in the DVS kinetic plot (FIG. 10B), suggesting that additional mass loss may be observed if held for a longer period at 0% RH or if the analysis was carried out at a higher temperature; and (2) between 10 and 30% RH, a second stepwise 1.2% wt. (0.44 mole equiv. water) mass loss/increase was observed which was found to be reversable. The DVS kinetic plot showed no deliquesce or re-crystallization events (FIG. 10B). XRPD analysis (Example 1, XRPD Method A) of the post-DVS sample exhibited no change in solid form after exposure to the DVS humidity conditions, with Form 2 inupadenant hydrochloride being retained.

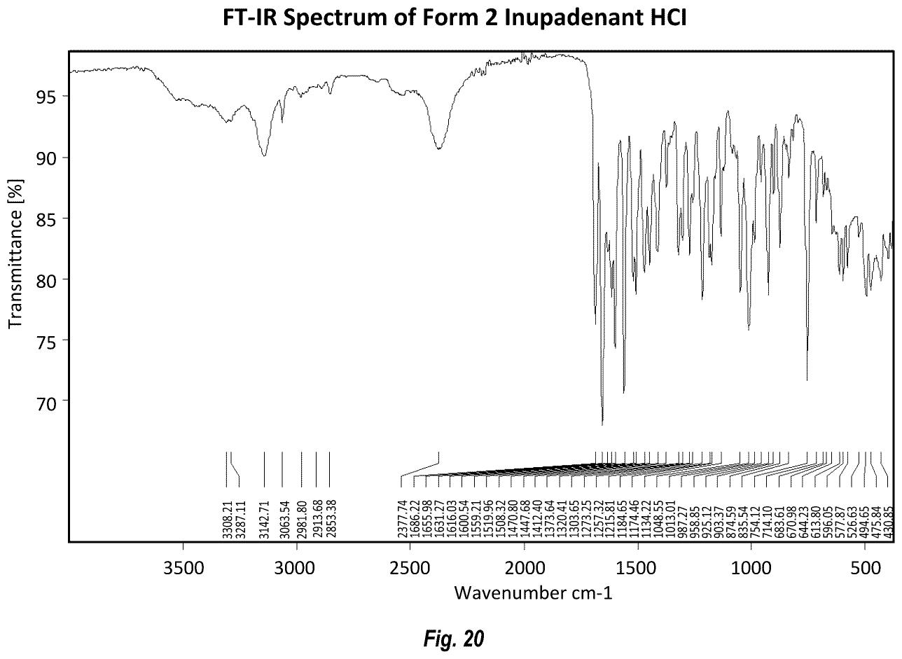

An FT-IR spectrum of Form 2 inupadenant hydrochloride was collected (Example 1) and is shown in FIG. 20. Tabulated characteristics of the FT-IR spectrum in FIG. 20 are provided below in Table 8, which lists peak wavenumber (cm⁻¹) and transmittance (%). The following stretches were observed: N-12 stretches ˜3300 cm⁻¹, aromatic C—H stretches ˜3000 cm⁻¹, C═O stretches ˜1700-1600 cm⁻¹, and aromatic C═C stretches ˜1660 cm⁻¹.

TABLE 8
FT-IR Data of Form 2 Inupadenant Hydrochloride

| | Transmittance |
|---|---|
| Wavenumber (cm⁻¹) | (%) |
| 3308.21 | 92.70 |
| 3287.11 | 92.90 |
| 3142.71 | 90.00 |
| 3063.54 | 92.80 |
| 2981.80 | 94.90 |
| 2913.67 | 95.70 |
| 2853.38 | 95.20 |
| 2377.74 | 90.60 |
| 1686.22 | 76.10 |
| 1655.98 | 67.90 |
| 1631.27 | 82.20 |
| 1616.03 | 78.50 |
| 1600.54 | 74.30 |
| 1559.21 | 70.60 |
| 1519.96 | 79.80 |
| 1508.32 | 78.70 |
| 1470.80 | 80.50 |
| 1447.68 | 81.10 |
| 1412.40 | 82.20 |
| 1373.64 | 87.50 |
| 1320.41 | 81.90 |
| 1303.65 | 83.10 |
| 1273.25 | 81.90 |
| 1257.32 | 86.20 |
| 1215.81 | 78.20 |
| 1184.65 | 81.70 |
| 1174.46 | 81.10 |
| 1134.22 | 83.50 |
| 1048.55 | 78.90 |
| 1013.01 | 75.70 |
| 987.27 | 83.00 |
| 958.85 | 87.90 |
| 925.12 | 78.70 |
| 903.37 | 86.90 |
| 874.59 | 82.50 |
| 835.54 | 88.30 |
| 754.12 | 71.40 |
| 714.10 | 84.60 |
| 683.61 | 86.70 |
| 670.98 | 87.20 |
| 644.23 | 83.70 |
| 613.80 | 80.30 |
| 596.05 | 79.80 |
| 577.87 | 80.90 |
| 526.63 | 83.40 |
| 494.65 | 78.60 |
| 475.84 | 79.00 |
| 430.85 | 79.80 |

HPLC purity analysis (Example 1, HPLC Method A) showed Form 2 inupadenant hydrochloride to have a purity of 99.6100 (by relative area).

HPLC-CAD analysis (Example 2, HPLC Method B) showed Form 2 inupadenant hydrochloride to contain 5.8% w/w chloride (0.9 equiv.), confirming a mono-hydrochloride salt.

KF analysis (Example 1) showed Form 2 inupadenant hydrochloride to have an average moisture content of 3.55% (1.3 mole equiv. water).

### Example 9. Variable Temperature XRPD Study of Form 2 Inupadenant Hydrochloride

Form 2 inupadenant hydrochloride was prepared according to the method described in Example 3.

A first VT-XRPD analysis (Example 1) of Form 2 inupadenant hydrochloride showed small changes in the XRPD pattern when heated to 100° C. (beyond the TGA mass loss, see Example 8), indicating slight structural changes upon dehydration (FIG. 6). Further changes were noted when heating past the endothermic event with an onset ca. 142° C., indicating a form change and a likely solid-solid phase transition at this temperature (FIG. 7). When heated past the larger endothermic event (ca. 229° C.), the material was observed to have melted. Upon cooling to 25° C., no recrystallization was observed in the diffractogram (FIG. 21). Tabulated characteristics of the XRPD patterns in FIG. 21 at 25° C., 100° C., 135° C., 162° C., and 200° C., are provided below in Tables 9-13, which lists diffraction angle (20) and relative intensity (expressed as a percentage with respect to the most intense peak).

TABLE 9
XRPD Data of Form 2 Inupadenant Hydrochloride at 25° C.

| Diffraction Angle | Relative Intensity |
|---|---|
| [°2θ] | [%] |
| 5.3 | 12.1 |
| 6.5 | 2.6 |
| 8.9 | 78.2 |
| 9.3 | 90 |
| 12.6 | 4.2 |
| 13.3 | 2.5 |
| 14.5 | 19.5 |
| 14.8 | 21.8 |
| 15.4 | 62.8 |
| 16.1 | 49.1 |
| 16.5 | 19.1 |
| 16.7 | 11.5 |
| 17.4 | 44.7 |
| 18 | 88 |
| 19.3 | 13.4 |
| 19.8 | 36.5 |
| 21.5 | 16.8 |
| 22.2 | 47.8 |
| 22.3 | 62.4 |
| 22.6 | 36.5 |
| 23.1 | 35.3 |
| 23.4 | 43.3 |
| 23.9 | 22.1 |
| 24.1 | 8.7 |
| 24.3 | 18.9 |
| 24.9 | 42.9 |
| 25.4 | 12 |
| 25.9 | 75.7 |
| 26.6 | 100 |
| 26.8 | 52.9 |
| 27.5 | 25.9 |
| 28.2 | 22.3 |
| 29.4 | 20.3 |
| 30.4 | 15.8 |
| 31 | 9.7 |
| 31.6 | 15.7 |
| 32.2 | 26 |
| 32.9 | 11.4 |
| 33.7 | 9.2 |
| 34.4 | 8 |

TABLE 10
XRPD Data of Crystalline Inupadenant Hydrochloride at 100° C.

| Diffraction Angle | Relative Intensity |
|---|---|
| [°20] | [%] |
| 5.4 | 11.6 |
| 6.7 | 3.8 |
| 7.6 | 4.1 |
| 9 | 32.6 |
| 9.5 | 84.3 |
| 12.6 | 7.4 |
| 13.6 | 5.9 |
| 14.6 | 21.6 |
| 15.2 | 53 |
| 15.8 | 81.2 |
| 16.1 | 39.4 |
| 16.6 | 29.3 |
| 17.4 | 44 |
| 17.6 | 62.4 |
| 18.2 | 83.1 |
| 19.1 | 19.3 |
| 19.9 | 50.5 |
| 20.7 | 18.1 |
| 21.5 | 35.9 |
| 22.8 | 60.6 |
| 23.6 | 60.1 |
| 24.2 | 47.4 |
| 24.8 | 69 |
| 25.7 | 100 |
| 26.6 | 97.7 |
| 27.1 | 62.8 |
| 27.5 | 85 |
| 28.2 | 45.5 |
| 29.2 | 34.8 |
| 29.8 | 51.8 |
| 30.5 | 41.5 |
| 31.8 | 51.7 |
| 32.3 | 50.5 |
| 33 | 56.5 |
| 33.9 | 48 |
| 35 | 55 |
| 35.6 | 51.3 |

TABLE 11
XRPD Data of Crystalline Inupadenant Hydrochloride at 135° C.

| Diffraction Angle | Relative Intensity |
|---|---|
| [°2θ] | [%] |
| 5.3 | 11.8 |
| 6.8 | 4.4 |
| 7.6 | 3.3 |
| 9.1 | 31 |
| 9.5 | 79.8 |
| 12.6 | 7.1 |
| 13.5 | 6.5 |
| 14.5 | 20.5 |
| 15.2 | 59.9 |
| 15.9 | 81.5 |
| 16.2 | 39.3 |
| 16.5 | 28.8 |
| 17.6 | 69.7 |
| 18.1 | 86.3 |
| 19.1 | 21.4 |
| 19.8 | 56.8 |
| 20.7 | 19.3 |
| 21.6 | 39.9 |
| 22.5 | 59.5 |
| 22.8 | 62.7 |
| 23.6 | 64.9 |
| 24.1 | 57.3 |
| 24.7 | 77.3 |
| 25.7 | 100 |
| 26.4 | 97 |
| 27.4 | 96 |
| 28.1 | 49.3 |
| 29.2 | 39.9 |
| 29.8 | 56.4 |
| 31.8 | 54.3 |
| 32.2 | 56.9 |
| 33 | 61.9 |
| 33.9 | 56.3 |
| 34.9 | 61 |

TABLE 12
XRPD Data of Crystalline Inupadenant Hydrochloride at 162° C.

| Diffraction Angle | Relative Intensity |
|---|---|
| [°20] | [%] |
| 5.3 | 18.5 |
| 6.7 | 4.5 |
| 8.8 | 26.3 |
| 9.5 | 60.2 |
| 12.4 | 4.8 |
| 13.4 | 8.2 |
| 14.9 | 40.4 |
| 15.4 | 76.2 |
| 16 | 25.2 |
| 17 | 12.1 |
| 18 | 100 |
| 18.8 | 14.9 |
| 19.3 | 24.4 |
| 20.4 | 7.9 |
| 21.1 | 17 |
| 21.9 | 31.8 |
| 22.7 | 40 |
| 23.5 | 5.9 |
| 24.5 | 61.6 |
| 25 | 56.8 |
| 25.8 | 33.1 |
| 26.1 | 44.3 |
| 26.6 | 42.1 |
| 27.6 | 16.2 |
| 28 | 18 |
| 29.2 | 27.7 |
| 30.6 | 8.7 |
| 31.2 | 11.8 |
| 32.2 | 20.9 |
| 33.4 | 12.6 |

TABLE 13
XRPD Data of Crystalline Inupadenant Hydrochloride at 200° C.

| Diffraction Angle | Relative Intensity |
|---|---|
| [°20] | [%] |
| 5.3 | 19.3 |
| 6.7 | 5 |
| 8.8 | 24.7 |
| 9.4 | 65.9 |
| 12.5 | 5.2 |
| 13.3 | 9.1 |
| 14.9 | 35.3 |
| 15.5 | 72.7 |
| 15.8 | 22.6 |
| 17.1 | 19.8 |
| 17.6 | 22.9 |
| 18 | 100 |
| 18.1 | 75.3 |
| 19.3 | 22.7 |
| 20.3 | 8.3 |
| 21.2 | 21.1 |
| 22 | 26.9 |
| 22.5 | 41.2 |
| 23.6 | 15.3 |
| 24.4 | 49.4 |
| 25.2 | 68.8 |
| 26.3 | 29.5 |
| 26.8 | 46.6 |
| 27.5 | 15.1 |
| 28 | 15.2 |
| 29.3 | 28.2 |
| 30.5 | 6.2 |
| 31.2 | 11.1 |
| 32.4 | 14.5 |
| 33.4 | 13.5 |

A second VT-XRPD analysis (Example 1) of Form 2 inupadenant hydrochloride showed small changes in the diffractogram when the sample was heated to 170° C. When the sample was cooled to 25° C., some evidence of reversion back to the low temperature diffraction pattern (e.g., pattern initially collected at 25° C.) was observed (FIG. 22).

### Example 10. Variable Humidity Study of Form 2 Inupadenant Hydrochloride

Form 2 inupadenant hydrochloride was prepared according to the method described in Example 3.

VH-XRPD analysis (Example 1) of Form 2 inupadenant hydrochloride showed no evidence of a phase transition between 30-90% RH (FIGS. 23A, 23B3, and 23C).

Some peak shifts were observed at <20% RH, with a potential form change observed at 2% RH (FIGS. 8, 23A and 23B). This phase transition is likely due to loss of water from the crystal lattice.

Tabulated characteristics of the XRPD patterns in FIGS. 8, 23A, 23B, and 23C, at initial ambient humidity, 20% RH, and 2% RH, are provided below in Tables 14-16, which lists diffraction angle (2θ) and relative intensity (expressed as a percentage with respect to the most intense peak).

TABLE 14
XRPD Data of Form 2 Inupadenant Hydrochloride
at Initial Ambient Humidity

| Diffraction Angle | Relative Intensity |
|---|---|
| [°20] | [%] |
| 5.5 | 3.1 |
| 9.1 | 28.1 |
| 9.5 | 46.3 |
| 13.5 | 3.6 |
| 14.9 | 15.7 |
| 15.6 | 35.4 |
| 16.3 | 49.8 |
| 16.7 | 13.6 |
| 17.5 | 36.1 |
| 18.2 | 100 |
| 19.4 | 13.1 |
| 20 | 27.9 |
| 21.7 | 26.4 |
| 22.6 | 79.1 |
| 23.2 | 33.9 |
| 23.7 | 41.4 |
| 24.1 | 26 |
| 24.4 | 18.9 |
| 25.1 | 29.3 |
| 26 | 50.8 |
| 26.8 | 72.1 |
| 27 | 60 |
| 27.7 | 26.6 |
| 28.6 | 22.5 |
| 29.5 | 30.7 |
| 30.6 | 22.1 |
| 31.1 | 18.8 |
| 31.7 | 31.2 |
| 32.4 | 49.4 |
| 33.1 | 30.2 |
| 33.9 | 27.7 |

TABLE 15
XRPD Data of Crystalline Inupadenant Hydrochloride at 20% RH

| Diffraction Angle | Relative Intensity |
|---|---|
| [°20] | [%] |
| 5.4 | 1.8 |
| 9.1 | 33.8 |
| 9.5 | 56.3 |
| 13.5 | 3.8 |
| 15 | 21 |
| 15.7 | 48.3 |
| 16.3 | 47.8 |
| 16.7 | 16.7 |
| 17.7 | 57.8 |
| 18.3 | 100 |
| 20 | 35.8 |
| 21.7 | 44.4 |
| 22.4 | 57.4 |
| 22.6 | 92.7 |
| 23.3 | 45.5 |
| 23.7 | 51.2 |
| 24.0 | 31.9 |
| 24.6 | 24.9 |
| 25.2 | 45.8 |
| 26.2 | 71.6 |
| 27 | 94.2 |
| 27.3 | 73.3 |
| 27.9 | 36.8 |
| 28.5 | 39.9 |
| 29.5 | 39.2 |
| 30.8 | 23.5 |
| 31.9 | 47.2 |
| 32.4 | 64.1 |
| 33.3 | 39.8 |
| 34 | 37.9 |

TABLE 16
XRPD Data of Crystalline Inupadenant Hydrochloride at 2% RH

| Diffraction Angle | Relative Intensity |
|---|---|
| [°20] | [%] |
| 5.5 | 2.6 |
| 9.2 | 20.9 |
| 9.6 | 58 |
| 12.9 | 3.6 |
| 13.5 | 3.4 |
| 14.8 | 10.7 |
| 15.3 | 28.7 |
| 16 | 51.7 |
| 16.3 | 38.7 |
| 16.8 | 17.4 |
| 17.7 | 46.8 |
| 18.3 | 96.4 |
| 18.4 | 100 |
| 20.1 | 28.5 |
| 21.7 | 37 |
| 22.7 | 75.8 |
| 23.8 | 51.7 |
| 24.3 | 26.2 |
| 25.2 | 38.4 |
| 26.1 | 60.5 |
| 26.9 | 76.4 |
| 27.4 | 63 |
| 27.9 | 37.6 |
| 28.5 | 27.4 |
| 29.9 | 29.6 |
| 30.8 | 21.4 |
| 32 | 36.9 |
| 32.5 | 47.2 |
| 33.3 | 35.8 |
| 34 | 31.3 |

### Example 11. Solubility Assessment of Form 2 Inupadenant Hydrochloride

Approximately 10 mg of the Form 2 inupadenant hydrochloride (prepared in accordance with the method provided in Example 3) was added to 2 mL vials. 0.5-1 mL of solvent was added to each vial and the samples were manually shaken at ambient temperature. Gentle heating was used to encourage dissolution of Form 2 inupadenant hydrochloride. The solvents assessed in this solubility study were 1,4-dioxane, water, tert-butyl alcohol, THF, ethyl acetate, acetone, methanol, and DCM.

Form 2 inupadenant hydrochloride was found to be insoluble in all the solvent systems assessed, even at 20 mg/mL with additional gentle heating.

### Example 12. Stability Assessment of Form 2 Inupadenant Hydrochloride

A 7-day stability assessment of Form 2 inupadenant hydrochloride was carried out as follows: (1) 20 mg Form 2 inupadenant was added to 3×2 mL glass vials; (2) the samples were then stored under the following conditions—(i) 40° C./75% RH—open vial, (ii) 80° C.—closed vial, and (iii) ambient light—open vial; (3) Solids were stored for 1 week under the above conditions with XRPD analysis (Example 1, XRPD Method A) and HPLC purity analysis (Example 1, HPLC Method A) being carried out post-storage.

XRPD analysis of the solids recovered from the 7-day stability assessment showed no changes to the solid form after exposure to the stability assessment conditions (Table 17).

HPLC purity analysis showed no changes to solid purity when Form 2 inupadenant hydrochloride was exposed to the 7-day stability conditions (Table 17).

TABLE 17
Form 2 Inupadenant Hydrochloride 7 Day Stability Assessment

| | | HPLC (Purity, |
|---|---|---|
| Conditions | XRPD Analysis | % area) |
| Input Form 2 | Form 2 inupadenant | 99.61 |
| inupadenant | hydrochloride | |
| hydrochloride | | |
| 40° C. + | Form 2 inupadenant | 99.64 |
| 75% RH | hydrochloride | |
| 80° C. | Form 2 inupadenant | 99.66 |
| | hydrochloride | |
| Ambient | Form 2 inupadenant | 99.66 |
| | hydrochloride | |

### Example 13. Stability Assessment of Amorphous Inupadenant Hydrochloride

A 7-day stability assessment of amorphous inupadenant hydrochloride was carried out as follows: (1) 20 mg amorphous inupadenant was added to 3×2 mL glass vials; (2) the samples were then stored under the following conditions—(i) 40° C./75% RH—open vial, (ii) 80° C.—closed vial, and (iii) ambient light—open vial; (3) Solids were stored for 1 week under the above conditions with XRPD analysis (Example 1, XRPD Method A) and HPLC purity analysis (Example 1, HPLC Method A) being carried out post-storage.

XRPD analysis of the solids recovered from the 7-day stability assessment showed amorphous inupadenant hydrochloride crystallized into Form 2 inupadenant hydrochloride after exposure to all of the stability assessment conditions (Table 18).

HPLC purity analysis showed no changes to solid purity when Form 2 inupadenant hydrochloride was exposed to the 7-day stability conditions (Table 18).

TABLE 18
Amorphous Inupadenant Hydrochloride
7 Day Stability Assessment

| | | HPLC (Purity, |
|---|---|---|
| Conditions | XRPD Analysis | % area) |
| Input amorphous | Predominantly amorphous | 99.61 |
| inupadenant | inupadenant hydrochloride | |
| hydrochloride | | |
| 40° C. + | Form 2 inupadenant | 99.64 |
| 75% RH | hydrochloride | |
| 80° C. | Form 2 inupadenant | 99.66 |
| | hydrochloride | |
| Ambient | Form 2 inupadenant | 99.66 |
| | hydrochloride | |

### Example 14. Salt Disproportionation Study of Form 2 Inupadenant Hydrochloride

A salt disproportionation study of Form 2 inupadenant hydrochloride was carried out as follows: (1) 20 mg Form 2 inupadenant hydrochloride was weighed into a 2 mL vial and 1 mL unbuffered water was added; (2) The mixture in the vial was then agitated at ambient temperature for 24 hours; (3) the solids were then analyzed by XRPD (Example 1, XRPD Method A); (4) the solids were then dried under vacuum at 40° C. for 24 hours; and (5) the dried solids were then analyzed by XRPD (Example 1, XRPD Method A).

XRPD analysis of Form 2 inupadenant hydrochloride after slurring in water showed no changes in the solid form.

### Example 15. Dissolution Rate Assessment of Form 2 Inupadenant Hydrochloride

A dissolution rate assessment of Form 2 inupadenant hydrochloride was carried out in pH 1.2, 0.2 M HCl/KCl buffer using the following method:
Approximately 200 mg Form 2 inupadenant hydrochloride was weighed out accurately in quadruplicate and each aliquot placed into separate 20 mL glass vials.
To each vial was added 10 mL of pH 1.2, 0.2 M HCl/KCl buffer.
This buffer had been previously checked for miscibility with the method diluent and no issues were observed.
Each vial was placed on a thermostatically controlled shaker at ca. 37° C.
Ca. 1000 μL of each sample was drawn from the slurry layer by automated pipette at 15, 30, 60, 120, 240 and 1440 minute timepoints.
These aliquots were spun down through a centrifuge filter, diluted 300 μl:300 μl with the method specified diluent and placed in total recovery vials. The solids were retained for XRPD analysis.
The solubility of the material in the buffer was determined by HPLC analysis (duplicate injections) (Example 1, HPLC Method C) against verified reference solutions of the source material at known concentrations.

Results obtained for the dissolution rate assessment carried out in a range of pH buffers were as follows:

TABLE 19
Form 2 Inupadenant Hydrochloride Dissolution Rate Assessment

| | Conc. mg/mL in |
|---|---|
| Timepoint (min) | pH 1.2 HCl/KCl |
| 15 | 0.0010 |
| 30 | 0.0013 |
| 60 | 0.0014 |
| 120 | 0.0023 |
| 240 | 0.0029 |
| 1440 | 0.0035 |

It was observed that the solubility of Form 2 inupadenant hydrochloride in pH 1.2 0.2 M HCl/KCl buffer was low, with a slow rate of increase when agitated at 37° C. overnight.

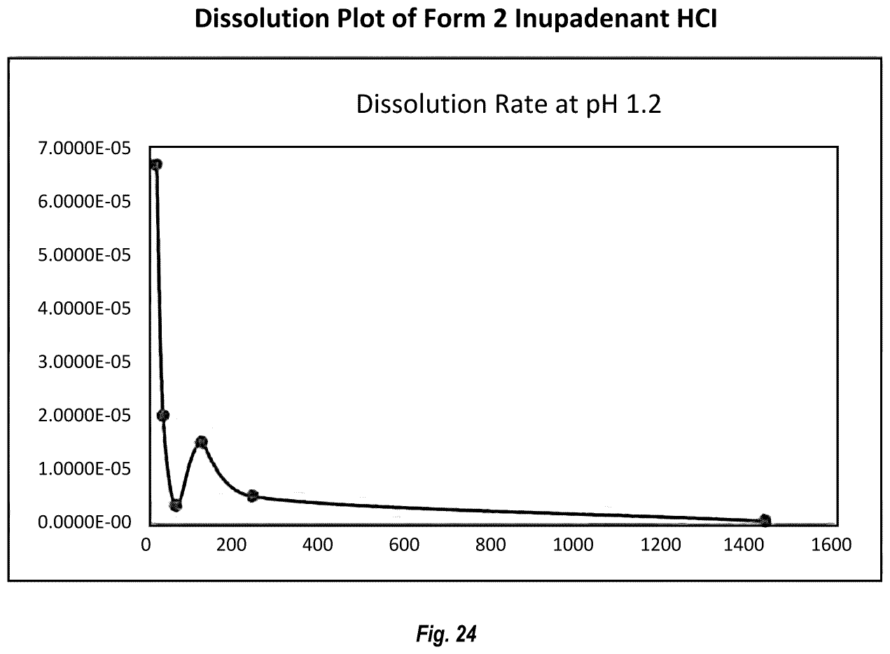

The dissolution rate for the pH 1.2 and pH 3.5 results was calculated as follows:

$${R}^{D} = \frac{Δ C}{Δ T}$$

Where R^{D} is the dissolution rate, AC is the change in concentration in mg/mL and ΔT is the number of minutes over which the change occurred. Plots of the dissolution rate of Form 2 inupadenant hydrochloride in pH 1.2 HCl/KCl buffer is shown in FIG. 24.

For the pH 1.2 HCl/KCl buffer, the solids were recovered from each timepoint solution extraction and analyzed by XRPD. All solids recovered were consistent with Form 2 inupadenant hydrochloride.

### Example 16. Process for Manufacturing Exemplary Form 2 Inupadenant Hydrochloride Pharmaceutical Compositions

In the following example, the manufacturing process is outlined for all exemplified Form 2 inupadenant hydrochloride pharmaceutical compositions. The corresponding amounts of ingredients are provided in the formulas disclosed in Table 20.

Gelucire 44/14 and/or the PEG (e.g., PEG 400 or PEG 1000) were heated to 60° C. until molten. The molten Gelucire 44/14 and/or the molten PEG were then weighed into a suitable pre-warmed vessel and the temperature maintained at 60° C. The BHT, copovidone, and Form 2 inupadenant hydrochloride were then added to the molten Gelucire 44/14 and/or PEG mixture and mixed until fully wetted. The temperature of the mixture was maintained at 60° C. throughout the mixing process. The mixture was then homogenized at 2810 rpm for at least 30 minutes at 60° C. After homogenization, the mixture was then low shear mixed at 160 rpm for at least 30 minutes at 60° C. to provide the pharmaceutical composition. Finally, the temperature was maintained at 60° C. while filing capsules with the pharmaceutical composition.

TABLE 20
Formulas of Exemplary Inupadenant Hydrochloride Pharmaceutical Compositions

| Formulation | Composition |
|---|---|
| 1 | 5.1% w/w Form 2 inupadenant hydrochloride, 87.4% w/w Gelucire 44/14, |
| | 6.3% w/w PEG 400, 1.15% w/w Copovidone, 0.05% w/w BHT |
| 2 | 5.1% w/w Form 2 inupadenant hydrochloride, 87.4% w/w, Gelucire 44/14, |
| | 6.3% w/w PEG 400, 1.15% w/w Copovidone, 0.05% w/w BHT |
| 3 | 5.1% w/w Form 2 inupadenant hydrochloride, 86.25% w/w, Gelucire 44/14, |
| | 6.30% w/w PEG 400, 2.3% w/w copovidone, 0.05% w/w BHT |
| 4 | 5.1% w/w Form 2 inupadenant hydrochloride, 86.15% w/w Gelucire 44/14, |
| | 6.30% w/w PEG 400, 2.3% w/w copovidone, 0.15% w/w BHT |
| 5 | 5.1% w/w Form 2 inupadenant hydrochloride, 81.1% w/w Gelucire 44/14, |
| | 12.6% w/w PEG 400, 1.15% w/w copovidone, 0.05% w/w BHT |
| 6 | 5.1% w/w Form 2 inupadenant hydrochloride, 81.0% w/w PEG 400, 1.15% |
| | w/w copovidone, 0.15% w/w BHT |
| 7 | 5.1% w/w Form 2 inupadenant hydrochloride, 79.95% w/w Gelucire 44/14, |
| | 12.6% w/w PEG 400, 2.3% w/w copovidone, 0.05% w/w BHT |
| 8 | 5.1% w/w Form 2 inupadenant hydrochloride, 79.85% w/w Gelucire 44/14, |
| | 12.6% w/w PEG 400, 2.3% copovidone, 0.15% w/w BHT |
| 9 | 5.1% w/w Form 2 inupadenant hydrochloride, 87.4% w/w Gelucire 44/14, |
| | 6.3% w/w PEG 1000, 1.15% w/w copovidone, 0.05% w/w BHT |
| 10 | 5.1% w/w Form 2 inupadenant hydrochloride, 87.3% w/w Gelucire 44/14, |
| | 6.3% w/w PEG 1000, 1.15% w/w copovidone, 0.15% w/w BHT |
| 11 | 5.1% w/w Form 2 inupadenant hydrochloride, 86.25% w/w Gelucire 44/14, |
| | 6.3% w/w PEG 1000, 2.3% w/w copovidone, 0.05% w/w BHT |
| 12 | 5.1% w/w Form 2 inupadenant hydrochloride, 86.15% w/w Gelucire 44/14, |
| | 6.3% w/w PEG 1000, 2.3% w/w copovidone, 0.15% w/w BHT |
| 13 | 5.1% w/w Form 2 inupadenant hydrochloride, 81.1% w/w Gelucire 44/14, |
| | 12.6% w/w PEG 1000, 1.15% w/w copovidone, 0.05% w/w BHT |
| 14 | 5.1% w/w Form 2 inupadenant hydrochloride, 81.0% w/w Gelucire 44/14, |
| | 12.6% w/w PEG 1000, 1.15% w/w copovidone, 0.15% w/w BHT |
| 15 | 5.1% w/w Form 2 inupadenant hydrochloride, 79.95% w/w Gelucire 44/14, |
| | 12.6% w/w PEG 1000, 2.3% w/w copovidone, 0.05% w/w BHT |
| 16 | 5.1% w/w Form 2 inupadenant hydrochloride, 79.85% w/w Gelucire 44/14, |
| | 12.6% w/w PEG 1000, 2.3% w/w copovidone, 0.15% w/w BHT. |
| 17 | 5.1% w/w Form 2 inupadenant hydrochloride, 81.05% w/w Gelucire 44/14, |
| | 12.6% w/w PEG 1000, 1.15% w/w copovidone, 0.1% w/w BHT. |

### Example 17. A Process for Manufacturing Pharmaceutical Compositions of Inupadenant Hydrochloride

Inupadenant hydrochloride (e.g., Form 2 inupadenant hydrochloride with the XRPD pattern shown in FIG. 4) drug product is formulated as solid oral capsule dosage forms using a common blend as two unit doses: a low-strength 20 mg dose and a medium-strength 40 mg dose (both freebase equivalent). Multiple capsules may be dosed in order to achieve higher doses. The dosage form is a conventional immediate-release capsule manufactured using known pharmacopeial excipients and by conventional mixing and capsule-filling processes. The manufacturing process differs only at the capsule filling stage for the capsule strengths where different capsule fill weights and different colored capsules are required.

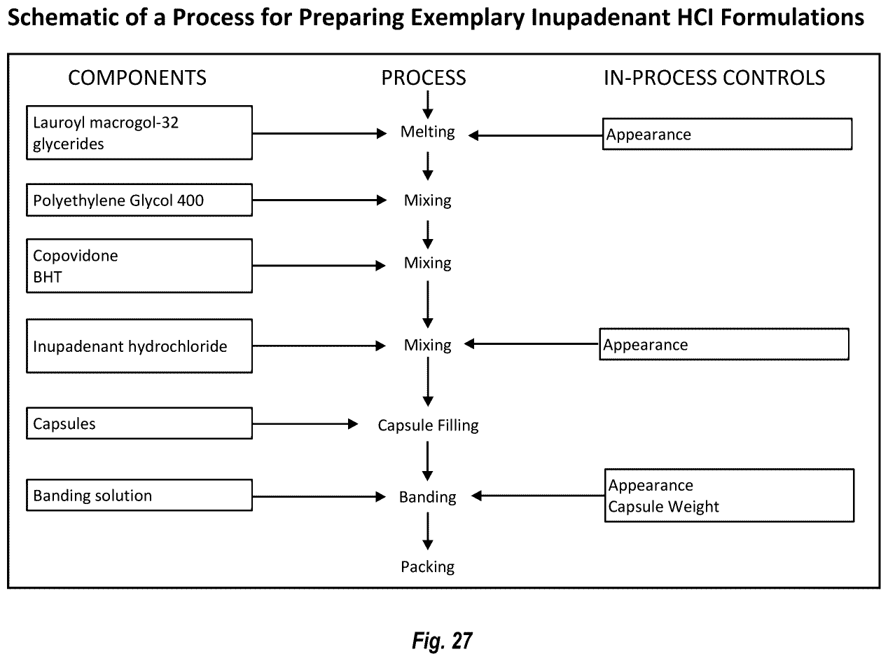

The process is schematically described in FIG. 27 and comprises the following steps:
i) lauroyl polyoxyl-32 glycerides is melted at a temperature not less than 50° C. but not exceeding 80° C.;
ii) Polyethylene Glycol 400 is added to the lauroyl polyoxyl-32 glycerides and mixed together
iii) Copovidone and BHT, are then added and mixing is continued;
iii) the inupadenant hydrochloride (e.g., a crystalline hydrochloride salt of inupadenant) is then added gradually under continuous mixing using a suitable mixer to produce a visually uniform distribution of the drug substance with no observable lumps or agglomerates;
iv) mixing is then continued for at least 30 minutes to ensure that the drug substance is homogeneously distributed as determined visually;
v) the blend is then maintained in the molten state with continued mixing and is filled into appropriately sized gelatin capsule shells to the target capsule fill weight; and
vi) the capsules are banded with banding solution.

The quantitative composition for the two dosage strengths of Form 2 inupadenant hydrochloride is shown in Table 21.

TABLE 21
Quantitative Composition of Form 2 Inupadenant
Hydrochloride 20- and 40-mg capsules

| | Composition | Composition | Composition for 20 | |
|---|---|---|---|---|
| | for 20 mg | for 40 mg | and 40 mg capsules | |
| Component | capsule (mg) | capsule (mg) | (% w/w) | Function |
| Inupadenant HC1 ^{a} | 21.21 (20 | 42.42 (40 | 5.1 | Active |
| | free base) | free base) | | Ingredient |
| Lauroyl macrogol- | 305 | 610 | 73.4 | Wetting/ |
| 32 glycerides | | | | Dissolution |
| | | | | aid |
| Polyethylene glycol | 70 | 140 | 16.8 | Wetting/ |
| 400 | | | | Dissolution |
| | | | | aid |
| Copovidone | 19 | 38 | 4.6 | Dissolution |
| | | | | aid |
| Butylated | 0.42 | 0.83 | 0.1 | Antioxidant |
| hydroxytoluene | | | | |
| (BHT) | | | | |
| Opaque gelatin | 1 white capsule | 1 yellow | — | Encapsulation |
| capsule | (size 1) | capsule | | |
| | | (size 00) | | |
| Total per capsule^{b} | 415.63 mg | 831.25 mg | 100.00 | |

^{a} 5.1% w/w drug loading for inupadenant hydrochloride is equivalent to a loading of 4.8% w/w as the free base of inupadenant.
^{b}Capsules are banded using clear gelatin banding solution (21.69% w/w gelatin, 0.92% w/w polysorbate 80 and 77.39% w/w purified water. Water is removed during manufacture leaving trace amounts of gelatin and polysorbate 80, average band weight may vary but is typically 4 mg for both the 20 mg and 40 mg capsules.

### Example 18. Investigation of In Vivo PK Parameters in Dog for Multiple Formulations of Inupadenant Hydrochloride

The influence of various parameters on in vivo pharmacokinetic performance for compositions of inupadenant free base and inupadenant hydrochloride salt were investigated in three studies, where dogs were administered oral compositions containing inupadenant, with and without pentagastrin to stimulate stomach acid production.

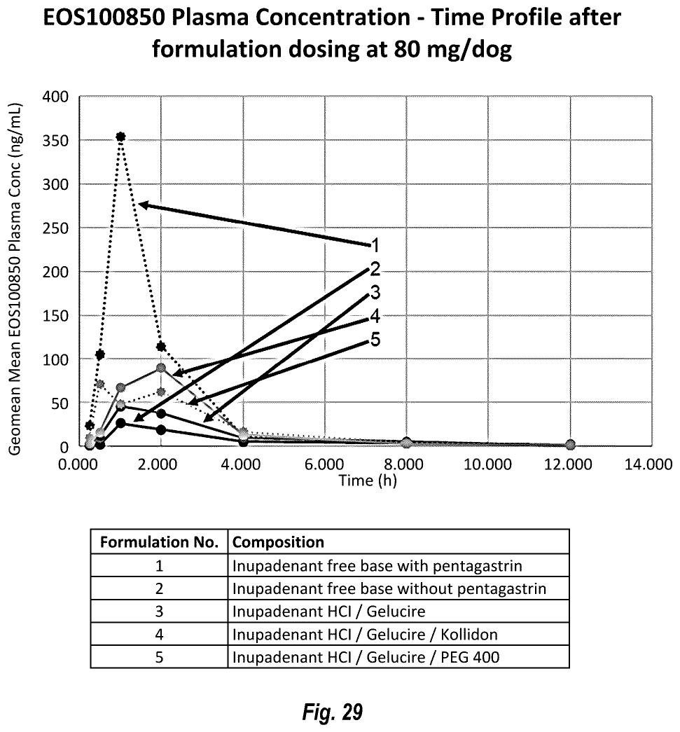

In the first study, the clinical inupadenant free base formulation (also comprising Gelucire 44/14, PEG 400, and BHT), with and without pentagastrin, was compared to three formulations of inupadenant hydrochloride in lauroyl macrogol-32 glycerides (Gelucire) only, lauroyl macrogol-32 glycerides (Gelucire) and Copovidone (Kollidon® VA 64-1%) and Gellucire and PEG 400. The inupadenant hydrochloride formulations were tested without administration of pentagastrin. The results, summarized in Table 22 and FIG. 28 and FIG. 29, show that the inupadenant hydrochloride formulations increase exposure and reduce variability between animals compared with the inupadenant free base formulation in the absence of pentagastrin, with no animals in the inupadenant hydrochloride group showing undetectable levels (unlike the inupadenant free base formulation). The overall exposure for the inupadenant hydrochloride formulations is lower (by 2-fold or more) than the exposure obtained with inupadenant free base in the presence of pentagastrin, but the variability is lower in most cases, suggesting the potential for a slightly different, flatter PK profile. Despite the promising performance of the Gellucire-only formulation in dissolution studies, addition of PEG and Copovidone (Kollidon®) appears important for in vivo performance.

TABLE 22
Comparison Between Inupadenant Free Base “clinical” Formulation
and Form 2 Inupadenant Hydrochloride Formulation.

| | Geomean AUCt |
|---|---|
| Formulation (1 × 80 mg) dose | (ng · h/ml)/(% CV) |
| Free base clinical + | 500/(44) |
| pentagastrin | |
| Free base clinical − | 110/(113) |
| pentagastrin | |
| HCI salt/lauroyl macrogol-32 glycerides (Gelucire) − | 164/(141) |
| pentagastrin | |
| HCI salt/lauroyl macrogol-32 glycerides (Gelucire)/Copovidone | 257/(44) |
| (Kollidon ®) − pentagastrin | |
| HCI salt/lauroyl macrogol-32 glycerides | 250/(32) |
| (Gelucire)/polyethyleneglycol (PEG 400) − | |
| pentagastrin | |

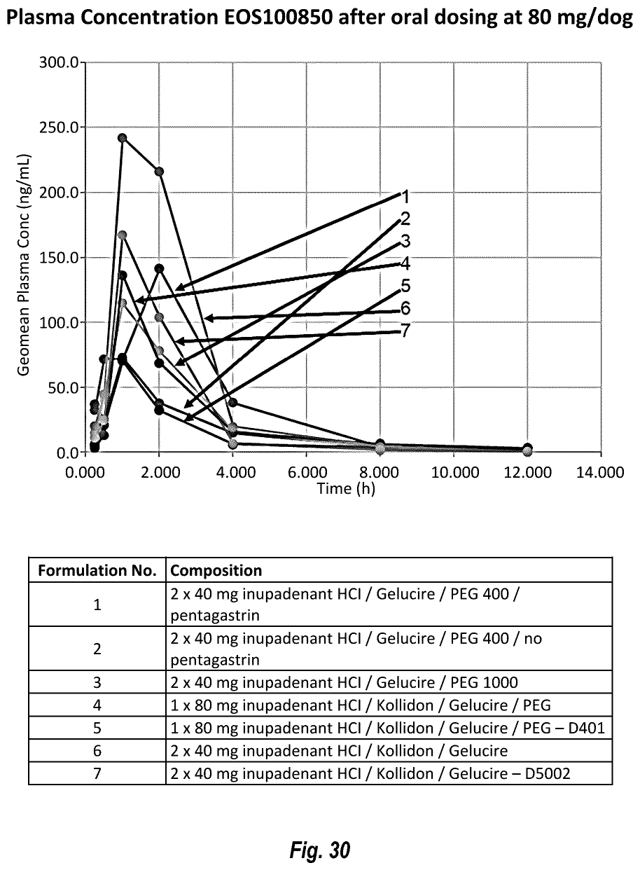

A second dog PK study analyzed the effect of excipient combinations and drug load on the pharmacokinetic profile of the inupadenant hydrochloride composition. In this study, the drug loading was reduced by 50%, from 10% inupadenant in the first study to ˜5% inupadenant in the second study. The results summarized in Table 23 and FIG. 30 show that a combination of PEG and copovidone (Kollidon®) is beneficial, a decrease in drug load results in an increase in performance and that the substitution of PEG 400 with PEG 1000 does not have any negative effects.

TABLE 23
Effect of Drug Loading and Excipient Combinations on Inupadenant PK.

| | Geomean AUCt |
|---|---|
| Formulation (inupadenant hydrochloride) | (ng · h/ml)/(% CV) |
| 2 × 40 mg lauroyl macrogol-32 glycerides (Gelucire)/polyethylene | 510/(36) |
| glycol (PEG400) + pentagastrin | |
| 2 × 40 mg lauroyl macrogol-32 glycerides | 243/(88) |
| (Gelucire)/polyethyleneglycol (PEG 400) − pentagastrin | |
| 2 × 40 mg lauroyl macrogol-32 glycerides | 333/(40) |
| (Gelucire)/polyethyleneglycol (PEG 1000) − pentagastrin | |
| 1 × 80 mg copovidone(Kollidon ®)/lauroyl macrogol-32 glycerides | 379/(181) |
| (Gelucire)/polyethyleneglycol (PEG 400) − pentagastrin | |
| 1 × 80 mg copovidone (Kollidon ®)/lauroyl macrogol-32 glycerides | 201/(43) |
| (Gelucire)/polyethyleneglycol (PEG 400)* − pentagastrin | |
| 2 × 40 mg copovidone (Kollidon ®)/lauroyl macrogol-32 glycerides | 614/(190) |
| (Gelucire)/polyethylene glycol (PEG 400) − pentagastrin | |

*Data recalculated after eliminating an outlier animal with abnormally high exposure

A third dog PK study compared two different doses (40 mg and 120 mg) of the inupadenant hydrochloride formulation with lauroyl macrogol-32 glycerides (Gelucire)/copovidone (Kollidon®)/polyethylene glycol (PEG) against the inupadenant free base clinical formulation (also comprising Gelucire 44/14, PEG 400, and BHT), with and without administration of pentagastrin. The results are summarized in Table 24.

TABLE 24
Comparison Between Pentagastrin and Pentagastrin-Free Administration of the
Inupadenant Hydrochloride Formulations in Dogs at 40 and 120 mg Doses.

| | Geomean AUCt |
|---|---|
| Formulation | (ng · h/ml)/(% CV) |
| HCI salt: 1 × 40 mg copovidone (Kollidon ®)/lauroyl macrogol-32 | 327/(41) |
| glycerides (Gelucire)/PEG + pentagastrin | |
| HCl salt: 1 × 40 mg copovidone (Kollidon ®)/lauroyl macrogol-32 | 44/(107) |
| glycerides (Gelucire)/polyethyleneglycol (PEG) − pentagastrin | |
| HCI salt: 3 × 40 mg copovidone (Kollidon ®)/lauroyl macrogol-32 | 694/(59) |
| glycerides (Gelucire)/polyethyleneglycol (PEG) + pentagastrin | |
| HCl salt: 3 × 40 mg copovidone (Kollidon ®)/lauroyl macrogol-32 | 343/(92) |
| glycerides (Gelucire)/polyethylene glycol (PEG) − pentagastrin | |
| Free base 1 × 40 mg - Clinical formulation − pentagastrin | 11/(123) |
| Free base 3 × 40 mg - Clinical formulation − pentagastrin | 198/(76) |

Based on the above results, the hydrochloride salt formulation of inupadenant is likely to give more reproducible exposure in humans, with greater dose proportionality.

Of note, PEG 1000 was replaced with PEG 400 in the final composition due to the superior consistency in the in vitro dissolution experiments.

### Example 19. First in Human (FIH) Clinical Trial Evaluation for an Inupadenant Free Base Formulation

Inupadenant free base was evaluated in a first-in-human (FIH) clinical study. The inupadenant free base formulation used in Examples 18 and 19 (“Clinical inupadenant free base formulation”) contains inupadenant 10%—lauroyl macrogol-32 glycerides EP (Gelucire 44/14) 71.90%—Polyethylene glycol 400 18%—Butylated hydroxytoluene 0.10% (% w/w). Study IO-001 (NCT03873883) is an interventional, multicenter, open-labeled, phase 1/1b study, designed to assess the safety, tolerability, the maximum tolerated dose (MTD), recommended phase 2 dose (RP2D) and PK, pharmacodynamics (PD), and antitumor activity of inupadenant free base when administered as monotherapy or in combination with pembrolizumab and/or chemotherapy in participants with advanced cancers. The compound was administered orally at doses of 20 mg and 40 mg once daily (QD) or 40, 80 and 160 mg twice daily (BID), depending on the study arm. The compound was administered with 250 mL of an authorized acidic beverage, under fasted conditions.

Data was available for 71 advanced solid tumor patients enrolled in the study. No dose-limiting toxicity (DLT) or dose related safety signals were observed during the monotherapy dose escalation. PK analysis demonstrated acceptable dose-proportionality through 80 mg BID, however, plasma exposures obtained with the 160 mg BID cohort showed no further increase. Thus, the overall PK data obtained for IO-001 indicate that the inupadenant free base formulation reached an absorption-limited plateau with highest exposures achievable at 80 mg BID. A total of 6 partial responses (PRs) have been observed in the study as of 7 Sep. 2021. In addition, several stable diseases (SDs) have been observed in the different cohorts including an ongoing durable SD (˜15 months) in a heavily pretreated NSCLC patient in the monotherapy arm (inupadenant 80 mg BID).

### Example 20. Human Clinical Trial Evaluation of a Composition Comprising Form 2 Inupadenant Hydrochloride

Study A2A-004 is an interventional, multicenter, open-label clinical trial evaluating a dose escalation and food effects for a composition comprising inupadenant hydrochloride (Inupadenant hydrochloride) in patients with advanced solid tumors at dose-levels of inupadenant HCl ranging from 40 mg BID to 320 mg BID. As of 25 Aug. 2021, 4 patients have been enrolled in the study with no notable safety findings and 3 patients have evaluable PK data. Notably, these initial PK results indicate that 40 mg BID inupadenant HCl formulation is able to achieve similar plasma levels to 80 mg BID inupadenant free base formulation. Similar exposure was observed between inupadenant HCl 40 mg BID and inupadenant 80 mg BID free base formulation (the monotherapy RP2D for free base), with less variability, and no safety/tolerability issues have been observed with inupadenant HCl 40 mg BID dose.

### Example 21. Clinical Trial to Evaluate Inupadenant Hydrochloride in Patients with Metastatic Non-Small Cell Lung Cancer (mNSCLC) or Locally Advanced, Unresectable NSCLC

A pharmaceutical composition comprising inupadenant (e.g., a pharmaceutical composition comprising inupadenant hydrochloride) is evaluated in an interventional, multicenter, randomized, placebo-controlled, phase 2 study evaluating the efficacy of inupadenant in non-small cell lung cancer. NSCLC includes non-small cell carcinoma not otherwise specified (<5%), squamous cell carcinoma (25%-30%), and nonsquamous carcinoma (adenocarcinoma, large cell, and undifferentiated carcinoma; 70%-75%.

The clinical study evaluates the safety and efficacy of inupadenant in combination with carboplatin and pemetrexed as a second-line therapy in adult patients with metastatic non-small cell lung cancer (mNSCLC) or locally advanced, unresectable NSCLC of nonsquamous pathology, with two primary goals:
Assess the safety and tolerability of the combination of inupadenant HCl and carboplatin/pemetrexed in an open-label, safety run-in phase, and determine the appropriate combination dose to continue to the randomized portion of the study
Assess the efficacy and safety of inupadenant HCl combined with carboplatin/pemetrexed compared to a control arm receiving placebo and carboplatin/pemetrexed.

The dose-finding part (Part 1) evaluates safety with a starting dose of inupadenant HCl at 40 mg twice daily (BID) combined with the standard approved doses of platinum chemotherapy (carboplatin area under the curve 5 mg/ml per min [AUC5] and pemetrexed 500 mg/m2 every 3 weeks [Q3W] for 4 cycles, followed by pemetrexed maintenance therapy). Each cycle covers 3 weeks. This dose is tested in a modified (3+3) escalation with a minimum of 6 evaluable participants, allowing for the enrollment of additional backup participants in case of non-evaluability of any participants in the starting cohort. Two additional dose cohorts (e.g., 80 mg BID and 160 mg BID) may be considered in the dose escalation if the starting dose is well tolerated. In addition, a 20 mg BID dose may be considered based on the available safety and PK data.

In Part 2, 150 patients are randomized 1:1 to inupadenant or placebo, both in combination with carboplatin and pemetrexed. Tumor response is determined according to RECIST 1.1 criteria and safety findings are reviewed by a Safety Review Committee (for Part 1) and a Data Monitoring Committee (for Part 2).

Key eligibility criteria include 1) Metastatic NSCLC (Stage IV) or locally advanced, unresectable (Stage III) NSCLC of nonsquamous pathology that has relapsed or progressed after prior anti-programmed death (PD)-ligand (L)1 therapy, 2) Stage IV patients should have received only 1 line of anti-PD-(L)1 therapy in the metastatic setting, without concomitant chemotherapy (immuno-oncology/immuno-oncology combination therapy is allowed); Stage III patients should have received single-agent durvalumab therapy post-chemoradiation), 3) have measurable disease as defined by RECIST 1.1 criteria and 4) Eastern Cooperative Oncology Group status <1. The primary endpoints are recommended Phase 2 dose to be used in combination with carboplatin and pemetrexed in Part 2 of the study (for Part 1) and progression-free survival between the active arm (inupadenant and carboplatin and pemetrexed) and the control arm (placebo and carboplatin and pemetrexed) (for Part 2). Secondary endpoints include change in tumor size, objective response rate, overall survival, and adverse events. Correlative aims include assessing blood and tissue biomarkers for association with clinical benefit.

### Example 22. Dissolution of Exemplary Form 2 Inupadenant Hydrochloride Pharmaceutical Compositions

Form 2 inupadenant hydrochloride compositions were prepared according to Tables 25 and 26.

TABLE 25
Formulas of Exemplary Form 2 Inupadenant
Hydrochloride Pharmaceutical Compositions

| Formulation | Composition |
|---|---|
| 1 | Form 2 inupadenant hydrochloride in 0.89% w/w Soluplus, 88.51% w/w |
| | Gelucire 44/14 |
| 2 | Form 2 inupadenant hydrochloride in 0.89% w/w, 88.51% w/w Gelucire |
| | 44/14 |
| 3 | Form 2 inupadenant hydrochloride in 0.89% w/w Kollidon, 88.51% w/w |
| | Gelucire 44/14 |
| 4 | Form 2 inupadenant hydrochloride in 0.89% w/w HPMC 606, 88.51% w/w |
| | Gelucire 44/14 |
| 5 | Form 2 inupadenant hydrochloride in 0.89% w/w Pluronic 127, 88.51% w/w |
| | Gelucire 44/14 |
| 6 | Form 2 inupadenant hydrochloride in 0.09% w/w SDS, 89.31% w/w |
| | Gelucire 44/14 |
| 7 | Form 2 inupadenant hydrochloride in 89.4% w/w Gelucire 44/14 |

TABLE 26
Formulas of Exemplary Form 2 Inupadenant
Hydrochloride Pharmaceutical Compositions

| Formulation | Composition |
|---|---|
| 1 | Form 2 inupadenant hydrochloride in 0.89% w/w Kollidon VA 64, 88.51% |
| | w/w Gelucire 44/14 |
| 2 | Form 2 inupadenant hydrochloride in 2.23% w/w Kollidon VA 64, 87.77% |
| | w/w Gelucire 44/14 |
| 3 | Form 2 inupadenant hydrochloride in 4.47% w/w Kollidon, 85.53% w/w |
| | Gelucire 44/14 |

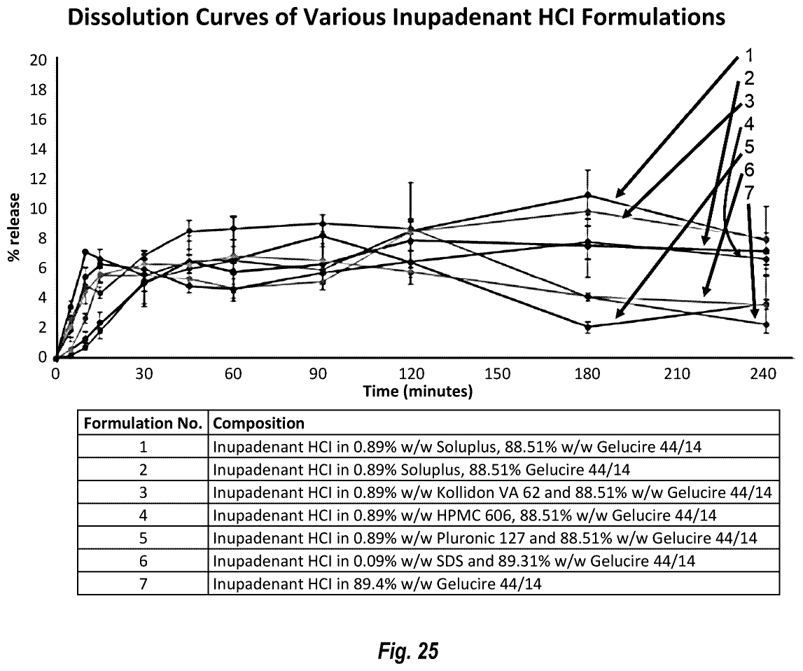

The dissolution profiles of the exemplary inupadenant hydrochloride pharmaceutical compositions listed in Table 25 (FIG. 25) were determined in fasted state simulated intestinal fluid (FaSSIF).

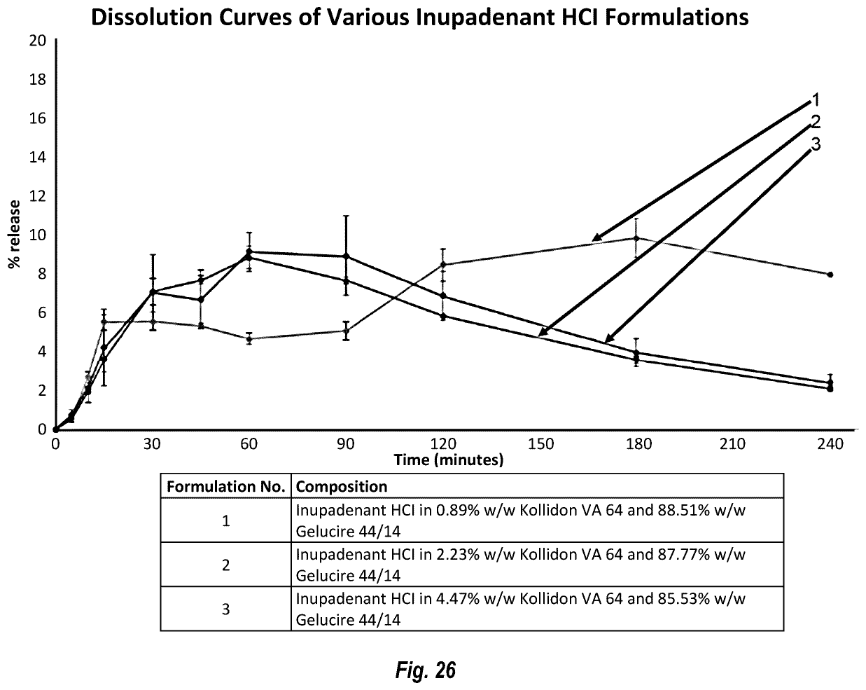

The dissolution profiles of the exemplary inupadenant hydrochloride pharmaceutical compositions listed in Table 26 (FIG. 26) were determined in fasted state simulated intestinal fluid (FaSSIF).

### INCORPORATION BY REFERENCE

This application refers to various issued patents, published patent applications, journal articles, and other publications, all of which are incorporated herein by reference.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. Amorphous inupadenant hydrochloride.

2-9. (canceled)

10. Crystalline inupadenant hydrochloride.

11. Form 2 inupadenant hydrochloride.

12. Inupadenant hydrochloride hydrate.

13. The inupadenant hydrochloride hydrate of claim 12 in crystalline form.

14-15. (canceled)

16. The crystalline inupadenant hydrochloride of claim 11, having an XRPD pattern comprising one or more peaks at about 8.9° and about 9.3° 2θ.

17. The crystalline inupadenant hydrochloride of claim 16, further comprising a peak at about 14.5° 2θ.

18-60. (canceled)

61. A pharmaceutical composition comprising:
amorphous inupadenant hydrochloride and/or crystalline inupadenant hydrochloride; and
at least one pharmaceutically acceptable excipient.

62. The pharmaceutical composition of claim 61,
wherein the crystalline inupadenant hydrochloride comprises Form 1 inupadenant hydrochloride.

63. The pharmaceutical composition of claim 61, wherein the crystalline inupadenant hydrochloride comprises Form 2 inupadenant hydrochloride.

64-183. (canceled)

184. A process for manufacturing a capsule pharmaceutical composition comprising the steps of:
(a) heating a lipid carrier and PEG to form a molten mixture;
(b) adding crystalline inupadenant hydrochloride, copovidone, and BHT to the molten mixture to form an intermediate composition;
(c) homogenizing the intermediate composition;
(d) low shear mixing the intermediate composition to form the pharmaceutical composition; and
(e) filing the capsule with the pharmaceutical composition.

185. A method for treating a cancer in a patient in need thereof, the method comprising administering to the patient a pharmaceutical composition comprising crystalline inupadenant hydrochloride and at least one pharmaceutically acceptable excipient.

186-193. (canceled)

194. A method for treating non-small cell lung cancer (NSCLC) in a patient in need thereof, the method comprising administering to the patient the pharmaceutical composition comprising crystalline inupadenant hydrochloride and at least one pharmaceutically acceptable excipient, in combination with pemetrexed and carboplatin.

195-229. (canceled)

230. Form 1 inupadenant hydrochloride.

231. The Form 1 inupadenant hydrochloride of claim 230, having an X-ray powder diffraction (XRPD) pattern comprising a peak at about 9.6° 2θ.

232. The Form 1 inupadenant hydrochloride of claim 231, wherein the XRPD pattern further comprises a peak at about 21.5° 2θ.

233. (canceled)

247. Milled inupadenant hydrochloride.

248-254. (canceled)
